(19) United States
(12) Patent Application Publication
(10) Pub. No.: US 2003/0027153 A1
(43) Pub. Date: Feb. 6, 2003

(54) Methods and compositions for diagnosing and treating neuropsychiatric disorders such as schizophrenia

(76) Inventors: Joanne M. Meyer, Framingham, MA (US); Rory Barrington-Martin, Wayland, MA (US); Alexander Parker, Natisk, MA (US)

Correspondence Address: DARBY & DARBY P.C., 805 Third Avenue, New York, NY, 10022, US

(21) Appl. No.: 09/935,464

(22) Filed: Aug. 23, 2001

Related U.S. Application Data
(63) Continuation-in-part of application No. 09/757,300, filed on Jan. 9, 2001.

(51) Int. Cl. C12Q 1/68; C07H 21/04; C12P 19/34

(52) U.S. Cl. 435/6; 435/91.2; 536/23.2

(57) ABSTRACT

This invention relates to methods and compositions for diagnosing and treating neuropsychiatric disorders, such as schizophrenia, schizoaffective disorder, bipolar affective disorder, unipolar affective disorder and adolescent conduct disorder. In particular, the invention provides novel variants of CADPKL nucleic acid sequences, as well as novel CADPKL polypeptides encoded by these variant sequences. The variant CADPKL nucleic acid sequences provided by this invention, as well as the variant polypeptides they encode are ones that statistically correlate with the presence of a neuropsychiatric disorder in individuals. The invention therefore also provides methods and compositions for using these variant nucleic acids and polypeptides to diagnose and treat such neuropsychiatric disorders.

This is a continuation-in-part of U.S. Ser. No. 09/757,300, filed on Jan. 9, 2001 and incorporated herein by reference in its entirety.

Numerous references, including patents, patent applications, figures, database references, and various publications, are cited and discussed in the description of this invention. The citation and/or discussion of such references is provided merely to clarify the description of the present invention and is not an admission that any such reference is “prior art” to the invention described herein. All references, patents, and patent applications cited and discussed in this specification are incorporated herein by reference in their entirety and to the same extent as if each reference was individually incorporated by reference.

### 1. FIELD OF THE INVENTION

The present invention relates to compositions and methods which may be used to diagnose and treat neuropsychiatric disorders, including schizophrenia, schizoaffective disorder, bipolar disorder, unipolar affective disorder and adolescent conduct disorder. In particular, the invention relates to a particular gene, known as the Calcium/Calmodium dependent protein kinase like gene or CADPKL, and its gene products. The CADPKL gene is demonstrated herein to be associated with neuropsychiatric disorders (including schizophrenia, schizoaffective disorder, bipolar disorder, unipolar affective disorder and adolescent conduct disorder). The invention therefore relates to novel use of the CADPKL gene, its gene products and antibodies thereto for diagnosing and treating such disorders.

The invention further relates to particular polymorphisms of the CADPKL gene, including particular single nucleotide polynorphisms (SNPs) and microsatellite markers, which co-segregate with neuropsychiatric disorders in individuals. The polymorphisms are useful, therefore, in the methods for treating and diagnosing such disorders.

### 2. BACKGROUND OF THE INVENTION

There are only a few psychiatric disorders in which clinical manifestations of the disorder may be correlated with demonstrable defects in the structure and/or function of the nervous system. The vast majority of psychiatric disorders, however, involve subtle and/or undetectable changes at the cellular and molecular levels of nervous system structure and function. This lack of discemable neurological defects distinguishes “neurospychiatric disorders” (for example, schizophrenia, attention deficit disorder (ADD), schizoaffective disorder, bipolar affective disorder (BAD) and unipolar affective disorder) from neurological disorders in which anatomical or biochemical pathologies are manifest. Hence, identification of causative defects in neuropathologies of neuropsychiatric disorders is needed so that clinicians may diagnose, evaluate and prescribe appropriate treatments for these disorders.

Schizophrenia is one example of a particularly serious and debilitating neuropsychiatric disorder that affects approximately 1% of the worldwide population. Currently, individuals may be evaluated for schizophrenia and other neuropsychiatric disorders using the criteria set forth in the most recent version of the American Psychiatric Association's Diagnostic and Statistical Manual of Mental Disorders (DSM-IV).

There is compelling evidence from family, twin and adoption studies for a significant genetic basis to schizophrenia and other neuropsychiatric disorders (McGuffin et al., Lancet 1995,346:678-682). This has initiated searches directed towards identification of the genetic component or components of neuropsychiatric disorders using such methods as linkage analysis, association studies of candidate genes and mapping of cytogenetic abnormalities in psychiatric patients. However, while such techniques have been applied successfully to monogenetic disorders, neuropsychiatric disorders apparently result from combined effects of multiple genes and environmental factors (see, McGuffin et al., supra). Such effects have complicated efforts to identify genetic components for these diseases. As a result, although ongoing sequencing efforts such as the Human Genome Project have lead to the discovery of many novel genes, little data is available to indicate which, if any, of these genes may be involved in a neuropsychiatric disorder.

One such gene, which is discussed in detail in the present specification, is referred to herein as the Calcium/Calmodulin Dependent Protein Kinase Like gene or CADPKL. CADPKL was first predicted within a Bacterial Artificial Chromosome (BAC) clone (clone RP1-272L16) sequenced by the Human Genome Project and available on GenBank (Accession No. AL023754.1; GI No.4007152). The CADPKL gene has also been isolated and sequenced from a cDNA clone (see GenBank Accession No. AL049688.1, GI No. 4678721). At least two ESTs corresponding to CADPKL are also known to exist and have been deposited in the GenBank dbEST database (Accession Nos. AL134342 and R05661; corresponding to GI Nos. 6602529 and 756281, respectively).

Calcium/Calmodulin protein kinases with substantial sequence similarity to CADPKL are known to play important roles in a variety of intracellular signaling cascades (see, for example, Hawley et al., J Biol. Chem. 1995,270:27186-27191). For example, the human Calcium/Calmodulin-Dependent Protein Kinase 1 (CAMK1) gene (SEQ ID NO:36) is the human gene most similar to CADPK1. An alignment of these two polypeptide sequences is shown in FIG. 1. Amino acid residues in italicized font correspond to consensus sequences that are largely conserved across the serine/threonine and tyrosine protein kinase superfamilies, indicating the CADPKL is, itself, a protein kinase.

CAMK1 is known to be a key element of the calmodulin-dependent protein kinase 1 cascade, and is expressed in a variety of tissues. Known substrates of CAMK1 include the Synapsin 1 and Synapsin 2 polypeptides, which have themselves been shown to be critical for processes such as axonogenesis, synaptogenesis, and formation and organization of synaptic vesicles (see, in particular, Chin et al., Proc. Natl. Acad. Sci. U.S.A. 1995, 92:9230-9234; Li et al., Proc. Natl. Acad. Sci. U.S.A. 1995, 92:9235-9239).

In addition, a rat homolog of CADPKL, referred to as CAMK1-γ, has also been cloned and is known in the art (see, Yokokura et al., Biochem. Biophys. Acta. 1997, 1338:8-12). Analysis of CAMK1-γ expression by RT-PCR has demonstrated that this protein is only expressed in the rat brain. Similarly, CADPKL cDNA (including partial cDNAs such as CADPKL ESTs) have, to date, only been isolated in libraries obtained from human brain tissue.

Thus, there is at best only some indirect evidence, from expression patterns and sequence homologies, indicating that CADPKL might play a role in the formation and/or organization of the human brain, and/or in cell signaling processes within the human brain. However, there is currently no direct evidence known in the art to directly link CADPKL with abnormal neurological activity. In particular, there is no data suggesting that CADPKL may be involved or associated with abnormal neurological activity such as a neuropsychiatric disorder (e.g., schizophrenia, attention deficit disorder, schizoaffective disorder, bipolar affective disorder and unipolar affective disorder).

There continues to exist, therefore, a need to identify specific genes, as well as specific genetic defects, mutations and polymorphisms, that are associated with neuropsychiatric disorder such as schizophrenia, schizoaffective disorder, bipolar affective disorder, unipolar affective disorder and adolescent conduct disorder.

There further exists a need for compositions and methods to treat and/or diagnose these and other neuropsychiatric disorders, e.g., by identifying and/or correcting specific genetic defects, mutations and polymorphisms that are associated with such neuropsychiatric disorders. For example, it would be beneficial to identify polymorphic regions within genes that are associated with one or more neuropsychiatric disorders, such as schizophrenia, schizoaffective disorder, bipolar affective disorder, unipolar affective disorder and adolescent conduct disorder. It is also desirable to identify polymorphic regions within a gene, such as CADPKL, that are associated with the response of the CADPKL gene or its gene product to one or more inhibitors of a neuropsychiatric disorder (e.g., schizophrenia, schizoaffective disorder, bipolar affective disorder, unipolar affective disorder or adolescent conduct disorder). Further, it is desirable to provide prognostic, diagnostic, pharmacogenomic and therapeutic methods utilizing such polymorphic regions, e.g., to diagnose and/or treat neuropsychiatric disorders.

The present invention overcomes these and other problems in the art.

### 3. SUMMARY OF THE INVENTION

The present invention demonstrates that the CADPKL gene is associated with neuropsychiatric disorders such as schizophrenia, schizoaffective disorder, bipolar affective disorder, attention deficit disorder, adolescent conduct disorder, etc. In particular, the invention provides polymorphisms, including single nucleotide polymorphisms (SNPs) and microsatellite repeats, that statistically correlate with a neuropsychiatric disorder in individuals. The invention further provides CADPKL polypeptides that are encoded by such variant nucleic acids and/or comprise one or more amino acid residue substitutions, insertions or deletions. The invention also provide antibodies that specifically bind to the variant CADPKL polypeptides described herein, as well as nucleic acids which may be used in the methods of the invention to detect a variant CADPKL nucleic acid or to detect a polymorphism in a CADPKL gene. For example, in one embodiment, the invention provides oligonucleotides sequences which maybe used, e.g., to amplify a CADPKL nucleic acid (for example, a specific locus on a CADPKL gene) having or suspected of having a polymorphism that correlates to a neuropsychiatric disorder.

Methods are also provided, as part of the present invention, which use the nucleic acids, polypeptides and antibodies described herein to diagnose or treat a neuropsychiatric disorder. For example, the invention provides methods to evaluate individuals for a neuropsychiatric disorder by detecting a variant CADPKL nucleic acid or polypeptide, such as one of the variants described herein, that statistically correlates to a neuropsychiatric disorder. The invention also provides therapeutic methods for treating a neuropsychiatric disorder by administering a compound that modulates (e.g., enhances or inhibits) the expression or activity of either a CADPKL nucleic acid (e.g., a CADPKL gene) or a CADPKL gene product (e.g., a CADPKL polypeptide). In one preferred embodiment, the compound modulates the expression or activity of a variant CADPKL nucleic acid or gene product, such as one of the variants described herein.

### 4. BRIEF DESCRIPTION OF THE DRAWINGS

FIG. 1. An alignment of the CADPKL polypeptide sequence (top row, SEQ ID NO:5) and the CAMK1 polypeptide sequence (bottom row, SEQ ID NO:36). Amino acid residues that are conserved in the two sequences are indicated on the middle row. Those amino acid residues that are largely conserved across the serine/threonine and tyrosine protein kinase superfamilies are indicated in bold-faced, italicized type. The shaded boxes indicated regions corresponding to the ATP-binding domain (amino acid residues 27-35 of SEQ ID NO:5), the “active site” (amino acid residues 42-44 of SEQ ID NO:5), the phosphorylation site (amino acid residues 177-178 of SEQ ID NO:5) and the putative calmodulin binding domain (amino acid residues 282-309 and 312-322 of SEQ ID NO:5), respectively.

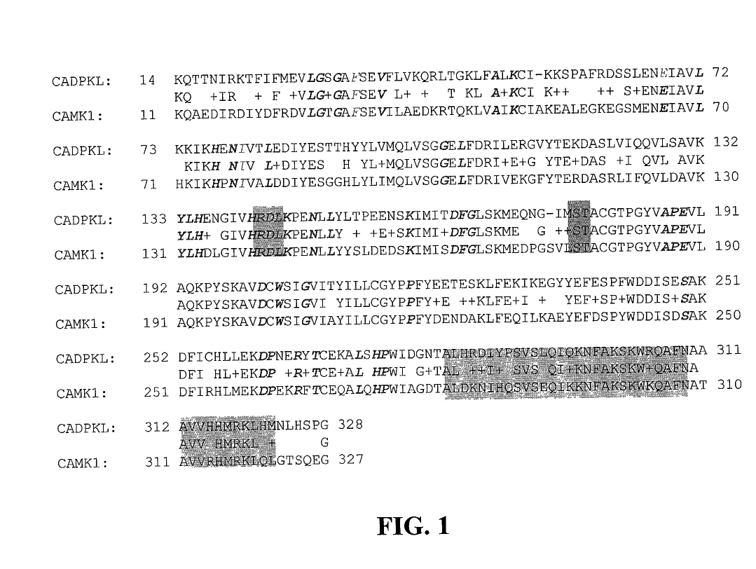

FIG. 2. CADPKL mRNA expression in human brain regions, normalized to the expression level in Locus Ceruleus (LC). See Example 3 for more details.

FIG. 3. CADPKL mRNA expression in selected bodily tissues, normalized to the expression levels in pancreas. See Example 3 for more details.

### 5. DETAILED DESCRIPTION OF THE INVENTION

The present invention relates to a gene that is referred to herein as the Calcium/Calmodulin Dependent Protein Kinase Like gene or the CADPKL gene.

The CADPKL gene has been previously described in the art. In particular, CADPKL was identified as an “in silico” gene prediction based on the human genomic DNA sequence contained in the bacterial artificial chromosome (BAC) RPI-272L16. The human genomic DNA sequence contained in this BAC comprises the sequence on human chromosome 1q32.1-32.3, which is set forth in SEQ ID NO:1. The sequence has also been deposited in the GenBank database (Bensa et al., Nucleic Acids Res. 1000, 18:15-18) and has been assigned the Accession No. AL023754.1 (GI No. 4007152).

The DNA sequence set forth in SEQ ID NO:1 comprises at least ten exons which may be transcribed and spliced together to form a CADPKL mRNA. These ten exons are delineated by the nucleic acid residues of SEQ ID NO: 1 set forth herebelow in Table 1.

TABLE 1

| | | | |
|---|---|---|---|
| Exon 1 | 129416-129534 | Exon 6 | 142317-142392 |
| Exon 2 | 134442-134570 | Exon 7 | 143439-143551 |
| Exon 3 | 137673-137747 | Exon 8 | 144310-144388 |
| Exon 4 | 139995-140133 | Exon 9 | 145924-146011 |
| Exon 5 | 140779-140902 | Exon 10 | 146251-148353 |

The protein encoding region of the CADPKL gene begins with the “start” (i.e., ATG) codon located at nucleotide residue 129443 of SEQ ID NO:1, and ends at the “stop” (i.e., TGA) codon at nucleotide residues 146718 of SEQ ID NO:1. Thus, the protein coding sequence of the human CADPKL gene comprises the contiguous sequence of nucleic acids 129443-129534; 13442-134570; 137673-137747; 13995-140133; 140779-140902; 142317-142392; 143439-143551; 144310-144388; 145924-146011; and 146251-146718 of SEQ ID NO:2. This protein coding sequence is set forth here in SEQ ID NO:2.

The predicted amino acid sequence encoded by the above-described CADPKL gene and, in particular, by the protein coding sequence set forth in SEQ ID NO:2, has also been deposited in the GenBank database, and has been assigned the Accession No. CAA19296.1 (GI No. 4007153). The polypeptide sequence is set forth here in SEQ ID NO:3.

A CADPKL cDNA has also been isolated, and its nucleotide sequence has been deposited in the GenBank database and assigned the Accession No. AL049688.1 (GI No. 4678721). This CADPKL cDNA sequence is set forth here in SEQ ID NO:4. The predicted amino acid sequence of the gene product encoded by the CADPKL cDNA has also been deposited in the GenBank database (Accession No. CAB41259.1; GI No. 7678722) and is set forth here, as SEQ ID NO:5.

Further, partial CADPKL nucleic acid sequences have been identified in at least two publicly available ESTs. These EST sequences, which have been deposited in the GenBank database and assigned the Accession Nos. R05661 and AL134342 (GI Nos. 756281 and 6602529, respectively), are set forth here in SEQ ID NOS. 6 and 7, respectively. Still other ESTs corresponding to partial CADPKL nucleic acid sequences have also been identified and are described in prior patent applications identified here below and incorporated by reference in their entirety. In particular, the following Table identifies each CADPKL EST by the identification number along with the particular patent application(s) where each clone and corresponding EST is disclosed.

| Clone ID No. | Prior Patent Application | SEQ ID NO. |
|---|---|---|
| juhXhN5ae08t1 | U.S. prov. app. Serial No. 60/193,481 | 46 |
| | (filed March 29, 2000) | |
| jthsa066c12t2 | U.S. prov. app. Serial No. 60/101,133 | 47 |
| | U.S. Serial No. 09/397,206 | |
| | (filed September 18, 1998) | |
| mine16109human_c1 | U.S. Serial No. 09/277,214 | 49 |
| | (filed March 26, 1999) | |
| jlhbaa144c09t1 | U.S. prov. app. Serial No. 60/092,406 | 50 |
| | U.S. Serial No. 09/3 54,899 | |
| | (filed March 10, 1998) | |
| cbhsa066c12jtcbt1 | U.S. prov. app. Serial No. 60/208,647 | 48 |
| | (filed May 30, 2000) | |

In addition, the multigene family that CADPKL belongs has recently been supplemented with a novel member (Verploegen et al., Blood 2000;96:3215-23). An EST which is a partial nucleic acid sequence of this novel member is also known. This EST is encoded by clone ID No. jthea053b05t1 and is described in U.S. Ser. Nos. 60/152,109 and 09/652,814, filed Aug. 31, 1999, both of which incorporated by reference herein in their entireties. In particular, this EST is about 72% sequence identity to CADPKL at the nucleic acid level.

The present invention relates, more specifically, to novel polymorphisms within the CADPKL gene, as well as to variant CADPKL nucleic acids that contain one or more of these polymorphisms. The CADPKL polymorphisms of the invention include single nucleotide polymorphisms (SNPs) at specific nucleic acid residues, as well as deletions or insertions of nucleotides at specific nucleic acid residues within a CADPKL nucleic acid. The polymorphisms also include variant regions of a CADPKL nucleic acid that are referred to herein as “microsatellite repeats” or microsatellite regions.

The variant CADPKL nucleic acids of the invention therefore include CADPKL nucleic acids containing one or more of these polymorphisms. Specifically, and without being limited to any theory or mechanism of action, at least two versions or “alleles” of the CADPKL gene are believed to exist. The first of these alleles is referred to herein as the “reference” or “wild-type” CADPKL allele. The reference allele has been arbitrarily designated and corresponds to the CADPKL gene contained in the genomic sequence that has been deposited in GenBank (Accession No. AL023754.1; GI No. 4007152) and is set forth here in SEQ ID NO:1. The other CADPKL alleles, which are referred to here as “variant” CADPKL alleles or “allelic variants”, differ from the wild-type allele by at least one nucleic acid residue. More particularly, the variant CADPKL alleles of this invention contain at least one of the CADPKL polymorphisms described herein, such as one or more SNPs and/or one or more microsatellite repeats.

It is noted that the terms “wild-type” (or “reference”) and “variant” CADPKL nucleic acids refer, not only to genomic CADPKL nucleic acids (e.g., the wild-type genomic CADPKL nucleic acid set forth in SEQ ID NO:1), but also to CADPKL nucleic acids derived from such genomic sequences and/or corresponding to portions thereof. Thus, for example, wild-type CADPKL nucleic aicds of the invention also include the wild-type CADPKL cDNA sequence (e.g., the sequence set forth in SEQ ID NO:4) and/or wild-type CADPKL protein coding sequences (e.g., the sequence set forth in SEQ ID NO:2). Likewise, the variant CADPKL nucleic acids of the invention include nucleic acids derived from a CADPKL genomic sequence of the invention and/or corresponding to a portion thereof, which also contain one or more polymorphisms. Thus, variant CADPKL nucleic acids of this invention include variant CADPKL genomic sequence, variant CADPKL cDNA sequences, variant protein coding sequences, variant ESTs, and the like.

The invention also relates to fragments of the variant CADPKL nucleic acids. In particular, the invention relates to nucleic acids having the sequence of a CADPKL allelic variant that contains at least one polymorphism. Such portions or fragments of a CADPKL nucleic acid are preferably at least five nucleotides in length. For example, fragments of a variant CADPKL nucleic acid may be at least 10, at least 15, at least 20, at least 25, at least 30, at least 50 or at least 100 nucleotides in length. As a more specific example, a portion or fragment of a variant CADPKL nucleic acid that is 21 nucleotides in length may contain a polymorphic site such as an SNP (i.e., the nucleotide that differs from the reference nucleotide at that site) and twenty additional nucleotides which flank the polymorphic site. These additional nucleotides may be on either or both sides of the polymorphic site.

As a more specific (but not limiting) example, Table 2 infra specifies SNPs of the CADPKL gene that are among the polymorphisms of the present invention. In particular, these polymorphisms are ones which were discovered to be associated with neuropsychiatric disorder (including schizophrenia, as well as schizoaffective disorder, bipolar disorder, unipolar affective disorder and adolescent conduct disorder), as described in the Examples infra. In more detail, Table 2 provides, in the left hand column, a “Polymorphism ID” by which each SNP is identified in this specification. Column 2 (under the heading “Residue No.”) specifies the nucleotide residue in the references CADPKL genomic sequence (SEQ ID NO:1) which is the location of the variant site in the SNP. Column 3 (under the title “Mutation”) specifies the identity of the variant nucleotide in the SNP. For example, the first SNP recited in Table 2 (i.e., cadpkl5) is located at nucleic acid residues number 140766 of SEQ ID NO: 1. This nucleotide is a thymine (T) in the wild-type (“WT”) sequence. However, in those CADPKL alleles having this particular SNP the nucleotide is a guanine. This polymorphism is therefore indicated by the entry (“T/G”) in column 3 of the Table. The nucleotide sequence flanking each polymorphism is provided in column 4 of the Table. Specifically, the sequence of the 10 nucleotides flanking either side of the SNP is provided (i.e., 10 nucleotides 5′ of the polymorphism and 10 nucleotides 3′ of the polymorphism) with the variant nucleotide indicated in lower-case letters. Finally, column 5 provides the SEQ ID NO. in the accompanying Sequence Listing for each flanking sequence provided in the Table.

TABLE 2
SNPs IN CADPKL GENOMIC SEQUENCE (SEQ ID NO:1)

| Polymorphism | Residue | Mutation | | |
|---|---|---|---|---|
| ID | No. | (WT/SNP) | Flanking Sequence | SEQ ID NO. |
| cadpk15 | 140766 | T/G | ACTACATATTgTTTCTCCTAC | 37 |
| cadpk16 | 142239 | T/C | ACCTCTTCTCcAAGCCTGGCC | 38 |
| cadpk17 | 143457 | A/G | GATACCCCCCgTTCTATGAAG | 39 |
| cadpk19a | 146041 | G/T | GGGTGGGAAAtCTGTTCTGGG | 40 |
| cadpk19b | 146125 | G/C | TTGGAGCTCCcTGTACCCTCT | 41 |
| cadpk110 | 146320 | G/A | CAGCCCGGGAaTCCGCCCAGA | 42 |
| cadpk0 | 117978 | A/G | ATGCACAAGCgTTTTTCTGGA | 77 |
| cadpk112d | 147997 | C/T | ACAGGCAGCTtCCCATGGTGG | 78 |
| cadpk112e | 148151 | A/T | AATAGAAAGAtGTTCATGAG T | 79 |
| cadu2 | 117926 | T/C | CAATTTCACAcACAC ATGCAC | 80 |
| cadu3a | 117533 | C/A | AGGACTGAGAaAGGTTTGGGG | 81 |
| cadu3b | 117584 | A/G | GACATATCAAgGATACTGAGT | 82 |
| cadu3c | 117642 | C/T | AAGGCCCTTTtTCCCAGTTCT | 83 |
| cad11a | 147192 | G/A | CTGCCCCATCaACTCTTCTTC | 84 |
| cadpk8 | 144444 | G/A | CCAAAACCATaCTGACTCATT | 85 |
| cadpkd1 | 128813 | A/G | TGCTAAATACgTATTGGTTAA | 86 |
| cadpkd2 | 128947 | C/T | ACAAAAACAGtACAATACTCA | 87 |
| cadpki | 127923 | G/A | GCCATTAGCTaTTGGAGGGGG | 88 |
| cadpkj | 127747 | T/C | CAAGACCCCAcAGAGTCTACA | 89 |
| cadpkk | 127700 | A/T | ATTGTAGAGGtACAAACTTTC | 90 |

Many of the SNPs identified in Table 2, supra, are found in exons of the CADPKL genomic sequence. These SNPs may also generate variant CADPKL gene products (for example, variant CADPKL mRNAs or variant CADPKL cDNAs derived therefrom) that have one or more polymorphisms relative to a wild-type or reference CADPKL gene product (e.g., a wild-type CADPKL mRNA or a wild-type CADPKL cDNA).

In addition, some of the variant CADPKL nucleic acids of this invention encode variant CADPKL polypeptides having one or more amino acid residue substitutions, insertions or deletions. Thus, the present invention also provides allelic variant and mutant CADPKL polypeptides. The terms allelic variant and mutant, when used herein to describe a polypeptide, refer to polypeptides encoded by variant alleles of a gene. Preferably, an allelic variant of a polypeptide will have one or more sequence polymorphisms (for example, one or more amino acid residue substitutions, insertions or deletions) relative to a polypeptide encoded by the wild-type gene (i.e., the “wild-type” polypeptide). Thus, an allelic variant of a CADPKL polypeptide is a polypeptide encoded by an allelic variant of a CADPKL gene. Similarly, a “wild-type” or “reference” CADPKL polypeptide, as the term is used herein, is a CADPKL polypeptide encoded by a wild-type CADPKL nucleic acid.

As noted above, the wild-type CADPKL gene has been arbitrarily designated and corresponds to the CADPKL genomic sequence deposited in GenBank (Accession No. AL023754.1; GI No. 4007152) and set forth in SEQ ID NO:1. Similarly, a wild-type CADPKL cDNA is also known (GenBank Accession No. AL049688.1; GI No.4678721) and set forth here in SEQ ID NO:4. These wild-type CADPKL nucleic acids encoded polypeptides have the amino acid sequences set forth in SEQ ID NOS:3 and 5, respectively. Thus, the terms “wild-type” and “reference” CADPKL polypeptide may refer either to a polypeptide having the amino acid sequence set forth in SEQ ID NO:3, or to a polypeptide having the amino acid sequence set forth in SEQ ID NO:5.

Tables 3A and 3B specify variant CADPKL nucleic acids and polypeptides, respectively, that are obtained from allelic variants of the CADPKL genomic sequence. In particular, Table 3A, infra, specifies SNPs in variant CADPKL protein coding sequences (e.g., CADPKL cDNA sequences) corresponding to SNPs recited in Table 2, supra. Variant CADPKL nucleic acids having these SNPs therefore are also associated with neuropsychiatric disorders such as schizophrenia, schizoaffective disorder, bipolar disorder, unipolar affective disorder and adolescent conduct disorder.

The left hand column of Table 3A specifies the “Polymorphism ID” by which each SNP in the Table is identified. In particular, these ID's are identical to the Polymorphism ID's specified in Table 2, supra, for corresponding SNPs in the CADPKL genomic sequence.

Each polymorphism recited in Table 3A is identified based on one or more changes in the variant CADPKL nucleotide sequence from a reference CADPKL nucleotide sequence. Thus, Column 2 in Table 3A (under the heading “Reference SEQ ID NO.”) specifies the reference CADPKL nucleotide sequence according to its SEQ ID NO. in the accompanying Sequence Listing. Column 3 (under the heading “Residue No.”) specifies the nucleotide residue in the reference sequence which is the location of the variant site in the SNP, and Column 4 (under the headling “Mutation”) specifies the identity of the variant nucleotide in the SNP.

Thus, for example, the first two SNPs recited in Table 3A correspond to the SNP “cadpkl7” recited in Table 2, supra, and therefore have the same Polymorphism ID. These SNPs are identified in Table 3A with respect to the reference CADPKL nucleotide sequences provided in SEQ ID NOS:2 and 4, and are located at nucleic acid residue position 654 and 671, respectively, of those sequences. The variant nucleotide of the SNP is a guanine (G), whereas there is an adenine (A) in that position of the wild-type (WT) or reference CADPKL nucleic acid (i.e., in SEQ ID NOS:2 and 4).

TABLE 3A
SNPs IN CADPKL CODING SEQUENCES

| | Reference | Residue | Mutation |
|---|---|---|---|
| Polymorphism ID | SEQ ID NO. | No. | (WT/SNP) |
| cadpk17 | 2 | 654 | A/G |
| cadpk17 | 4 | 671 | A/G |
| cadpk110 | 2 | 985 | G/A |
| cadpk110 | 4 | 1002 | G/A |

Similarly, Table 3B specifies variant CADPKL polypeptides encoded by variant nucleic acids having an SNP recited in Table 3A, supra. The left hand column in Table 3B specifies the polymorphism ID of the corresponding SNP that encodes the variant CADPKL polypeptide. Column 2 (under the heading “Reference SEQ ID NO.”) specifies the reference CADPKL polypeptide according to its SEQ ID NO. in the accompanying Sequence Listing. Column 3 (under the heading “Residue No.”) specifies the amino acid residue of the reference sequence that is the location of the variant amino acid residue (i.e., an amino acid residue substitutions, insertion or deletion) encoded by the SNP, and column 4 (under the heading “Mutation”) specifies the identity of the variant amino acid residue in the wild-type (WT) or reference CADPKL polypeptide, and in the variant polypeptide encoded by the SNP.

TABLE 3B
AMINO ACID SUBSTITUTIONS
ENCODED BY CADPRL SNPs

| | Reference | | Mutation |
|---|---|---|---|
| Polymorphism ID | SEQ ID NO. | Residue No. | (WT/SNP) |
| cadpk110 | 3 | 329 | Val/Ile |
| cadpk110 | 5 | 329 | Val/Ile |

The various aspects of the invention are set forth, infra, in more detail. In particular, Section 5.1 sets forth and defines certain terms as they are used herein to describe the present invention. The CADPKL nucleic acids and polypeptides of the present invention invention, are the described, in detail, in Sections 5.2 and 5.3, respectively. In particular, these sections describe the variant CADPKL polypeptides and nucleic acids which may be used in, and are therefore considered part of, the present invention. Exemplary methods by which a skilled artisan may express such CADPKL nucleic acids and polypeptides, as well as exemplary methods for generating antibodies that specifically bind to such CADPKL polypeptides are also provided, in Sections 5.4 and 5.5, respectively. Finally, Section 5.6 provides novel uses of the CADPKL nucleic acids and polypeptides of the invention, e.g., for diagnosing and/or treating neuropsychiatric disorders such as schizophrenia. These methods include, for example, diagnostic applications (e.g., by detecting variant CADPKL nucleic acids and polypeptides of the invention) and screening assays, as well as therapeutic methods and pharmaceutical preparations.

### 5.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the devices and methods of the invention and how to make and use them.

General Definitions.

The term “neuropsychiatric disorder”, which may also be referred to as a “major mental illness disorder” or “major mental illness”, refers to a disorder which may be generally characterized by one or more breakdowns in the adaptation process. Such disorders are therefore expressed primarily in abnormalities of neurological activity. Currently, individuals may be evaluated for various neuropsychiatric disorders using criteria set forth in the most recent version of the American Psychiatric Association's Diagnostic and Statistical Manual ofMental Health (DSM-IV). Exemplary neuropsychiatric disorders include, but are not limited to, schizophrenia, attention deficit disorder (ADD), schizoaffective disorder, bipolar affective disorder, unipolar affective disorder, and adolescent conduct disorder.

The term “neurological activity” herein includes, but is not limited to, thought, feeling and/or behavior producing either distress or impairment of function (i.e., impairment of mental function such as dementiar, senility, depression or mania to name a few).

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested for the presence of a CADPKL polypeptide, or for the presence of a CADPKL nucleic acid, e.g., to evaluate a gene therapy or expression in a transgenic animal or to identify cells that express CADPKL. The term sample may also refer to a biological material which can be tested for a particular variant or polymorphism of a CADPKL nucleic acid, or for a polypeptide encoded by a particular variant or polymorphism of a CADPKL nucleic acid. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture. In a preferred embodiment, samples are obtained from brain tissue or from other tissues of the nervous system.

Non-human animals include, without limitation, laboratory animals such as mice, rats, rabbits, hamsters, guinea pigs, etc.; domestic animals such as dogs and cats; and, farm animals such as sheep, goats, pigs, horses, and cows, and especially such animals made transgenic with human CADPKL.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “aberrant” or “abnormal”, as applied herein refers to an activity or feature which differs from (a) a normal or activity or feature, or (b) an activity or feature which is within normal variations of a standard value.

For example, an “abnormal” activity of a gene or protein such as the CADPKL gene or protein refers to an activity which differs from the activity of the wild-type or native gene or protein, or which differs from the activity of the gene or protein in a healthy subject, e.g., a subject not afflicted with a disease associated with a specific allelic variant of a CADPKL polymorphism. An activity of a gene includes, for instance, the transcriptional activity of the gene which may result from, e.g., an aberrant promoter activity. Such an abnormal transcriptional activity can result, e.g., from one or more mutations in a promoter region, such as in a regulatory element thereof An abnormal transcriptional activity can also result from a mutation in a transcription factor involved in the control of gene expression.

An activity of a protein can be aberrant because it is stronger than the activity of its native counterpart. Alternatively, an activity can be aberrant because it is weaker or absent related to the activity of its native counterpart. An aberrant activity can also be a change in an activity. For example an aberrant protein can interact with a different protein relative to its native counterpart. A cell can have an aberrant activity due to overexpression or underexpression of the gene encoding CADPKL. An aberrant CADPKL activity can result, e.g., from a mutation in the gene, which results, e.g., in lower or higher binding affinity of a ligand or substrate to the protein encoded by the mutated gene.

An “abnormal” or “aberrant” feature is a feature which differs substantially from a normal feature or value for a CADPKL gene or protein. For instance, an abnormal nucleotide or amino acid sequence is a sequence which differs from the wild-type sequence due to, e.g., polymorphisms in the respective sequences. Similarly, an abnormal level of a CADPKL gene, cDNA, mRNA, polypeptide, or protein, is a concentration or a total amount of a CADPKL gene, cDNA, mRNA, polypeptide, or protein, in a sample, cell, or subject, which differs from a reference value. Moreover, an abnormal tissue distribution of CADPKL cDNA, mRNA, polypeptide, or protein in a subject is a tissue distribution which differs from the tissue distribution of CADPKL cDNA, mRNA, polypeptide or protein in a “normal” or “healthy” subject. Such aberrant tissue distribution can be the result of, e.g., an abnormal transcriptional activity from the CADPKL promoter region.

The term “molecule” means any distinct or distinguishable structural unit of matter comprising one or more atoms, and includes, for example, polypeptides and polynucleotides.

Molecular Biology Definitions.

In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, New York (referred to herein as “Sambrook et al., 1989”); DNA Cloning: A Practical Approach, Volumes I and II (D.N. Glover ed. 1985); Oligonucleotide Synthesis (M. J. Gait ed. 1984); Nucleic Acid Hybridization (B. D. Hames & S.J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently caries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecules or both. The other molecule is referred to as a “receptor”. In preferred embodiments, both a ligand and its receptor are molecules (preferably proteins or polypeptides) produced by cells. In particularly preferred embodiments, a ligand is a soluble molecule and the receptor is an integral membrane protein (i.e., a protein expressed on the surface of a cell). However, the distinction between which molecule is the ligand and which is the receptor may be an arbitrary one.

The binding of a ligand to its receptor is frequently a step in signal transduction within a cell. Exemplary ligand-receptor interactions include, but are not limited to, binding of a hormone to a hormone receptor (for example, the binding of estrogen to the estrogen receptor) and the binding of a neurotransmitter to a receptor on the surface of a neuron.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” and expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiation transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors. For example, CADPKL may be expressed in PC 12, COS-1, or C₂C₁₂ cells. Other suitable cells include CHO cells, HeLa cells, 293T (human kidney cells), mouse primary myoblasts, and NIH 3T3 cells.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

An “allele” refers to any one of a series of two or more genes that occupy the same position or locus on a chromosome. Generally, alleles refer to different forms of a gene that differ by at least one nucleic acid residue. Thus, as used here, the terms “allele” and “allelic variant” refer, not only to different forms of genomic sequences, but may also refer to different forms of sequences that are encoded by or otherwise derived from allelic variants of the genomic sequence. For example, the term allelic variant may refer to mRNA sequences that are encoded by allelic variants of a genomic sequence, or to cDNA sequences that are derived from such variant mRNA sequences. As it is used herein, the term allelic variant can also refer to protein or polypeptides sequences which are derived from (e.g., encoded by) allelic variants of a particular gene.

Allelic variants are usually described by comparing their nucleotide or (in the case of variant polypeptides) amino acid sequences to a common “wild-type” or “reference” sequence. Thus, a “wild-type” or “reference” allele of a gene refers to that allele of a gene having a genomic sequence designated as the wild-type sequence and/or encoding a polypeptide having an amino acid sequence that is also designated as a wild-type sequence. The wild-type allele may be arbitrarily selected from any of the different alleles that may exist for a particular gene. However, the allele is most typically selected to be the allele which is most prevalent in a population of individuals. Thus, for example, the wild-type CADPKL genomic sequence has been arbitrarily selected, here, as the genomic sequence deposited in GenBank (Accession No. AL023754. 1; GI No. 4007152) and set forth here in SEQ ID NO:1.

The term “polymorphism” refers, generally, to the coexistence of more than one form of a gene (e.g., more than one allele) within a population of individuals. The different alleles may differ at one or more positions of their nucleic acid sequences, which are referred to herein as “polymorphic locuses”. When used herein to describe polypeptides that are encoded by different alleles of a gene, the term “polymorphic locus” also refers to the positions in an amino acid sequence that differ among variant polypeptides encoded by different alleles.

The polymorphisms of the present invention include “single nucleotide polymorphisms” (SNPs) and microsatellite repeats. The term SNP refers to a polymorphic site occupied by a single nucleotide, which is the site of variation between allelic sequences. Typically, the polymorphic site of an SNP is flanked by highly conserved sequences (e.g., sequences that vary in lees than 1/100 and, more preferably, in less than 1/1000 individuals in a population). The polymorphic locus of an SNP may be a single base deletion, a single base insertion, or a single base substitution. Single base substitutions are particularly preferred.

A “microsatellite repeat” or “microsatellite”, as the term is used herein, refers to a short sequence of repeating nucleotides within a nucleic acid. Typically, a microsatellite repeat comprises a repeating sequence of two (i.e., a dinucleotide repeat), three (i.e., a trinucleotide repeat), four (i.e., a tetranucleotide repeat) or five (i.e., a pentanucleotide repeat) nucleotides. Microsatellites of the invention therefore have the general formula (N₁, N₂, . . . Nᵢ)ₙ, wherein N represents a nucleic acid residue (e.g., adenine, thymine, cytosine or guanine), i represents the number of the last nucleotide in the microsatellite, and n represents the number of times the motif is repeated in the microsatellite locus. In one embodiment the number of nucleotides in a microsatellite motif (i) is about six, preferably between two and five, and more preferably two, three or four. The total number of repeats (n) in a microsatellite repeat may be, e.g., from one to about 60, preferably from 4 to 40, and more preferably from 10 to 30 when i=2; is preferably between about 4-25, and more preferably between about 6-22 when i=3; and is preferably between about 4-15, and more preferably between about 5-10 when i=4. A CADPKL nucleic acid of the invention may comprise any microsatellite repeat of the above general formula. However, the following motifs are particularly preferred: CA, TC, and, AATTG; as well as all complements and permutations of such motifs (for example, TG, GA, and CAATT.

The term “locus” refers to a specific position on a chromosome. For example, the locus of a CADPKL gene refers to the chromosomal position of that gene.

The term “linkage” refers to the tendency of genes, alleles, loci or genetic markers to be inherited together as a result of their location on the same chromosome. Linkage may be measured, e.g., by the percent recombination between two genes, alleles, loci or genetic markers.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant.

“Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined, e.g., by the BLAST or FASTA algorithms. Preferably, function-conservative variants of a given polypeptide have at least 75%, more preferably at least 85% and still more preferably at least 90% amino acid sequence identity to the given polypeptide and, preferably, also have the same or substantially similar properties (e.g., of molecular weight and/or isoelectric point) or functions (e.g., biological functions or activities) as the native or parent polypeptide to which it is compared.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origina (see, Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (e.g., BLAST, FASTA, CLUSTAL, etc.).

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of CADPKL, or to detect the presence of nucleic acids encoding a CADKL polypeptide. In particularly preferred embodiments, oligonucleotides are used to detect the presence of CADPKL nucleic acids having a particular polymorphism, such as an SNP or a microsatellite repeat. In a further embodiment. an oligonucleotide of the invention can form a triple helix with a CADPKL DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention provides antisense nucleic acids (including ribozymes), which may be used to inhibit expression of a CADPKL gene or its gene product. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, triple helix interactions, ribozymes and RNase-H mediated arrest. Antisense nucleic acid molecules can be encoded by a recombinant gene for expression in a cell (e.g., U.S. Pat. Nos. 5,814,500; 5,811,234), or alternatively they can be prepared synthetically (e.g., U.S. Pat. No. 5,780,607). Other specific examples of antisense nucleic acid molecules of the invention are provided infra.

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotidemimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2′ position: OH, SH, SCH₃, F, OCN, O(CH₂)nNH₂ or O(CH₂), CH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃; SO₂CH₃; ONO₂;NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 5× SSC, can be used, e.g., 5×SSC, 0.1% SDS, 0.25% milk, and no formamide; or 30% formamide, 5× SSC, 0.5% SDS). Moderate stringency hybridization conditions correspond to a higher Tₘ, e.g., 40% formamide, with 5× or 6× SCC. High stringency hybridization conditions correspond to the highest Tₘ, e.g., 50% formamide, 5× or 6× SCC. SCC is a 0.15 MNaCl, 0.015 MNa-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) ofnucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2× SSC, at 42° C. in 50% formamide, 4× SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6× SSC/0.05% sodium pyrophosphate, or other conditions that afford equivalent levels of hybridization.

### 5.2. CADPKL Nucleic Acids

In general, a CADPKL nucleic acid molecule of the present invention include: a nucleotide sequence that encodes a CADPKL polypleptide as defined, ifra, in Section 5.3; the complement of a nucleic acid sequence that encodes a CADPKL polypeptide; and fragments thereof. Thus, in one preferred embodiment the CADPKL nucleic acid molecules of the invention comprise a nucleotide sequence that encodes the amino acid sequence set forth in SEQ ID NO:3 or in SEQ ID NO:5. For example, a CADPKL nucleic acid molecule of the invention may comprise the particular nucleic acid sequence set forth in SEQ ID NO:2 or, alternatively, in SEQ ID NO:4. In other embodiments, a CADPKL nucleic acid molecule of the invention may comprise a genomic sequence, such as SEQ ID NO: 1, that contains the sequence of a CADPKL gene. The genomic CADPKL nucleic acids of the invention may also comprise sequences of one or more introns or exons of a CADPKL gene, such as the introns and exons defined in Table 1, supra, for the CADPKL gene contained in SEQ ID NO:1.

The CADPKL nucleic acid molecules of the invention also include nucleic acids which comprise a sequence encoding one or more fragments of a CADPKL polypeptide. Such fragments include, for example, polynucleotides encoding an epitope of a CADPKL polypeptide; e.g., nucleic acids that encode a sequence of at least 5, more preferably at least 10, 15, 20, 25 or 50 amino acid residues of a CADPKL polypeptide sequence (e.g., of the polypeptide sequence set forth in SEQ ID NO:3 or in SEQ ID NO:5).

Alternatively, a CADPKL nucleic acid molecule of the invention may comprise larger fragments of a full length CADPKL nucleic acid (for example, a fragment of a full length CADPKL mRNA or a cDNA derived therefrom). Exemplary partial CADPKL nucleic acids are known in the art and are provided here in SEQ ID NOS:6 and 7. In particular, these partial CADPKL nucleic acids correspond to EST sequences which have been deposited in the GenBank database and assigned the GenBank Accession Nos. R05661 (GI NO:756281) and AL134342 (GI NO:6602529). Other exemplary partial CADPKL nucleic acids are provided here in SEQ ID NOS:46-50, and are also described in U.S. patent application Ser. Nos. 60/193,481; 60/101,133; 09/397,206; 60/208,647; 60/152,109; 09/652,814; 09/277,214; 60/092,406; 09/354,899. Preferably, partial CADPKL nucleic acid molecules such as these are between about 100 and 1000 micleotides in length, and are more preferably at least 150, 200, 250, 300, 350, 400, 450 or 500 nucleotides in length.

The CADPKL nucleic acid molecules of the invention also include nucleic acid molecules that comprise coding sequences for modified CADPKL polypeptides (e.g., having amino acid substitutions, deletions or truncations) and for variants (including analogs and homologs from the same or different species) of a CADPKL polypeptide. In preferred embodiments, such nucleic acid molecules have at least 50%, preferably at least 75% and more preferably at least 90% sequence identity to a CADPKL coding sequence (e.g., the coding sequence set forth in SEQ ID NO:2 or in SEQ ID NO:4) or to a genomic sequence (for example, SEQ ID NO:1) that contains all or part of a CADPKL gene. Alternatively, nucleic acid molecules of the invention may also be ones that hybridize to a CADPKL nucleic acid molecule, e.g., in a Southern blot assay under defined conditions. For example, in specific embodiments a CADPKL nucleic acid molecule of the invention comprises a nucleotide sequence which hybridizes to a complement of a CADPKL nucleic acid sequence, such as any of the coding sequences set forth in SEQ ID NO: 1, 2 or 3, under highly stringent hybridization conditions that comprise, e.g., 50% formamide and 5× or 6× SSC. In other embodiments, the nucleic acid molecules hybridize to a complement of a CADPKL nucleic acid sequence (e.g., to any of the coding sequences set forth in SEQ ID NO:1, 2 or 3) under moderately stringent hybridization conditions (for example, 40% formamide with 5× or 6× SSC), or under low stringency conditions (for example, in 5× SSC, 0.1% SDS, 0.25% milk, no formamide, 30% formamide, 5× SSC or 0.5% SDS). Alternatively, a nucleic acid molecule of the invention may hybridize, under the same defined hybridization conditions, to the complement of a fragment of a nucleotide sequence encoding a full length CADPKL polypeptide.

In other embodiments, the nucleic acid molecules of the invention comprise fragments of a full length CADPKL nucleic acid sequence. For example, in preferred embodiments, such CADPKL nucleic acid fragments comprise a nucleotide sequence that corresponds to a sequence of at least 10 nucleotides, preferably at least 15 nucleotides and more preferably at least 20,25, or 30 nucleotides of a full length coding CADPKL nucleotide sequence. In specific embodiments, the fragments correspond to a portion (e.g., of at least 10, 15, 20, 25 or 30 nucleotides) of a CADPKL coding sequence (e.g., as set forth in SEQ ID NO:2 or4) or of a genomic sequence (such as SEQ ID NO:1) containing a CADPKL gene or a portion thereof. In other preferred embodiments, the CADPKL nucleic acid fragments comprise sequences of at least 10, preferably at least 15 and more preferably at least 20, 25 or 30 nucleotides that are complementary and/or hybridize to a full length coding CADPKL nucleic acid sequence (e.g., in the sequences set forth in SEQ ID NOS:1-2 and 4), or to a fragment thereof. Suitable hybridization conditions for such oligonucleotides are described supra, and include washing in 6× SSC/0.05% sodium pyrophosphate. Because the melting temperature of oligonucleotides will depend on the length of the oligonucleotide sequence, suitable hybridization temperatures will vary depending upon the oligonucleotide molecules used. Exemplary temperatures will by 37° C. (e.g., for 14-base oligonucleotides), 48° C. (e.g., for 17-base oligonucleotides), 55° C. (e.g., for 20-base oligonucleotides) and 60° C. (e.g., for 23-base oligonucleotides).

Nucleic acid molecules comprising such fragments are useful, for example, as oligonucleotide probes and primers (e.g., PCR primers) to detect and amplify other nucleic acid molecules encoding a CADPKL polypeptide, including genes that encode variant CADPKL polypeptides such as CADPKL analogs, homologs and variants. Oligonucleotide fragments of the invention may also be used, e.g., as antisense nucleic acids, triple helix forming oligonucleotides or as ribozymes; e.g., to modulate levels of CADPKL gene expression or transcription in cells.

For example, Table 4 in the Examples infra describes several specific nucleic acids, comprising the nucleotide sequences set forth in SEQ ID NOS:8-35 and 51-76, that may be used to amplify regions of a CADPKL gene or genomic sequence as described in the Examples. In particular, these sequences are used in the Examples to amplify particular segments of the CADPKL genomic sequence set forth in SEQ ID NO: 1 and identify nucleic acid mutations or polymorphisms (including microsatellite repeats and single nucleotide polymorphisms) which correlate with and are therefore associated with a neuropsychiatric disorder. The nucleic acids of the present invention therefore include ones which comprise any of the nucleotide sequences set forth in Table 4, infra, and in SEQ ID NOS:8-35 and 51-76.

The “primers” and “probes” of the invention are nucleic acid sequence which can be used for amplifying and/or identifying a CADPKL gene sequence. Primers can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of a CADPKL gene, and which by hybridization or absence of hybridization to the DNA of a subject will be indicative of the identity of the allelic variant of the polymorphic region of the CADPKL gene.

Numerous procedures for determining the nucleotide sequence of a nucleic acid molecule, or for determining the presence of mutations in nucleic acid molecules include a nucleic acid amplification step, which can be carried out by, e.g., the polymerase chain reaction (PCR). Accordingly, in one embodiment, the invention provides primers for amplifying portions of a CADPKL gene, such as portions of exons and/or portions of introns. In a preferred embodiment, the exons and/or sequences adjacent to the exons of the human CADPKL gene will be amplified to, e.g., detect which allelic variant of a polymorphic region is present in the CADPKL gene of a subject. Preferred primers comprise a nucleotide sequence complementary a specific allelic variant of a CADPKL polymorphic region and of sufficient length to selectively hybridize with a CADPKL gene. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a CADPKL gene. In an even more preferred embodiment, the primer is capable of hybridizing to a CADPKL nucleotide sequence and has a nucleotide sequence of any sequence set forth in any of SEQ ID NOS: 8-35,37-42, and 51-90, complements thereof, allelic variants thereof or complements of allelic variants thereof For example, primers comprising a nucleotide sequence of at least about consecutive nucleotides, at least about 25 nucleotides or having from about 15 to about 20 nucleotides set forth in any of SEQ ID NOS:8-35, 37-42, and 51-90, or complements thereof are provided by the invention. Primers having a sequence of more than about 25 nucleotides are also within the scope of the invention. Preferred primers of the invention are primers that can be used in PCR for amplifying each of the exons of a CADPKL gene.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of the nucleotide sequence shown in Table 4A (SEQ ID NOs:8-35 and 51-76). A reverse primer can be a primer having a nucleotide sequence or a portion of the nucleotide sequence that is complementary to a nucleotide sequence shown in Table 4A (SEQ ID NOs:8-35 and 51-76).

The nucleic acid molecules of the invention also include “chimeric” CADPKL nucleic acid molecules. Such chimeric nucleic acid molecules are polynucleotides which comprise at least one CADPKL nucleic acid sequence (which may be any of the full length or partial CADPKL nucleic acid sequences described above), and also at least on non-CADPKL nucleic acid sequence. For example, the non-CADPKL nucleic acid sequence may be a heterologous regulatory sequence (for example, a promoter sequence) that is derived from another, non-CADPKL gene and is not normally associated with a naturally occurring CADPKL gene. The non-CADPKL nucleic acid sequence may also be a coding sequence of another, non-CADPKL polypeptide, such as FLAG, a histidine tag, glutathi one S-transferase (GST), hemaglutinin, β-galactosidase, thioreductase, or an immunoglobulin domain or domains (for example, an Fc region). In preferred embodiments, a chimeric nucleic acid molecule of the invention encodes a CADPKL fusion polypeptide of the invention.

CADPKL nucleic acid molecules of the invention, whether genomic DNA, cDNA, mRNA or otherwise, can be isolated from any source including, for example, cDNA or genomic libraries. Preferably, the cDNA library is a library generated from cells, tissue or organ, such as brain, which expresses a CADPKL gene of the invention. For example, the CADPKL EST nucleic acid sequences set forth in SEQ ID NOS:6 and 7 are both ones that were isolated from a human brain cDNA library. Methods for obtaining particular genes (i.e., CADPKL genes and nucleic acids) from such libraries are well known in the art, as described above (see, e.g., Sambrook et al., 1989, supra).

The DNA may be obtained by standard procedures known in the art from cloned DNA (for example, from a DNA “library”), and preferably is obtained from a cDNA library prepared from cells or tissue with high level expression of the gene or its gene product (for example, from brain cells or tissue). In one embodiment, the DNA may be obtained from a “subtraction” library to enrich the library for cDNAs of genes specifically expressed by a particular cell type or under certain conditions. In still other embodiments, a library may be prepared by chemical synthesis, by cDNA cloning, or by the cloning of genomic DNA or fragments thereof purified from the desired cell (see, for example, Sambrook et al, 1989, supra; Glover, D. M. edl, 1985, DNA Cloning: A Practical Approach, MRL Press, Ltd., Oxford, U.K. Vols. I and II).

Clones derived from genomic DNA may contain regulatory and intron DNA regions in addition to coding regions. Clones derived from cDNA generally will not contain intron sequences. Whatever the source, the gene is preferably molecularly cloned into a suitable vector for propagation of the gene. Identification of the specific DNA fragment containing the desired CADPKL gene may be accomplished in a number of ways. For example, a portion of a CADPKL gene exemplified infra can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe, such as an allelic variant from another individual, will hybridize thereto. In a specific embodiment, highest stringency hybridization conditions are used to identify a homologous CADPKL gene.

Further selection can be carried out on the basis of properties of the CADPKL gene product; such as if the gene encodes a protein product having the isoelectric electrophoretic, amino acid composition, partial or complete amino acid sequence, antibody binding activity or ligand binding profile of a CADPKL polypeptide as disclosed herein. Thus, the presence of the gene may be detected by assays based on the physical, chemical, inununological or functional properties of its expressed product.

Other DNA sequences which encode substantially the same amino acid sequence as a CADPKL gene may be used in the practice of the present invention. These include, but are not limited to allelic variants, species variants, sequence conservative variants, and functional variants. In particular, the nucleic acid sequences of the invention include both “function-conservative variants” and “sequence-conservative variants”. Nucleic acid substitutions may be made, for example, to alter the amino acid residue encoded by a particular codon, and thereby substitute an amino acid sequence in a CADPKL polypeptide for one with a particularly preferable property.

CADPKL Polymorphisms.

The present invention also provides, in preferred embodiments, variant CADPKL nucleic acids including variants which comprise one or more single nucleotide polymorphisms (SNPs). As an example, and not by way of limitation, Table 2, infra, discloses several single nucleotide polymorphisms (SNPs) of the CADPKL genomic sequence set forth in SEQ ID NO:1. Table 3A discloses similar SNPs of the CADPKL cDNA sequences set forth in SEQ ID NOS:2 and 4. In addition, the Examples, infra, demonstrate that these SNPs are ones which correlate with a neuropsychiatric disorder. Accordingly, CADPKL nucleic acid molecules which comprise one or more of these SNPs are particularly preferred embodiments of CADPKL nucleic acids of the present invention.

The polymorphic sequences of the invention can advantageously be used as primers to amplify an allelic variant of a CADPKL gene, i.e., nucleic acids which are capable of selectively hybridizing to an allelic variant of a polymorphic region of a CADPKL gene. Thus, such primers can be specific for a CADPKL gene sequence, so long as they have a nucleotide sequence which is capable of hybridizing to a CADPKL gene. Preferred primers are capable of specifically hybridizing to any of the allelic variants listed in Table 4A (SEQ ID NOS: 37-42 and 77-90). Such primers can be used, e.g., in sequence specific oligonucleotide priming as described further herein.

The CADPKL nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of a CADPKL gene which is polymorphic (SEQ ID NOS: 37-42 and 77-90). In an even more preferred embodiment of the invention, the probes are capable of hybridizing specifically to one allelic variant of a CADPKL gene having a nucleotide sequence which differs from the nucleotide sequence set forth in SEQ ID NOS 1, 2 and/or 4. Such probes can then be used to specifically detect which allelic variant of a polymorphic region of a CADPKL gene is present in a subject. The polymorphic region can be located in the promoter, exon, or intron sequences of a CADPKL gene.

For example, preferred probes of the invention are those probes listed in Table 2, wherein the bold nucleotides represent the location of the nucleotide polymorphism. For each probe listed in Table 2, the complement of that probe is also included in the Table as a preferred probe of the invention. Particularly preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a CADPKLgene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In another preferred embodiment of the invention, the isolated nucleic acid, which is used, e.g., as a probe or a primer, is modified, such as to become more stable. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published December 15, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the nucleic acid of the invention maybe conjugated to another molecule, e.g., a peptide, hybridization triggered cross-linking agent, transport agent, hybridization-triggered cleavage agent, etc.

The isolated nucleic acid comprising a CADPKL intronic sequence may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acidmethylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides maybe synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The invention also provides other variants of a CADPKL nucleic acid, including nucleic acids having variant microsatellite repeats. A “microsatellite repeat” or “microsatellite”, as the term is used herein, refers to a short sequence ofrepeating nucleotides within a nucleic acid. Typically, a microsatellite repeat comprises a repeating sequence of two (i.e., a dinucleotide repeat), three (i.e., a trinucleotide repeat), four (i.e., a tetranucleotide repeat) or five (i.e., a pentanucleotide repeat) nucleotides. Microsatellites of the invention therefore have the general formula (N₁, N₂, . . . Nᵢ)ₙ, wherein N represents a nucleic acid residue (e.g., adenine, thymine, cytosine or guanine), i represents the number of the last nucleotide in the microsatellite, and n represents the number of times the motif is repeated in the microsatellite locus. In one embodiment the number of nucleotides in a microsatellite motif (i) is about six, preferably between two and five, and more preferably two, three or four. The total number of repeats (n) in a microsatellite repeat may be, e.g., from one to about 60, preferably from 4 to 40, and more preferably from 10 to 30 when i=2; is preferably between about 4-25, and more preferably between about 6-22 when i=3; and is preferably between about 4-15, and more preferably between about 5-10 when i=4. A CADPKL nucleic acid of the invention may comprise any microsatellite repeat of the above general formula. However, the following motifs are particularly preferred: CA, TC, and, AATTG; as well as all complements and permutations of such motifs (for example, TG, GA, and CAATT. As a specific, non-limiting example, Table 7, infra, identifies several novel microsatellite repeats in a CADPKL nucleic acid, as well as some known microsatellite repeats (e.g., D 1S471 and D 1S491) that may be associated with a neuropsychiatric disorder. These variant CADPKL nucleic acids are also considered part of the present invention.

Accordingly, the nucleic acid molecules of the present invention include CADPKL nucleic acid molecules having one or more of the polymorphisms described in Table 2 and Table 3A (SEQ ID NOS:37-42 and 77-90). In preferred embodiments, the nucleic acid molecules of the invention include specific CADPKL allelic variants, which differ from the reference or wild-type CADPKL nucleic acid molecules described supra (i.e., nucleic acid molecules having the nucleotide sequence set forth in SEQ ID NO:1, in SEQ ID NO:2, or in SEQ ID NO:4).

The genes encoding CADPKL derivatives and analogs of the invention can be produced by various methods known in the art. The manipulations which result in their production can occur at the gene or protein level. For example, the cloned CADPKL gene sequence can be modified by any of numerous strategies known in the art (see, e.g., Sambrook et al., 1989, supra). The sequence can be cleaved at appropriate sites with restriction endonuclease(s), followed by further enzymatic modification if desired, isolated, and ligated in vitro. In the production of the gene encoding a derivative or analog CADPKL, care should be taken to ensure that the modified gene remains within the same translational reading frame as the CADPKL gene, uninterrupted by translational stop signals, in the gene region where the desired activity is encoded.

Additionally, the CADPKL-encoding nucleic acid sequence can be mutated in vitro or in vivo, to create and/or destroy translation, initiation and/or termination sequences, or to create variations in coding regions and/or form new restriction endonuclease sites or destroy preexisting ones, to facilitate further in vitro modification. Modifications can also be made to introduce restriction sites and facilitate cloning the CADPKL gene into an expression vector. An technique for mutagenesis known in the art can be used, including but not limited to, in vitro site-directed mutagenesis (Hutchinson et al., J. Biol. Chem. 1978, 253:6551; Zoller & Smith, DNA 1984, 3:479-488; Oliphant et al., Gene 1986, 44:177; Hutchinson et al., Proc. Natl. Acad. Sci. U.S.A. 1986, 83:710), use of TAB linkers (Pharmacia), etc. PCR techniques are preferred for site directed mutagenesis (see, Higuchi, 1989, “Using PCR to Engineer DNA” in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible cloning vectors include, but are not limited to, plasmids or modified viruses. The vector system must, however, by compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pKK plasmids (Clonetech), pET plasmids (Novagen, Inc., Madison, Wis.), pRSET or pREP plasmids, pcDNA (Invitrogen, Carlsbad, Calif.), pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (i.e., “linkers”) onto the DNA termini. These ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell (for example, E. coli) and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequence from the yeast 2m plasmid.

### 5.3. CADPKL Polypeptides

The present invention relates to a polypeptide referred to herein as the Calcium/Calmodulin Dependent Protein Kinase Like polypeptide or CADPKL. A CADPKL polypeptide is, in general, a polypeptide that is encoded by a gene which hybridizes to the complement of a CADPKL nucleic acid sequence as described in Section 5.2, supra. Typically, a full length CADPKL polypeptide comprises a sequence of approximately 450 to 480 amino acid residues and, more preferably, comprises a sequence of 460 to 476 amino acid residues.

In one specific embodiment, a CADPKL polypeptide is a polypeptide from a human cell or tissue and, more preferably, from a human brain cell or tissue. For example, a human CADPKL polypeptide of the invention may comprise the amino acid sequence set forth in SEQ ID NO:3 or, alternatively, the amino acid sequence set forth in SEQ ID NO:5.

In other embodiments, CADPKL polypeptides of the invention also include fragments of a full length CADPKL polypeptide. For example, the CADPKL polypeptides also include polypeptides comprising the amino acid sequence of an epitope of a full length CADPKL polypeptide, such as an epitope of the full length CADPKL polypeptide set forth in SEQ ID NO:3 or in SEQ ID NO:5. An epitope of a CADPKL polypeptide represents a site on the polypeptide against which an antibody may be produced and to which the antibody binds. Therefore, polypeptide comprising the amino acid sequence of a CADPKL epitope are useful for making antibodies to a CADPKL polypeptide. Preferably, an epitope comprises a sequence of at least 5, more preferably at least 10, 15, 20, 25 or 50 amino acid residues in length. Thus, CADPKL polypeptides of the invention that comprise epitopes of a full length CADPKL polypeptide preferably contain an amino acid sequence corresponding to at least 5, at least 10, at least 15, at least 20, at least 25, or at least 50 amino acid residues of the full length CADPKL sequence. For example, in certain preferred embodiments wherein the epitope is an epitope of the full length CADPKL polypeptide set forth in SEQ ID NO:3, a CADPKL polypeptide of the invention preferably comprises an amino acid sequence corresponding to at least 5, at least 10, at least 15, at least 20, at least 25 or at least 50 amino acid residues of the sequence set forth in SEQ ID NO:3. In other embodiments wherein the epitope is an epitope of the full length CADPKL polypeptide set forth in SEQ ID NO:5, a CADPKL polypeptide of the invention preferably comprises an amino acid sequence corresponding to at least 5, at least 10, at least 15, at least 20, at least 25 or at least 50 amino acid residues of the sequence set forth in SEQ ID NO:5.

The CADPKL polypeptides of the invention also include analogs and derivatives of the full length CADPKL polypeptides (e.g., of SEQ ID NOS:3 and 5). Analogs and derivatives of the CADPKL polypeptides of the invention have the same or homologous characteristics of CADPKL polypeptides set forth above. For example, a CADPKL polypeptide derivative may be a functionally active derivative; i.e., it may be capable of exhibiting one or more functional activities associated with a full length, wild-type CADPKL polypeptide of the invention such as one of the polypeptides set forth in SEQ ID NOS:3 and 5.

CADPKL chimeric or fusion polypeptides may also be prepared in which the CADPKL portion of the fusion polypeptide has one or more characteristics of a CADPKL polypeptide described above. Such fusion polypeptides therefore represent embodiments of the CADPKL polypeptides of this invention. Exemplary CADPKL fusion polypeptides include ones which comprise a full length, derivative or truncated CADPKL amino acid sequence, as well as fusions which comprise a fragment of a CADPKL polypeptide sequence (e.g., a fragment corresponding to an epitope or to one or more domains). Such fusion polypeptides may also comprise the amino acid sequence of a marker polypeptide; for example FLAG, a histidine tag, glutathione S-transferase (GST) or the Fc portion of an IgG. In other embodiments, a CADPKL polypeptide may be expressed with (e.g., fused to) a bacterial protein such as β-galactosidase. Additionally, CADPKL fusion polypeptides may comprise amino acid sequences that increase solubility of the polypeptide, such as a thioreductase amino acid sequence or the sequence of one or more immunoglobulin proteins (e.g., IgG1 or IgG2).

CADPKL analogs or variants can also be made by altering encoding nucleic acid molecules, such as by substitutions, additions or deletions. For example, analogs or variants of a CADPKL polypeptide may be made by using any of the variant or polymorphic CADPKL nucleic acids described infra to encode a variant CADPKL polypeptide. Preferably, such altered nucleic acid molecules encode functionally similar molecules (i.e., molecules that perform one or more CADPKL functions or have one or more CADPKL bioactivities). Thus, in a specific embodiment, an analog of a CADPKL polypeptide is a function-conservative variant.

A CADPKL analog or variant polypeptide is also, preferably, one that is encoded by a CADPKL nucleic acid that is associated with a neuropsychiatric disorder, such as schizophrenia, schizoaffective disorder, bipolar affective disorder, unipolar affective disorder and adolescent conduct disorder. For instance, the Examples infra describe various mutations to the CADPKL gene that encode an analog CADPKL polypeptide. Such analog CADPKL polypeptides therefore represent exemplary, specific embodiments of analog CADPKL gene products of the present invention. In particular, the Examples describe many variant CADPKL polypeptides encoded by CADPKL genes with these mutations. These particular, variant CADPKL polypeptides comprise one or more amino acid residue substitutions, including the specific substitutions provided in Table 6B of the Examples, infra. Thus, CADPKL polypeptides (e.g., having the polypeptide sequence set forth in SEQ ID NO:3 or 5) comprising one or more of these specific amino acid substitutions represent exemplary embodiments of analog CAPDKL gene products of the present invention.

Amino acid residues, other than ones that are specifically identified herein as being conserved, may differ among variants of a protein or polypeptide. Accordingly, the percentage of protein or amino acid sequence similarity between any two CADPKL polypeptides of similar function may vary. Typically, the percentage of protein or amino acid sequence similarity between different CADPKL polypeptide variants may be from 70% to 99% or higher, as determined according to an alignment scheme such as the Cluster Method and/or the MEGALIGN algorithm. “Function-conservative variants” also include polypeptides that have at least 50%, preferably at least 75%, more preferably at least 85% and still more preferably at least 90% amino acid sequence identity as determined, e.g., by BLAST or FASTA algorithms. In one embodiment, such analogs and variants of a CADPKL polypeptide are function-conservative variants which have the same or similar properties, functions or bioactivities as the native polypeptide to which they are compared.

In another preferred embodiment, such analogs and variants of a CADPKL polypeptide are ones which are associated with a neuropsychiatric disorder, such as schizophrenia, schizoaffective disorder, bipolar affective disorder, unipolar affective disorder and adolescent conduct disorder. It is further noted that the analogs of the CADPKL polypeptides of the present invention include, not only homologs and variants of the full length CADPKL polypeptides (e.g., variants of a CADPKL polypeptide comprising the amino acid sequence set forth in SEQ ID NO:3 or 5), but also include variants of modified CADPKL polypeptides (e.g., truncations and deletions) and of fragments (e.g., corresponding to particular domains, regions or epitopes) of a full length CADPKL polypeptide. In yet other embodiments, an analog of a CADPKL polylpeptide is an allelic variant or mutant of a CADPKL polypeptide. The term allelic variant and mutant, when used to describe a polypeptide, refers to a polypeptide encoded by an allelic variant or mutant gene. Thus, the allelic variant and mutant CADPKL polypeptides of the invention are polypeptides encoded by allelic variants or mutants of the CADPKL nucleic acid molecules of the present invention (see, Section 5.3, infra).

In yet other embodiments, an analog of a CADPKL polypeptide is a substantially homologous polypeptide from the same species (e.g., an allelic variant) or from another species (e.g., an orthologous polypeptide); preferably from another mammalian species such as mouse, rat, rabbit, hamster, guinea pig, primate (e.g., monkey or human), cats, dogs, sheep, goats, pigs, horses, cows, etc. However, an analog of a CADPKL polypeptide may be from any species of organism, including chickens, Xenopus, yeast (e.g., Saccharomyces cerevisiae) and bacteria (e.g., E. coli) to name a few. For example, the rat homolog of CADPKL has been cloned and is also known in the art (see, Yokokura et al., Biochem. Biophys. Acta. 1997, 1338:8-12). Thus, this homolog is a particular example of the CADPKL analogs and homologs of the present invention.

In a specific embodiment, two polypeptide sequences are “substantially homologous” or “substantially similar” when the polypeptides are at least 35-40% similar, as determined by one of the algorithms disclosed herein. Preferably, two substantially homologous polypeptide sequences are at least about 60% similar, and more preferably at least about 90 or 95% similar in one or more highly conserved domains or, for allelic variants, across the entire amino acid sequence.

In other embodiments, variants of a CADPKL polypeptide (including analogs, orthologs, and homologs) are polypeptides encoded by nucleic acid molecules that hybridize to the complement of a nucleic acid molecule encoding a CADPKL polypeptide; e.g., in a Southern hybridization experiment under defined conditions. For example, in a particular embodiment analogs and/or homologs of a CADPKL polypeptide comprise amino acid sequence encoded by nucleic acid molecules that hybridize to a complement of a CADPKL nucleic acid sequence, for example a complement of the coding sequence set forth in SEQ ID NO:2 or the cDNA sequence set forth in SEQ ID NO:2, under highly stringent hybridization conditions that comprise, e.g., 50% formamide and 5× or 6× SSC. In other embodiments, the analogs and/or homologs of the CADPKL polypeptide may comprise amino acid sequences encoded by nucleic acid molecules that hybridize to a complement of a CADPKL nucleic acid sequence (e.g., the complement of the coding sequence set forth in SEQ ID NO:2 or of the cDNA sequence set forth in SEQ ID NO:4) under moderately stringent hybridization conditions (e.g., 40% formamide with 5× or 6× SSC), or under low stringency conditions (e.g., in 5×SSC, 0.1% SDS, 0.25% milk, no formamide, 30% formamide, 5× SSC or 0.5% SDS).

In still other embodiments, variants (including analogs, homologs and orthologs) of a CADPKL polypeptide can also be identified by isolating variant CADPKL genes; e.g., by PCR using degenerate oligonucleotide primers designed on the basis of amino acid sequences of a CADPKL polypeptide (for example, the polypeptide sequence set forth in SEQ ID NO:3 or 5).

Derivatives of the CADPKL polypeptides of the invention further include, but are by no means limited to, phosphorylated CADPKL, myristylated CADPKL, methylated CADPKL and other CADPKL polypeptides that are chemically modified. CADPKL polypeptides of the invention may further include labeled variants; for example, radio-labeled with iodine or phosphorous (see, e.g., EP 372707B) or other detectable molecule such as, but by no means limited to, biotin, a fluorescent dye (e.g., Cy5 or Cy3), a chelating group complexed with a metal ion, a chromophore or fluorophore, a gold colloid, a particle such as a latex bead, or attached to a water soluble polymer.

Chemical modification of a biologically active component or components of CADPKT, nucleic acids or polypeptides may provide additional advantages under certain circumstances. See, for example, U.S. Pat. No. 5,179,337 issued Dec. 18, 1970 to Davis et a. Also, for a review see Abuchowski et al. in Enzymes as Drugs (J. S. Holcerberg and J. Roberts, eds. 1981), pp.367-383. A review article describing protein modification and fusion proteins is found in Francis, Focus on Growth Factors 1992, 3:4-10, Mediscript: Mountview Court, Friern Bamet Lane, London N20, OLD, UK.

Polymorphic CADPKL Polypeptides.

The present invention provides isolated polymorphic CADPKL polypeptides, such as CADPKL polypeptides which are encoded by specific allelic variants of CADPKL genes, including those identified herein. Accordingly, preferred CADPKL polypeptides of the invention have an amino acid sequence which differs from SEQ ID NOs:3 or 5. In one embodiment, the CADPKL polypeptides are isolated from, or otherwise substantially free of other cellular proteins. The term “substantially free of other cellularproteins” (also referred to herein as “contaminating proteins”) or “substantially pure or purified preparations” are defined as encompassing preparations of CADPKL polypeptides having less than about 20% (by dry weight) contaminating protein, and preferably having less than about 5% contaminating protein. It will be appreciated that functional forms of the subject polypeptides can be prepared, for the first time, as purified preparations by using a cloned gene as described herein.

Preferred CADPKL proteins of the invention have an amino acid sequence which is at least about 60%, 70%, 80%, 85%, 90%, or 95% identical or homologous to an amino acid sequence of SEQ ID NOS.:3 or 5. Even more preferred CADPKL proteins comprise an amino acid sequence which is at least about 97, 98, or 99% homologous or identical to an amino acid sequence of SEQ ID NO.:3 or 5. Such proteins can be recombinant proteins, and can be, e.g., produced in vitro from nucleic acids comprising a specific allele of a CADPKL polymorphic region. For example, recombinant polypeptides preferred by the present invention can be encoded by a nucleic acid, which is at least 85% homologous and more preferably 90% homologous and most preferably 95% homologous with a nucleotide sequence set forth in SEQ ID NOS: 1, 2, or 4, and comprises an allele of a polymorphic region that differs from that set forth in SEQ ID NOs:1, 2, or 4. Polypeptides which are encoded by a nucleic acid that is at least about 98-99% homologous with the sequence of SEQ ID NOs: 1, 2, and 4 and comprise an allele of a polymorphic region that differs from that set forth in SEQ ID NOs: 1, 2, or 4 are also within the scope of the invention.

In a preferred embodiment, a CADPKL protein of the present invention is a mammalian CADPKL protein. In an even more preferred embodiment, the CADPKL protein is a human protein, such as a CADPKL polypeptide comprising an amino acid sequence from SEQ ID NO: 3 or 5 in which amino acid 329 is an isoleucin residue.

CADPKL polypeptides preferably are capable of functioning in one of either role of an agonist or antagonist of at least one biological activity of a wild-type (“authentic”) CADPKL protein of the appended sequence listing. The term “evolutionarily related to”, with respect to amino acid sequences of CADPKL proteins, refers to both polypeptides having amino acid sequences which have arisen naturally, and also to mutational variants of human CADPKL polypeptides which are derived, for example, by combinatorial mutagenesis.

Full length proteins or fragments corresponding to one or more particular motifs and/or domains or to arbitrary sizes, for example, at least 5, 10, 25, 50, 75 and 100,amino acids in length are within the scope of the present invention.

Isolated peptidyl portions of CADPKL proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a CADPKL polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) CADPKL protein.

In general, polypeptides referred to herein as having an activity (e.g., are “bioactive”) of a CADPKL protein are defined as polypeptides which mimic or antagonize all or a portion of the biological/biochemical activities of a CADPKL protein having SEQ ID NOs:3 or 5, such as the ability to bind a substrate pr ligand. Other biological activities of the subject CADPKL proteins are described herein or will be reasonably apparent to those skilled in the art. According to the present invention, a polypeptide has biological activity if it is a specific agonist or antagonist of a naturally-occurring form of a CADPKL protein.

Assays for determining whether a CADPKL protein or variant thereofhas one or more biological activities are well known in the art.

Other preferred proteins of the invention are those encoded by the nucleic acids set forth in the section pertaining to nucleic acids of the invention. In particular, the invention provides fusion proteins, e.g., CADPKL-immunoglobulin fusion proteins. Such fusion proteins can provide, e.g., enhanced stability and solubility of CADPKL proteins and may thus be useful in therapy. Fusion proteins can also be used to produce an immunogenic fragment of a CADPKL protein. For example, the VP6 capsid protein of rotavirus can be used as an immunologic carrier protein for portions of the CADPKL polypeptide, either in the monomeric form or in the form of a viral particle. The nucleic acid sequences corresponding to the portion of a subject CADPKL protein to which antibodies are to be raised can be incorporated into a fusion gene construct which includes coding sequences for a late vaccinia virus structural protein to produce a set of recombinant viruses expressing fusion proteins comprising CADPKL epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a CADPKL protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of a CADPKL polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of CADPKL proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the CADPKL polypeptides of the present invention. For example, CADPKL polypeptides can be generated as glutathione-S-transferase (GST-fusion) proteins. Such GST-fusion proteins can enable easy purification of the CADPKL polypeptide, as for example by the use of glutathione-derivatized matrices (see, for example, Current Protocols in Molecular Biology, eds. Ausubel et al. (N.Y.: John Wiley & Sons, 1991)).

The present invention further pertains to methods of producing the subject CADPKL polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant CADPKL polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant CADPKL polypeptide is a fusion protein containing a domain which facilitates its purification, such as GST fusion protein.

Moreover, it will be generally appreciated that, under certain circumstances, it may be advantageous to provide homologs of one of the subject CADPKI, polypeptides which function in a limited capacity as one of either a CADPKL agonist (mimetic) or a CADPKL antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of CADPKL proteins.

Homologs of each of the subject CADPKL proteins can be generated by mutagenesis, such as by discrete point mutation(s), or by truncation. For instance, mutation can give rise to homologs which retain substantially the same, or merely a subset, of the biological activity of the CADPKL polypeptide from which it was derived. Alternatively, antagonistic forms of the protein can be generated which are able to inhibit the function of the naturally occurring form of the protein, such as by competitively binding to a substrate or ligand.

The recombinant CADPKL polypeptides of the present invention also include homologs of CADPKL polypeptides which differ from the CADPKL proteins having SEQ ID NOS.:3 or 5, such as versions of those protein which are resistant to proteolytic cleavage, as for example, due to mutations which alter ubiquitination or other enzymatic targeting associated with the protein.

CADPKL polypeptides may also be chemically modified to create derivatives by forming covalent or aggregate conjugates with other chemical moieties, such as glycosyl groups, lipids, phosphate, acetyl groups and the like. Covalent derivatives of CADPKL proteins can be prepared by linking the chemical moieties to functional groups on amino acid side-chains of the protein or at the N-terminus or at the C-terminus of the polypeptide.

Modification of the structure of the subject CADPKL polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the CADPKL polypeptides described in more detail herein. Such modified peptides can be produced, for instance, by amino acid substitution, deletion, or addition. The substitutional variant may be a substituted conserved amino acid or a substituted non-conserved amino acid. For example, it is reasonable to expect that an isolated replacement of a leucine with an isoleucine or valine) an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, WH Freeman and Co.: 1981). Whether a change in the amino acid sequence of a peptide results in a functional CADPKL homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

### 5.4. Expression of CADPKL Polypeptides

A nucleotide sequence coding for CADPKL, for an antigenic fragment, derivative or analog of CADPKL, of for a functionally active derivative of CADPKL (including a chimeric protein) may be inserted into an appropriate expression vector, i.e., a vector which contains the necessary elements for the transcription and translation of the inserted protein-coding sequence. Thus, a nucleic acid encoding a CADPKL polypeptide of the invention can be operationally associated with a promoter in an expression vector of the invention. Both cDNA and genomic sequences can be cioned and expressed under control of such regulatory sequences. Such vectors can be used to express functional or functionally inactivated CADPKL polypeptides. In particular, the CADPKL nucleic acids which may be cloned and expressed according to these methods include, not only wild-type CADPKL nucleic acids, but also mutant or variant CADPKL nucleic acids. These include, for example, a CADPKL nucleic acid having one or more mutations or polymorphisms that are associated with a neuropsychiatric disorder, such as CADPKL nucleic acids having one or more of the polymorphisms specified in Table 5 and in Table 6A of the Examples, infra. In addition, nucleic acids that encode a variant CADPKL polypeptide, for example a variant CADPKL polypeptide associated with a neuropsychiatric disorder and/or having one or more of the amino acid substitutions disclosed in Table 6B of the Examples, infra) may be cloned and expressed according to the methods described here.

The necessary transcriptional and translational signals can be provided on a recombinant expression vector.

Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of a CADPKL protein may be controlled by any promoter/enhancer element known in the art, but these regulatory elements must be functional in the host selected for expression. Promoters which may be used to control CADPKL gene expression include, but are not limited to, cytomegalovirus (CMV) promoter (U.S. Pat. Nos. 5,385,839 and 5,168,062), the SV40 early promoter region (Benoist and Chambon, Nature 1981,290:304-310), the promoter contained in the 3′ long terminal repeat of Rous sarcoma virus (Yamamoto, et al., Cell 1980, 22:787-797), the herpes thymidine kinase promoter (Wagner et al., Proc. Natl. Acad. Sci. U.S.A. 1981, 78:1441-1445), the regulatory sequences of the metallothionein gene (Brinster et al., Nature 1982, 296:39-42); prokaryotic expression vectors such as the b-lactamase promoter (Villa-Komaroff, et al, Proc. Natl. Acad. Sci. U.S.A. 1978,75:3727-3731), or the tac promoter(DeBoer, et al., Proc. Natl. Acad. Sci. U.S.A. 1983, 80:21-25, 1983); see also “Useful proteins from recombinant bacteria” in Scientific American 1980,242:74-94. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985, 315:338-340; Kollias et al., Cell 1986, 46:89-94), hematopoietic stem cell differentiation factor promoters, erythropoietin receptor promoter (Maouche et al., Blood 1991, 15:2557), etc.

Indeed, any type of plasmid, cosmid, YAC or viral vector may be used to prepare a recombinant nucleic acid construct which can be introduced to a cell, or to tissue, where expression of a CADPKL gene product is desired. Alternatively, wherein expression of a recombinant CADPKL gene product in a particular type of cell or tissue is desired, viral vectors that selectively infect the desired cell type or tissue type can be used.

In another embodiment, the invention provides methods for expressing CADPKL polypeptides by using a non-endogenous promoter to control expression of an endogenous CADPKL gene within a cell. An endogenous CADPKL gene within a cell is a CK-2 gene of the present invention which is ordinarily (i.e., naturally) found in the genome of tht cell. A non-endogenous promoter, however, is a promoter or other nucleotide sequence that may be used to control expression of a gene but is not ordinarily or naturally associated with the endogenous CADPKL gene. As an example, methods of homologous recombination may be employed (preferably using non-protein encoding CADPKL nucleic acid sequences of the invention) to insert an amplifiable gene or other regulatory sequence in the proximity of an endogenous CADPKL gene. The inserted sequence may then be used, e.g., to provide for higher levels of CADPKL gene expression than normally occurs in that cell, or to overcome one or more mutations in the endogenous CADPKL regulatory sequences which prevent normal levels of CADPKL gene expression. Such methods of homologous recombination are well known in the art. See, for example, International Patent Publication No. WO 91/06666, published May 16, 1991 by Skoultchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 1988,67:31-40), pCR2.1 and pcDNA 3.1+ (Invitrogen, Carlsbad, Calif.), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant CADPKL protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 1992, 7:980-990). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), or can be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovii-us, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV 1) vector (Kaplitt et al. , Molec. Cell. Neurosci. 1991, 2:320-330), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as thevector described by Stratford-Perricaudet et al. (J. Clin. Invest. 1992,90:626-630; see also La Salle et al., Science 1993,259:988-990); and a defective adeno-associated virus vector (Samulski et al., J. Virol. 1987, 61:3096-3101; Samulski et al, J. Virol. 1989, 63:3822-3828; Lebkowski et al., Mol. Cell. Biol. 1988, 8:3988-3996).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), Transgene (Strasbourg, France; adenoviral, vaccinia, retroviral, and lentiviral vectors) and Invitrogen (Carlbad, Calif.).

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner et al., Proc. Natl. Acad. Sci. U.S.A. 1987, 84:7413-7417; Felgner and Ringold, Science 1989, 337:387-388; Mackey et al., Proc. Natl. Acad. Sci. U.S.A. 1988, 85:8027-8031; Ulmer et al., Science 1993, 259:1745-1748). Useful lipid compounds and compositions for transfer of nucleic acids are described in International Patent Publications WO 95/18863 and WO 96/17823, and in U.S. Pat. No. 5,459,127. Lipids may be chemically coupled to other molecules for the purpose of targeting (see, Mackey et al., Proc. Natl. Acad. Sci. U.S.A. 1988,85:8027-8031). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce the vector in vivo as a naked DNA plasmid. Naked DNA vectors for gene therapy can be introduced into the desired host cells by methods known in the art; e.g., electroporation, microinjection, cell fusion, DEAE dextran, calcium phosphate precipitation, use of a gene gun, or use of a DNA vector transporter (see, e.g., Wu et al., J. Biol. Chem. 1992, 267:963-967; Wu and Wu, J. Biol. Chem. 1988, 263:14621-14624; Hartmut et al., Canadian Patent Application No.2,012,311, filed Mar. 15, 1990; Williams et al., Proc. Natl. Acad. Sci. U.S.A. 1991, 88:2726-2730). Receptor-mediated DNA delivery approaches can also be used (Curiel et al., Hum. Gene Ther. 1992, 3:147-154; Wu and Wu, J. Biol. Chem. 1987,262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1998,321:893; WO 99/01157; WO 99/01158; WO 99/01175).

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (see, e.g., Wilson, Nat. Med. 1995, 1:887-889). In that regard, it is advantageous to employ a viral vector that is engineered to express a minimal number of antigens

### 5.5. Antibodies to CADPKL

Antibodies to CADPKL are useful, inter alia, for diagnostics and intracellular regulation of CADPKL activity, as set forth below. According to the invention, CADPKL polypeptides produced, e.g., recombinantly or by chemical synthesis, and fragments or other derivatives or analogs thereof, including fusion proteins, may be used as an immunogen to generate antibodies that recognize the CADPKL polypeptide. In particular, the CADPKL polypeptides which may be used to generate antibodies include not only wild type CADPKL polypeptides, but also variant CADPKL polypeptides that comprise one or more amino acid residue substitutions, insertions or deletions. For example, in one preferred embodiment, a variant CADPKL polypeptide associated with a neuropsychiatric disorder (for example, a CADPKL polypeptide having one or more of the amino acid substitutions set forth in Table 6B of the Examples, infra) may be used to generate antibodies that specifically recognize (i.e., bind to) a variant CADPKL polypeptide.

Such antibodies include but are not limited to polyclonal, monoclonal, chimeric, single chain, Fab fragments, and an Fab expression library. Such an antibody may be specific for (i.e., specifically binds to) a human CADPKL polypeptide of the present invention or, alternatively, for a CADPKL ortholog from some other species of organism, preferably another mammalian species such as another primate (e.g., ape or monkey) mouse, rat, etc. The antibody may recognize a mutant form of CADPKL (e.g., one which is associated with a neuropsychiatric disorder, such as a CADPKL polypeptide having one or more of the amino acid substitutions set forth in Table 6B), a wild-type CADPKL, or both.

Various procedures known in the art may be used for the production of polyclonal antibodies to CADPKL polypeptide or derivative or analog thereof. For the production of antibody, various host animals can be immunized by injection with the CK-2 polypeptide, or a derivative (e.g., fragment or fusion protein) thereof, including but not limited to rabbits, mice, rats, sheep, goats, etc. In one embodiment, the CK-2 polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the CK-2 polypeptide, or fragment, analog, or derivative thereof, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridomatechnique (Kozbor et al, Immunology Today 1983,4:72; Cote et al, Proc. Natl. Acad. Sci. U.S.A. 1983, 80:2026-2030), and the EBV-hybridoma technique to produce human monoclonal antibodies (Cole et al., in Monoclonal Antibodies and Cancer Therapy, Alan R. Liss, Inc., 1985, pp. 77-96). In an additional embodiment of the invention, monoclonal antibodies can be produced in germ-free animals (International Patent Publication No. WO 89/12690). In fact, according to the invention, techniques developed for the production of “chimeric antibodies” (Morrison et al., J. Bacteriol. 1984, 159:870; Neuberger et al., Nature 1984,312:604-608; Takedaetal., Nature 1985,314:452-454) may also be used. Briefly, such techniques comprise splicing the genes from an antibody molecule from a first species of organism (e.g., a mouse) that is specific for a CADPKL polypeptide together with genes from an antibody molecule of appropriate biological activity derived from a second species of organism (e.g., from a human). Such chimeric antibodies are within the scope of this invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

According to the invention, techniques described for the production of single chain antibodies (U.S. Pat. Nos. 5,476,786, 5,132,405, and 4,946,778) can be adapted to produce CADPKL polypeptide-specific single chain antibodies. An additional embodiment of the invention utilizes the techniques described for the construction of Fab expression libraries (Huse et al., Science 1989, 246:1275-1281) to allow rapid and easy identification of monoclonal Fab fragments with the desired specificity for a CK-2 polypeptide, or its derivatives, or analogs.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a CK-2 polypeptide, one may assay generated hybridomas for a product which binds to a CADPKL polypeptide fragment containing such epitope. For selection of an antibody specific to a CADPKL polypeptide from a particular species of animal, one can select on the basis of positive binding with CADPKL polypeptide expressed by or isolated from cells of that species of animal.

The foregoing antibodies can be used in methods known in the art relating to the localization and activity of the CADPKL polypeptide, e.g., for Western blotting, imaging CADPKL polypeptide in situ, measuring levels thereof in appropriate physiological samples, etc. using any of the detection techniques mentioned above or known in the art. Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations will decrease binding stability.

In still other embodiments, anti-CADPKL antibodies may also be used to isolate cells which express a CADPKL polypeptide by panning or related immunoadsorption techniques.

In a specific embodiment, antibodies that agonize or antagonize the activity of a CADPKL polypeptide can be generated. In particular, intracellular single chain Fv antibodies can be used to regulate (inhibit) CADPKL activity (Marasco et al., Proc. Natl. Acad. Sci. U.S.A. 1993, 90:7884-7893; Chen., Mol. Med. Today 1997, 3:160-167; Spitz et al., Anticancer Res. 1996, 16:3415-22; Indolfi et al., Nat. Med. 1996,2:634-635; Kijma et al., Pharmacol. Ther. 1995, 68:247-267). Such antibodies can be tested using the assays described infra for identifying ligands.

### 5.6. In Vivo Testing Using Transgenic Animals

Transgenic animals, including transgenic mammals, may be prepared for evaluating the molecular mechanism(s) of CADPKL and, particularly, for evaluating the molecular mechanism(s) of disease and disorders, for example neuropsychiatric disorders (e.g., schizophrenia, schizoaffective disorder, bipolar affective disorder, unipolar affective disorder and adolescent conduct disorder), that are associated with CADPKL. Such animals provide excellent models for screening and/or testing drug candidates for such disorders.

Thus, human CADPKL “knock-in” animals, including human CADPKL “knock-in” mammals, can be prepared for evaluating the molecular biology to this system in greater detail than is possible with human subjects. It is also possible to evaluate compounds or diseases in “knockout” animals, e.g., to identify a compound that can compensate for a defect in CADPKL activity. Both technologies permit manipulation of single units of genetic information in their natural position in a cell genome and to examine the results of that manipulation in the background of a terminally differentiated organism. Transgenic mammals can be prepared by any method, including but not limited to modification of embryonic stem (ES) cells and heteronuclear injecion into blast cells.

A “knock-in” animal is an animal (e.g., a mammal such as a mouse) in which an endogenous gene is substituted with a heterologous gene (Roamer et al., New Biol. 1991, 3:331). Preferably, the heterologous gene is “knocked-in” to a locus of interest, either the subject of evaluation (in which case the gene may be a reporter gene; see Elegant et al., Proc. Natl. Acad. Sci. USA 1998, 95:11897) of expression or function of a homologous gene, thereby linking the heterologous gene expression to transcription from the appropriate promoter. This can be achieved by homologous recombination, transposon (Westphal and Leder, Curr Biol 1997,7:530), using mutant recombination sites (Araki et al., Nucleic Acids Res 1997, 25:868) or PCR (Zhang and Henderson, Biotechniques 1998, 25:784).

A “knockout animal” is an animal (e.g., a mammal such as a mouse) that contains within its genome a specific gene that has been inactivated by the method of gene targeting (see, e.g., U.S. Pat. Nos. 5,777,195 and 5,616,491). Aknockout animal includes both a heterozygote knockout (i.e., one defective allele and one wild-type allele) and a homozygous mutant. Preparation of a knockout animal requires first introducing a nucleic acid construct that will be used to suppress expression of a particular gene into an undifferentiated cell type termed an embryonic stem cell. This cell is then injected into a mammalian embryo. In preferred embodiments for which the knockout animal is a mammal, a mammalian embryo with an integrated cell is then implanted into a foster mother for the duration of gestation. Zhou, et al. (Genes and Development, 1995, 9:2623-34) describes PPCA knock-out mice.

The term “knockout” refers to partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of: (1) DNA from some portion of the gene (exon sequence, intron sequence, and/or promoter sequence) to be suppressed; and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise: (1) a full or partial sequence of one or more exons and/or introns of the gene to be suppressed; (2) a full or partial promoter sequence of the gene to be suppressed; or (3) combinations thereof. Typically, the knockout construct is inserted into an embryonic stem cell (ES cell) and is integrated into the ES cell genomic DNA, usually by the process of homologous recombination. This ES cell is then inj ected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

Generally, for homologous recombination, the DNA will be at least about 1 kilobase (kb) in length and preferably 3-4 kb in length, thereby providing sufficient complementary sequence for recombination when the knockout construct is introduced into the genomic DNA of the ES cell (discussed below).

Included within the scope of this invention is an animal, preferably a mammal (e.g., a mouse) in which two or more genes have been knocked out or knocked in, or both. Such animals can be generated by repeating the procedures set forth herein for generating each knockout construct, or by breeding two animals, each with a single gene knocked out, to each other, and screening for those with the double knockout genotype.

Regulated knockout animals can be prepared using various systems, such as the tet-repressor system (see U.S. Pat. No. 5,654,168) or the Cre-Lox system (see U.S. Pat. Nos. 4,959,317 and 5,801,030).

In another series of embodiments, transgenic animals are created in which: (i) a human CADPKL gene(s) is(are) stably inserted into the genome of the transgenic animal; and/or (ii) the endogenous CADPKL genes are inactivated and replaced with their human counterparts (see, e.g., Coffman, Semin. Nephrol. 1997, 17:404; Esther et al., Lab. Invest. 1996, 74:953; Murakami et al., Blood Press. Suppl. 1996, 2:36). In one aspect of these embodiments, a human CADPKL gene inserted into and/or expressed by the transgenic animal comprise a wild-type CADPKL gene. For example, the wild-type human CADPKL gene may be a gene that encodes a polypeptide having the amino acid sequence set forth in SEQ ID NOS: 3 and/or 5. The wild-type human CADPKL gene may be a gene that encodes a nucleic acid gene product having the sequence set forth in SEQ ID NOS: 1, 2, and/or 4. In another aspect of these embodiments, the human CADPKL genes inserted into and/or expressed by the transgenic animal comprise a mutant or variant CADPKL gene. For example, a CADPKL gene having one or more of the polymorphisms described in the Examples infra may be inserted into and/or expressed by a transgenic animal of the invention. In a particularly preferred aspect of these embodiments, the polymorphism or mutation is one that is associated with a neuropsychiatric disorder such as schizophrenia, schizoaffective disorder, bipolar affective disorder, unipolar affective disorder and adolescent conduct disorder.

Such transgenic animals can be treated with candidate compounds and monitored for neuronal development, neurodegeneration, or efficacy of a candidate therapeutic compound.

### 5.7. Applications and Uses

Described herein are various applications and uses for the CADPKL gene and its gene product, including particular applications and uses for the CADPKL nucleic acids and polypeptides of the present invention, and for antibodies directed against these CADPKL nucleic acids and polypeptides. As described supra, the present application provides, for the first time, data showing that CADPKL is associated with neuropsychiatric disorders such as schizophrenia, attention deficit disorder (ADD) schizoaffective disorder, bipolar disorder (BAD), unipolar affective disorder and adolescent conduct disorder. In particular, the invention provides several variant CADPKL nucleic acids and variant CADPKL polypeptides that are encoded by these variant CADPKL nucleic acids (see, for Example, Tables 2-4, supra). The Examples, infra, further provide data demonstrating that the variant CADPKL nucleic acids and polypeptides of the invention are associated with neuropsychiatric disorders. Accordingly, the present invention also provides particular applications which use the CADPKL polypeptides and nucleic acids of the invention (including the variant CADPKL polypeptides and nucleic acids provided in the Examples, infra), e.g., to diagnose and/or treat neuropsychiatric disorders, including specific neuropsychiatric disorders such as schizophrenia, ADD, schizoaffective disorder, BAD, unipolar affective disorder and adolescent conduct disorder.

In particular, the methods of the present invention include diagnostic methods, e.g., to identify individuals who have a neuropsychiatric disorder (for example, schizophrenia, ADD, schizoaffective disorder, BAD, unipolar affective disorder or adolescent conduct disorder), or to identify individuals who have a predisposition to and/or an increased risk of developing such a disorder. For example, in preferred embodiments, the invention provides methods for determining whether an individual has a CADPKL gene comprising one or more of the variant CADPKL nucleic acid sequences described herein which is associated with a neuropsychiatric disorder. In other preferred embodiments, the invention provides methods for determining whether an individual expresses a variant CADPKL nucleic acid (for example, a CADKPL mRNA) or a variant CADPKL polypeptide that is associated with a neuropsychiatric disorder. By determining whether an individual has or expresses a CADPKL nucleic acid or polypeptide associated with a neuropsychiatric disorder, the individual is identified as one who has such a disorder or, alternatively, as one who has a predisposition to and/or an increased risk of developing such a disorder. Such diagnostic and prognostic applications are described, in detail, in Subsection 5.6.1, infra.

Other applications and methods for using the CADPKL nucleic acids and polypeptides of this invention are also provided. In particular, Subsection 5.6.2 describes pharmacogenomic methods by which the variant CADPKL nucleic acid and/or polypeptide sequences of this invention may be used, e.g., to design therapies or treatments for an individual that are most likely to be affective. Subsection 5.6.3 describes methods for using a CADPKL nucleic acid or polypeptide of this invention to treat a disease or disorder associated with CADPKL, particularly a neuropsychiatric disease or disorder such as schizophrenia, schizoaffective disorder, bipolar affective disorder, unipolar affective disorder and adolescent conduct disorder. Subsection 5.6.4 describes other exemplary applications and methods for using CADPKL nucleic acids and polypeptides and, in particular, polymorphisms and variants of the CADPKL gene and its gene product. These methods include, for example, forensics methods, paternity testing, and kits.

### 5.7.1. Prognostic and Diagnostic Assays

The present methods provide means for determining if a subject has (diagnostic) or is at risk of developing (prognostic) a disease, condition or disorder that is associated with a CADPKL allele, e.g., neuropsychiatric disorders such as schizophrenia, ADD, schizoaffectiove disorder, BAD, unipolar affective disorder, and adolescent conduct disorder, or a neuropsychiatric disease or disorder/disorders resulting therefrom.

The present invention provides methods for determining the molecular structure of a CADPKL gene, such as a human CADPKL gene, or a portion thereof. In one embodiment, determining the molecular structure of at least a portion of a CADPKI, gene comprises determining the identity of the allelic variant of at least one polymorphic region of the gene (determining the presence or absence of one or more of the allelic variants, or their complements, of SEQ ID NOs.:1, 2,4,6-7 and/or 46-50). A polymorphic region of the CADPKL gene can be located in an exon, an intron, at an intron/exon border, or in the promoter of the gene.

The invention provides methods for determining whether a subject has, or is at risk of developing, a disease or condition associated with a specific allelic variant of a polymorphic region of a CADPKL gene. Such diseases can be associated with an abnormal neurological activity, such as, e.g., those associated with the onset of a neuropsychiatric disorder such as schizophrenia, schizoaffective disorder, bipolar disorder, unipolar affective disorder and adolescent conduct disorder. An aberrant CADPKL protein level can result from an aberrant transcription or post-transcriptional regulation. Thus, allelic differences in specific regions of a CADPKL gene can result in differences in the encoded protein due to differences in regulation of expression. In particular, some of the identified polymorphisms in the human CADPKL gene may be associated with differences in the level of transcription, RNA maturation, splicing, or translation of the gene or transcription product.

Analysis of one or more CADPKL polymorphic region in a subject can be useful for predicting whether a subject has or is likely to develop aberrant neurological activities or disorders resulting therefrom, such as neuropsychatric disorders or diseases, e.g., schizophrenia, ADD, schizoaffectiove disorder, BAD, unipolar affective disorder, and adolescent conduct disorder.

In preferred embodiments, the methods of the invention can be characterized as comprising detecting, in a sample of cells from the subject, the presence or absence of a specific allelic variant of one or more polymorphic regions of a CADPKL gene. The allelic differences can be: (i) a difference in the identity of at least one nucleotide or (ii) a difference in the number of nucleotides, which difference can be a single nucleotide or several nucleotides. The invention also provides methods for detecting differences in CADPKL genes such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. Examples of probes for detecting specific allelic variants of a polymorphic region located in the CADPKL gene are nucleic acid sequences comprising a nucleotide sequence from any of SEQ ID NOS: 37-42 or 77-90, as set forth in Table 2, supra. For instance, a probe for detecting a specific allelic variant in intron 4 is set forth in SEQ ID NO:37; a probe for detecting specific allelic variants of the polymorphic region located in intron 5 is set forth in SEQ ID NO:38; a probe for detecting specific allelic variants of the polymorphic region located in exon 7 is set forth in SEQ ID NO:39; a probe for detecting a specific allelic variant located in intron 8 is set forth in SEQ ID NO:85; probes for detecting specific allelic variants of the polymorphic region located in intron 9 are set forth in any of SEQ ID NOS:40-41; and probes for detecting specific allelic variants of the polymorphic region located in exon 10 is set forth in SEQ ID NO:42, and any of SEQ ID NOS:78, 79, and 84. Probes can also be used for detecting polymorphic variants of regions preceding, i.e., located upstream from, the coding sequence of the CADPKL gene, such as the promoter region. For instance, probes for detecting specific allelic variants of the polymorphic region located in the region located upstream to exon 1 are provided in SEQ ID NOS:77, 80-83, and 86-90. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ promoter region can be determined in a single hvbridization experiment.

In other detection methods, it is necessary to first amplify at least a portion of the CADPKL gene prior to identifying the allelic variant. Amplification can be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart. Preferred primers, such as primers for amplifying each of the exons of the human CADPKL gene, are listed in Table 4A in the Examples, infra.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a CADPKL gene and detect allelic variants, e.g., mutations, by comparing the sequence of the sample sequence with the corresponding wild-type (control) sequence. Exemplary sequencing reactions include those based on techniques developed by Maxam and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S Pat. No.5,605,798 and International Patent Application No. PCT/US96/03651 entitled DNA Diagnostics Based on Mass Spectrometry by H. Köster;. Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a CADPKL gene in DNA from a subject can be shown by restriction enzyme analysis.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a 5-LO allelic variant with a sample nucleic acid, e.g, RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gets to determine whether the control and sample nucleic acids have an identical nucleotide sequence or in which nucleotides they are different. See, for example, Cotton et al (1988) Proc. Natl Acad Sci USA 85:4397; Saleeba et al (1992) Methods Enzymod. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of CADPKL allelic variant. For example, single strand conformation polymorphism (SSCP) maybe used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region inpolyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of the CADPKL gene. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and can be used to detect specific allelic variants of a polymorphic region of a CADPKL gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms (SNPs) in a CADPKL gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient.

Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis (“GBA”) is described by Goelet, P. et al. (PCT Appln. No. 92/15712). The method of Goelet, P. et al uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2.650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al, Anal. Biochem. 208:171-175 (1993)). These methods differ from GBAO in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer.J. Hum. Genet. 52:46-59 (1993)).

For determining the identity of the allelic variant of a polymorphic region located in the coding region of a CADPKL gene, yet other methods than those described above can be used. For example, identification of an allelic variant which encodes amutated CADPKL protein can be performed by using an antibody specifically recognizing the mutant protein in, e.g., immunohistochemistry or immunoprecipitation. Antibodies to wild-type CADPKL protein or mutated forms of CADPKL proteins can be prepared according to methods known in the art and are also described here in Section 5.5, supra. Preferred antibodies specifically bind to a human CADPKL protein comprising any of the amino acid substitutions set forth in Table 3B. Alternatively, one can also measure an activity of a wild-type or mutant CADPKL protein, such as enzymatic activity or binding activity. Enzymatic assays are known in the art and involve, e.g., obtaining cells from a subject, and performing experiments with a substrate, labeled or unlabeled, to determine whether the conversion rate of the substrate differs from a control value. Alternatively, a ligand to the CADPKL protein can be mixed with both wild-type and mutant CADPKL protein to evaluate whether ligand binding of the mutant protein differs from ligand binding to the wild-type protein.

Antibodies directed against wild type or mutant CADPKL polypeptides or allelic variant thereof, which are discussed above, may also be used in disease diagnostics and prognostics. Such diagnostic methods, may be used to detect abnormalities in the level of CADPKL polypeptide expression, or abnormalities in the structure and/or tissue, cellular, or subcellular location of a CADPKL polypeptide. Structural differences may include, for example, differences in the size, electronegativity, or antigenicity of the mutant CADPKL polypeptide relative to the wild-type polypeptide. Protein from the tissue or cell type to be analyzed may easily be detected or isolated using techniques which are well known to one of skill in the art, including but not limited to western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of CADPKL polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the CADPKL polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-CADPKL polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the exteit of enzymatic reaction of a substrate in comparison with similarly prepared standards.

Detection may also be accomplished using any of a variety of other immunoassays. For example, byradioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a gene or gene product or polymorphic variants can be used to monitor the course of treatment or therapy.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA, e.g., DHPLC, sequencing and SSCP.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific CADPKL allelic variant.

Sample nucleic acid for using in the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) ofpatient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

### 5.7.2. Pharmacogenomics

Knowledge of the identity of the allele of one or more CADPKL gene polymorphic regions in an individual (the CADPKL genetic profile), alone or in conjunction with information on other genetic defects contributing to the same disease (the genetic profile of the particular disease) also allows a customization of the therapy for a particular disease to the individual's genetic profile, the goal of “pharmacogenomics”. For example, subjects having a specific allele of a CADPKL gene may or may not exhibit symptoms of a particular disease or be predisposed to developing symptoms of a particular disease. Further, if those subjects are symptomatic, they may or may not respond to a certain drug, e.g., a specific CADPKL therapeutic, such as, e.g., an inhibitor of CADPKL activity or binding, but may respond to another. Thus, generation of a CADPKL genetic profile, (e.g., categorization of alterations in CADPKL genes which are associated with the development of a particular disease), from a population of subjects, who are symptomatic for a disease or condition that is caused by or contributed to by a defective and/or deficient CADPKL gene and/or protein (a CADPKL genetic population profile) and comparison of an individual's CADPKL profile to the population profile, permits the selection or design of drugs that are expected to be safe and efficacious for a particular patient or patient population (i.e., a group of patients having the same genetic alteration).

For example, a CADPKL population profile can be performed by determining the CADPKL profile, e.g., the identity of CADPKL alleles, in a patient population having a disease, which is associated with one or more specific alleles of CADPKL polymorphic regions. Optionally, the CADPKL population profile can further include information relating to the response of the population to a CADPKL therapeutic, using any of a variety of methods, including, monitoring: 1) the severity of symptoms associated with the CADPKL related disease, 2) CADPKL gene expression level, 3) CADPKL mRNA level, 4) CADPKL protein level, 5) eosinophil level, and/or 6) leukotriene level, and dividing or categorizing the population based on particular CADPKL alleles. The CADPKL genetic population profile can also, optionally, indicate those particular CADPKL alleles which are present in patients that are either responsive or non-responsive to a particular therapeutic. This information or population profile, is then useful for predicting which individuals should respond to particular drugs, based on their individual CADPKL profile.

In a preferred embodiment, the CADPKL profile is a transcriptional or expression level profile and step (i) is comprised of determining the expression level of CADPKL proteins, alone or in conjunction with the expression level of other genes known to contribute to the same disease at various stages of the disease.

Pharmacogenomic studies can also be performed using transgenic animals. For example, one can produce transgenic mice, e.g., as described herein, which contain a specific allelic variant of a CADPKL gene. These mice can be created, e.g., by replacing their wild-type CADPKL gene with an allele of the human CADPKL gene. The response of these mice to specific CADPKL therapeutics can then be determined.

### 5.7.3. Methods of Treatment

The present invention provides for both prophylactic and therapeutic methods of treating a subject having or likely to develop a disorder associated with specific CADPKL alleles and/or aberrant CADPKL expression or activity, e.g., disorders or diseases associated with aberrant neurological functions, such as neuropsychiatric diseases or disorders.

The CADPKL nucleic acid molecules, polypeptides and antibodies of the present invention may be used, for example, in therapeutic methods to treat disorders, such as neuropsychiatric disorder (including, for example, schizophrenia, schizoaffective disorder, bipolar affective disorder, unipolar affective disorder, attention deficit disorder, and adolescent conduct disorder). In addition, compounds that bind to a CADPKL nucleic acid or polypeptide of the invention, compounds that modulate CADPKL gene expression, and compounds that interfere with or modulate binding of a CADPKL nucleic acid or polypeptide with a binding compound (e.g., with a natural ligand such as calmodulin) may be useful, e.g., in methods for treating such neuropsychiatric disorders.

For example, in a preferred embodiment, compounds that specifically bind to variant CADPKL nucleic acid of the present invention or, alternatively, compounds that specifically bind to a variant CADPKL gene product encoded by such a nucleic acid molecule may be used to inhibit the expression or activity of that variant CADPKL gene or gene product, while not inhibiting the expression or activity of a wild-type CADPKL gene or its gene product.

Prophylactic Methods.

In one aspect, the invention provides a method for preventing in a subject, a disease or condition associated with a specific CADPKL allele and/or an aberrant CADPKL expression or activity, such as a neuropsychiatric disorder, e.g., schizophrenia, and medical conditions resulting therefrom, by administering to the subject an agent which counteracts the unfavorable biological effect of the specific CADPKL allele. Subjects at risk for such a disease can be identified by a diagnostic or prognostic assay, e.g., as described herein. Administration of a prophylactic agent can occur prior to the manifestation of symptoms associated with specific CADPKL alleles, such that a disease or disorder is prevented or, alternatively, delayed in its progression. Depending on the identity of the CADPKL allele in a subject, a compound that counteracts the effect of this allele is administered. The compound can be a compound modulating the activity of a CADPKL polypeptide, e.g., an inhibitor. The treatment can also be a specific diet, or environmental alteration. In particular, the treatment can be undertaken prophylactically, before any other symptoms are present. Such a prophylactic treatment could thus prevent the development of an aberrant neurological function or aberrant neuropsychiatric profile such as those displayed in , e.g., schizophrenia, schizoaffective disorder, bipolar disorder, unipolar affective disorder and adolescent conduct disorder. The prophylactic methods are similar to therapeutic methods of the present invention and are further discussed in the following subsections.

Therapeutic Methods.

The invention further provides methods of treating subjects having a disease or disorder associated with a specific allelic variant of a polymorphic region of a CADPKL gene. Preferred diseases or disorders include those associated with aberrant neurological function, and disorders resulting therefrom (e.g., neuropsychiatric diseases and disorders, such as, for example, schizophrenia, schizoaffective disorder, bipolar disorder, unipolar affective disorder and adolescent conduct disorder).

In one embodiment, the method comprises (a) determining the identity of the allelic variant; and (b) administering to the subject a compound that compensates for the effect of the specific allelic variant. The polymorphic region can be localized at any location of the gene, e.g., in the promoter (e.g., in a regulatory element of the promoter), in an exon, (e.g., coding region of an exon), in an intron, or at an exon/intron border. Thus, depending on the site of the polymorphism in the CADPKL gene, a subject having a specific variant of the polymorphic region which is associated with a specific disease or condition, can be treated with compounds which specifically compensate for the allelic variant.

In a preferred embodiment, the identity of one or more of the nucleotides of a CADPKL gene identified in Table 2 can be determined.

In a particularly preferred embodiment, it is determined that a subject has A/G (WT/SNP) at position 143457 at position 146442 of SEQ ID NO:1.

If a subject has one or more of the polymorphisms of the invention (Table 2), that subject can have or be predicted to be at risk for developing a neuropsychatric disorder, e.g. schizophrenia. The neuropsychiatric disorder can be prevented from occurring or can be reduced by administering to the subject a pharmaceutically effective amount of a compound found to inhibit the activity or binding of the CADPKL polypeptide, or modifies the transcription or expression of the CADPKL gene.

Generally, the allelic variant can be a mutant allele, i.e., an allele which when present in one, or preferably two copies, in a subject results in a change in the phenotype of the subject. A mutation can be a substitution, deletion, and/or addition of at least one nucleotide relative to the wild-type allele (i.e., the reference sequence). Depending on where the mutation is located in the CADPKL gene, the subject can be treated to specifically compensate for the mutation. For example, if the mutation is present in the coding region of the gene and results in a more active the CADPKL protein, the subject can be treated, e.g., by administration to the subject of a CADPKL inhibitor, such that the administration of an inhibitor prevents aberrant neurological function associated with the CADPKL protein. In addition, wild-type CADPKL protein or nucleic acid coding sequence/cDNA can be administered to compensate for the endogenous mutated form of the CADPKL protein. Nucleic acids encoding wild-type human CADPKL protein are set forth in SEQ ID NOs:2 and 4. Furthermore, depending on the site of the mutation in the CADPKL protein and the specific effect on its activity, specific treatments can be designed to compensate for that effect.

Yet in another embodiment, the invention provides methods for treating a subject having a mutated CADPKL gene, in which the mutation is located in a regulatory region of the gene. Such a regulatory region can be localized in the promoter of the gene, in the 5′ or 3′ untranslated region of an exon, or in an intron. A mutation in a regulatory region can result in increased production of CADPKL protein, decreased production of CADPKL protein, or production of CADPKL protein having an aberrant tissue distribution. The effect of a mutation in a regulatory region upon the CADPKL protein can be determined, e.g., by measuring the protein level or mRNA level in cells having a CADPKL gene having this mutation and which, normally (i.e., in the absence of the mutation) produce CADPKL protein. The effect of a mutation can also be determined in vitro. For example, if the mutation is in the promoter, a reporter construct can be constructed which comprises the mutated promoter linked to a reporter gene, the construct transfected into cells, and comparison of the level of expression of the reporter gene under the control of the mutated promoter and under the control of a wild-type promoter. Such experiments can also be carried out in mice transgenic for the mutated promoter. If the mutation is located in an intron, the effect of the mutation can be determined, e.g., by producing transgenic animals in which the mutated CADPKL gene has been introduced and in which the wild-type gene may have been knocked out. Comparison of the level of expression of CADPKL in the mice transgenic for the mutant human CADPKL gene with mice transgenic for a wild-type human CADPKL gene will reveal whether the mutation results in increased, decreased synthesis of the corresponding protein and/or aberrant tissue distribution of the protein. Such analysis could also be performed in cultured cells, in which the human mutant CADPKL gene is introduced and, e.g., replaces the endogenous wild-type gene in the cell. Thus, depending on the effect of the mutation in a regulatory region of a CADPKL gene, a specific treatment can be administered to a subject having such a mutation. Accordingly, if the mutation results in increased CADPKL protein levels, the subject can be treated by administration of a compound which reduces CADPKL protein production, e.g., by reducing gene expression or translation or a compound which inhibits or reduces the activity of the CADPKL protein.

Furthermore, it is likely that subjects having different allelic variants of a CADPKL polymorphic region will respond differently to therapeutic drugs to treat diseases or conditions, such as those associated with neuropsychiatric disorders.

A correlation between drug responses and specific alleles of CADPKL can be shown, for example, by clinical studies wherein the response to specific drugs of subjects having different allelic variants of a polymorphic region of a CADPKL gene is compared. Such studies can also be performed using animal models, such as mice having various alleles of human CADPKL genes and in which, e.g., the endogenous CADPKL gene has been inactivated such as by a knock-out mutation. Test drugs are then administered to the mice having different human CADPKL alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

Monitoring Clinical Therapies.

The ability to target populations expected to show the highest clinical benefit, based on the neurological activity or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g., since the use of CADPKL as a marker is useful for optimizing effective dose). In situations in which the disease associated with a specific CADPKL allele is characterized by an abnormal protein expression, the treatment of an individual with a CADPKL therapeutic can be monitored by determining CADPKL characteristics, such as CADPKL protein level or activity, mRNA level, and/or transcriptional level. This measurement will indicate whether the treatment is effective or whether it should be adjusted or optimized. Thus, CADPKL can be used as a marker for the efficacy of a drug during clinical trials.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of a CADPKL protein, mRNA, or genomic DNA in the preadministration sample; (iii) obtaining one or more post-administration samples from the subject; (iv) detecting the level of expression or activity of the CADPKL protein, mRNA, or genomic DNA in the post-administration samples; (v) comparing the level of expression or activity of the CADPKL protein, mRNA, or genomic DNA in the preadministration sample with the CADPKL protein, mRNA, or genomic DNA in the post administration sample or samples; and (vi) altering the administration of the agent to the subject accordingly. For example, increased administration of the agent may be desirable to increase the expression or activity of CADPKL to higher levels than detected, i.e., to increase the effectiveness of the agent. Alternatively, decreased administration of the agent may be desirable to decrease expression or activity of CADPKL to lower levels than detected, i.e., to decrease the effectiveness of the agent.

Cells of a subject may also be obtained before and after administration of a CADPKL therapeutic to detect the level of expression of genes other than the CADPKL gene, to verify that the therapeutic does not increase or decrease the expression of genes which could be deleterious. This can be done, e.g., by using the method of transcriptional profiling. Thus, mRNA from cells exposed in vivo to a CADPKL therapeutic and mRNA from the same type of cells that were not exposed to the therapeutic could be reverse transcribed and hybridized to a chip containing DNA from numerous genes, to thereby compare the expression of genes in cells treated and not treated with a CADPKL therapeutic. If, for example a CADPKL therapeutic turns on the expression of a proto-oncogene in an individual, use of this particular therapeutic may be undesirable.

### 5.7.4. Other Uses

The identification of different alleles of CADPKL can also be useful for identifying an individual among other individuals from the same species. For example, DNA sequences can be used as a fingerprint for detection of different individuals within the same species (Thompson, J. S. and Thompson, eds., Genetics in Medicine, WB Saunders Co., Philadelphia, Pa. (1991)). This is useful, for example, in forensic studies and paternity testing, as described below.

Forensics Applications.

Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, DC, 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorpbic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation: p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies: P(ID)=x⁴+(2xy)²+(2yz)²+(2xz)²+z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus: cum p(ID)=p(ID1)p(ID2)p(ID3) . . . p(IDn).

The cumulative probability of non-identity form loci (i.e., the probability that two random individuals will be difference at I or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

Paternity Testing. The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the puntative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father) is given by the equation (see WO 95/12607): p(exc)=xy(1−xy), where x and y are the population frequencies of alleles A and B of a biallelic polymorphic site.

(At a triallelic site p(exc)=xy(1−xy)+yz(1−yz)+xz(1−xz)+3xyz(1−xyz)), where x, y, and z and the respective populations frequencies of alleles A, B, and C).

The probability of non-exclusion is: p(non-exc)=1−p(exc).

The cumulative probability of non-exclusion (representing the values obtained when n loci are is used) is thus:

Cum p(non-exc)=p(non-excl)p(non-exc2)p(non-exc3). . . p(non-excn).

The cumulative probability of the exclusion for n loci (representing the probability that a random male will be excluded: cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

Kits.

As set forth herein, the invention provides methods, e.g., diagnostic and therapeutic methods, e.g., for determining the type of allelic variant of a polymorphic region present in a CADPKL gene, such as a human CADPKL gene. In preferred embodiments, the methods use probes or primers comprising nucleotide sequences which are complementary to a polymorphic region of a CADPKL gene (e.g., SEQ ID NOS:37-42). Accordingly, the invention provides kits for performing these methods.

In a preferred embodiment, the invention provides a kit for determining whether a subject has or is at risk of developing a disease or condition associated with a specific allelic variant of a CADPKL polymorphic region. In an even more preferred embodiment, the disease or disorder is characterized by an abnormal CADPKL activity. In an even more preferred embodiment, the invention provides a kit for determining whether a subject has or is at risk of developing a neuropsychiatric disease such as, e.g., schizophrenia, schizoaffective disorder, bipolar disorder, unipolar affective disorder and adolescent conduct disorder.

A preferred kit provides reagents for determining whether a subject is likely to develop a neuropsychiatric disease such as, e.g., one of the aforementioned disorders/diseases.

Preferred kits comprise at least one probe or primer which is capable of specifically hybridizing under stringent conditions to a CADPKL sequence or polymorphic region and instructions for use. The kits preferably comprise at least one of the above described nucleic acids. Preferred kits for amplifying at least a portion of a CADPKL gene, e.g., the 5′ promoter region, comprise two primers, at least one of which is capable of hybridizing to an allelic variant sequence. Even more preferred kits comprise a pair of primers selected from the group set forth in Table 4A below (SEQ ID NOS: 8-35 and 51-76).

The kits of the invention can also comprise one or more control nucleic acids or reference nucleic acids, such as nucleic acids comprising a CADPKL intronic sequence. For example, a kit can comprise primers for amplifying a polymorphic region of a CADPKL gene and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of a CADPKL gene of an individual who does not have a neuropsychiatric disease, aberrant neurological activity, or a disease or disorder associated with an aberrant neurological activity.

Yet other kits of the invention comprise at least one reagent necessary to perform the assay. For example, the kit can comprise an enzyme. Alternatively the kit can comprise a buffer or any other necessary reagent.

The present invention is further illustrated by the following examples which should not be construed as limiting in any way. The contents of all cited references (including, without limitation, literature references, issued patents, published patent applications) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular CloningA Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### 6. EXAMPLES

The invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification, and can be made without departing from its sprit and scope. The invention is therefore to be limited only by the terms of the appended claims along with the full scope of equivalents to which the claims are entitled.

### Example 1

### Detection and Identification of CADPKL Sequence Variations Associated with Neuropsychiatric Disorders

This example describes experiments in which genetic sequences from populations, refferred to herein as the Sib pair and Kuusamo populations, were analyzed and CADPKL polymorphisms were identified. The Sib pair and Kuusamo populations are populations of individuals that contain both individuals who are phenotypic for a neuropsychiatric disorder (e.g., schizophrenia), and individuals with no neuropsychiatric disorder phenotype. The polymorphisms described here were found to co-segregate with, and are therefore associated with, neuropsychiatric disorders (for example, schizophrenia, schizoaffective disorder, bipolar affective disorder, unipolar affective disorder, adolescent conduct disorder) within these populations. The variants include novel CADPKL nucleic acid variants and novel CADPKL polypeptides that are described here for the first time, and represent novel CADPKL nucleic acids and polypeptides that can be used in methods described supra, e.g., to diagnose and treat neuropsychiatric disorder such as schizophrenia, schizoaffective disorder, bipolar affective disorder, unipolar affective disorder, adolescent conduct disorder, etc.

PCR Amplifcation.

CADPKL genomic sequences were amplified according to standard PCR protocols described supra, using oligonucleotide primers described below.

Denaturing High Performance Liquid Chromatography (CHPLC) Analysis.

CADPKL genomic sequences were analyzed for genetic variants using standard DHPLC protocols that have been previously described (see, in particualr, Spiegelman et al., Biotechniques 2000,29:1084-1092). Briefly, the technique detected mutations based on the presence of heteroduplexes from individuals who are heterozygous for CADPKL SNPs. Heteroduplex molecules occurred in PCR products that contained mismatched nucleotides from wild-type and mutant CADPKL sequences. In the absence of a mutation, wild-type homoduplexes were formed. The DHPLC analysis consisted of visualization of variation among chromatograms corresponding to heteroduplex and homoduplex samples. Specifically, the variation is dependent on differential melting characteristics of hetero-versus homoduplexes.

Identification of Microsatellite Repeats.

Microsatellite repeats within CADPKL sequences were identified by two independent methods. First, known public microsatellite sequences and their flanking amplimers were collected from mapping data in the human Genome database. These known microsatellites included the microsatellite repeats referred to here as d1s471 and d1s491. Although such microsatellite repeats may have been publicly known, they have not previously been associated with neuropsychiatric disorders such as schizophrenia.

In a second method, CADPKL nucleic acid sequences within the GenBank databases were searched to identify novel microsatellite repeats, and PCR primers were designed using the program OLIGO 4.0 to amplify the sequences flanking those microsatellites. The upstream amplimers were synthesized with a fluorescently labeled dye and the downstream amplimers were synthesized with a specific seven nucleotide repeat added to the 5′ end of the amplimer. This specific repeat promoted amplification of non-template adenylation, resulting in cleanermorphology of allele peaks. The sequence lengths of the microsatellite markers were then confirmed by polyacrylamide gele electrophoresis. Individuals from the Sib pair and Kuusamo populations were then genotyped with the microsatellite markers. In particular, genetic samples from individuals suffering from a neuropsychiatric disorder (e.g., schizophrenia) were genotyped, as well as genetic samples from control individuals who were not suffering from and did not exhibit symptoms of a neuropsychiatric disorder.

DNA Sequencing.

DNA samples were sequenced using standard nucleic acid sequencing techniques described supra.

Results. PCR amplification products of the CADPKL genomic sequence that contain exon (including intron/exon junction), 5′-UTR, 3′-UTR and regulatory (e.g., 5′-promoter) sequences of the CADPKL gene, as well as genomic sequences from regions of human chromosome 1 in the vicinity of the CADPKL gene were generated from genetic samples obtained from individuals of the Sibpair and Kuusamo populations. The genetic samples included DNA samples obtained from individuals suffering from a neuropsychiatric disorder, as well as samples from control individuals who were not suffering from and did not exhibit symptoms of a neuropsychiatric disorder.

The PCR products were analyzed for polymorphisms using DHPLC. In particular, aliquots of PCR products amplified from the genomic DNA samples of appropriate individuals were heat denatured and electrophoresed in polyacrylamide gels, and variant nucleotides were detected by mobility shifts in the gel. If a variant nucleotide was detected, the remaining PCR product from the select individual(s) was(were) sequenced to confirm and identify the polymorphism.

In more detail, Table 4A, below, lists both the forward and the reverse primer used to amplify a segment of the human CADPKL gene (or a genomic sequence in the vicinity of the human CADPKL gene) where one or more polymorphisms were identified that correlate with a neuropsychiatric disorder. Table 4B indicates the nucleic acid residues of the CADPKL genomic sequence (SEQ ID NO:1) that are amplified by each primer. Each primer pair is identified in Tables 4A-B by the name of the polymorphism identified in the amplified region. These primer sequences represent exemplary oligonucleic acid sequences which are part of the present invention. In particular, the oligonucleic acid sequences shown in Table 4A, below, may be used in the methods of the invention, e.g., to detect polymorphisms and genetic variants associated with a neuropsychiatric disorder such as schizophrenia, schizoaffective disorder, bipolar affective disorder, unipolar affective disorder, adolescent conduct disorder.

TABLE 4A

| | Forward Primer | | Reverse Primer | |
|---|---|---|---|---|
| Polymorphis | | Seq. ID | | Seq. ID |
| ID | Sequence | No. | Sequence | No. |
| cadpk15 | agaagggaagaatgggggag | 8 | gagacggatgaattggctgg | 9 |
| cadpk16 | cagtccaacaggtgagtcatcg | 10 | gggaacgagaaggggtaagc | 11 |
| cadpk17 | tgggagcttgggggagca | 12 | actttccttggcagcctgttc | 13 |
| cadpk19a* | cctgcccactccctggatga | 14 | gctgcgttgaaggcttgcta | 15 |
| cadpk19b | cctgcccactccctggatga | 14 | gctgcgttgaaggcttgcta | 15 |
| cadpk110 | cacaaggcaaagggaaagttta | 16 | ccattgaccaggcagttgag | 17 |
| cadpk110-2 | cctgacccaattaccctgcc | 18 | ccccctcatccagaactcatc | 19 |
| 272116ca2p | caaaaagtaggattgtagccctgc | 20 | gtttcttctaccatccccactttcagaacc | 21 |
| 272116tc1p | cctctctgtgaaatggcattgac | 22 | gtttcttaatgcctggtcaaataccgtagg | 23 |
| 272116ca4p | agccaaaactgacaccaggaag | 24 | gtttcttggaaatggcttggtcttggtc | 25 |
| d1s471 | gatgggcactgtgttactgg | 26 | gtttcttgctttgatggaaatagtattatgc | 27 |
| 272116tc2 | tgaaataaatgtgctctgggctc | 28 | gtttcttccagcctgcctccactcag | 29 |
| d1s491 | cacaggacggtcgatggttc | 30 | gtttcttgctgtcagcaagaantgtgaaagt | 31 |
| 272116aattg7p | caaagatgctctccttccctgtc | 32 | gtttcttcagccatttagggacctgcc | 33 |
| 272116ca6p | ttacccctttctcgttccctcc | 34 | gtttcttagatgtaggaacagagggtccacc | 35 |
| cadpk0 | tgtcatcacccattcaggataatg | 51 | ttaagcaaggagaccctctaaagc | 52 |
| cadpk112d | ctgggtctgtgctgtttgtcg | 53 | gaagtgtgctggctgggtctc | 53 |
| cadpk112e | tcgttgcctgcccatctg | 55 | ggtgtgctggactttcaaggag | 56 |
| cadpk1mp | ggagtcatctggagagtttatgcc | 57 | tgttcactttcttgagtgtgacaatg | 58 |
| cadu2 | tcccagttctgtaagagataacaagc | 59 | cggctctgctcaccctcc | 60 |
| cadu3a | agagggagcacagagtttccg | 61 | cagaacatctacactggctgacatg | 62 |
| cadu3b | agagggagcacagagtttccg | 61 | cagaacatctacactggctgacatg | 62 |
| cadu3c | agagggagcacagagtttccg | 61 | cagaacatctacactggctgacatg | 62 |
| cad11a | ggcttggttatctttatcttttctgc | 63 | catcactcacactctggcatgg | 64 |
| cadpk8 | agtggactctagaccccagcc | 65 | tcaggacaagcagattccagg | 66 |
| cadpkd1 | cagtacccatcggcaccttg | 67 | cacagtgctgggcaaatagt | 68 |
| cadpkd2 | ctatttgcccagcactgtgc | 69 | acctctctcccacctgttatgg | 70 |
| cadpki | ctctgttcctttggatattccactc | 71 | aaatggtgtctcactcatcactcc | 72 |
| cadpkj | gaggaacaaactttctttttgttcaa | 73 | ataaccttccttccccactcg | 74 |
| cadpkk | tcaggttggcctccaaaacta | 75 | ccttccttccccactcgag | 76 |

*The same primer pairs were used to amplify the genomic region containing the SNPs cadpk19a and cadpk19b, and the genomic regions containing SNPs cadu3a, cadu3b, and cadu3c, respectively.

TABLE 4B

| | Amplified | | Amplified |
|---|---|---|---|
| | Nucleic Acids | Polymorphism | Nucleic Acids |
| Polymorphism ID | (SEQ ID NO:1) | ID | (SEQ ID NO:1) |
| cadpk15 | 140637-141065 | cadpk112d | 147798-148088 |
| cadpk16 | 142060-142460 | cadpk112e | 147961-148309 |
| cadpk17 | 143358-143687 | cadpklmp | 130958-131300 |
| cadpk19a | 145857-146267 | cadu2 | 117649-118038 |
| cadpk19b | 145857-146267 | cadu3a | 117473-117725 |
| cadpk110 | 146172-146519 | cadu3b | 117473-117725 |
| 272116ca2p | 22701-27854 | cadu3c | 117473-117725 |
| 272116tc1p | 48936-49313 | cad11a | 147085-147454 |
| 272116ca4p | 68586-68774 | cadpk8 | 144410-144494 |
| d1s471 | 78230-78548 | cadpkd1 | 128772-128918 |
| 262116tc2 | 98970-99216 | cadpkd2 | 128900-128989 |
| d1s491 | 104192-104499 | cadpki | 127872-127964 |
| 272116aattg7p | 122683-123008 | cadpkj | 127696-127790 |
| 272116ca6p | 142443-142783 | cadpkk | 127679-127786 |
| cadpk0 | 117819-118281 | — | — |

The polymorphisms and other nucleic acid variants which were found to correlate with neuropsychiatric disorders include both single nucleotide polymorphisms (SNPs) and microsatellite repeats. Table 5, below, summarizes SNPs identified in the CADPKL genomic sequence (SEQ ID NO:1). In particular, column 3 (under the title “Residue No.”) in Table 5 specifies the nucleotide residue in the CADPKL genomic sequence set forth in SEQ ID NO: 1 where each SNP is located. Column 4 (under the title “Mutation”) in Table 5 specifies the identity of the SNP. For example, the first SNP recited in Table 5 (i.e., cadpkl5) is located at nucleic acid residue number 140766 of SEQ ID NO:1. This nucleotide is a thymine (T) in the wild-type (WT) sequence. However, in those nucleic acids having this particular SNP, the nucleotide is a guanine (G). This polymorphism is indicated in Table 5, below, by the entry “C/T” in column 4.

TABLE 5
SNPs IN CADPKIL
GENOMIC SEQUENCE (SEQ ID NO:1)

| | | Mutation | |
|---|---|---|---|
| Polymorphism ID | Residue No. | (WT/SNP) | P-Value |
| cadpk15 | 140766 | T/G | >0.05 |
| cadpk16 | 142239 | T/G | >0.05 |
| cadpk17 | 143457 | A/G | 0.0213 |
| cadpk19a | 146041 | G/T | — |
| cadpk19b | 146125 | G/C | — |
| cadpk110 | 146320 | G/A | — |
| cadpk0 | 117978 | A/G | — |
| cadpk112d | 147997 | C/T | — |
| cadpk112e | 148151 | A/T | — |
| cadu2 | 117926 | T/C | — |
| cadu3a | 117533 | C/A | — |
| cadu3b | 117584 | A/G | — |
| cadu3c | 117642 | C/T | — |
| cad11a | 147192 | G/A | — |
| cadpk8 | 144444 | G/A | — |
| cadpkd1 | 128813 | A/G | — |
| cadpkd2 | 128947 | C/T | — |
| cadpki | 127923 | G/A | — |
| cadpkj | 127747 | C/T | — |
| cadpkk | 127700 | A/T | — |

Many of the SNPs identified in Table 5, above, are found in exons of the CADPKL genomic sequence (see, in particular, Table 1, infra). Thus, these SNPs may also generate an altered, transcribed gene product (e.g., an altered mRNA or an altered cDNA derived therefrom). These altered CADPKL cDNA sequences are specified in Table 6A, below, with respect to the CADPKL protein coding sequence set forth in SEQ ID NO:2, and also with respect to the CADPKL cDNA sequence set forth in SEQ ID NO:4.

TABLE 6A
SNPs IN CADPKL CODING SEQUENCES

| | | | Mutation | |
|---|---|---|---|---|
| Polymorphism ID | SEQ ID NO. | Residue No. | (WT/SNP) | P-Value |
| cadpk17 | 2 | 654 | A/G | 0.0213 |
| cadpk17 | 4 | 671 | A/G | 0.0213 |
| cadpk110 | 2 | 985 | G/A | — |
| cadpk110 | 4 | 1002 | G/A | — |

Certain SNPs identified in Table 6A, above (i.e., cadpkl7) are silent mutations and merely change the located at the site of the altered base into one that encodes the same amino acid residue as the wild type sequence. Accordingly, the SNPs do not alter the amino acid sequence of the protein encoded by the nucleic acid molecule. However, other SNPs identified in Table 6A (in particular, cadpkl10 and capkl10_{—}2) change the codon where the SNP is located into a codon for a different amino acid residue. Thus, nucleic acid molecules which comprise these SNPs encode an altered CADPKL gene product. Specifically, the CADPKL polypeptides encoded by these SNPs comprise amino acid residue substitutions. The specific amino acid residue substitutions encoded by each of these SNPs are indicated in Table 6B, below.

TABLE 6B
AMINO ACID SUBSTITUTIONS
ENCODED BY CADPKL SNPs

| | | | Mutation |
|---|---|---|---|
| Polymorphism ID | SEQ ID NO. | Residue No. | (WT/SNP) |
| cadpk110 | 3 | 329 | Val/Ile |
| cadpk110 | 5 | 329 | Val/Ile |

Thus, for example, a CADPKL nucleic acid containing the SNP cadpkl10 or cadpkl10_{—}2 may encode an altered or variant CADPKL polypeptide. For example, a genomic coding sequence (such as SEQ ID NO:2) may encode a variant of the polypeptide set forth in SEQ ID NO:3 in which the amino acid residue at position 329 of this sequence is isoleucine (Ile or I) rather than valine (Val or V). Similarly, a CADPKL cDNA sequence (for example, SEQ ID NO:4) may encode a variant of the polypeptide set forth in SEQ ID NO:5 in which the amino acid residue at position 329 of this sequence is Ile rather than Val.

In addition to the above-described SNPS, other polymorphic markers were also identified which evidence allelic association with a neuropsychiatric disorder such as schizophrenia. A “microsatellite” or “microsatellite repeat”, as the term is used herein, refers to a short sequence of repeating nucleotides within a nucleic acid. Typically, a microsatellite repeat comprises a repeating sequence of two (i.e., a dinucleotide repeat), three (i.e., a trinucleotide repeat), four (i.e., a tetranucleotide repeat) or five (i.e., a pentanucleotide repeat) nucleotides. Thus, for example, a dinucleotide repeat of guanine and thymine may be indicated by (GT)ₙ, which denotes a dinucleotide sequence of guanine and thymine that repeat n times within a nucleic acid. Microsatellite repeats frequently vary in length on different alleles of a gene or on different alleles of a genomic sequence. Accordingly, polymorphisms of a microsatellite may be readily identified by using PCR primers to unique sequence upstream and downstream of a microsatellite (for example, the PCR primers identified in Table 4, above) to amplify a region containing a microsatellite, and determining the length (e.g., by mobility on an agarose or other gel) of the amplified nucleic acid.

Table 7, below, identifies several microsatellite repeats in the CADPKL genomic sequence set forth in SEQ ID NO:1. Specifically, Table 7 indicates, for each microsatellite repeat, the location (i.e., the nucleotide residue number in SEQ ID NO:1) of each microsatellite, along with the repeat motif (e.g., (GT)ₙ) and the number of repeats n in wild-type and mutant CADPKL sequences. It is understood that the number of repeats specified for each microsatellite in Table 7 may be, in preferred embodiments, approximate.

Polymorphisms in the length of these repeats may show an allelic association with a neuropsychiatric disorder such as schizophrenia. Regions of the CADPKL genomic sequence containing these microsatellite repeats may be amplified, e.g., using the PCR primers identified in Table 4, above, for each polymorphism.

TABLE 7
MICROSATELLITE REPEATS IN THE
CADPKL GENOMIC SEQUENCE (SEQ ID NO:1)

| | | | Repeat (n) | | P- |
|---|---|---|---|---|---|
| Polymorphism ID | Residue No. | Motif | Wild-type | Mutant | Value |
| 272L16CA2P | 27701 | (CA)ₙ | 21 | 15-27 | 0.0002 |
| 272L16TC1P | 48936 | (CT)ₙ | 13 | 12-25 | 0.0312 |
| 272L16CA4P | 68586 | (GT)ₙ | 15 | 15-17 | >0.05 |
| D1S471 | 78230 | (GT)ₙ₁ (AG)ₙ₂ | n1 = 21 | n1 = 22-31 | >0.05 |
| | | | n2 = 6 | n2 = 22-31 | |
| 272L16TC2P | 98970 | (CT)ₙ | 16 | 16-32 | 0.0044 |
| D1S491 | 104192 | (CA)ₙ | 15 | 10-18 | >0.05 |
| 272L16AATTG7P | 122683 | (ATTGG)ₙ | 30 | 27-32 | 0.0074 |
| 272L16CA6P | 142443 | (CA)ₙ | 12 | 9-12 | 0.0201 |
| cadpklmp | 130958 | (CA)ₙ | 20 | 17-26 | — |

### Example 2

### Expression of CADPKL in Human Tissues

Materials and Methods.

Expression assays were carried out via real-time PCR with FRET detection, commonly referred to as the TaqMan assay, according to methods already known in the art (see, in particular, Livak et al., PCR Methods and Applications 1995, 4:357-362). The assays were performed using an ABI 7700 Sequence Detection instrument, with the following oligonucleotide reagents:

| | |
|---|---|
| Forward Primer | |
| TGGAGAATGAGATTGCTGTGTTG | (SEQ ID NO:43) |
| Reverse Primer | |
| CATCTATGAGAGCACCACCCACT | (SEQ ID NO:44) |
| Probe | |
| TCAAGCATGAAAACATTGTGACCCTGG | (SEQ ID NO:45) |

Independent control experiments demonstrated that the assay was specific for CAPDKL mRNA and did not detect CADPKL genomic DNA sequences.

Results.

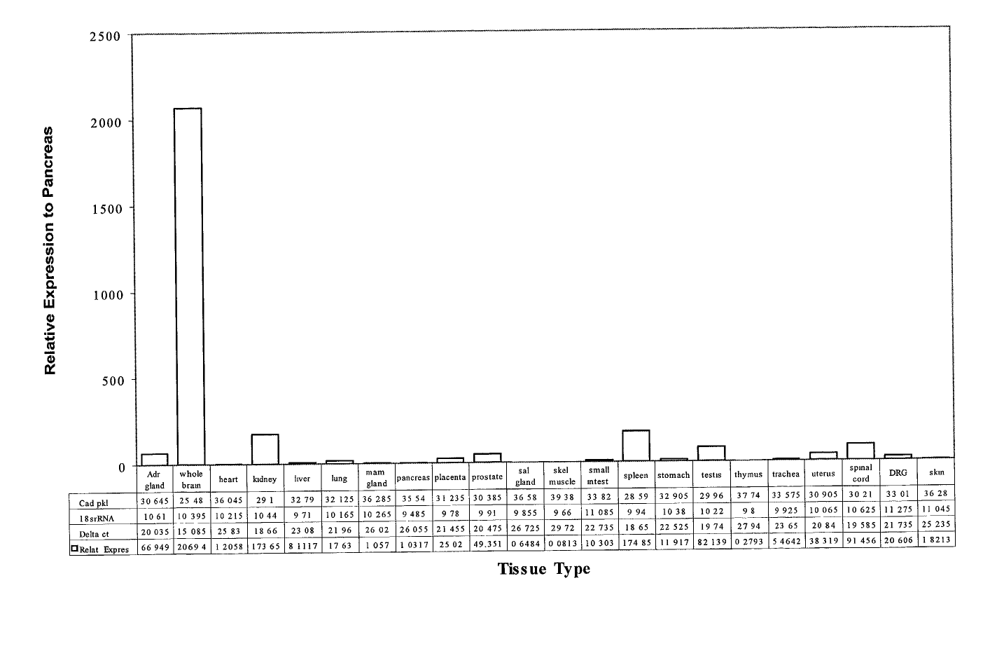

Two different expression profiling experiments were conducted to identify tissues where the CADPKL gene is normally expressed. First, a broad spectrum of tissues derived from a single individual of no specific phenotype (i.e., who was not known or believed to be suffering from or susceptible to any neuropsychiatric disorder) was analyzed for CADPKL mRNA content using the TaqMan assay described above. The CADPKL expression levels measured for these different tissues are indicated in FIG. 3, which shows that the CADPKL gene is predominantly expressed in the brain.

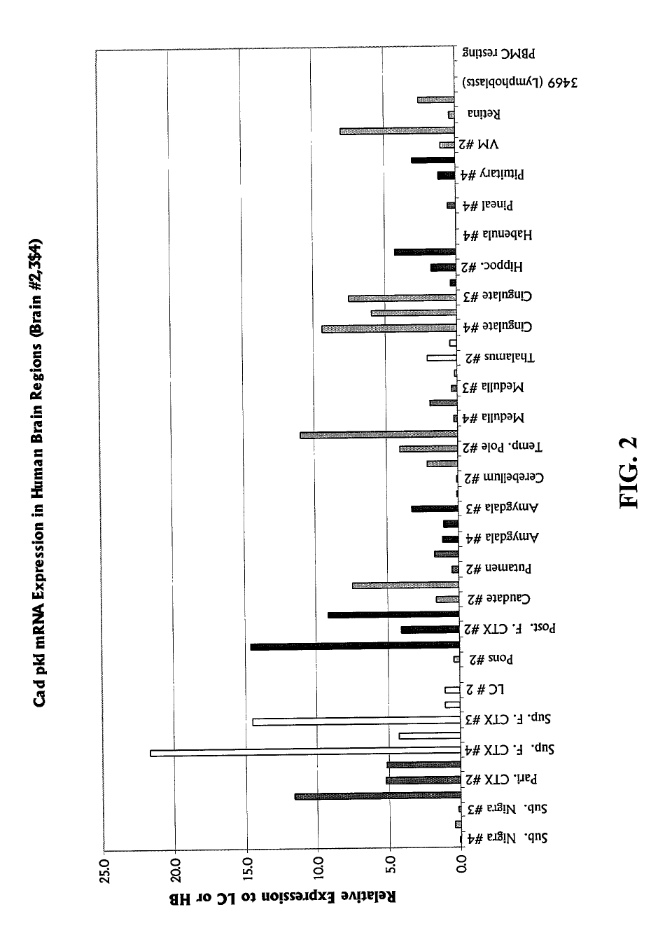

In a second experiment, various brain tissues were dissected from three different human cadavers (referred to herein as Brains 1-3), also of no specific phenotype. These tissues were also examined for levels of CADPKL mRNA expression using the TaqMan assay, and the results are shown in FIG. 2 for each of Brains 1-3, respectively. These results show that within the brain the CADPKL gene is expressed primarily in the cerebral cortex and in tissues of the limbic system (in particular, the hippocampus and the cingulate gyrus). Thus, the CADPKL gene is normally expressed in areas of the brain that are believed to be associated with neuropsychiatric disorders such as schizophrenia, etc.

What is claimed is:

1. An isolated nucleic acid which comprises a nucleotide sequence of a polymorphic region of a CADPKL allelic variant, wherein the CADPKL allelic variant has a nucleotide sequence that differs from a reference nucleotide sequence selected from the group consisting of SEQ ID NO:1, SEQ ID NO:2, SEQ ID NO:4 and complementary sequences thereof.

2. The isolated nucleic acid of claim 1 wherein the polymorphic region is located in a 5′ promoter region.

3. The isolated nucleic acid of claim 1 wherein the polymorphic region is located in an intron.

4. The isolated nucleic acid of claim 1 wherein the polymorplic region is located in an exon.

5. The isolated nucleic acid of claim 1 which comprises a nucleotide sequence selected from the group consisting of SEQ ID NOS:37-42, SEQ ID NOS:77-90, and complementary sequences thereof.

6. A kit for amplifying or determining the molecular structure of at least a portion of a CADPKL nucleic acid, which kit comprises:
a probe or primer capable of hybridizing to a polymorphic region of a CADPKL nucleic acid; and
instructions for use.

7. The kit of claim 6, wherein the CADPKL nucleic acid is from a human CADPKL gene.

8. The kit of claim 7 wherein the probe or primer is capable of hybridizing to a polymorphic region of a CADPKL allelic variant, which CADPKL allelic variant has a nucleotide sequence that differs from a reference nucleotide sequence selected from the group consisting of SEQ ID NO:1, SEQ ID NO:2, SEQ ID NO:4 and complementary sequences thereof.

9. The kit of claim 8 wherein the polymorphic region is located in a 5′ promoter region.

10. The kit of claim 8 wherein the polymorphic region is located in a 3′ untranslated region.

11. The kit of claim 8 wherein the polymorphic region is Located in an intron.

12. The kit of claim 8 wherein the polymoprhic region is located in an exon.

13. The kit of claim 8 wherein the polymorphic region comprises a nucleotide sequence selected from the group consisting of SEQ ID NOS:37-42, SEQ ID NOS;77-90, and complementary sequences thereof.

14. The kit of claim 8 wherein the probe or primer is a single stranded nucleic acid.

15. The kit of claim 8 wherein the probe or primer is labeled.

16. The kit of claim 8 wherein the probe or primer has a nucleotide sequence from about 15 to about 30 nucleotides in length.

17. The kit of claim 16 wherein the probe or primer comprises a nucleotide sequence selected from the group consisting of SEQ ID NOS:8-35, SEQ ID NOS:37-42, SEQ ID NOS:51-90, and complementary sequences thereof.

18. A kit according to claim 16 which comprises a first primer and a second primer, wherein the first and second primers are selected from the group consisting of SEQ ID NOS:8-35, SEQ ID NOS:37-42, SEQ ID NOS:51-90, and complementary sequences thereof.

19. A kit for determining whether a subject is at risk of developing a neuropsychiatric disorder, which kit comprises:
a probe or primer that is capable of hybridizing to a polymorphic region of a CADPKL nucleic acid; and
instructions for use.

20. The kit of claim 19, wherein the neuropsychiatric disorder is schizophrenia, schizoaffective disorder, bipolar disorder, unipolar affective disorder and adolescent conduct disorder.

21. A method for detecting a CADPKL allelic variant, which method comprises contacting a sample CADPKL nucleic acid with a probe or primer complementary to a polymorphic region of a CADPKL allelic variant so that the CADPKL allelic variant is detected in the sample CADPKL nucleic acid.

22. The method of claim 21 wherein the nucleotide sequence of the CADPKL allelic variant differs from a reference nucleotide sequence selected from the group consisting of SEQ ID NO:1, SEQ ID NO:2 and SEQ ID NO:4.

23. The method of claim 21 further comprising determining the identity of the CADPKL allelic variant.

24. A method according to claim 23 which comprises determining the identity of at least one nucleotide of the sample CADPKL nucleic acid.

25. The method of claim 24 wherein the sequence of the polymorphic region of the sample CADPKL nucleic acid is determined.

26. The method of claim 23 wherein the identity of the CADPKL allelic variant is determined by restriction enzyme analysis.

27. The method of claim 23 wherein the identity of the CADPKL allelic variant is determined by single-stranded conformational polymorphism.

28. The method of claim 23 wherein the identity of the CADPKL allelic variant is determined by allelic specific hybridization.

29. The method of claim 21 wherein the identity of the CADPKL allelic variant is determined by primer specific extension.

30. The method of claim 21 wherein the identity of the CADPKL allelic variant is determined by an oligonucleotide ligation assay.

31. The method of claim 21 wherein the CADPKL allelic variant is an allelic variant of a human CADPKL gene.

32. The method of claim 21 wherein the nucleotide sequence of the probe or primer is from about 15 to about 30 nucleotides in length.

33. The method of claim 32 wherein the probe or primer comprises a nucleotide sequence selected from the group consisting of SEQ ID NOS:8-35, SEQ ID NOS:37-42, SEQ ID NOS:51-90, and complementary sequences thereof.

34. A method according to claim 21 which further comprises contacting the sample CADPKL nucleic acid with a second probe or primer, wherein each probe or primer has a nucleotide sequence selected from the group consisting of SEQ ID NOS:8-35, SEQ ID NOS:37-42, SEQ ID NOS:51-90, and complementary sequences thereof.

35. The method of claim 34 which comprises hybridizing the two probes or primers to the sample CADPKL nucleic acid.

36. The method of claim 21 wherein the probe or primer is a single stranded nucleic acid.

37. The method of claim 21 wherein the probe or primer is labeled.

38. A method for determining whether a subject has or is at risk of developing a disease or disorder associated with a specific CADPKL allelic variant, which method comprises identifying, according to the method of claim 21, the CADPKL allelic variant in a nucleic acid sample from the subject.

39. The method of claim 38 wherein the disease or disorder is a neuropsychiatric disorder.

40. The method of claim 39 wherein the neuropsychiatric disorder is selected from the group consisting of schizophrenia, schizoaffective disorder, bipolar disorder, unipolar affective disorder and adolescent conduct disorder.

41. The method of claim 40 wherein the neuropsychiatric disorder is schizophrenia.

42. A method for selecting an appropriate drug for administration to an individual, which method comprises determining the molecular structure of at least a portion of the CADPKL gene of the individual.

43. The method of claim 42 wherein the molecular structure is determined according to a method that comprises determining the identity of an allelic variant of at least one polymorphic region of the CADPKL gene of the individual.

44. A method for treating a subject having a disease or disorder associated with a specific allelic variant of a polymorphic region of a CADPKL gene, which method comprises:
(a) determining the identity of the allelic variant; and
(b) administering, to the subject, a compound that compensates for the effect of the specific allelic variant.

45. The method of claim 44 wherein the compound is a CADPKL protein activity inhibitor.

46. The method of claim 44 wherein the polymorphic region is located in an exon.

47. The method of claim 44 wherein the polymorphic region is located in an intron.

48. The method of claim 44 wherein the specific allelic variant is a mutant allele.

49. The method of claim 44 wherein the polymorphic region is located in a promoter region.

50. The method of claim 44 wherein the sequence of the specific allelic variant comprises a nucleotide sequence selected from the group consisting of SEQ ID NOS:8-35, SEQ ID NOS:37-42, SEQ ID NOS:51-90, and complementary sequences thereof.

51. The method of claim 44 wherein the specific allelic variant is associated with a neuropsychiatric disorder.

52. The method of claim 51 wherein the neuropsychiatric disorder is selected from the group consisting of schizophrenia, schizoaffective disorder, bipolar disorder, unipolar affective disorder and adolescent conduct disorder.

53. The method of claim 44 wherein the compound modulates CADPKL protein activity levels.

54. An isolated CADPKL nucleic acid which comprises a microsatellite repeat, and which is amplifiable from a genomic DNA using PCR and any primer pair disclosed in Table 4A.

55. The isolated CADPKL nucleic acid of claim 54, wherein the microsatellite repeat comprises a motif selected from the group consisting of CA, CT, GT, AG, ATTGG, and all complements and permutations of said motif.

56. The isolated CADPKL nucleic acid of claim 54, wherein the microsatellite repeat comprises a repeat motif selected from the group consisting of (GT)ₙ, (GT)ₙₗ (AG)ₙ₂ (CT)ₙ, (CA)ₙ, (ATTGG)ₙ, and all complements of said repeat motif.

57. A method for detecting a CADPKL allelic variant, which method comprises contacting a sample CADPKL nucleic acid with a probe or primer complementary to a microsatellite repeat of a CADPKL allelic variant so that the CADPKL allelic variant is detected in the sample CADPKL nucleic acid.

58. A method for determining whether a subject has or is at risk of developing a disease or disorder associated with a specific CADPKL allelic variant. which method comprises identifying, according to the method of claim 57, the CADPKL allelic variant in a nucleic acid sample from the subject.

59. The method of claim 58 wherein the disease or disorder is a neuropsychiatric disorder.

60. The method of claim 59 wherein the neuropsychiatric disorder is selected from the group consisting of schizophrenia, schizoaffective disorder, bipolar disorder, unipolar affective disorder and adolescent conduct disorder.

61. The method of claim 60 wherein the neuropsychiatric disorder is schizophrenia.

62. A kit for amplifying or determining the molecular structure of at least a portion of a CADPKL nucleic acid, which kit comprises:
a probe or primer capable of hybridizing to the isolated nucleic acid of claim 54; and
instructions for use.


SEQUENCE LISTING (90 sequences)

SEQ ID NO: 1 - DNA, 157875 bp, Homo sapiens
gatcatccaagcctgtgtccttccctctagagtgagctcagttcactcagaatcctgaaattccctgcacatatgactccaggcctgcttccgtggcacaaatcattatctttcctggttcttcaccctaagagtagactgtgctattgacattttcaccctaggcctaggggtggccaaagcttggctgcttacaggaggtgtggatgaatctcacacgtgcaaacccaaaactctcacaactgtctacacatcctctcctattatgggatggagttgcaatgcaaacaagggagtgtggacaatgggccagggtccaggagtcagtacttcacatgctacaacatttcaccatgtaactctggggagtctaaacactttaaatttgaatctgagcttccaggtcattatgaaggtatatttcttaaatcagaaggcaaaaatgcaagtttctttagcttgatttatgagttttaaatatttagacatttagcattaggatctccattcatattctcaccccagagcatgtcaatcttaggaaagagacaaaaggcagggagaccagtggaagagctcttgcaggcactgagatgacaaaaggaaagccagattagtggttaggaactttcaaaggagaaacagaatggagacatttcaggagtagaagtgataagttcatctcctagtgggagaatgggtttgggaatggcctcagtgatgtctcagagtcctctacctggatgtgtggattagaaagaaacagtgggaaaagacacaggttttgggagaggaatatatactttggttctacacatgctgtgtttgtggggccagcaggctatccacaaggtgaggactgtggcttgagatatagatttggaaaatttaagacatgaagttaatgtaatcagggtggaagaagaaggctgagcagcaattcagaggaagagccgcaaatatgttgtaccagtcaggataagctgaagtgacaaccccaaaatctcaggaactaacaacattaaaggtctgtttctcactcatacacgtgcactgtgggtcagctagggctctgcccttgtgtcttagtccattttgtgtttctgtaacagaatacctgagagtgggtaatttataaagaaaagaggcttatctagctcacagttctacaggctgggaagttcatggtaatggcactggcttctggcaagaatttttcctgctgcaacgtaacatgctgaaaggtcaaaggggtaagagagaggtacgaaggggccaaatgctaagggcatcctgggtttataacaacttacattctcaggagttaatatattcctgagagaggaagaacacacccctgtgagatggtatgaatctcttcatgaaggatccacttcatggcccaaatacctcccactgggccccatgatgtcccaatgcatcagcactggcaattaaactttaacatgagttttggtggagacaaacttcacccaaactacagcaccatgctttctctccaggatccgggccagtggaagagcatggctggtggtgggtaactgaggaaaaaggtagcctggttgctatgacctgaatgtgtgtgtcccccaccaaattcatgtgttgaaatcccaagtcccagtggaatggtattagaagatgggggactttggtgagtgctccctcatgaatgggattagtgtccttataaaaggagccccagagagctgccccaccccttctactgtgtgaagatgcagcaagaagttactaagaggaataagccctcaccagacaccaaatctgccagcacctggatctcggacttcctaacctccagaaatgtgaggaataacagttgttcataaggtattttcattatagtgccctaaacaaagagagtggcaatccacacattggctcttacagccccttcttggaaatggcataggaccctgttaggcaaagcaagttacatggtaaaaactgccatcagtggagcagggaggtacaaccttttccagggagaggcagtggttatttttgaatattctagaaaactaagtcatctcatctttctccccatcctttccctgttccataactgaagctatctaagatgggagtggggttcagggctacttttaaaataactatagtagttaaccagattattttggagggaagagaaggattaaggtgccaatgttccaaatgaggacaaatgtacatgcaaatgtataaagatgaaaagagcattcatccagtcagtaggcacttatttatcatctacgaagtactgagtattggaaaaaaatctttctacaaacccaataaatgaatgtagggaaggaaaaaaaatccccttaccctcttatttttagtggctggtcttgcaaaataaactgacaaaggcaatttaacaggagaaaagcatacatgcttatttaaaattaaaagttttatatggcacaagagagcttcataaagaagagaaaagcctccaaagaagcagttaggcttggaggcttttatgccattcagacaatgaatgatcaatttgtagagaagtgataagacaaaggaaaggcgtttctgggctttttagggatggtaaactgtgggaaggtaaatacatgggtgacactcaatgatagataacggttactttgcgaggtttgtttttgtaaattattttcagtgccatctcatctctggtgataatgttgtcttcccctaactggtacaaaaagtagggtgggtggaataccttcacaagaagaatttatgttccactttcaggaagatagggagagagcagaaaactcatcctgtgtctgctttgtctcaattgcctctagctcaaaataattcatatgccagggtggcatatttgggagcagtatattctggtctgctacatgagctaatacacccaatacaactgatgacttatagtcataagagaaaatacaagttttatccaaattgctttatataaattatacaaaagtttttatataattgctttatatatatgaaccacacctcctcttttcctcagtgtgatagttgttactcctgtctatcttagtctgtttggatggctgtaacaaaatagcctagactaggtgtcttacaaacaacaaaaatgtctcacagttctgggggctgggaagtccagataaaggcactggcagatctggtgtcttgtgaaggcccattttctggttcatatgtagcatcttcttactgtatcctcacatgttggatgcagtgaggggtctctctcaggtctctttaataaaatctcattcatgagggctcttacccctgtcctattcatctccaaggctccacctcttaatactatcatcttgggggttaggatttcaacatataaatttgggaggagaggctgttttgaatgagctactttgagcttctacattaacttagctgttgtttccagcagtcaccagagatgccaataacaccaggagaagatccacctgcatgaatcttctcaaggcaggcctcaccatcaggagtctgatcgcaggtgcaactgcaatgacgcaaaaacaatgtgaaggtttaagactttttagtacttacagaccctgggggtgtcaatggcatgcctggaggccacataccagggaatgcaggcagagggagagagaagggacccatgggccagtgtttttattgggtacagggtgttatccaaacaggttgcccaaggggagttgtaatgggtcagttgaacacaagtgagctctagttccaggaggccacactgtgactgagaggtgatcacattaagttcttgggcaaattgtttaaatggtcagtttaaaggaaatggcaggaaaatgttgagcccagcctgttaggcaagaaagatgcctctaagtattatctctggccaccagctggggccatttgggtgggatatagtactaggaactgtgtcaaggatgactgaaccctgcttctaatataagaaagttaaacgtattttaaaatagatgctgaggcaacataaaattataaacacttactacaaacattctgacattctgatttaccccccaaattcatttccttcttgcatgcaaaatacatttcttccatccgaatagcccccaaagtcttaactcattccagcatcaacgtctgaattccaaagtctcatctaaatgtcatctcaatatgatatttagatgagatgagatatgggtgagacttaaggtatgattcattctgagccaattccccatccccctccagctataagcctgtgaaatcaaactagttttgtacctacaatatataattgtggaatgggcataggacagtcattcccattatacaagggagaaataggaaagtagaaagaagtgacatatctcaagcaagtccaaaatccaacaaggccaagaacattcagtcttacagccctaaaacaatctttgttgagttgctggtctgccctctaaaaccactagagtggaggtatgccttctggacctactggggtggctgtctttcccctttggatttgagcagccctgctcccatggctttgcggcatgcaacccacactgcacccctcaagggttggaataatgtgcctgcagctttcctaggccagaatgaagtgctgatgtctttaccagcctggggtcatgattcaccttaatgaatttgctgggcattgccctagttctttgcagtagccctacctccacagctgctctttgcctgggtccttcagctttgggggcatcttttggagtctgggtggaggcagtcaagcccctacacctttgcagggtgcagcacaaaccataccaggccccctggagccacacttggggcagttgaggagcatagaaatggagtgtgaagagcagagcccatgatgaggcagcactaagcaggaggtgtgctctgctttcctttaaaattgttctgctctgcaggcgcctgcactctgggactgtgatgggagggacagccctgatgatctccaagatacctttagagtcgttcttctattgtcttagacaataagtcctggcctctatttagacgggtgattcatactaattttctatccattttggccacatctttcattttctttctcaaacaggctttctcattttttccaatatagataggctgtgaattttttagatttttaagttctgctgtcattttgattagtctttaaatcattccttttttccccctcacgtttactataagcaatcaagagaaactaagccataccttcaagactgcttaaaaatagcttcagctaagtgagaccctcgtctctacaaaaaaaaattagccacatgtggtggcacacacctgtaatcctagctcctcagaaggctgaggcaggaggacaacttgagcccaggcgtggaggctgcagtgagctatcataccaaagcactccagcctgggcaacagagtaagatcctaccccaaaaataaaaattaaaaaaatagcttcagctaaatatccaatttcattctttacaaattctaccttccacaaaacactaggagaggaacaaaattcagccaagttgttctttgccagtgtatataataggtcacctttccttcagtttctaataacatgttcctcatttccatctgagacctcatccaaatggcctttactgtccatatttctattagcattctgttcataacctataagctatcctgtaagaatgctgaggctttctttatagctctcctcctctcctgagccctcaccagaatcaccctcactagccaattcacagcaactgtaggcttttctaaatcatgcacttcaaaactattccaggctctattcacttacctagttccaaacctgcttcgacattttaggtatttgttactatagcacccctacttctcaataccaattttctgtcttagtctttcaagctgctacaacaaaataccaccaactgggtggtgttacacgtgtctgtgtgaagagaccaccaaaaaggctttgtgtgagcaataaagctttttaatcacctgggtgcaggcggactgagtctgaaaaaggagtctgcaaagggagataggggtggggcagttttataggatttgggtaggcagtggaaaattacagttaaaagtggttctttcttgtgggcaggggcgggggtcactaggtgcagggtgggaagatcatgagacgcattgtccaggggaggaatgtcacaaggtcgaattgattagttagggtgggacaggaacaaatcacaatggtgaaatgtcaccagttaaggcaggaactggctgtttcacttcttttgtggttcttcagttgctccaggccatctggatgtatacatgcaggtcacagggtttatgatggcttagcttggtctcagaggcctgacaggtggcttataaacagtggaaatatatttctcacagttctggaggctgagaactccaaggtcaaggcagatttggtgtctggtgagaacccattttccggaaatagattatagatggtgccctctcactatgctcacatggtagagggaataaggggtctctctcaagccttttttataaaggtattaatcccattcatgaggtctccaacctcatgacctaatcacttctcaaagatctagcttcctaataccattgccttaggggttaggttttcaacctacaaatgggattgggggactcaaactttcagaccatatcactgtcattttacagaagaggagtgtgaagttgaaggatttaagaaacttcccaaggttatatcactgaccagggccagagcctggattccatccatttctaattctgaccccctattgaaccatctatccttagcattcagagaactgcccaaagtatggaagaaataagcatggagggtatagaacaaaggctggaaaagagggagctagtaaattggatacactaattcactttagattataaagtcccctttccagtggactcctgaagacaatgatgttttaactttcaaatggtagatgaccaagcattgaggagagcagctggactgacattctgaggtgagtaaaaggtgggagacagtggggagttgtcttggtggagacatcatatcagcctttgaggtcccaccaagtaaccgtcttctgtccttgtctatcctaatcccctaagcagtttcagaccctaagtcatgaaaaactttgcaacaccttaaaataccacattctgctttctcttacatgtgttgcaccattatctccctttggagattttaaacttcctgatggctgggacagtatcttacatatctttgttgctcaatggtttgcacttggtaccatctccgtgtgtctgcctgagttgagaatttttcctcaaataagtccccctacaggcaaggagagttgtccttgattagaaaaaactttaatggctatattttgaagaagggttaaggggacttctcctgggaggagtgtaattggaaagatggaaagattatttccagaacattagggattgcagtatttgaagaatcaaagactgtctgctctgtcaactcaagtatgctcctttgcatttaaaagcaatgccatttacttacaaattatagtggctttgatgcaaatgaaccactccaggacagtttcctctcccccagattagctaagaaatgctggggaaattgaccaaggatgcagaatgctaagcctgacctaaggcctattaaaatcaagttcctggtttaattatttatattgctgaaataagaggtaattagaatccagtaaagtggcaagaggaggccagcccaggattttgcttttatttgtatggattctgggtctggatatagcagacagagcatacttggcccagataatgtggagaaattgctgagagcagaggcagcgtcctaggccagacccaacccaaactcataggagaattgcagtaaattactgaacccctttggccctcagtttctctgtgcaatagggagatccattcctgccctgtttatgagagaaccagtgagggaatattaacatagtggctagtattcatcaatttacaatttataaagtgtttttgcatccattatctcatttgatctctacaagaccctgtgaagtaggaatttttatcatctgcattttcagcaggaaaactgggtctcttggaagttaaattgcccaagtcacacaatcaattagtaatggtggcatgctgcttctaactccaaatcccattaggctcttcttctcccctcatagatagaataatgatgagaagataagacatgacttgtaatgttacagtaggtagttaggcagacatgagcagggcaggagagggtgctccctgccaccaggaaggtcagccgaccatcaggtgatggtcaggttgttgtcaactgtctctaaaataattggccacagccagcacaagggaaaggcagtctcccaataaatagaaaacacctgacactagtgatcagcagctgcctgataagatctcaagagttgggtgagtgggctcaagcatgtacagtaagaggcaaaatggtggagtttaactggtatatgacatttttctaggaacacttgactggcaagggaaaaacactcaagtgagcatgcacacatcttcagtaaacatactacccatgcagcccctcctgagtgctggcaggccattgtgcatgcagacagcctaccccaagggaagaatcaggggagaagagatgcaatcccccgggaagcatgccaacatataaaaccccaagtcaaaactcaaaccacacagttgatctctcaagttgtccacttggccctcttctgagtgtactttacttcctttcactcctgctctaaaacttcttaacaaactttcactcctgctctaaaacttgccttggtctctccctctgcctcatgcctcttggttgaattctttcttctgaagatgcaagaattgaggttgttgcagacctgtacaaattcactgctgctaacagtaataacaaccccatattaactttttaacagccaagcatggctcaaagagctatccatatgctaattcatttattcttcccaacaactcattgaggtaggcatatttaccaatgaggaaactgagacacagagaagccaagatcacacagttaataggtagtgaagcagagatttaaatttaaaaaaactggttctagaatacacgttcttaagccctggtaattatttcaattattttacctatttcctatggcacagcaggagaatgaacagggcttagtagttacctgcacagcactatttgaaggatgaaagaatatccgtcttatccatccccaacccagaaaatctttcctattaaccaccgtgttatcagacattttaccattaaacaattaaaggtctgggctgggtgcagtggctcattcctgtgatcccagcactttgggaggctgaggtgggtgggtcacctgaggtcaggagttccagaccagcttggccaacatgtgaaaccctgtctctactaaaaatacaaaaattagctgggcatgatggtgcatgcctgcaatcccagctactcgggaggctgagataggagaatcgcttgaacctgggaagcggaggttgcagtgaggtgagaccgtgccactacactccagtctgggtgatagagcaagactctgtctcaaaaagaaaaacaaaaaaaaaactaaaggtctagacaaagttattagttctcctggtttaaatgcctacaccctgccctcccatccccgtgtgcatgcatacagaatgaagtctaaacttctagcagcaagccttccctgctctcatcccctctttcctcttagctcctgctcctctctgcttcacaccctacctcaagacccatcaaacctgggcatgttcctcgaaagggctgccgtctttcacataccacatacaagtgagtcttcacgtttagttctctcttccttgttacctgttgaacacctatttatataagttctttgcttctctcttagttgaccttcagtgcccctcctccatgctcccatagcaacctgggcattttctttacatagggcataagtgagcaagtatactgagagccaactgaaggaaagggttatttttgctcatgtctgtattatccatttcagtgcctggtacttagcagatatttgataaagtttccctaggccaataatagagtgtttatcaacactctgtaataaaccagattgtcagtatggttttttccacaattgaaatagctttataattttcctccttattaaaataatgcattctctttgcaaacatattctgttataacaagaaaatgtaaggaagaaaataaacatcaccgatcctatcaccactagtgatagcttctccttttagctagttttctgtaattgcagctggctgagaaagaatggagcctaagacatgttcattcatcagagccaaaaggaaatgagagacctaaatcacagagcactgctgtggaaaggcatccatgagtttaacactcaatggatatgagaaaagcacaaggtgactggttgctgaagaaagagtaccaggtggaataggacagcaaagctatggtgacagaatgaagagattgacttctttgctgccttcagagaacaaaaccaatacacagagaaaatctgccaggaagtcagcctttgattcaatttaaacaagttcctaataatcagagagatctctaggaagaatatattgctctctaagatagttaagctgcctgtccccaaaggtgttcgagtagttgccaagaaatattcattcacttagtcaatcagtcagtcagtcattcagtcagcattaattgagcacccatcacttgtcaaatgatgttctaggtgctggggaacgaaagtcaaatgaggcccagtggtccttttcctaaagtggccattcctcaagagcaggttgctactactacagatgacagggtagatcagatgacttctgacattctttctaactacagcaaccagtgagtcttacttctgagaagtctggccctagcaaacctcacaaaagcattccagcttgaatgtgggtagaggaaaaaagatgagaagatccctgggacatacttctctcctccaggtccaagggggcctattgcttctcctctatgggaggagattacaagtgtctggatttccagttaggttgaggaaaatccacttttcaaaaggtttggatttgtacagtgttttacttaaataatgagggccaactcagcttatgaccaaagagttatttcataaaataatctgcagttgagttcttcaaaactataagctccctttatgcctttacaattggatttgacctcaccaacaaataattctatcaattttgaggaggtcatctatttgagaaatcaaggctcccatgtttaattccctggacctgcatttgctcagcttaaccctgtcaaactggtgtagcagctcttcccctattcttgctgtgggatcaggattccttttcaccattgtagctgacctgggacctctgcagacagacatcctcacaggagatccagacagtggtaaagaaaagagttacaacctgggcaagccactggacttttggctgatgaggaccttaggggcagatgatgtggtactcctccttgtatgagtggggaatgtcaggcacattgtcccctggcaattgccctgatccagcccacttcctagaaacccaaatggtacccctccagtggtcctctctgcccaactggctggcactcctggtggctgttccactccaactactcaggcttagcagaaatcctgatttcagataaccctcctttttccttcttttccagtctttcttcaattccagaacatatccacattaccctcccactccaaatttttgttttattattttctttcattaaacttttaagtgtaattttaatgctatcctaaataattagtgtgtatacacaggcaaacacacaaacacacacacacacacaataaggaggtttgctttaggggaaggatggcttggtcttcaaaattacaatggctagcagggaaaatgtctgtatttcattctaaggaaggtcacctccctgtctataagagaaattgacttctcctaatgcagcaaagaaaaaggcaaaaagtaagttgaattgttgacattaaaaaaaaatagattggacataaatattcggctgcatgtctgtgaaggctccagccaaagtggtttaatgatgctggctcagagaagggctgggagcactccaagactggtcagggagacagtagaagccaccagaaaaaatggtagaagacaccagcagaggtggcacctgggtggcccttacttgactgtcaacttcagagcatttcccccaacctctgccattttgttctggtttcctagattaaatggcctggtggagttctactggaagccccagataggcagctggtaagctgtctgagtgttcttagcttaaggtaacagagggaagactagagaggtacatgaaaaacctttgcaatggagattcttcttttgaagctgagatttgaattctcatcaggagagcagccggatgtgcacattattctctgcaagagtactagctaaaaagggtaagttgaggctaaaatccaggcctgccacgggactacatttattccaaacaagcttcttttctaagtcccataagggcactgaaagggttagcaacccctcctaaggagctgctctgagattatgctgctcctgtcatgccaaccctcatggtccacaggcatgtctcctgcccataaaaaaggctggaagcagcaggaaaaaataccgtcatcaccatgttcacatcctccccagcactcccttactcctctctatagacagtggctacttgagatcgaagtcttccagggagaaagcatcggggcctgagttgtctagaaaatttgttttcttttcagctgttgctgtccaactgtgagcaagtcacccaatcttgttaaaccttggtttcttcattcctaaaatgaaatcaatcccagtcctacctacctccaagtttactgtgacaattaaatgaaatggttcccctgccctcaatctagtttccagaattcttaggaagcagtgctagagagagctttgagaaacaatcagaccagcctccatcctgttctctgccccatcctacccgacttcaagccagtgataactcaatctttgcaatgtgccaagaccctgctccttaaaatgctggagggtattttaccaaagaaaatgtccctaggggtttgcaggagtggccctaagcagtcacaggcccggggccattttccgtctctcagcagcatgcatctaggaagtggagggagaggagagaggggaggaagggctgccagaggatgcttaaatatacctgcctgtcatctgctgccctctgcccaggccatatcctgagatgatgtggggaaaggctgacctgtccccttagcagtgttcagaggctagctcagatgttgttgtcacaatgcacatatggtgggtccctttgcccttcaacccagcgccccttgcagagcaaatcccatggtgagatcattatttgcatcaaccttcggctgccagtctggtgctcccaaagctgtctatctagaagcccatgggactaaagatttagtagccgatcaggcaccaggtcttccaatttacagttcagagaaaagaaaccctattaatctggcaccttttattccattgtaaattccggccatatttaactcagccgccatcaaggtttccctactatctgccaacagcaggcccataatataaagcttgccaattcaaggagaaattaagatcctgcttagtattcaaagggtgatccctttcactagcttcctttccgaagttccccaacatatccattgaggcaggggcagaacatatctgatctagcttagggaagcccttttggtcatgcagctggagaaaaagaggcctggtttccttccaccttgtaaaaaacaaactcagcctgcactgtctgtgatgatcagacagtcccttcctctcataaagcctacagaacaaaaatgactctccaaggcctcagggctcacaagtggcagagccagggccagaggagagaaaaaaaaaaaaaagaaaaatgtatgtatgtacttatttagtgagtaactcccatgttcaagacactgttacatattcttttctattaaatacacccctgcaagcccagtgttattagctccatctaacaaagaagaaactgaggctcagatcacccagctactaaggcttaggcctgcaattagtctctgtctccagaggcaatttattcagagtcaacaagtattttctgagaggctgttatgtgtcaggcacagtgctagaagaagggattatagaggtgaacatgagatctttgtcttctgttctaaggaaaagagacagaaataaagaaataaatgagaaatgtgtgagagtgatgagtactctgcagaaaatgaaaattgggtcatgtgatagagaggggcaacattagattgaatagtcagggaaggccactatgaggaagtgccatttaaagagacatctaatggcaagaaaaagccagttcagacaaaaggggagtaaaggagaatgtaagcagaaggaacggcccctgcaaagagcccaaggtagaagtgatggcggctggcttctatctaggaaacaggcagaggggcagcgtgaggagacggaggagtgtcaggtcagtccccagcagtagacaagggccagagtaaggagttgagattttattccaagtggaatgagaaatcattggaaggttttaagtgggaaactgacgtcatctttatgatttaaaaagagccactggcccatacggcttgggtgagagtgataagatgtcactgttttcctggttaatagtttctgataaattctctcaactggccaagtgagccagctctacagtgttatctaagccccttccctggctcctgaaatacttaccagtaagctttccttcaggcaaagaaaacaatggtcctttaaaaataagcacatgaagaaatgggaacccactgaacctgagtcagccctgacacacagaagcagttggcctgacaggttattcccagttcatgaacctgaggtaaggagacccaggttgccatgtattgatgaggggaggacagaggaagagaaacttagatcacgaattgaaccaggagtccttcaaaatccagtagccctaaaagcctgcctgaccacccaaactttagtgtgtttgaaaaccagcagtaagttgactaagtagcttcccatctcttgatggaaactggcagctgtaagttcggtctgcagctcaagtccctggtgggccacggaaagcaaagttctccttctgtctcatggagcatccaaccctcttcatctcctcaaagggttgcttagatatgatacctggaccactgggccatctgctttagaactacatggagcactgctgattgcccagtagggccagggaatgtgaatttgcaagcatggcaccattctagtgctcaccaatatttgtggattgctggaagcccagctgcaggtggagctcctggtggaggaggcttccaggtattgctcaggttgactcctgctcctaacagttcacagctccacccttttcgaaacgagaattatttgtcctcaaaagccctgtgagaaggtaaggaggactaggatcctttcctttctggaggtcacgaaatagagaaaagggtttggtgacttatgaaaggccacgtagcaggtagagagtaaaacttggatttgaatcaagtcttatgaattcaaaccctgtggtatttcaaacgatagaaaccatctgtcccagaaacagggagcaatgcacccgctttacagcactgggaggaggctgtggggaccctggaggcagagtcgctgtttggaaagcaccatgcagaaaaagaaatgctgtgttctgaggagaacttaattgatctgatttatctcaacatggataaggaactatcaagctcctaccagtacatggagtcctggttcactgtcctctccctgaggcaggttttaaaaagttataaaagactgcatttttatctctcaacgcctaccctggtagattaatctctttcctgatcacactgatgcccttcccccagccccttcccagcacagagagagaggtactgaaagtttttctcaatgtggggaggaggcaaaaggtaagaagatggggatttggggtggaatttctgtttgttcctgagccaagaatgcttgtcccactctaggcgcttcagggaaaggagtggaactagttgtaaggggcttcaaagaaaaatcacaactgattggcaagtctgaattctgtccaacttggagtgaatctcaggggtattagtgggatgtgacagagatttttagctcctcctgagggctatcagtctagccagactaaatcaatgatcctcaaaccctaaagtagataataatcacaacgaattttgcttaaaattcagattcctaggcccaaccccaaagactctgactcaagaggcttgggatgggaaatgaaaatctaccctttagcaatgaccccccagatcattctaataaggtggtctacaagatcataccttgagaacaacttgaccacctggaccagatccagggctaggattcccagatacccttcctggcacaactttacctagacagagaaggattacttcaagtaggagcctaacaactcccacacctcccttctggtcccagagcagaactgctcctcagtgatatgatgagaagacttcctcttcccagcatctaaattccatttcctgattcctttctaccctgtgacactatccaacttgactcctttggtgccttgtgaaatggttgtctatggattataggttatataggaagaggaatgaggccaggagggagcaatggcctagaaaggtggagaaggtcagtgttctacaaggaccatgatgagactgatgaggaggtagaatggagagataaaacagtccctggtgatagcaggatccagatgtctccactgagagggtctggaatgaacttctgtgtgacaagcagagactctgaggctgtggaattaagcatatcatccacatggacattgaggttgcccaggcaatgataagaaatggtttgcagaggaagaccataagccagaagctgaatacttcaatgaagagatggccatggccagcagatcagtaggtgacaacaagctgtgggtgtcatagcctggcgaaggagcaatgttgaacaataatggttgaaagaaggaatgaatgtgtagagaatttcaacctgatctccaggctggagctgaaggaagggtaggtggaaagagaaacaaagaaagtgagtaacttccaaggagaaggttgcaaaagaattggtcctatcaggtggaagccagaagtcaattaacacagtgttgtacagtgctttaggaatcaaatagacctgaattctggcctgactctgccaatttctaactgtgcaacctggggccattcaattcattcaattcatctcccattgtctcattttccttatctgtgtgaaatggagataaaaacactgcctaccttataagagtttcataagggattgataaattaacgtatgtggctgattagcacagtacctcacacatattaatgcccaataagttagctcttatttaatgaatggatgaaataatctagaaatagtgtggggctgagaaaaaaattgctgattttgacagagcctagctagatgcttatcattccatttcctcttcctgggcacacaggaagcctcctttgcagtttgattggctcttatgtgaaataagacctttctatgaagtaattttgaatgtcttctgaggaacacatatcaggcacaagaaagcaaccaaaggaactataagtgcagaacaaatagaaaaagagagtccagttctctcctttgaattggaaacaggccctgcttcccctccttaactcctcctgtgggtaagactttgtgttctacagatgggttacagttgaaagatagtagagaatgaaatcgacctagagcaccactgataagagctgatacagcaagacgcctacaacacgagtcatcatcttcatggatgaagttgccaatgccaagtgccattcccagcacacatagccgtattctcacccaatcttctaaatctctgataggtcctattttcttgaaggctgtggagcatgttgacaagtatgggtgggggacagggcagagctgtgccactcattcaggttacccatacctacagtcttgacctcattagtgacatattgtaataggctgaacacaaccacaaaattttgatggagtatacaaatggaaaccatgaaatggtcaaattctgtatttatttatttaagaaatatccctagagttccttctagatgcaagacaccatgctaggtgctaagggttgttagcaaacaacccagacaaggtcccaatcttcctggaagataattaaagcaagcgattgcagtgaggttctaaggaggttaggatagggaaggtgttacatagatttgatgaaaatctaattgccattttcttttcatctgttccctgtggtttgaataaccctgtacaagccttgtgtgtgagagagagtctgaattttatcatctttatgatggaacatcacacctctccccataagaattgaattattgccaagagattctcagtctatcttcccttctgaaggcctcaatgtgctgaatggtgagagaatttgtttaccaaagtaaacaaagctgaaagtccatttcaggcagacaaaaatcaccagctgggggcatatctctgtggctcattctgaatcccagctgtctgcatgccttctgaatgtgacctgaaccctttaaaatgatccccaccctatccctgctctcctgtctcttatcagtatggctaacagggcacactgtaatcttctctcttatgtttctcaaaaatgtggcctgagtaccaacagcatctctagccactggatacttgtttaatgcagaatcacaggccctaacctacacctcctgaatcagaatccaaattttaacacaattcccagatgctttaatgtgcattaaaatttggggcacattggctttgaatataagctgatttctacccagcttcttctaccaaactgcacacaatcttttgttctaaaaataagcacctactatgtgctgacaccctgctaggtgcaggagttacactgttgaaccctcagaatgacaagaaaaacatctgtctgaaatctttttggaatgaagtaagttatacagtaataagaagtggcagcgactggcccagagacaagtccacacagtagaacatgagaactgctctgacatagatgacaaaaggggctgtgggaacaaggaaggagttaagttggcataagcagtttaataagactccacaaaggaggcaatgcttgggcttaacatggaaggatgagtaagggcggagtgggtggaacagcaagaggataaaattgctggccaagggataggaatgatggatgtcaaagcaagaggaggcctgttctgctcttggctggaggcccaagctctgcgctcatcatgggaaagctctctgccacttggggctctgccaacaatcactatgaggccttgggcaaatctctgctcccctctgggccttatgttcatctaaaacttacaggttggaccagcacttggagattttcaaatgtagagttccttcaagaacttcagcaatggctaaaaaaaaaaaatggaaagaggcaggactctgggcctggccattagaacatctgccaagttgtattcattttccatgtggggtaactgcttagctttaagggggtggaggggaatgtggcgtgagattgcacatctttatgtcagagatcagaatatgccaccccaaaatatgatgttggaggggtgcaggacaagccaccccaaattatgtcgctttagtattttgattattttgagctaaaaacacttgaaaaatagcagatgcagggagaggctttcactgaagtctccttatctgcctgaagacagatccttcaaaaagaattcaattggcataaatccctttgctgggagtttcatcaaccagggaaggttggctcttatcacaggagaggaggctatagacgatccttgacttaggatggtttgatttatgatttctcgactttagggtgatgcaaaggtgatataggcattcagtagtattttgagtacccatacaaccattctgtgctttcactttcagtatggtatttagcaaattacatgagatactcaacactttattataaaacaggctttgtattagatgatttgacccaactgtaggctaatgtaagctgagcgcatgtaatgcaggctaggctaagctatgatgttcagtagcttcagtaggttaggcaaactgaacgcattttcaacttatgatacatttatcaggatgtaaccccatcttaagtcaaagagcatctgtagaagttgacactaaacccagacaaactttgtcacaaaatatcatatcctatttattcttttaaaagcccattcatcttttataaaaatcacttactttccctcaggtggcctacatccctctccccctcccctattaagatggatatacagctttttaaatatcaccacttttgggggtatttgcttttccccccaggatgcccctgtgcatctaacattaaacattaatacatttgtatgcctattcttctgttaatatgcctgttgtcagtttacttcatagacctagctattgaacctaaaggataaaggaaaagtctttcttccctacactttcaaagtgtgaaaacaactgaagggggtgacttctgagacctcaagccagtgctaatacttaatgcttatccagttctgcaataagtctccaggttgtcacgcaaatcagactttcagctctatggtcacaggcctcctctcactgtgtgtcttatagctataatttctatatcttttaaagagcagaatccctttcctaaatgatattacacaggaaatccacaatatgaataactgatcaagtgaatacacagatttctagataggataaatcttcattatttggcaatactttatgtggagtctgcttgtccctgtcaccaccatccccagcccttctgtagattcccagggttgagagtgaaaatgactcagaggaagtggttcaagctgagatgaagtgtgcagtaagtcctggagctatggaagcaaagagcttccctagaggaaggtgtagtgggacctaggagcagtggcttgaaaagatttggggaatggaaaaagggaaaaggacaggaccagatgaaagaagacaaacaaacaggaagacaagaacagaatgaaagcttcctcaaaggccaattaagtgatccgaaggaaggctatatgaagccttagcaactgctgcatacacagccaccttttaaagaaagtggcccctatccatccttttccacttgctgactattataggctgaattatgccccctaataagatatgttgaagctctgactcatagcacctcagaatatgacattatttgaaactagggtcattgtggatgtaattagttaaaatgaggtcatcccagagtaagatgggcccttaattcattatgacagatatccttcaaagacagaagagtctcagagacccatagggagaatgccatgtggtgacagaggtagagattgacatgctgctgctgtaacaccaaagatcactggccaccaccagaagccaggaagagacaagggaaatgtctgtccagagtctcagaggaagcagggccatactgacacccaattttggacttaaagcttccagaactgtgagaaaatacatttctgttgttttaagcagcccagttggtggtactttgttatgacagccctaggaagtcaattcatttgctgtggcacaagcagtagtaggttttgctagagagatcatccctgctactcagcagatcagttgggcatggacgcagacaaagacaactgggtgcctctgagccagcctgtcactctttgtccagttgggacatttccacagagtggaggcaaagcaatccaatgattccctggctgtggagccacataaatgcagataaagagaggaaaagagagagagagagagaaggaaagaatgagaaagagtgcagattaagatccagctcaacccttgtcatgatttgccaaacagaatccaatcctagtcaaatcttcagctagaaggaggaggtatccactacagagaagggatctttggcgtaacagagggatattccccagaattccccagaagggaatatgatgtgggcaggcactgcaaaacagacctgctgcaggggtgaaacattgcccttataataactgcaaagcctgagcttgcaggcaagaagaaaagaaagggaagcaaatgtatataaagaagtgtggaggaacagagggatgtagaatgcccaaccatcccagattgccttgcactgagggggttcccgggacataagactttcaggtttaatactgggacaaccctgaataaacgaggatgaattggccagccaaagacagtaaaagagtaaagtggaagaagcaagttttgcatggtgaaatatattgtcagcacccagaacatgtttacagaagtgacctaagccaatacagatcaatctagaccaggagagcccatggtcccactgcaacctggctagatgggcctgtcagtggattcaggggaacttgcactgaggattcactcagaacctctatgctgaccaagggcccttgggatggcaatggtaggtgacatgaggatcagatccctttctgcagtctggaaggcttgccctgtccagcaatcctctctaaggggatgagctcttcacactgaactggaggatttaccttcttgtccagtggggttggcctgccatattcctcagccctacagggaaccctgtaagtaatgagtccaaggccctgtctacagagatacttttttatatctccttgtcttttaccccacacctgccacaaattttatatgaaatattcccaaggatgcatgaaaaatgtcattcaccaacttatccccaccaggtgggagaaggcctcctgtttatgtttccatgtccttccaggcatatgcccatcatagcactttcagagtatattagggtcattgattttatttttcaccttctcagcttgtgatcaatttccttcatagtagagcccataccttgctcatctttgaaatcctagagcccaggaaaacacctggtatgtaggagtttctcatgcactgtctggcaagtaaactaattaattaattagttcatgttgatcaagatgatccttctctttgtgtcaagtgtctaacacttgctagatctaagacccagcactttcttggtttttaatatatcagtcattaagccatctgcatgcagatgagaggtagaaaacacttcagagccctgcagcctcagagggtcacctgagtgagcagtcaatgcggccagctctaaagggcttatgtcaaggcagtcacaagggcttgggaaggagatgcagacacctgtgcccattacaattatcctacttctactctcacctgttactgcggttctaaacaaagagaataaaataaaataaaactgagtcaaggaaaagaccagctgctgagctccagacagaagctcaataaaagataaacagaatggtataaattggctaagagaacattggctggaatactctatgacattaattctatcttattaagaaggcccttgatgatgaggtggtagacctgggtctgatctccattccactactatatatctccagtcaggtggtctagcctctctgagcctctattagcccaactctagcttgctaataaccttctgctccaggtccgtcgaatagacatctgaaccaacacatgtccgacattttgacctcttctaacgaggcgccctagagaaattcaaagcagagaattttctctgccctaaaatgactcccagagctccaaggacaattattttagcaaacctattgatggacttaaagtttatgaacccgaaagctaatgtaccttcctctgtaaaaagcaaggcactgctagatttttgtctgcataaaaactcaaacactcacctgggaagtctgttagtaaataaagcttatcttttgcaagatgcaagcattctacttctagtttggcactgttcaaaattcttttgttctgaaatttgaaaaaggataggcctaatgtttcactgacctcctgtgggcataaggaagaagtacactaattcctcagatctggatatcctggtgtggtctaaggaagaacgtcttaaatcttacctacctgatggaaaattcatacagaataagattacacatgctatttcatctcttctcccaggatccctcaggcccgtcataagttcctaatctaacgggtacttaaatccattagatagatttctacaatggcagagcaggcagtgtgcacaggaatcatatgaggaattgattgaaacatagattcctgagttccatttaagagattctgactttgtgggtctggggtggacctgggaatttgtattattttactaagcaaatgattttgattctgatgcaaacagcactgtggattttacttcggaaaataccaactcgccaaatcccctcatgttataaataaggaaattaagacattgagaagttaaatgttttgcccaaacccaaacggctaattagaggcaaatctggacttggcactaacttctgattctgtcatttgcaatcaaaatagtcaccatttaagggcacttattattcatcaggtgctgtgctaaatgcattggctcatataatccttccatcaattctgcggagaagacgggtagtattactaactccattttataggtgaggaaacagaggcttagagaggtgaaatgacacattcaggtaccaaaaagcagaacaagactcaaactcagtgcatgtgactttaaactaaagctctcccccactgccccactctgcctcagaagcatagcacagactttaatcttttgtggccctctgaaaagcaggagtccccccaaatttgaaatgtaatgatgcacatgtgacatccaaggacacactttagcaaaccttctctcttccctcaagagagagaatcttcaagatggggcatctttgtaaatctcagaaaaggaatcttcttcaccacaccctgaagcaacttgaactcagtatcagctggggcctttcaatcaactggaagatctgtgctgaggttgtaatcagagcctcttcaacaagcatcagaccactgaaactcaggaatacagagagctgttcaactaggggatgtcttggagaaaaataaagactcttcagaaagatgggttcaaaatccagtcggggaagtcactgacaagcaccgtttaaatgaatcaaaggaagaagcaggattattctcatgcacctatttctctcattaagccttgtattagttatccgttgctatatgacaaattatcctaaaagttagcagcttgaaacaacatttattatctcacacaatttctgaggttgagaaattttggagcgacttagttaagtggttctggctagcctctgaagttgcagtcaagctgtaggctgagctgcaatcttctgaagactggaggatctgcttccaagctcatgcatgtggttattgggtagcctcagtctctcaccagctgaacacctcagttccttgccacatgggctattccacagggctgccttagtgtcctcaagacatggaggtggtctccctcagagtgagtgatagaaaaagagagaagccacaatggaagttctgcttttttataacataaactcagaaggaaaatgcatattctgttggtcacagagaataaccctgctgccatgtgggaggggttgtgaaaggaaaataaatattgcggcctcaaaatcactcaagctgggaactgcttagggcaaacctgtctcccattctattcaaagtcacccctctgctcactgagataaatgcgtatctgattacctcctttggagaggctaatcagaagctcaaaagagtgcaaccatttgtctcttatctacctatgacctggaagctctctccccgctgagtcttcctgcctttgcttctagttgtcccacctttccagactgaaccaatgctcatcttacatatgttgattgatagctcgtgtctccctaaaatatataaaaccaaactgctctgaccaccttcaacacatgtcatcaggacctcctgaggctctgtcacaggcatacatcctcaactttgacaaaataaactttctaaattaactgagacctgtctctgattttgaggtttcacgggatctatacaatagtataaatatcaagaggtggagacttgtggaggactgttttgagtctggctactgcaaactccttctcctgctcacctttccttgtccatacaggtccaggtaactcctccatgggctacacactatgctaagcaccagtgaggtagttgtgacaacagccagtctcttgcatcttggagttttctatttaatgtaagaaatagacattagacaagtaatcacacaagtaaatgtacaactaaaaatctgtgaagaaactgagaaggaaaagtatgatacatgatataagagtataacatgtggtgggggagctgtaatttgcactgaggaagttaaagaaagctttcctaaataagtaaaattttaaattaagtcttcaaggccaaataggattaagctaaaacaagaatgggagaaagaccatcctggcaggaaaacagcatgaacgtatttcctataaaggggaagatcatgacacatccagggatatgaaaagccagtgtagctagagctcagtgggcaaggaggcagtgagttcctccctcctggaacactctctccactccttgtctcttctctaaagtatacccttagagctcaaatgaagcctcacctcctctacaagttcttacctactccaaccgtcatctctcatgccttgttcaaccctacacaacctaactctaatgccttgaaatttcagagtagtacatatcacactgtgctattttatagatttttttctttctcagtacaataaactctctttgcaagaaactataactagtttctatatccttgtgtacctaatgtaatgcataacatataatagacattcaatacgctcaaataaatgatcaaaaaccccatgcaaaaagtaggattgtagccctgcttgcatgcttgtacatatacatgactgtgcttttttttttctaaaggcatgcatcacacacacacacacacacacacacacacacacacacacacatctaaagagcaggttctgaaagtggggatggtagacaggaaaactaaaatctggtgggattagaccatcctgagtgattttaatccattcaaaggaagtaaggacttgtaaatatttgaatgataaaagattaaacacacacggttagatttgctctctccaaaaatcttactaatattacagcaaagggatttttaaacataactttaaagacaaacaatacaaaatgatgcaagcaacaacattttataagttgggctactactcagataaagtagcttagtgatttaacaaacttgaaaaagttaaaccctaagctagtggtagagaaatccagcaaataaatcaatgtatactgaggaacttccaaaggctctggaattggcagcaaggcaattctggaagtggggatgaaagtgaaactgaaaataggagaaagggtctaaagtcagcttagaagcagttgctcctagaaacatgccttgaatgacatgcaaggcaattcatagaaaaactattactatggtaccatacttacaaagctcaaaacaggcaaaactgaaaacatacattgtttatagatatatacatatatgattaaactatattttaaaagcagggaaatgattaatacaaaattcatgacagtaattacctctgtgtcaggatggtgaaggaggcagggaggtggaaaagggaagaaacgcagaggtaggtacaaaaatatttggaatgcctttgttcttaattgatgttcattttgttaacatgcttcataatttacatatgtgttattaatatggtttgcatataatatttgtatattttatcttgtataaatattacattgtgtaaaaatataaacagacagccagatctctttcttccaagtgtctgggtgactgcctgttccccatccaacatgagctgaagatatcatctctaaagagagtaaaaccaagggtatttggaataacagagagggttgaagggtagaagacaggtgccatactttaatcaagcctaaaatgaatattgtgttccgtgaattgtgatcttcagccttctccttccaatcagtttccagcatgcttgcagccaggcctataaggtagatatttgaaagatgtttcttggagaatcaggccggcccaaaggaaagggctagattttcacatgggaggtttctcaacaaatggtccacactgaacacattatagtgaggctcaatagtcaacaaactgaacctatgtgttcagggatttaagtttgctttttagtactcattcataaatattaacaaatgaccagggacatctaaatatctgagggagtcctctagtggaaaaaataaacaacaaaatcaaggagaaaataacaacttgaaaaaacagaaaattcaaggaaaacaaaagtataaagttgtgtatcaccaatgttctcaggaagataacaagacagatattgtatccatgaaacataagcagcatactatagtgaaggaaagctaagtaaacagaaaagtgctcttggaaattaaaaacataattgcacaaataaaatattcaatataagacttcaaatgttaaattcagaaaatttcttagggtatataagccagaggggagggaaagaggagagagagagagagagagtacacaagggaaaacagaataaaaatgtctttctttgagctgctacaacagaataccatagactaggtggcttatgaacttatttcttacagttctagaggctagtaagtccaagatcaaggtgctggcagattcacggccttcttctcactgtaacgtcacatggcaaaagagggtgagtgagctctctgtgtcctttttagaagggaactaatcccattcataaaggcagagccctcatgacctaatcaccttccaaagacccccacctacaaataccatcacgttgggggttgaattttaacatatgaatttggagaggacacagatgttctgcctatagcaagaaagacgaagaaaattagaaaacttattccagaaggtccaatattcaaatggaagaaattctagaaaaagacaaccaagaatatgaaacatgcatgaaacaaatgaagagaatttaccaaaactaaaagacataagtttccaaattgaaaagttccaccaaataatcataaaaatagaagtggataaggacaagctcataccatcatagaaacatggtattttagagcactcaagaaaaaggagatatccgaaatgtctttggactttttgatttttttccttttttttttttatttttgagacagggtctcactatgttgcctaggctggtctcacactaccaggctcaagtgatctttcaacctcagcctctcaagaagctgggacaaggcacacaccatcatgagtggcctggaatttttaagagcaatacttagagctaaaaatgtgtgggaagttcatcaaaattctgaaggaaagtattttcaacctggaattctatacccaacaaaagtatcaattcacaattaagaatcagagtacaataatgacattttaagacatccaaggtttaagaaatttacttcctaggcatcctttcttaaaaagctatcagataacataaaattgactgccttttgcatagcaattgttgtagaatttttcaccctaaaattctgcagtctgtgagagatttaaacctaaggctctaaggcaacggttcttaaatatttttaatttcaggatccctttaaattattaaaattcattgagtattccaaagaatttgggtttctatggactatatgtactgaaatttaccatattaaaaataaaaactaagaaaaataaaaaatgttaaatatttattttaaaatagcataataaacctattacatgttaataaaacatgtttttatacaaatatgtgttcaaaaaatagaaaaaatagtacattgttttaatttttttaatttttattttagtttttgggacagggtctcactctgtcacccaggctggagtgcagtggtgtgatcccagctcacttcaatgtccacctcccagaatcaaacatcttcccacctcagcccccccaagtagctgagactacgggcacacatcaccacacctggctaatttttgtattttttgtagagataagattttgtcatattgtccaggctggtctcgaactcctgggctcaagcagcgatccgcccaccttgacatcccaaagtgctgggactacaggctcaagccaccacaactggcctttaatttttgcaaatgtgttcaaagtctgacttaatagaagatatttggatttacatatctgcttctttttacctcaaacatttattacttcttgtgctggaaacattccaaatcttcatttctagctattttgaagtatacaacagtatagttactgttaactattgtcatcctcctatgctatcaaacactagaacttattccttttatctaattatatttttgtacccattaaccaaccttttttcatctccctctccattctaccctttccaatctctggtatccaccatgccactctctacctccattaaatcaattttatttaacactcatgtatcagtcagaacatgcaatatttgtgtttctgtctctggcttatttcatttaacatactgtcctccaggctcatctatgttgccacaaatgacaggatctcattcttttttatggttgataataatctattgtgcatatatactacatcttttcttctttctcccattgataatgaacacttaggttgattctacatcttggctgttatgaatagtgctgcaataaacatgggggtgcagatatctttttgacatactgattcccttttcttaggatatttatgcaccagtggggttgctggatcataagatagttctatttgtagttttttgaggaaactacatactgttgtccataatagctgtactaatttacatttccaccaacagtgtatgagagttcccttttctctgcatcttcagctgtactttttttgtgtttttgatggtaatagctgttctaactgggataagaaacatggaaatcgataagaacatggaaatcgttccttttttgtgtgttttcttaaatttcttccagaactgctttatagtttttcttgtagagatattttaccctttggttaaatttattcctagttatgttttttgtagatattgtaaatgagattgctttcttgatttctttttctgctacttcattgttggggtatagaaatgctactgatttttgtatagtgatgtcttagtccatcatgtgttgctataaaggaatgcctgaagataagttatttataaagaaaagaagtttctttggcttatgaatctgcaggctgtacaagaagcacggcactggcatctgctcttgtgagggcctcaagctgcttccattcatggtgaaaggggaaggagagctgatgtgtagagatcacatgatgaaggaggaagcaagagagcaggcagcaggtgccaggtttttttaacaaccagctttcatggaaactagtagagtgagaactcattaccacaaggacagcaccaaaccattcatgagggattcccctcatacccaaatacctcccaagaggcctcatctccaacactggggatcaaatttcaacatgagatttggaggagtcaaaccaaactatggcagtttattttgtatcctgaaacttcactaaatttctttatcacttctaagagatttctggtgaaacttgtggattttctatacatgacaccatgttttctgcaaatggacaatttgactttttcttttccaatttgtatgctgcttatttctttctcttacctaattggtctggctaggatttccagtactatcttgaataaaagtattggaagtgaacatctttgtcttgttccaaatcttagaggaaaagctttcaacttttccccattcattatagtgttggctgtgggtttgttatagataggctttattgtgttgaggtatgttccttgtatactaacttattgaaagttttttatcatcaggggatggtgaattttatcatacgatgtttctgcatctattcagatgatcatacaattcttgtctatcattttgttaatgtgatgtatcacatttattaatatgcatatgttaaaccatctttgcatccttggataattcccacttgatcatggtgaatgatcattttatgggctgctgaatttgctattattttgttgaggatttttgcccctatgttcatcagggatttgcaatcagggtaatgtaggctacatagaataaattcggaagaattctctcccctgaaattttctggaagaatttgaggagaattggtgttggttcttccttaaatgttcgctagaattgtgcaatgaagtgattagatcctaggcttttctttgatgggagacattttattacagatttaatctctttactcataatttgtctgttcagattttctatttcttcttggttctatcttggtaggttgcaagatacattgtccacgaatttatcaacttctgctaggttttttttttgtttttttttttttttttgagatggagtctgactttgttgcccaggctggagctggagtgcagtgccgtgatctcagctcactgcaagctgcgcctcccaggttcacgccattctcctgcctcagcctcccgagtagctgggactacacaggcgcccgccaccatgcctggctaattttttgtattttttttagtagagatggggtttcactgtgttagccaggatggtctcgatcccctgacctcgtgatctgcccgcctcggcctcccaaagtgctgggattacaagcgtgagccactgcacccgaccgatttctgctaggttttctaattaattggcaattagttgttcatagtagtctatgattctttgtatttgtgcaatgttaattgtaatgtctcctttttagtttttgactttattcatttggatctttcacctttttttttcttggttaatctttctaatggtttgtcaattttgtttatcttttcaaaaaagcaactttttctttgatcttttaaaattttttagactctatattgttgatttctgctcctatctttattatttccttcctcctttaatttcggattttggtttttctcggttgtctaggcccttgaaatgccttattaggttgtttatttgcaatctgtctacttttataatagagccatttattgctattaaattttctttttaagactacttttgctgtatcctataagtttgggtatattgtgtatctattttcatttgtttcaagaaattatttaatttccttctaaatttattaattgacccatttgtcatttaggagcatgttgtttaattttcgtttatttatatggttccaaagttgttcttggtattaacttttagttttattccatgtgatcagaaaagatatttgatataatttcaattattttacctcttttgaggcttgttttgtggcctaacatgtactctatcccagagaatattccctgtgctgatgaaaaaaaaatgagtattctgtagctgttggattaaaagttctgaaaatgtctatttggtccatttggtctaaaatgcagtttaaattcaatgcttctttgctgattgtctgtgtacatgatctgtccaatgctgtgggtggagtgttgaagtccccaacctaaattggagtctatctctccatttaggtctaatcatatttgctttatatatctgagtgttccagtcttaaggtgtatatatatttacaactgttatatactcttgctaaattggctcttttattattatataattgctttctttgtatgtttttagtttttgacttaatgtctattttatgtaatgtaagtatagcagctactcctgctcacctctgatttccatctgcatgaaatatctttttccatctcttcactttcagtctatatgtgtctataggtgaagtgagtttatttaagacagcatatagttgggtcatatttcttattctccattttgtatctagtctatcaatgagttcataattttatgtgttttcatgatagtgattatcatctttttgtttccagatgtaggactaccttgatcgaacatttcttctaagactgtctagtagtgatgaatttcctcattttttgcttgtttgagaaagtctttatttcttcctcatttctgaaggatagctttactaggctttgtattcttggctggcaattttttatttcagccctttgaatatataatcctattctctactgacctgtaaggtttctttagagaaatctgttgttagtgtaatggagattcccctatatatgattttatgcttttctcttgcagttttacaattatctctcttttttacttctgacaatttgaatataatgtgtcttatggaggtcctttttgggttgaatctatttggagacttttgagcttcctggatctggatgtccatatttctccccagacttgcaaagttttcagctattattttattaactaggttttctacatttttcttcacttctttttctagaatacccataatacaaatatttgttcacttaatagtgttccacaagtcttgtaagctttcttcattcatattctttcttatttttctttctctgagagtgtgacttcaaatgatctatatttgagttcagaaatttttctcctgctcgatcaagcctgctactgaaactctctattgtactcttcatttcattcactgaattcttcagcttcagatggttattttcaattctttttctggcaattcattgattttcttttccttgggatctgttactagagagttattatgtgcctttggtggtataacagttctttacttttttgtgtttcttttgtctctgcattgatgtctgtgcatctggtagaacaactgtctcttccaaactttctagtgtcacttttatagagaaagacttgcacctgcagttaaacattacagtgctggttggaagagatgtggtgactgtttttgcataggttcagtggtgtagtctttgtgcagcttcttcagctacattcaacataagcaataactgtggatgcctcaggctgtagaagtttatggcagtcatagcaattgtgtaggttattaatatccttggtgtcaagggcttttggggtcctcttattgtcatttttcccacgatgggaagacttagccaaggggatcctttttagtgtcaggtctgacatggttctacaagcagctgcagcagcactgggttccaggtgcaggtgtccagagtggctttggggctgggatcctagcctgagggtctcatgaacctattgtggcatctgggttttccagtgcaggcttgctccctgtggcagggatataggttgcccacaaagccaggatctgtgattctgaggcacctcctagcagctcagacccagggatccagatcgtaactgtgattctcccccaggaggggtagggtacaacaccggaagggggagtgctctggagctttgggcccagggaccagggtatagctgcaattcaggaacttgagctaataggtcacagtagcaactcaggtcccaagagatgaggtactgtgtagtggtaattctagactctgtggtggtgggtttcagcagtatcccagactctgtgaagccaggtgcagcagcaacaagtaccccaggatggtgaagctcagctattattagagcgctgggatgaggatgatggagacaggaaacagcacaatgatgactgcactccccagagagtgcagtatcttagcagcttagcctcaaggaggctagtccagctccagggaagcagggtgttaagagttgtttggcttgtaaggtgaggtctgtcagctcaatcactgctctgtttccctgggatgtgaggtaccacatcagctcagccctgggatgcacagctgcacagcttggccagggtactgattccccagaaggtaatatgctgcttcagttcatgtctgtgagggcatgactattctgggaagcccaggcaccatttctgggggatgcagggtgctgcttcagctggagtacatgaccactctggaaagccaaggtgctgtttcttgggacacaggatgctgcttcagcttaggcaccagggaggcatgactgctctgagtggccaaggtactgtttttcctggaggctgggtgctgcttcacctatggcctgagaggatgagggcaggggtaggtggagcagcttcatctctgcttggctccacaaggaagtcacagctttgctcacagcatggcttggggatgttgtgccactgggctggagtggtttgttggtggcttagcctcaggaattaaggggctacccaccccaagagcaagacacactccagccatagttccaatcccaagaaggtgtagcaaaccataagagatggggcatagcgttggctccttctctttgaggcacagctatgtggaatccagacggatccctcaaatgggtgtggtgcccctgaggactgcaggtgaaccagaatggtgaggcctgtaggtatgaaggtgtggatggggttgctaggatcctcttctttatctccttgccttagagagaagtttctcctgattcccagctgatccggttggggatggggtggtggaggcctggtgtttcctttcattctctatgtgactatcctgagtttctgtgctctccagggtttctgttaccccttggacacactctgtggctctccctcagttatttttattaaaatagtattgtttcttcactgttctagctgtctttgtgagcaggatgagcgctatgggcttctagccaaccatcttctgaacttatcttcacatctcctttactcaatcttttcagatatgttattggtgaagtaaatgaagaaaatccagccacacaacatgagaagaatttcaatagtcttttgcaataattgtggctattctttgattctacacaaaaatgcaataaataatagttcttcaaaggttacttgtgatgtggaatctgaagtcatatcaatgaactttatgtactctgttatactaaaatatactggtctatcttgtactttggttcttttatccatgcatgattttgtaacagaatgcactggttacttggaaaatattggttcatattgcaggtcttccaaacattgacacctttgattatacaacgtcaaaaaatcacagacattaatatcatccaatcagaaaaaaatattttaaggatgggagagttttccagctcacagcagcagataaaagttttccaaaattctaattttcatttgaaagctcattttattatcagttgttttccttgaactgacttgtccatttcatccatttttagaaaatatctgccaaaaactgaagtctgaatagtgatagtttgtctattagtcattctttcaagtaaaaatggtgctcaatagaaaaggctgtcagttcagcccacaactcaaacaatcacatggctgcttttcctcaggacattaatggtagtgtggtatacagcagaaatgctttatgaatatttcttattttgtcacataaaatactttttagaacctaatgaaggaattcttaaggaaaaccaatgcttttttattcatagcaaatatatggccatgaaaaatacaatgatcaggaacaatttggtgcattgcttgattcatgctaaggtgccagcagttatacacaacacaatttataaacaatcagtgcaaatgtcaacacagtgaaaaggaaaaacgtcttagcattagtatgaaaaatggtttaacctcacagatgtcctaaaagagtcttagagacatttggggctccaagcaccacactttgagcaccattgtcctaaggtatccattatatataatgaattaatctctcccatatctagaaggacaccagttttctaccatcctcgggagaatgggggaaaatgccaatcaaaacacattctgtgatcttgtggttcaaatgagggattccttttgaaaaagattgctactgtgtgcatgtatatatttgatcaaaataaaatgaaaatcaattaagggaggtaaattgaagccagcagactgcaagcctaaactcctgcacacagagactcagccacagctgccagctctcctcacatgctctcctttgccatctcaccaccatcaggaatgacgaggaaacagatgaagctaccaagttcaggagcaagaatctgtggtatggactcactttagtaatcatgtagcctagaggtcaagactggaaatgaagagacctggtgcttatgctgtatggtatgatgcagtagaaagatcactggctgtgttaagcctgattcaaagcccagttcaaaggttcaaatcccagttctgtcacttactagctatgggatgcctgccaatgcatttcaacctttctctctcctcatctgtaaaaccttcataaggttttaaaactattaaattttttaaaataactataaaaataaagactacatgactgtggacagagattatctattattataacattatatgcacaaaatataagtacaggtccctgacctcaagcagcttatgacttagctggggagtcagcataacttacacaaaagaagaggtgacagcaccaggccctgtataatcagatactaattccaaggtcctgactaaagggattcaggaagtgaggaaccactctcgcctttaaccatatctgtcatacaatcttgggatatctatcattccccagtacccaggtaatttcctgataactctcaggaaaaccagggaaaaaaaaaaagctaactaggcagacacagcagagacagaggcagctgcctacacaggaaatggaaaacaagccttccaaaggccatctgccctgatgggctcttctccaccataaagacactctttaggttaaagcattgcatcctgaaagcttgtgtttaaatgggaaaaattagcttgaatgggtgaccattaactaaagtctaagtggctttcccagtaggattattctagagagcagacttcccttgtctcctatgatcagggaaaggtcagaagaggccttggtccagtggaggcctcagctgggctactagcccctgggtctccatggaacacccagggctctgaaatttgaggacaatatgagagggctaatctagtaaacatcatctccattttacatattaccaagggggctagctcctaagaggagagaaaagagaagttccactaaggtctgggcctggatttcccactctataagtacagtcctgctttggtgtcagacatttcagaaatccttcaaacactcccagaagccacaaagaaaaggagctcccttccctagaaggtgggttttctgtatatgatgagggagagtattgggctcacatatatgccacaccaacaatatgactttcactcagaggtcaccaggaaagatgaaacagccactcctcaatgggaaacacaatttaaatatcgtttgtgcccatgtcccaccatacgctgctgagcaagagtaattaaaatagaggggaaacctgctgacaagttaataacagccctaaaatctcaaaggggtaactggaaaatagctcatcgttaacataaaacagaaagccagccctcaggaaatgcccccaacatacaaaggagatgaaaggaatagaacagtgagggatctgaaacttttctcactccatagacctgtgagtttttgctcatgtctgcttttatcttgtttctccccagggacactggttggtagactcacccatatcgagctcttggtatcaaccacagggatgctgtctctgccttgaagtctctggaatacagactcttcagaaactgagctctgctaagtggaggtcaaacagtctggctggagatctttcttccttctgcatttagaggtgaattcagatgttcagcagcgctcccagcaagacagcgtatggtggacatgctcacctcaggctcagcactccaacagtatggacagtttcagagctactggctgggtgtttagctggaggaataaggtagggtgagaggaccccagagaaattgcctgcatgctacaggtaatcaaagcctggtctctgtctcctatcccagaatatgctcttataaacctgcaacaactcctcagtgccaatttctacaagacaaactctttagggtctcacaagaccttctacaactttgctccatctttcttgcctgaccctgctgcaccctccccaactctccctttcctccagccaggcacaactggtcccctcctcttgaacattctagcctttgcaaattttatttcccctgctatagaagcttctttcttccttttttttccagaacaatttcaatgtatctgtcaagaaccagctctgtgaacttgcccctttatgagtgctttgccttttgtttcctggagtatattcacattgcatcgaaagtatctatttatctaacgataatgatagtcagcactattaattgaatgacagttgtgtgctagtcaagtacattcacattgcattgaaagcatctgcttaataataatccaataacaatgagaatacaataatgataattagcatcattaattgaatgccaatcatgttctagttactgggttagacacttacatatataatctcaacacctcataaaagcctttaagtgagtattattatccctatttcgagaagttcaaaaagattatgggacttgctcaaaaatttatagccagtaaccagggcagaattcaaaccaggatagcctgtgctatcctcaataagacagaactcctcaatggcaggaactatgctttattcattcctgaacccttagattaatcaggcccttaaataaatatttgcaaaaggaagaaatagggacatatcggggaactttattcatgttaattgccaacaccagttgaatgtttatcattagccaggcaccttgcttagccctttacttgccttcttttcatttaatttttgccaaaatacctatgagataggcactattattatcggtgtctatcatagtacctgacacaaaatagatgctcaattaatacatgttgaatgaattaatctccattttagagatgtggtaactgaggcttagaaatactaagtatttggggctcacacctgtaatcccagcactttgtggggctgaggtgacagaaccagcctgggcaacatggtgagacactgtctttacaaaaaagatttttttaattagctgggtgtagtggcacacacctgtagccgcagctactggagaggctgaggtggaaggatcacttgagcccagaagttcaaggctgcagtgagtcatgattgtgccactgcactccagcctgggcaacagactgagaccctgtctcaaaaaaaaaaaaaaaaaaaaaacatatacaccatggaatactatgcagccataaaaaatgaagagttcatgtcctttgtagggacatggatgaaactggaaaccatcattctcagcaaactatcgcaaggacaaaaaaccaaacactacatgttctcactcataggtgggaattgaacagtgagaacacttggacacaggaaggggaacatcacccaccggggactgttgtggggtggggggaggggggagggatagcattaggagatatacctaatgctaaatgacgagttaatgggtgcagcacaccaatatggcacatgtatacatatgtaacaaacctgcacattgtgcacatgtaccctaaaagtataataataataaaataaaaaataaacaacaaagcattaagtatttgggagacaaagaatgggaagccgcacagctgagactagaatccaggtacagccttcctcaagcccatagtaggaaggtgggcaaacaacagtctctcttcctttattgctagtctggatgaagtgcagcatttgagatttccaaatagggtagaagagttttctgagttaagccttctctgtagtatgctttgaaagctgatatatcatggaaatcacccccacactgtttgggaagcctggattctggtcttggctttttttgccacagcagaataacttcagtgagccacataagctcccagagtgcagttcctgcatcggtaaagtaaattgttattaggctcacatgacaattcagagggcggtagagaatgcagtgctagtgtgaatctatgacacctgtgataaagataattccatctctgattatgataatgaccaaggcctgataagggctcctgtgtgttcattactctgaggcaagacagagggtgcagtgagtgggcagttactccatacccacaactcccccagtgggcagctcccagaaagtccaattagcagccttgcaccctgcctggtcctagacaactctggatccccggagaagctgccgtcatctctctccctctctgattttattaggatcataattgtacatttctccacttacctgtggggatgaaagggcatcagtgtttccataatgcatgactctaggcgtgaggctggtgccccacgcatcacttctcattgcaatgatgcagcaccaaaaagccatccttcatcatgtggaaaacactccccatattgcagtgtctctatttgcaacagcctccaggatattcatgtgatagatcatatatttgcctgaatcaatatttccctgacactgggaagtccgccatccatccgtcccattttgtttgatggagctcttctgtgttgcctttactcaggctgacgggatcacaagccctccttccccacctgacagatgatgtccagatgaaggattgcttcataaattagcatcttgggagcagagcccccagaactcaagcaaccctgcacaacaatggagcagacagcaaggagacatggcatgaagggcaaaggcatagaacttgagctggctattaagtatatattacctttgtttttaatataaatttttaaattgtaagtttttgtgacttaatttatagtaatggccatgtttaacaattggctggccgggcgtggtggctcacggctgtaatctcagcactttgggaggccgaggtgggtggatcacgaggtcaggagtttgtgaccggcctggccaatatggtgaaactctgtctctactaaaaatacaaaaattagccaggcgtggtgctgcacacctgtagtcccagctactcaggaggctgaggcagaagaattgcttgaacctgggaggcggagattgcagtgagccgaggagatggggccactgcactccagcctgggcgacagagcaagactccgtttcaaaaaaaaaaaaaaattggctgaagatttcataaccggttcttgtgaggtggctcaagttggctccagtacaccactgcgtacctgccttccttcctggctccctgtgacctgtgacctgctcagttctggcctctttatccttcacctggatgattttaatagcctgctaatctgtgtctcagcttctagtctcctctattccaactgatatggtttgagtgtgtgctctaccaaatctcatgctgaaatgtcatccccagtattggaggtgtggcctggtggaaggtgttcggaccatgggggtggatccctcatgaatggcttagtgccatctccttggtgatgagttcatgtgagatccgattgtttaaaagtgtctggcacctcccactctctctttcttgctcccgctctcaccatgtgagatgcctgttcctgcttcacctcctcccatgattgtaagcagcccaaggccgcactagaagccaagcagatgcttcctgtgcagcctgcagaactgtgagtcaattaaacctcttttcttttaaattaaccagtctcaagtatttctttatagcaatccaagaatggcctaacacactcaccatccagttttatctttccaaaatgtagctcctagtctcccctgctcaaagacgttaactctccagtgactctgaatgaaacatatgctttttctaaacagccaatgtcctgtacgaagcctaagatccccaaactttctagttttatctctcaagcagtgatgtgcagctccctagcacttcagagactgacaagcgtgggtctgaagtttgcttttgtcatttattagctgggtgacctggggcaaggtatttaactttactgacgattactttccccacctgaaaaatggataaattaatagagttgttaggaggattaaataagatcatatgtaatactccaagaacagtgaccagtccattttaggcatccaataatagcaatggcatcagcttctttttatgaattcctcttatgtataaacacgggttctttatctcattttatctcattgttaccccaaactgatagatcattagagaagtttgtcagccttttctgttaaatgcagagtcacatctctcatgaaaatgctacactctgaagccaatctgttttacttcttactccttacaagtgcctaccttttccacatctaagcttttgctcctacactcctacacctctcaaaaactcagccatccactcaaaaataaaaagacaaccgaattaaaaagcatctgtgcctgacctgggcccaggatttgaatagacatatgtcccccaacaccaaaaaaaaaaaatacaaatagctaataagcacatgaaaagatgctcaacatcattagtcatcagggaaatgcaaatcaaaaccacaacgagataccacttcacaccctccaggatgtctatcatcaaaaagacagactataacaaggacactgataaattgaaacccttatttattgctggtgggaatgcggtaaggtgcagccactttgaaaaacagtttggcaattcctcaaaaagttagacatagggataatatatgacctagcaattccactcctaggtatacacccaagagaaatgaaaacatgtccacataaaaacatgcagatgaaagaactgcaccatttctcataataggcaaaaagtggaaacaacccaaatgttcattaatagatgaatgaataaacaaacatggggaatatctatacaatggaatatttttcggccatgaaaagaaatgaagtaccgacaccttttacaacatggacaaaccttgaaaacattatgctaagttgaagaagacagccacaaaaaactataattccattaatataaaatacctagaataggcacatctatagagacagaaagtagactagtggttgcctagggctggggagtgaggggtgaatggggctgactgctcatgactgcatgtttctgttagaggcgacaaaaatgttctaaacttagattgtgacaatggttgtgtacctctgtgaatatactaaaaaccactgcattgtatgctttaaatgagtgaattttatagtatttgaagtatatctcaataaagatgttttttaaaaaagaataccttgaaaaaaattccatccgtccatcaaagcttattgcagggggtggggggcccttcctagtgctcaccttcctactccaaagtggctgccattgctcctgctcttgagtcccagtattcactgagtgttgtttcttctgtcatttctcaccttttgtttcctattgtagttgtctctgtattacttttatctattacattaagtggcctcaattttgtgaatatgtaaaatggaacagattcagtagtgttttcataagatcactcacccttgtattctgttttttcacacagggaagaaaagaatggtgtttcagttcacacaagaagctgtgcctgacttgggcccagtcactagagcatgttgcagacaagaatgacatccatcatagtgtgtgtgtgtgtgtgtgtgtgtgtgtgtgtgtgtgtcgagtgtggtagctagtagatctccaaactgggtaaaatctgcagagccatgattgagttttgcaggctgggtcttctccatgtccccaggcccagggctgtacctcagctacaaatccaaggaatatgtgtggaatccttcccttggcattcagtttgagactctgtcaccaacataagatcaaggaaacctgctgacagggtggccaccaaggacaaaacgaaactaattcttcagtggttttaagtgtttctttcaccagggggcattttcctttggaacgacaagttgtctttgtttttatttataaacaaaaatttgctgccacttccagcttccctcagggtgagccctatgcacccagatgagcagagaaaggctggtttttttcctttgtcactgggccctccacggggacataggagactggtacaagtcctgtatttaggcacaaagtattgagccatgtcggcaagaccaggaggaggtgcaggataaggagagggtgtcatcgaccacaaaactgtgacacagaggcaaagaggcagaagtaggggaaggctagtccgctcagggcaaagggaggaggcctttacaaatgagagactcggcagcctctctggaaatgggaacttgtgctgaagttggctctcatggagagctcctggaaatcgaatttaactcttttcccccagtgaagatcaagcagagaaaacagaggcaatccattttcagagtgtggcccaagtagtcagttgctgccagggtgtcactgatgcctcatttgctggtcaccgacattgaggtgaaaatacacaacaattggcatatgtgcatgcacgcgctcaaaacgcacacacactacccccacttgcagctggcactgtgggtgctaaagctgatctacagatgcagggaacatggatgtgctgcttataagcctgagctgggagacaaacccatagtgctcttcttttggaaccagatgggaaaggcattcatgtatcgccacctaatggtacttacacctcattacagtcttggacctgttcaccatcaacaggtagatgggtccactgaggactcacaatgaaagatgtaaacttggacttcaacttccaaatgcatatcccaactctctggtcacagctcatggttgggccttaatgagtgtgtgcagggctggttaccagcgctgcctccgtgcccctattccacatcctgtaccttttggctgctctcagctatttctcttatacttcctaggatcccctttagcacaaagctttgatcccttttaacaggcactcctgaggactattactccaacagttatcactcacttttccagatgtagaatcaggtgccgaggacgggtctaccaagtgattctgcctagagccaacctgccacatctgccttcgccctatgctccaggttccagcctgccttcgtggataagccctgggtcaatgggatttgaaaaccaaaggatatttcatcccctgctgcaggctcaagggcttaagggctgacaactcgctgaagtctggggaggtctttttggcctctttcacctgccggttatatcaaacccctgaggttagctgagtggcccctgttgacttttgagagccccaggtgattggaaatcctgagtaaccaataaggaagaaagggcctttggggttgaccaaactctagagtgtttatattcgtgagagcaggctacaatggccctgcatctgccctcctctccctgggctcagcagttactctctccagcttctttcaacaaaaagagtgggtccttgcttccttttaaaggttggaagaacaaagtccagagatgtttagccagttgcccatgatcacacaatggagcccagtggagagaaagctaggacctgggtccctcaactcccaagccttggcacctccttctcaggctggcctggacttcagacacccctgatgaatgcccctctctgtgaaatggcattgacattgtaaaatgcactttggttggttggaaggtaaagagttgtcttaagtgacaggtccctgaatctactgtacctttgagtagagtgcagtatgagaagctgaaaccattggtgtttaagtaaatgtacaagctgttcacatgctctattctcttagctgcaatagtggaaggttctaatgccctaggtaggggtgggaaaacctggttgtgacaaacctctctctctctctctctctctctctcctctctttttctctcctactgtctcttttcttttctatctttcctcttccttctccatccctctttcaagttcttcctcagtaattccctacggtatttgaccaggcatttcttttaaaaaagaatattcaggaattttttaagcccaaatgcaactctttcccctgattttgtaaatactcctctacctaaacatcccaccctcccttaaagtcagctggaggaaagtcctaatcatctttcctctgaactggatggactcggggaggaacccatgaaagaattattcattctatcacacgaatatggtatgaaagcagtcctctctgtttttcccactgcatatggcatactccattaagaggtggatccaaatgttacccagccatagccaggaagaccagagagggttggacgggctaaagatgcttctacatgtgctaggcacaggagacaccagccatctgttaaggggctaatctaaactctgttcataaaatgctcaacagaaattgctggcaacaacacattgtcctttgattatgaatccagctgtccattctagaataaggaaaattgatcccctaataatacaactaacttttttggtacagaacttacctagtttttgaactgaaaggctcgtgtcctgagaaatttcttagtcttaggcaaactaaggtggtctctatagaccccaacgttgtgctgttgctgctgctgctgagatgatctccttcaccttttcttacacctttgcaataatcttccatagctgtgagtaacctagatgcatctcttcctgtacccatgaggtacctagcctgcaggatggtattctccctccgcacccctgctccatgtccaccaacctggttcaacttcttttgtccatcaataattccctggcttattctattcattttctagtttgaaatacttgacatctttgcttagaccctctcctgcattgcctatatgcaattaatcaccaagtcctattgacttttattttgagatgtttgtggaatctgcctttgtccatctttactaccatgacaataacaataatgataaaaataaactccaactttaaggaccaatcagagtggactcaagaaagaatggaaggaaagaaataggaatcaacacgtatataggattcagaatttctcaaaattatttgatgatgaaacttacttttcacagaacagctcttgggaccattgttccatgaaatacagtttggtaaaggctcatctaacccacaaatggactgttgtgcagaattagaaagaatgttcaaaaccatcaagacaagttctacaattttatacatgaaaactctgagacctcaggagataaagtggcccaagccacacagctcacttctgggagacccaggctttgaatcaagtactgagttccttactctccaccatccactactccctaccagatgccctgcattctgaaatagcctcctatcttcactgaagtcctactcgtaattcaaggtcaagcttactctttagctcctctacattctttgtcagtcatacaagcctacactgataccaatcttggctgaatttcaataacagttacttttatcattatgtttgctaccttgtattaggcactataaacaactagtccacgtaccactacatgcccaacttatgcccatggcattcattgctaatccaccagatcatccttttcaattgagcccagaaagagcttcagaatcattttcagcataatactctagatagccatagccagccagccatgattagcacgtgagatgaaacttctttaggcccaactttgttgtttaattatttcatgtgtgtgtcttaattgctttgccatgtgggaccttcctaaacagcaaaaacaatgtcttcgaattctttcacgtaactgttagctgcaagtatagttctttgccctaataagtgctcaataattgtttgaatgcctgaatatcaaatatgaaaatgagaataattaacatttatacaatgtttagttacagagtactttcacatgctataccacatttaattaattctaacaacagcactggaaaataagtataattatgctctttagttccttctagacagaaagctttcatggggatgaaagactgttccttagctcagagtagggaatcaaagtaggcggggcttagttcagagtgggcggggcttagctcagaataggccatcaatttgtagaattgaagtgtgaactctgaggatgaagaaacagggctgcaggggtgtgtaacttgcaccaagtcaccctactttacattgcgccacaccgcccagctaagtcttcgaaatgttcataaggtgcttgagcactgattcctttgcctgttcaatgtttgaattccactgtcactggcttttcttttttcctcgtaatgaagcaaaacctggtgggttaattaatccttcttgatgctgggcagcagtaataggatcaagaagaaagcctttctcctcctgtgctcccaaggatgcccagcgggagactgtgaggagatggaacacagccttcactctgttaattgttcaagagcaaatcacctgtcagcaggaaacagaaaaaaagacagcacgaaattcagagtcagaacaataaataacattcagtgctatacccttgagaaaaataaagctgccaacattctgtttcttagggaaaacaaaaatgctcagtgtttctgcagacctgtgaaatccagtagaaaatactgcagcctgcccagactggtcgtctcttttatcaactcagggtgcaccagcccaaaccccacagccagcaccactgtccagagagacaggcacatgtatgcaggaataaaaatgattacgcaaatataaaaatggatttcctaataagaaattccctttggtgaaagggacagaggagcatattacaatggcctcacactattctctctgtgtccactcccatactcctaaattcaccctaagtagctccctaaggtgagtcccccatgagctggcctctctaccactctatagcctcgtacctcaggcttttccagcaccaaattgctcctggtgtcactccggacactgctcacacctcagcctctgctccttcctgcctctccagcaggagtgacttttctgtcacctgctaaattctcctctacccttaaggatcatctcctgtcaccacttccccacctctagcctgtgcatctggagcactctgtgacactctactatttgctgtgaccaagcaaacagattcctgagccagataacccagattcaagtctattctctgctacttattgcttgtctgatcttggctgtgatctttcttgatggctgggtgtctctgttcctcatttataaaatgggatggagataataatagttcctacttcacagagttgtgtgaagattaaactgtttaatacatgtaaattacttagcccaatgcccattgtgagctactatggccattaataatttcaccataatcatcagcatatccatcttcttctttaagctatgagttatttgagggaaaggtccacatcttatccttccctttatgcccatcacttagatagttgctatggtttgtatgtggtttgtcctcaccaaaactcattaagtttgactgccagtgaggcagtgttggaggtgaggcctgatgggaggtgtttgggtcataggagtttcaccctcacaggtgccttggtgctgtttttgctgtagtgagttcttctcaagacacagggttagttgtagcagaaatggattagttctcacaagagtggttgttataaagtgagaacacacttcatgtttggccccttcctgtgtttacttcccctttgaccttccaccgtgttataacaaagcaccaaagtcctcaccagaagctagtgccatgcccctgaacttctcagcctgcagaaccatgagataaataaaccttctttgtttataaattgcccagtctcaagtattctgttatagcagcacaaaacagactaagatagaaaattggtgccaggagtgggatatcgctataaagaaacctgaaagtatagaagcagcttggaaatggttaagaggcaagagttgaaaaagtttggaggagcagaccagcaaaagcctgtattgctatgacttaaacattaagtgtgattctggtgagggctcagaagaagacaaaaagatgagggaaagattggaacttcttagagatcagttaagcagtcatgaccaaaatgctgatagaaaatggaccataaaggccattctgatgaggtctcagatggaaatgaggatattgatctggaaactggagcaatggtcagccttgttatatggttgcaaagaacgtggctatattttgtccatgccctagggttttgtggaaggccaaacttaagagcaataatatagcatatctgtggaaggaatttctaagcagcaaagcactcaggctgctgcatgtttacttctaactgcttacagtgaactgctaggaaaaaagggaagcaaagtagaaagatctggaaaattcacagcctggccatgtggcagagaattaaaaaaaacatttttcaagagagacattaagagtttggtggagcaacacttactaaagagattagcatggctaaaagggaatcaggtgcttatagtcaaaacaatggggaaaaggcttcaaaggcatgtttaaggtttttgagggcacccttcccattacagacccagaagcctaggaagacagaatggtttcaggggacaggtctggggcactgctgccctgtgctgcctcgggatgctagctcccctcaggatactactgcaatggctcaagtgaccccaggtgagaaaaagagactcatgtggccactccagagggtgcaagcagtaagccttggaatcatgtggtgttaactctttaggcctgcggaatgtgagagctatgaagtatgtcttcctccaccaagattccaaaggatatatggaaaagcctgtgggcccaagcaaagacttgtcacaggggaggagcgctgcagagagcctctactagggcaatgacaagaggaaatgtagagttggagctgccacagcgagtccccaccagggcaatgcctagtggagatacaagggcagggctgccactgggacccccagaattgtagagccactggaacatgcaacgtcagctcaaaaaagccacaggcatttgactccaacttatgagagcaaccaagtaggatatgcccagcaaagccatgagaacagtgctgtgcaaggccttgagagctcaccactcacactagtgtgcccagaatgtgggacatggagtcaaaggagatcattccagagggttaagatttaatgtttaccccgaagggttttatttttgcttggaacctgttacgtctttcatcttttggtgtatttctccattttgtaatgggaatatctaccctatgcctgttccaccattgtgtcttggaagtaaataacttgttttttattttatgaactcagaactaaaaggaacttgccttgaggctcagataagactttgatctttggacttttacactggtgctagaacaaattaagacttttgggaccactaagagggaatgattgtattttacctgtaagaaggacatgagttttggtcaggggatgggagttggccaggggtggaatgctatggggtggaatgttatggtttggacatggtttgtccccaccaaaactcatattgaagtttagttgctaatgtggccatactgggagatggggtctaatgaaaggtgtttaagtcacaggggctgcacctcatgggtcttgatgccattcttaaaacagtgagtgagttctcactctcataagactagattagctcttgggggaatggattagttcctgcaagaatggattgttataaagcatggatgcccttcatattaggctccttcatacatgcccacttcccacgaccttccaccatgttatgacacaacaagaaagaccttttcagaagccagcaccatgtccttgaacttcccagcctgcagaactgtgagctaaataaacttcttttctttataaagtatccagtatcaggtattctattatggcaacataaaacagactaagataataatgttcctgaacacctgtcttccctctcccctgcatgattattaacctttcctacccattgcttgtgacttgcagtgccttcctaaaggaagggtaccttgcccattgacgacaagcataattatgtacttgtcttggccaataaaatgtgaatgaaagtgacagtgttcccgttccacaaagacacttcaagaggcattccatgattttactagctcctttcgtccctctgccatgagaatggcatgtctcagataaggttgctcctccatcctagatcccagatgccaggaaacagcttggaacaggagtagaacgacagctgactggcaatgagcatgcaatatgaacaagaaataagtatttggttataagccactgggttttggggttgtacttatcacaacataacttagtgaaagctggtgaacacagcacccaataaaagtttgttgaataaatgaatatactgaaaaaacaatgaatgagtgagtgaattatgttttctccagtacctagctaggactatggttcttatgtacatcaccaaatcaactaatttaagcatgtgctcttttaatagtgactatattatctgacacttactttggatataaatacttttaactaaaaagtcaggcatgaccttgtagtcatccagacaccactcggaatatccatccccagaacccatgtggaagtaaagtggcagtgaagagaccaacacttcttgggcaactactgtttcaaacactttacatatgccagctcagcagagccccttcaagtatattttccagatgtggaaacaggctcagaaaccctgagaaggtcatcagagatataagtgatggtacacgtgtgaacgcagatctgtttgagtacaaagcccatgttttccattaaaggacgctataaccatcaaagtacttggagataagccagggaagtctggctaaccaacaacactcctcctatactacctatattcctcacctctttttcttattttttggcacatggcccaaaatgttaacagttggagattcaagtgagataatttgaaaatgcaacactcctggtgggattaaacagactatccatcattagtctcagtaaatgaattttttaaatcagattgttttctcatcacaaaatttaaatctgtccaagaaaagagaaaatgctagtttgggtataatagaagagggagaaagtggaaagagtagctcccatcaggggctgcttttctgtggggctgagcaggtaagataatgagtgaagaaggctgggtagggtattggcaagctggagtccacagacctgacctaaaggggacagctcctggtctatttcagtcaattactattaggtaggaatgccaggcttttagaagttggaaatctggacatgtatttggagtctccaaatgtttaaatgttggcaactaattcaagttgtttttttcaatactgtatattcgaacctttcttccaaaagttgttatatgggttctgacatatgtgtcacctatttgttactcacaggacacttgagagaattgcatgtttagagcagcctacatttagaggagataaagggaagggggaagtcagagcatattataagacccttgtgcttaggagggagaggagaaggggcagaggacagtgaaaggcaggtacagaatgtaaatggaaagtcactgaaaaacttcttttaatgcaattggcttggcctgacttcgttcttccctatttcagcactgcatcaaataaaagcaaattctgataaggaaaagccatttgttccagaataaacttcattactctgctaaatatctttactggatttcatttgatcctagtcattctcaattcaatattgcactcagccagcttaaccttttaaaaggagttttattgatggtgtgttatgcagacattaacatttctcaaaaaaaaaaacaagaaagaaagaaatagaaaaaattaaattgctctgcttagcgatcttaattatctggaataattagctccagtcaattctccaacaagagctctccagagaaatcagtgtttagggaaagggaaagaaaggaggagccaaacttgggatcaaagcaagtatttggcaagttatttggaaatatctaattaaccccgagagtgaagctgacaatcaaagccactttttttggtacttaattattaaaagcaaatgaagaaagtaaatattcagggaactggtctattgagcgttggaaaataggagccactttgtgccaaagtactcaataatccccaaatacattggcatgcatgctccatgcctttgttcctgctaatctctgtttaagatatttttccaccccatctctgcttagaaactcctactaggcctctatgatatcatccctaacccacgtttctcccataccagagaaaattattctcccctttttgtccatagtactcatactgttgtgccttcattaggtatttttctgcccccctagatggtaagcatagtgcatattcatctctgtatccccagtgcctagtgtgtctggaacagagtaggtgctttataagtggaagttgaatgaatctacagagcaacagctttttatgcagtctggtacatactggaaagtcaacaaatcttcccttgtttttctttcttgccttttttctccctcctcctctattctttctttctttctttcttctttctttcttccttcctttctttcttctttctttcttttttctctctctccctttctctttctttcttctttctctttctttctttctccttccttccttcctatttattttagttttaggttctaccattaaaggtattgatttcagagctgctgggcccaaacaacagtgttctcctatgtgagtctgtggttttgacttgtgattgactttcaataagccaatgcaaatttcagtggagtggctgagaaaccccagtgcccaggtagcatgggggtactcgatgcacaaccccattagaagagaaatttcttgatttgcagccactactcgggcttgcagtgtgagaaacttcagttcccagaagaagaatttaaagaagaaatgatgcacaaagcatctgctaatgtaaccctgggttattttttaaaactagcttgtcactgttggtacataaatcagtacagccattatggaaaacagtatggaagttcctcaaaatactaaaattagaactaacatatgattcagcaattccactactgggtatatattcaaaagaaaggaaatcaacatatcaaagagatatctgcacacttaacgtgtattgcagcactattcacaatagcctaaatatggaatcaacctaagtgtccaacagatgaatagagaaagaaaatgtatatgtacacaatagaatattactcagccataaaaaataatgaaatcctgtcatttgtagcaatgtgcatggaactggaggtcattaggttaagtgaaataagccaagcacagagacaaatattgcatcttctcacttatatgtgggagcttaaaaagtagatgccataaagataaagagcagactggtagttgctggaggctgggagagtggagggaagggggaatgaagagaagctgattaatgaatacaaatatataattatatagaagaaataagatatggtgttcaatggatcagcagggtgactatcattaatattaatctattgtacatttcaaaatagctagaatgtgcgccgggtgcggtggcttatgcctgtaattccaacatttttgaaaaccaaggcaggcagattgcttgagcccaggagtttgagatcagcctgggcaacatggcaaaaacccgtctctttaacaaatacaaaaattagtccacatggtggtgcacgcctctagttgcagctacttggagggctgaggcaggaggattgcttgcacctgggtagaggggagtccagcctgggtgacagagactctgtctcaaaaagaaaaaaaaacagctagaatgtttcaaaatttgaatgttcctagcaaaaaggaaagataaatatttaaggtgatggataacccaattgctcagaattgattatatgaatgtatcaaattatcacatgtacccccaaaatatgtacatctattatgtatcaataagaaaactagcttgtgtacctgccgtgaggaccatgacaatcctgggggccatttgtagaatattcatccccagactctgaactgagtcttgaagtctgagtaagaatttgctgggtgaagaaaagggtaacgttctttcaaaaggagagaccagcataaacaagggtcccggggggcagaacatcatgataggagtaggaatgcaggtgaggttttggttttctgcagaatattggtactctgctataacctgtaaatagagagtaagaaagtgaggcaggtagtgttaagaagagacattcatagtcattgctaatttctgagcttggatgtggctgttagttttagcctcagggtacactgccccctgcacttccaatctcactcaggtgtcctacaagcaccccagattcaataagtacaacaattgatgctttacatttctcgtaagctttcttctcctcttaacattctgttaattgcaccatcaatcctagttgtctaaggcaaaattcttggaaatcattcttgaacctctctttccctctctcccacacccaattaaccagcaaatcctgaaggcttagcctcctaacatttcctgcatctctgcattctgccctatttccattatcactgccttcatcatcttttcccctggacaatagccacccataaccccttacctccagccttgctcacctcaactccaagcttcctacctatcactcctctgccagcatgttcaggataaggttcaaactcccagacatgcatataaggacttctgagccctggccttgcctgccccttctgcttgatctctgtctactcacttctgcattgcactttccactttgataaatctgaatggcctggagttccatgacactatatgctatctctcatctctgtgtctttgctcctgctctcccctctcctgaaccctcctctcacttttcttcagctggctcttactaaactttcaacactcagttctggcatcttctcttccaggaaatcttccttcaaagtccagactcccctagtgtcctcctctgtgctcctgcagccccctgtgcacattcctgccctttacctcccaccatttcatggggattaagtaattacatgagatctgtctctccaggctctggcttcctcaaggacagctgccaccaggtcctatccatcttgtaagtccagttcttaacgtggtggcactcggctgttacacatatgtttgttgagctgcactccaggctgtgccctctgagatgactcttttggcaaatagaaaagctcagcctaagccaagatgtgcctgccccaagacacagcaaaggaaggtccagagaacagccaagggctccagagagttccagaaagtttttgctctgttttgtttataagtttctgaaaagtggaaagcatacaggctatagagtccaaacatatctgggttgaaatcttagtcctaccagtattagctaatgtaaccattggagaaatacgtcatctcggtgggtttcaggtaaattatctaaaatattaggattatttgcttaaatggaaaaactgtttttaaagcccctagcacagtaactagtacaatgtaagtgcccaataaatgttagttctctcttgtagcctccatatttctggagaaataggagatccccatggcctggaatagaataactttggttgccagaaacacaagatattcttgttttctacatgccttaaaatcttatcattttaaactctaattactagtactaattggctttcatctcttaagagcctattttatgccaggtactatgcaagatcctcaaggtacgttttcttacttattatttgtgatggtagcgatgttagtaaagttagctcttactgagtccttaactatgccctgctctgagtcctctccaagtattaactcattcagtcctcacaataactttaagaggtagatagagtatccccattttaaagatgagaaaactgagtctcagagttgttaatttgcccaaggtacatattatggaataatgctgggactagaattcatctgcacaacaccaaggactctaattttttcatcttgctataaagtatcttcagtattgtacacagttttattaaaagttaagatgatagctttattttaatacctttcctcataatgccctttccacttttggaaactttatgttgttcaatcagcaaatagtgtattgggtaagagaaacactaggttattttcaacctcaagctatttaatgagatcattggagtcaactggtcaactgttgtgacctacgagagagaatattttctggactgtgtctttattactgacagatcattgtggcaatgtgtaatattcaataactgacttgtaacataccatactgaattgtactacttgtacagattgaattacgaatgtgcttttataaaggccaactctttggtcccaggaaccctgaagacctcaagttaattgctcattcagcaagaatttactgaatgcctgataaatcccaggccttattctagtctgaggattctgagatgaataagtcactgtctcttctcctaaggtacccacagtctcactggggagattcacagaggaattgacttcacccagggtgacaagtaaagttcccctcttcaaatccaaagcaaggctctaatgtctcaggagtgttcttaatgcatcatccagaactcccttcattttatccttagatatatattggcctaaaaagcctcagcctggcatagtggagtggtttggggcagagtagactctcaatgtggttagagagcactgaggacccctgcttctatgtggactctcacatggctgagtctcctcagccccaggtgggaaaagggctggcctgaggttctactgctgctgagtcagatcctcctctcttgctagacaaagagcattttcctccccaacaacagcgtgtcagaccgagctccctgcaccatcctcccccgcaatctcccaagcacagtatcgacacttgagatggactggaaggccctgagcaaatattggctcttgagaggttaataatttacaaatgaactacagctgataaaaaaaaaaaatcagggcctttgctgctgggatatggtgcatgacatgcatgcagccagtcttctccagtgggcaacaaagtggcaggcaccagctggctcctttataaaggtttagagaggtgggccagaccttgttctttcccatataacaacgggagtcgtggggagcccgagcgtttggtcaaaccaccaatatttcttctgtacacagaagtccagtgagatgcataggcagcacgcttctcctgggggcagcttggcagctccattcttcatgccaggaaggacatcaggatttttattcaactccacaggcctgccacaccatcccaccagcctttgcaaaccacttctgtactgtgacttagacccccttggacaggcaggtgcctatgtttgatcccctgcagagagaaggaaacaaagcccactttttaaagctgttgttgattccttgggattttggtgggcacatacaggagtgagcaagatgaagcagaggaacagaaaaagcctgccagcaagcagcccgtgcatttaggagctgagaaaggagcccacaacaaatggacagaaccagaatcattctgtgtggaaaagaggtggtttagaagagagggggcggtggctgtgttcatcctggggagtagcatagcaaggaacaaacagacaaggttcaagaggatgaaatgaactcctccttcaggaaagaaattactaggcttggacatgaagaaactgttgtgttgcagaatggatatttctattccctgctctactccattgtctgggccagtacctatggaaggcagattaacaccagagagcatttagtaattttagtaattttctcaggtgaagaaccagaaactctttttacatagtagtccataaaagctatgctaagaagaaacccattctggcagtgagaaagtttgtgctgttttatcatcctctccccaccctgctacccagcccaaagtagaacccagaacagcacacacataatcacactcaacacacaaatacacacacacagaggacataatcacactcacacatacacacagaggacataatcacactcacacacacacacacagaggacataatcacactctcacacacacacaggacataatcacacacacacactcacactgcacatattacactcacactggacatacacacacacacactggcacacgcacatactcagatgtactcatacacgtgctcagacttgcacatacactcaaaagtaaccaaacacaagtgctcacacttgcattcacatacatgctcccaggtgcaaacacacacgctcccaggtgcaaacacacacacaaactcatacctgagcccatcaagaagagagctgggggaacaaatgaccatgcctgagcctggggagaacagatggagccaggcagagagtcctggtgggaggctttcagccctgcgcttccccacatctgcaatgtccttcgtgatcatgtccacacacctccaaatcttacccacatgtaggtgcccccagccagagaggctgctcagcccttgttcttgggaactgcttccactgcacaaagaggggtcttagcagccatgtttctggggacagttcccctggccatagctaatgagtttacagactggagtcctgacactctgcttgattaatcacattagatcacctggaaactgaatgtttcatcagagaaacaaagaatgtttggcactgaattacattgacaggggaactctctggggaagggccacagattccaccacagggtccctgaagatctttgcttctcacccctacctgagacttacttgttgagttttttcatgggttctgtgagatagtagagtctgctgcctgtgaacaccttttgttacaaaagctagactcactttaatttatttgcaaaaaaacaattaatataccatccattgtaattctattcaaatatccactcctgcccatgtctccagctagaagaaatctgttcatcctcagagcattcaaagcatttttatggcctcattttcttccctgtaccattcatttctctcttacccttagtactagatcatatgctcctttaaggtacaagttatgtttttttgttttgttttttgtttgagacagggtcttgttctgttatccaggatggagtgcagtggcatgatcatagctcactacagccttgacctcctggattcaagggatcctcccaactcagcctcccaagtagctgcgactataggtgcaccaccacagctggttaatttttcatttaaaaaaaatttagtagagacaaggtcttgccatgttgcccaggctggtgtcaaacccatgagctcaagcgatcctcccacctcagcctcccaaaagtgctgggattacaggtgtaagccatcacacctgaccagaagccatattttactttatctttttagcccacttagcaattagcactgtgccttccacacaatagtttttgataagcatttgttgtacgaatgtgcaaatgcatgattgaataagtgagctaataagtgatctcaggtgatgttaagtatgtcttttacttttcctccaggaatttatggggtcttggtctcagaagctaaatctcctcacatgtgaatcaattcattttgtttcaacatacatttgttgacatcaaacatgtgccagaccctgtacaaagaggacacaaaatgagtaagacatggactgtccttaaggaactcatgttctcactgcaccaagggtggaggttgtgaataaggcaaaggaacacagctccccgccccgttcaccctgtacacagccctgtacaccctgttcccgggaacagcagaggtaggaaagtcacaaattgtacatccaccccaatcttgcaacaagagtgtattaaaagccatttttcaaggctgagtaagagtttgtgacctgatagacgatggctggcaaactcatttcatcataaatatcacagcagataatatacttcaatacaagtgatgcatctgcctatgccaggatcctcagagtgagtaatgctcctccaatatgggcttctgaaagataaacaaattatgggctgtaattacctgacatttgagtttcctttttatgagattttcttcactctaatcacactcagagtgggaggatggaagatacaattggggcttaaagaagggaagaagaggggtctgctctctcagcagagaggcggatgccagataaacagctcctaaagaaggtgctggtgcttctgtccctctggaatgagaggaaaggctcagcaagtaggaagatgtgcagaaagtggagaaatgagaaaggagagagagtggtgatttctcaagtgtgtgcatttttagacgatgcctgtgacagaaaagaaattctcttctctgacgcatacatatcacataaagacataatgcatgctatgcagacagaattctacatcttcaaactctccccgctgcagagctcaggcggagattcacacactctctagcccactgtggaccttgaatttgacccttcctcctttctgaaggcagcatttctcatctccccttcaaaggtttgccttaaacttcacttttacaaatcctttacctcctgataggctcctgtctgccaacatcgattgttcctccaaaatgtttcctctatgggcctgttgttaatatttgtacttgaaaattaaacctctccttacacaggggaccagactctattgagtctgtggcttcttctgaagggtcagagaaatatttcaagatgggaaggaagtgtgccataaaaaataataaatcaacaaatgagagggtcacagtgtaatgtagtaatctctaaattggacgtcaggaaatctgggtttaattagaatcttcactgtttcttatgaaagtgatgtttctttgcttcaagtcaattttctcctcacttaaaatgggaatcacttatttggtaggttgctttaaggatccaacgtcataatatccataaaattccatgtcaccttctgggttccataaatatttatttcatcccagcctcctaagtctgaataataaaggactataaatgtgcacagcattctttgttgtacttttgaaggtcttttcacactaacaacattctccagaggcaggaaagactaaagcagttgccttttttttaattgatgaagccattgaaagaattcagggaggttaagagacttgcccagagctatgtggagactgaaaacccagattcttgaacctttctgtggtcctcaaagacagcatacttacctgggagggtcaggtcaaaagggggtcccttttatgaactatttaaaaactcctggcatttctgagcagagaagcagcccatgtggcaactgaggtgtgaatccagtggatatgaactttgcctcaaagaaaatgagccagctgacacctgatgccagaaatgcaatgggtgagatcaatggtttgaatctgaaaaaaggcagtggggggggtggggtgtgtggggggcgtgctatgcatagggagaagaagtatgcatgaacttagtgataaattcatttgtttcctagggtaccacaaattggtggcttaaaagaacatattatctcactgttctgggagctagagatccaaaatcaagatgtcagcagggtcatgttctctctgaaggtgctagggaagattccttccttgactctttgtagttttgggttattgccagcaattcatggtgttccttggcttgtagacacattgcttcagtctcttgtttccgtagtcacacagtgatctgccagggtgtgtctgtttttgtgtctcttctcctctccttatgaggataccatcatattggattcatggtgcacctgtaccagaatgacctcatcttaactgagatggccattaattaaataaccaattgaataattaaataaccaattaaataggtcatttgcaatgacccaatttccaaatagaaccacattctgaggctccaggtaggacacaaattttgggggaacactattcaacccagcacaataagaaaatgagagccaagctgaggaggaagaatgaaaacctgagaaagaaaactaggtgaagagggagactatgagacagaagacagacaaagtagagagtgagacagagaccagaggagacagaagtggaaagacagagtgaaaagatgagaaaggaagagaaagagaaggatgctagtgaagcacagatacacagacccctgcacacaccatgtgggagacacacaacaacttggtgtttaaagaatttcccccaaatatgcaccttctctcccactagatgcccactgggcagccctaaagctgttactgtagaaactgaagtagacatcctctttggggttaatagatggccccagtaagagagaccagttagtccgttggccctcacattcctacttcctcccatgactctggctccaggggcttccagttttcaacgttagaacctccgtgatggtttttcaaaaaaaagtctttttttcaaaaggccaattttcaggtggtgttatgtttatttacctcctatagagcagaaaatgggccactcaggagatgcgatgatgacacttcccccttgagaagcagctgttcctagcttctggcaagctgcttagggtcagactgtccccatttttcatccaaccagatgtcatccagcgttattctacagattccatgtcctggtcttttccaactcactggagttttccaaagatctccaaagaggtgctagccactttagtatcagccaaaactgacaccaggaaggaggaatcataaattagcagtaggaaggaggccttggggctggcagaacaccagtgtgcagtgtgtgtgtgtgtgtgtgtgtgtgtgtgtaagggggaggttttcatggagaaataggtggaggtagaaagatatccatcaatatagaccaagaccaagccatttccaagttacggggctctagaaccctctttgtcattggaaaaatcacgcattcactcagaacagaaagtcacataggacagccagaatttataagctaagatgtgctaagtctccccttgctttattttcttttgccatattgaaaatttagtgagtttggctttaggataatagtccccactccttccctatcatagaaaccaccatttaaaaagagaaagccacaagcttgcaggtctcctaagggaaatttcccatgccagcctgacctggccagtagtgtgacgggggagatgtgggtgaaaggacagaggtgggcagacaccaccatagcatcgtgctcagccgctgtgccatcccttcggagcagtgagaggaagagccctctactggcaggagcatcttctgggcttctgggctccccagtcctctcacctcccacagcaggaaaggcagtgaggtgcgcaagcccatctccagggacagccacagaaagagcagtgagttaggagacatttatctcacatggccacacaatggagcatatgccttaattgtcatagcagctgttattcattcgttgtttctcttatttactcattcatagttttttttttttttgtcaccacggttacagtggtctatgttaagtactgaaggtatcaagatgaaaaaaagctcagttcctaattttaattcaaagcaagagcaaagcaagaaaaaggaagtgtaggggctgagtgctcagggtcacaaactataaaggaatggattctttcacctgaaggcaggctttcccatcgcccccacagggctgcaggtgtttggggcctcttctaactctggatctctcctctgccaggtgtctagcacctccctgatgacctgatgacttaagactcccttagatcattctcacaatcaactatggcaccctaacccgtgggcagaataaaaacctcaggtcaaggtggtaaaagaattctggaaaggactggagatgtggaagtttgcagagggaggtggttattactgacctgcctcccctcctcagggttctaggttaaaagagaaggccagacagcccaagggtatgaaatacagtgaaataaacaaaccaaaacagacacgagttttaagcagaactaagtaaaatctatgattgcgctatgtacccatagttctcttgctagacagaatattacttgacctgagaaatacccagcctgtaaattttttgtctctaatatattcttgtggtgatgtcctcttttagcatctgaccagcaatattcttatttgaaagctgcagagactaactgtgggcttgtcatttgccatcggttccccacctttcccttgctctgctatgtcttgcagggatgacgaggccctcttgccagctggactctgccaatgtttctgccaatggtgcactggtggcagcctggggaaagacggggagggaggtcagggtagttatttcttccttgctttgcctcaggccatgcctctgactacattgcatcaggtaagcttcatctccactagacaaggcatgattgaagctcacattggttagcttcagcctcctcctcccactatgactccagtcctttgtaattaacaacattctgcaactgttcatctttaggtttcttcaccattgtttggcttttcagcttccccatcatccaccaatccaattcctgatatccaattctctccattgaaaaacctagaggggcttatgttttcctaagggaaacttaatacatttctgacttgtttagtagcaaagtaattaactagaaggatgttgggtcatttacagaattgccaggaggcagaggggctatgcagccagacacagagcaaagttcattccacggaaacgacagcaaggactcctccgcagccactgctggactccagaatgtgatgaccaccatgagaactccaccagctgtgatgctgagcctacctctgctgcccttggacactcactcccaaataaagttcagattgctctctctgattatctaacttgttaagtccacatcattttccccaccctagcttcagaagactggaaaattgagtttctgatatttgttaacagagtccagatccacatttcagtgagacttgcagggtcaagaattctccaaatatcagaaggagatcagatgcagaagagccaagagaaataaataaatatttgtcccaggaataagaccaggagttagaggctgagtttcagccctagggtcagcatgtgaagaagagaatctctctaagatcactatgttaaaggcaacagaaagactagaactctgggtgggagcttttgccctatgagtataatcacaggccttctcagtacccttcaaattgataatgggcaaccagtatttacaactataggatagcacagcttcctacccaggactgaatgtctacctcactaagagtacttaccagatttggggatcagtcctgaccctagccagagagaacttaaggaatctctggcttctgaatccactctgaggatccacattgattcctaagacatttcttatttctaggaactaggaactaggttcctaccttgtacctctgacccagctccggggtcttgcccttcttcctggaaccctgctgattcttaagaacaaaggtattaattaacatccccattcaatccctctgaagtgtgggtcttgcctcagcccagcagtttttgtggtgaggtcctctttgtctacctccagatttgatcttcctgtagtcgcccctgcctgagcaacagccagcctgaaattcctcctgttacctgctcttagaggcagcttgacataatagcaaaaggatggatttcctttacatcctggaaagtcagtcagacctgggttttaatcccagctctaacacttactgcttgtatgagcttggtgggcttctgtacttactgtttctcttgtggttctcaaaccttagtatgtaagtgtctaagtatctgctgtcctgttgtgtttgttgaaaaggcagattcctagtttccttctcttggagattccattccttaggtccaaggtgattgagatgcaggtggtttacagaccacactttgagaatcactgccccaccaggtttccaagtcctgttcattgtcaatgtggttgtcattcattcagtcagcacttcccagggttcaggtgggcaggggatgtgagtaagtacaaccacatcctcttatgttccagctggaggttccacttcacccagacctcctgtattagaatcttgggataaaaatggagctgacgtatgcaattcaccaaagttccacatgtatctgttatacaaacctctggttcaaaagccctgtattactgcttcagtaccagacttgactccagtccccttcctgccttaatttcccacctccccatcctgtgttccactcatacccttagcttccctgcagccttacttaagaatagggcatttgctcttcatagatgccatttcaatgagagttccctctcaagaaagctaagtctttttttattctccaagattttatctgtatccatttggcaggagagtagattctattatgatcattacccatatggacaagcagccaagatttaatatctgttggtcaataatagctttcataactaatagctcttgatgaccatctagaggggagtgtgaccttcttatcctcctggtcccctttgctggcactcacatatcctccaaaaactggggaccttgatgcccacatacataaggaccccccttctcccgagggagcacacagcacttataaccagagtggggaaatggaagactgacctctccatcctcaaagcccaacccatgtttcacctcctccatgaagccgttcctgattccagtgccctgtgctactttcccttctcagattttctaccacatgccatattacacagcttagcactggactgtgtcctactttggaatgctgttttttatttttattttttgcttgtacttcttgcttatctagcaatattgcagactctcttagaactaaatcttgattttagactttccttgaagaatcatggaggtgaaaagagaaacagatcaaggatctgctcaaggatgtcacattggagtgggagagggggctgaggacattgacctgcctgtggaggcagagtcttcaggcatctccctccagggtgaagctgaggtacagtattaaaggctgtcatcacctccaacatgactcaggtcacagttgctggaggaagacccactggtaggagaaaggaagtgcaaaggacagagcacaaagaatccaggtagctgttggtaaataccaacaagcagtcacaatccaaggaatgtccactatcagctagatgcaaagtaatggactagggcataatccaccagttttgtggatccaggaagaggcagagtaggggtcccaaggcaccagaatgggattttcattaggtaaccagcattagagagactgagctcatggacaggaaaatcatcaaaattacagagtggagccttcgagacagacacaaaagaactgtctcggaggctgactctgctcaaatcctcagcagcttctctggtcctcctgctccacctcatcaccccaggggatttctccaggcttgagaattgactccctctctctcctgtgctgagctctgtggcaggttctctggttcccaatatttcaagactccaactttgctccgctttcactcaagtttgcagtggcagaggctgtagaagtgaagtctggctggccctttctaatgccacagtgcccagaatcatgctaagtatatatatggtggccatccattcggatacatcaaataaatacagctcacttaagcaagtgcacaccagaagggtgtcaagaaacccgggagatggtgagccttcttcagaattcatctagatgtccccattatcctctctgtaacttcttacgttcctcttctcttaaagagacctgggctcaaatctgccatttactaacctggtaccttgggtaagttattatctttaaaaaatggagatagcagagcctatgaataagcagcctatgtccccagtctctgggggtgcactggctagcctgccactcccctacctcacacatcaaatggtaggatctgagaagtggtgctcagtgttaattgacttgaccacttgactatgactaaagatatcaagaggcaagcactcatttatagcaagtgggagaaacatcagtacattttttctgggcactggggatggcatagttcagtgtttgtgagatgggctggtagtttgtttgcaaggctaggcacaaactctcttgaaagacaaatcaccatataaatgctttagcatcagggcagcattagggcatgagtcatgctcaaaagatggatgaaaatgagggacagggataccaaggaggcaggattatgaaggagcaaggactcttttgctcaggccccctcccccagattctgtgtcaaggtttcagtatgtttttaatcactttactattacttataaaaaccaatagcaggtgcctgatggaagaagaaaataatgattcaagcttgagagtcattttccagttgatttcctttctcttcatttccttttgacctcagtagggaaggcatagcctcactattcaaagaaaccataactcagtcctcacttacggactttgtttaagaaagtgcttaatatttttcttcttatgtccaagggaactacattttaggtgtttaatcatctttacagcccccaaatatctgggatttatggaaagcatttaaactcagttgaaaaaaataaaaacagagtgtcctaaattcagggtaaatttttactcttgttatgggtgttaatttatgattcgacaatgatttcaaggctttacttcaaatcttctgaggcttatattttcttattattcaacctggttttattctcagtgtcaaatgaatcacacattcaatcacacagagcacaatgttcccccaaaacacataattcagagtcttagcctatggaattatgaaccccctgggaggaagatcaagggagtccttgtggtctttcattccccctggtgatgaaaatgccttatccttcaaaagactgatttttgaggggccaatgctcctgggagctaggtcttgatgccagagtgtctcagctgtcctaagtacagaatacggttcctggccactgcaataccatgaggagtattgggaaagtctaaactcaagattttattccaaaggaatgaagtagttttgagaagagttgatctagacttaccaaagtggccttaagtgaatcctccactcatgctttggagggggaagaagctgagacttggggcctcaactttagtagtcacgtcttctcactattaggtaagagacaagaagagtggttagaaccaatcagggagtgggggactcctggcaggttttggattgcagggaaaaaaaccctattacactccatccaaccatccttgctcctgcccagggctgcaggcactctagaaaacatctctgcagccaccatctcagggcagagcccaggccccatggcttatcctggctttgaacctccggctttcctgaatgctgtccctcacttagattctccctttctgactagcctctccaaactgaagccttgtttccatagcctgcctggatttggaatgcaactgctcacctagatgactttctcagtcccatattcctcacctgtaggtgggtaatggccttaccttcacactctacccaagtcctcaggatgcccacatctccactttcatagaagggagattagacagcctgtgactgacatttgctgagggagaatgaagtgtggcccaggctctcccgtgtgccacacttcctgtgtctctcaccttcaatccagcatgtgcccacggaagccactctggtctgctctcccagaggtgaactgcactctggctctcttcctccacctttctccatgagcactggcattcacatgtcctacaagggcctagtaggtcagagatatattgatttctgatcagccaaaaacagcagtaacttttcaaaagcatatttaagggagaatatcttaacaaagaaatgagacgtggaattataaaacttcaactagctggatggaggaatgatttgggagaaaaacattcagtggtattgctcaattcatattatatagaataaaaagacactagaagaaagaaataaactactatttatttagtacctattatctgcaagaccctatgatgcatttttcctcatacattacttaattttattctataaacagtttaccaaaatactgtcttacttttacaaaacagaattgaaatgtaaagagattaagaaaattaactcatgatactcagctagtaaattgcagaactggggttcaaatccaggcctatattttctcctaaatttaggagttttctattccaacgcttttgtcatagtaggctttcagcttcaggaagtcacttgaatctgatgctgacattcttttctgagcaggagctggcagtggtagacatgggacaccatcctccatcccaactctctcctccagccctgatgccacttgccacccaagtgaaagatacaggttgagctgcccacacttgttcttgccccaggcccactagagagcagactacctgacacactggccccttgtcaccttggaacaaggcctttcccacttatagagaccctagaactctcaggaaccccatattgctcttcactctgcagagctgttcttcagaggcaacggtgctgaagagaaggatctggatctgggcccctaatgaaactccccactagactactgcttcagcttaaccaaactggaaatttgagccaattcttctttattctgctacccatctcagaaagcaacttgcagaggtcaaggacagcagagagtcttcttagcttccaatcattctgcttcttactatgaacatgatccattcaagtgcaagaaacctgtaagttcagggctaagctgccattgaaatgtctgagtgcttaagaagcaggaaagaaaatgcatgatgcttttaagaaaccactattagcaaaacaatctcaaataaacccagaaagtcctgatagagctggatgactcctacccagcctcaggggtcattcatttaccaatttatgattcagagactatcactgctggctcagtctctctcctatcccaatcattgtccacctaccaaactgacctgttagtggtagatctgccaacagccactcatagagcaccagaaacaatgccctcctctctccctcccaccaccacttatcttctccagaaataaagggttaatgcaggaactcctaagacccacaatactcatgactcacactgtggttaaccaaaagaacctaccttcctcaagtccaagtcattacttctctgtacctctctggacaagcagagaggcaggtgtgagtcagtggatctaaaaggtgaagcataaagctattacaaattaataagaaaaagtcaagcacattgtaggggactgaattgtgtcccctaaaaatatatgttcaagtcctaatgccctagggccaatgaacacaacctgatttggaagtagggtctttgcagatgcagtcaatttaagatgaaatactgcattagagtatgccctaaatccaataattggtgtccttataaggagatgaagatttagagacacacagataagcatggagagaagacagttgtctgaaggcgacagaaattgagtaatgcagctgtaagccaaggaataccaaagaatgccagcaaaccccagaagctagaaagaggcaaggaagaattctttcttaagagtcttcagagggaacgtagccctaccaacaccttgatttcagatatctagcgtctagaactatgaaagaatatatttctgttgctttaagccaccaagtttgtagtaatttgttatggcagccctaggaaactaatatatgcaccatggggggtaaaatagcagaatgatatgtaaaagcaaatcatgaaagaagaaatgtaaatgacaataaaaaatgtaaaccattttcagcctcggtggaaagaaatatgacttaaaacaatgatatgtcctttttacttttcagatggctaagattaaaaagaattataatgcctggtgtcagcatggggggtgggaaatggacatctattcactgcttttgggaataggcgagaacaacaaattctagagaataatatggtaatatatatcaaaagctgtaaaatcattatattctaggaaataataaggtaaggtgcaaatatctatgcataatactatttccatcaaagcattacttatagcaaacttaaaaagggaaagaaatttaaagttcaaacaataggaattgttaaataatttatgttatgttcacacaataaaattactatgaagccattaaaattgatgatatcgattcatatttatcgacaagaagagatgttcaaatatattgccaagtgagaaaaatagcctaatctaatcttctgtgtgtgcctgcatgcctgtgtgtgtgtgtgtgtgtgtgtgtgtgtgtgtgtgtgtgtgtagagagagagagaactaaggatgggcactgtgttactggctgctaacgttatgaattatttgtgttttcaattctttaatttttccaaagtgaaaatcaatactttaaaaaattagtacatgtttcaaatgaaggagatggattggagagcttctgctgctgagctacatccctacaatgtgtccagaacaaagggtactagggcggcctgcctacacccctttgtccagcacaggaagccaattgttcagagcaggccaaaggtccacaatgggccgaggacaaacaaacatttcccttgtcttttcttacctcaacttgactctgctctgaacccttaagcacaccctcccagacctccctgtctcacctccaaatcaaccagctcttccttcagctcccattttgaaggttttctaccccctccttattacctacccctcacttttgacccgggcctagtaataaaatcatgtttttcttcccactttcacttggtagccctgctacctgtagaaatgtccactttcctcactacagcatgagctccatgaagtcatggggaactggtccacctgcaccgccagggcccaaggctgtacctgacccacttgtgtttattgggttgaagtgaatcatgcaattgctagaacccaaatggacctggaaatcaataccaatcaactttctcactttacagacaagggagtgaaagactcagaaaaccaaaatgactggccagtggccaaatcagacgagaggccagaacccctgactcctagatagcatttctcctactgcgtcacactgactctctaaagaagcaatggtcccttaggcctgggccacaggatccctctggagttcaggaggaaactgagtggctccctcagagccttcagcaagtttgatgatcccttccccagattcctcccacattctctttcctgtattcccaccctcaggcacttccaactgggtgagcttctggatcagctgcctttctctgacaccctgccctgctctttgccagccgtcctttccctggccgccctcccagcccagacccatttcattggtccctctctgatgtccttgtaccttctgaccccatttcttatcacccctccactaccctgcaccccagtctcaaccccaagtccactcctgggcccagcctcctgggtccagtcacggagaaaaagtgaactgttgctcccctcaggcctcacattctcctttccaaggccatgtttacacctaggttcctcccacccaggtctgttgagcgctcagcctgcccccaggtgacagctcagaacctctctgcttcagcccaaaggcaagtgctcctgatcagctttgctaaatgctccatcacccaaaagtggagaatcccttctcgcagccccctgcccttcccccatcatggcaaggcagaccacagccagctcaatctcctcccaaaggaagggccagcaccattgccccatggggccctaaggttcaggggctggggactggcttgcttgcttgcttgctttttctttctttctctttctttctttctctctctcttcccttctttctttccttctttctctctttccatctttctctctttccctctttccctctttccctctttctctctctctttctttctttcttttgacagggtcttgttctgtcacccaggctggagaacagtggtgcaaccttcgctcatggcagcctcgacctcctgggttcaagcagttctcccacctcagcctcctaagtaattggggctacaggcatgcaccaccacacctggttaattttttttttttttttttttggtagagacaaggtttcgcccaggctggtctccaactcctgggctcaagcgatccactcacctcagcctcccagagtgctgggattagaggcatgagccactgtgcctggccaaatactcacttttatctgctaggaaatgagcacttaatttctcagaagcttaccagggctcttaggggacaataaatactctctgttgccacttactccagctcaattttagcctgtgggcctgtggtggttacccctgggagaaaaaagaagagaaaagtggttgagagataactcaggaacaggaccaaaagcaaaaggtaagaattctataaacaagggcattgggttctccagctgagcgccacagaaaccaagttccttggggtcctggtgaggtggtaagtgggaggctgagctgatagggctgaaagccctcccctcaattcacctaaaggaactccacacttagatcacttacatattttgaataccttcagattgtatttgaaaaatggacaccattgttaaaatcaaggtttaaaaaacacttaactagggtaactcaaggaagatcccatcttaggactaaggcaatagtccagtttgcatgacgggaacctgtattcattcatccactaaacaaatactctgagacactactatacaccaggcaatgaatcaaggtcaggctaacctggcttactgcagagacagaaatatctgacacttcaggttaggactggttctgaggattgggagcctgccttggcttatgttccgggtgtctaacaagccatcccaaatatagggctggaaacaacaagcatttattttgttcacaaacatgcaacgtgagcagggtttggtggggacagctcgttttgctccatgcagagaatcatcagaagtcttgctcatgtgtttggcagttaatgctggcttctggctggggcctcagctggagctgtcagctgaaacatctaacatggctgtccatgtacttgtctgcctccccacagtgcagtggctgggttccaacagtgagcatcccagaagagagaagtggcagcttccagtttcttaaggcccagggctggacactggcacattgctacttcccctatattctaatagtgaagtagtcacagagcgcagattcaaggaaagggggcagagaccccacctcttggtgagaagagagtcaaagacctttggggtcctgtcttaaaattgccacagcctcaggggcctgtaatcccagctacttgggaggctgaggcagagaatagcttgaacccaggaggtggaggttgcagtgagccaacatcacgccactgcactccagcctaggcaacagagcaagactccatctcaaaaaaaaaaaaaaaagttgccacagcctgcaaatcgggaagaaatggctccagctatggggagggctgtaaacctactaacctgtactctttgtccagaaagtacagtagttaaaagcatgggttatggtaacactcacctctggacactcgtactgccctatgggacaacatgaaatttgcttatgccctctggcttatggcagcttgtcatcaattccatgatgcctaccttacccagttcctggtcccaagaaatacctaggccccagactcagcactctcaatatcttcctccattccttaactgatttggtttccaggtccccttgccaagctggtcagcatccctgggaaatgtctgtgcttatatacatttcttggaaaatgccaccccacactcaatgctcatgtccctgaatagtgccaggtgccagtgggcctcatctcccttgttgtggctactccatcaacagatacaggctgatgtcttcagcagtctcttggttcagttacacataccgaccttgcagtagcctcaaactttctcccctgtggtgctattaaaccctagcccttcattcataacaaattcaatttcacagttattaagaggatatcctgtgcttaggcagttgacagtttggcaccaaagagtgggattccacattacctacacgaggggaaagctgcagtgcactttccagtgttcctctcttaagggccctttagctaaaggatcttgttgccagaggtgcggcagtggcataggcaggaagtgaaatattacctgcacagtaatttccatcaagtgtaaaatatgcaacaagaccatgaaataatcttgagatattccaatatcagatttcacttctcctgccaaaccggccatccttctccctcctaagagaggacacatccaagaaaagatttggctcctaaactgacagttggtggcacgcggctagatttattgagaaacacctaattaagctccagagtaaactagccatcgaggtgtttaattggcagcatgtgactattaggggctaattaagttgttccctcgtactttaattaatgtttgtcatttggcctctgtatagctggtgcaaggttaaggaaaacagaagggacctgccggccagcccctagaccaagttactagctgtccccagcaccaaggagggctgagcagatgggacagcatgtggggcatgaagttcgttcactgtccaagcctcagaagttactttgaatgtacctaatgaacaattattgagcctcaactctgtgccagccaggcagggagacagagagggtaggagagagaatctggtgaagcctaggttcaaatcccacctctgcccctcccttgttgtataatcatgtgccaccaggcctttctgaacttcaattttcttatctgtaggatgaaaccaagaaacacctacctcatacagctattgttttaaaatggaaatcagatatgatttcccaactactcaactctgctgttgtagcacgcaaacggccatagagaacatgtaagtgagcggttgtggctgcattccaataaaatatttctgcatactgaaatttgaattgatatagttttcatgtgtcacaaaatattcttctttggatttttttctgccatttaaaaatgtaaaagctattcttagcccgtgggctatataaacttagtcaagagaccagaattttgtccgtgagctgtagtttgctaacacttgggttagacaaggggaagaaaaggataaaatactgtggcttgcaagtgaggtttaaaatatgatgctacagattaaatctcataaatgtttatggaaaatggcaggaactagatgggactgtggctgtcagggaaggctttggggaagaggcgagacttaggctaactgctgagacataggaagcagcaggctaagtggaggataggaaagcttcccaggcatagaaaataatagtagtagtaataataacaacagcttactaagtgtatatgatacggcaggctcggttcttagtgttttgcaagcatggtttagcttaatccttgtagcaaccctactaggaagtacattttatccccctcttaaaaaaaagaccagggccaagagggttaataatttgcccatatctgcacagtcagcggcagagtcagaatctcagcctaggatgtctgcctccaaagcccaccctccctcctactattcctcctccagcaacccctttgatctcggtcccctcccatttcctatgatcagatgctgccatgtccctggtgtgcctgtattcacgtaaataaccatttcctaaatacactcaatgaaaaatcaattacatagcaaagtggctgaggtttggataaacaatctaattgttccctctggggctgcaggctgagcagtccctgctgagagaagaaagagcgttaacatttattaagagctccgcctcaggcaccgatatgttttcttcctccttgaccactaggggtctgagcccattggccaagccccctacagaatcccagggaagctgctggtctcataccactggtagaaggtgagaattctgtgttcaaagtacacctctaacccaggtagacttgagactttgacccccaatagtgcctttcagatgccttactcaaagataaactcctggctgccttcctctgatggatttgaggatagtctctccagaagggccaccctgtttacaccctgacttgcccctcacagttctgttcccttctactcaaaatgttgcaattagacgtcccttattttgagtcaggacactgtccctggttgatatgcaaagatcccaggaagatgtattccatcaaagcttatcaatcattaacaccttgctgtgaattggttttcaggacttttcaaccagacaggtaattaaaattgagcaattggctagagaagatggaaagttcaagtttacatagggcacagtaggggaaggctgaagaacagcaaagtggcctctggcaagtgaagtcctaaaatgaaggtatggtacttcaaaagcctgagtcggagtggggcttatttccccatcttcccttccagccccctcttccccaccttagttattaaagtcagtcgtttgtggtgggtcagctggtgatgctcaaaaaaataagatctagttccaaacctgtgactcccaaataattacaacagcaggcaatgtggcctgtaggaagacaaaagcacatctaccagttctggagtaaagcgatccaaggtgtgaccatccaaccatggaagtcatgggataactgaaagaacaccaagcaaggagtcaaaagacaaagattctaatgctggttctgtcacatgtgtatttgctgcatgacattgcctaagttccttcccctctctgggccttggtttactcacccattgactcagttattcattcaactttgtgccagccttgtgccatgccctggggatgaatatagtgattaacagatacctttcctgttcatataaagttttcaatctaatatgagaaataaacgtgcaagcaataatttcacagataattgccctataagatgatgaagttggtctagatcaggggctttcaactctggctatagattaggaccaactaggggcttttagaaaaaaaaaaaaaaagatgactatctgggcattatctccaaagatgattattaaatttggtagggttcaggcatagatatttttctaatttccccagtgattgtggggaaccaaggctgacagccactgtgcaaccagggttgagagccaccaaggtagacaatatttagcatttcctcctgctataactttttatttgtctcttatattctatgagtaacctaagatacatattctcttatattccatgtcttatattatattcatatgtcttatattctatgagtaacctaagatacatatgtctacactgaaccccagaatgtaactatcctctggggatgtcctctggcttcgtgttctctggcttcatgttccggcccacaggctggtaccttgctcccttagagagactgcctgtgagggctacccttctgcctgtctccagagtgttccaaatgtcagccactccccaggccatcccatctgtggggccacatctctgtggccactctcctcatgccagtaggctctggattcatgctggaacttctgggccccttagacatgggttgagctcacttgttcagagagctccatgcatcagaataagaggaggaactgacagcctagcctgtccctatagatgccctgtggcaagactcatttcagatcccaagacttctctcatccctccctcctgccagggctgggatgaggggaaaacaggaggaacacctaggaggtgcccaccctcagggtcgggcaagcccagatcagcacccagagagggggtgccttcttatattttgcaccctggtcccctcactcacctcatccctgtcccagtgctgcctcctgccccaggttgccataaatattgcaggtccctatgaaagactaaagtaggacactgtcaaccccaagggctccatgaagttattctgtcactcagccagtatttattgtgtatctctaggttataaacgcaaagcagacagagttcctggctgtcaggaactcacagcataatggggaaggaaaacctataagcaagaaataacacaagactggccagaccaagtcctataacagagtaacatgtggaaggcagtggaggttgggattccttacaaagagagatggtacggaagccttcaaggagaaggcacaagttgactatcccttatccaaaatgcttgggaccagaagtgttttggattttgtatttattcagatttgggaatatttgcatatacataatgagatatcttggggataggactcaagtctaaacatgaaattcatttatatttcacatatatcttatacacatagcctgaaggtaattttatacagtattcttaaataattttgttcatgaaacaaagttttggttgtgttttgactgtgacccatcacacgaggtcaggcgtggaattttccacatgtggcaccatatcggtgctcaaaacatttcagattttgaaatattttggattttgaattttcagattagggatgtgcaacctgtagtaggtttacctgggacttggaactagggagagaaaacagaaagctgatagcaggcagaaaacagcatgagtcaaggtgtggtgtgttctgaggctagcagatggctgggcatcattagagggtgagagacttggaaagaatgatgggagatgaggtcacagaggaagaagaccagattttagagagccttgaataccctgtggaggagtgctgggctttgatctatggtacacggtgcatcttggaagattttgaggcagaggaggaaggtaatcagatttagcatcacaaataaagaattacatgttgggggaagtggacaagatcagcaatgggaaaagcaattgggagaccattttaacaacagctggcaagtaacggctgattttctaagtcaggtgcatcaaatgccagaggctgttatactgtgggctggaaaagtggacagcaccaggaagccagcccagacaggcaccctatcattccacaagcatctgccaaagtgctggcttccgcagaaagaaaacctaaaatcactggctcagatgttctctgggatggcagcctgcagagacaattgttagaaatgacatgcaaaacagtctccatgggtgttgtggcccacatcctggggtaccctcaagctaccgctgctttgacaagcttctgtcgggaagaaaatctcttctcttcatcccaggaagaaaagcacctgcctgttaggtagttcttcaggttggggattaaagggccctgctctatcagagcccctaaagaggaatgtccaaatcactttgaaaactcccttcctgccctattcatctgtggcttgaggattccatgagtataagatgcagacactggaaactttaactctcctagagttccctgctgaaataaatcccaggtgtctgtaatgaaagtactcattttgatggttcacttttctcgatccacactgagaacaccaagccctggccacggtcatctctcaccaggcccctgcctgctacagcctctgactcatcttccctgttttcagtgttgctctcgttcctcgtccctttttccatgaataaagtaagaatatggtttctgaagcacatcagtctgatctgatcacatccctgcctgaaatccttcctgctgccttcaggatacagtcaggctcctgtgaagggctcactgggacgcttcataaggctgccccatgacctccctaaaccccaagctcagcaattaaaaatcccttccaattccccaaatccatcgaactcccacctgcctctgagctctgtgcagaccgttcttgttgcttagagcagtgccagccaataaaaatagaatgggagccacataggcaatcgtgagttttctagtagccacagtgaaaaaaagtgaaagaaaggagtgatggtatcatgaataaccatattttataattcaatctatccagaatgttatcacttcaacatggaatcaatataaaaatgattaatgagatagtttacatttttttccataccaagtcttccaaatctggtgtgtattccacacttacagcacatctcagtgtggacttgccaagttttcaagtgctcgacagtcgcatgtggctaatggctaccatagtggaaacacaggcctagagccccgctgcttctaccccacccagctctctaccctgtggtttaggtctcagcagcaaggcccggcctctcgtcctgtctgtcctggtgctcctcctctgtgctcccgtagctctctgtcttagtccattcaagccacaataacaaattaccataggactgggcagcctgtaagcaatagaaatgtatttcccactgttctggaggcaagttcaagactggtcaggttctggtgaggactctcttccagctgtggactgcaaacttctcagtgtatcctcacttggtggaaccggtgagggtctctcttatgagtgtccttctgaggccccaccctcatgacctgctacttccccaaaggccctttaccctaataccatcaccttgggcttcagcatatgaatgttgtggggacacaaacatccagaccatagcactccctacagcccctcttgcaactctcacactgaattgcaaaagcctgtgttcttatctgaatctgagactgtcagctccatgagggcaaggactgtatttgtcttctttatcattcaacttcaacactgagcctagcagcaaactatatatgttgtatagaacaaatcagtatctgttgaaagaagaaaaatagtctcctgtgtcctcacctgtcactgggagatactaatatactttggaaattcccttaaagattcccttctacttgaccctctctctctccaccatctatgtgtacatgcacacatgctcgcactcattgcacacaaatgctcacactcattacatactctcacactcacacacttgcacagtagcattacgcacatatgccatacactctcgaaccctctcacactgcctcacactcactgaaacacgctcatgtgtacacacctctgcacatacacatactcacacatatttacacacgcactcacacatccatacgtacactcacacacattgcagaggttagtgagaggcccagagaggtcttcatttgctccaggtcacacagcacaactggggcaggcctggcactatatcccaaggtgactgcttaggtgaccacactgccccccactggactaacaggaccacaagatgtaaaagtgtgtctgggtacattaacagtgatttctgtttgccaccaaccagtgactaatgccatacctgatcgtcaatgaagtatatttctaggtttttgtgctattcagacaagttgccctggctgaggtgcctcagagaaaaaccccgctgatccccagggcaggcagagttttgctgacacagcagtgggcagccagggtttcatgctgacctgccaggacctggcaccttctcctcctcgacaaagaaggggctgaaccatctccattaaaaaaatcacatctctcccagtctcctagaaggaaagcaagacttttggcctaggtgaggggagctgtacatgatatcctagagcagtagtcttaaaactggggtacacagttgtgctgtgtgacctggagcaaatgaaagacctctctgagccttctaacaacttctgcaatgtgggtaagtgcaggtgtgtgtgagtgaagaaaaggtgagtgtgcgtacatgtgtggatgtgtgtgttttggtgtgtgagtgtgaaagtgtatgaaagagtttgtgagtgtatatggatgacggtgtgtacgtaggtgagcctgtgagtatacatccgtgtgtacaacagtgtttgtaggtgtgaaggtgtgtgtttgggaatgtttggtgtgtgtgagcttgtgagtgtgagtgtgtggaaataattccatccatgaggaccttctgaggtgtattcagatatggctattttgatccactgctaggagatactaagatactcaaaccgcatttactatttcctagtgtcctatcagtcccctttgctctccccatccctgcactttttaattgcctttctctcactttacaaaagaaaggagtttccctcacccattcgcaactttactgtgctttactctccagaatggaaaagttttcagggcaccaaacaaaaaggacaattaaaaacatttttcaggggtgtgtttccttattgataaattaaggcacaaaagctcatcttagggccttcagtccttccctgctttagtcaagagcaagctacaagctcagtaatgggcccttgtagaagtggtctttaatcagtcatcctatagagtagcggcagtggaatgttggcaatttggggtaaggacctcttctctgcattcctcaaggcttgtccaagaatgcattgcttttgacagggtcccaggagagcaaagaaaagacatcataaagaataacaaaggctaaaaaaaagtccttttttcctcacatattaaacataagcaactcttttcaaatattctcagtaccaaccctaggatggccaaaaacaaaagaagaaggaaagaaggaaggagggaaggaaaagggaagggaggaaaggctctttgaatagccaaagcagcacaggtttgtaatgttaattcttttaaacacaaataaaatgtattcagggctactaacacttacaagttaagccagataaaaccacagagagaaattgtatacgctttcttttgaatgcagtgtgcattattcaataggtacttaaaatctattttatcaaataatgtaatatgtattccagttacaatcaatcctgagactatttcatcttataaaatgtttaaaatttgtttctcctacaccttctattaatcaaaaaataaaattaaatgctagctactggaccaattcatctttagtaacatatacattgctttttcattaaatcagttggagagtttatctcaatgtctaatgaaattaaaggagttatttctgttttttatacaagattttaatttaattattcccaaaagccaactctaaaactcagttcacatatgcatgcatatttgcgacacttttattatgtattttcttttgtttcataaataacttgcatttttggcttcatataacatttaattgtgactaagtaatgagaaacagcatagagatttattcttgcttttataatttaccataaaatagattaattatattatatccttcatgattttaatcttgactgctttttagtaatctttttacaagattataagtaatccctcttatagtagtttttataatattccttcaaatatatgatgttgatttgaattaaaaatttaagtcagactctttttacagaaagtaagtctgatttgcagactggtttgacaagaaagactggctttgctaaagagattatgcataaggcatttttcataaattgaatgagctaaatctgagctatatggttttgacaaaaagacacttgaagcatgcaataaaacaaatgcatctggctttttaaaatgcattgaaaaaagtatattgaaatttacaatattttaatttttcaatcttttcttttcttttttttttgttttgagatggagtcttgctctgttgccaggctggagtgcagtggatctcagctcactgcaacttctgccttctgggttcaagtgattctcatgcctcagcctcccaagtagctgggattacaggcatgcgccaccacaccccactaattttttttgtatttttagtagagatggggtttcaccatgttggccaggatggtctcggtctcctgacctcgtgatccacccatctcggcctcccaaagtgctgggattacaggttgtgagccaccgtgcctggcctcaatcttttcttaataagttgaatgagacaagttgctgctaagttaaaaaaaaaaatgaataaaagatgtaattaatagtcattaaataaattttggaaaaggatttttggatattcccagaaattgagaaagggaatggttccaattatgggcaacaaataccttaggaagtctttttctcttttgacagaattgaaaaagaaaataatttttgatgataggtcactgtgtgatttggagcatttgaacccaaaaagatttcaaataattgatgaagttaccataataaaatatctgttcccatcaacttacacatgtaagcaaaattaatcaatgtgcacatttataaaagtgaaaccagttttcaataaagaatagaatggatttatatcctttatcattctagcaaaaaggaatatttattatctaattggaggatctcactaatctcattaagagatgtagtcccaataaatttctactatgtttaagttagcaatgacaaaatttatgttatatttatgttttttggcaatgtgtacactgaattataatgattattcattgaagaagtaattttgacatttggaaacttatggaaacatactaatagtaaaatttctgattacacacaacaatatgaatgaacttaacattactgaactgtacacttcaaaaagactgtcaagcataaaaacatatcacattagaataaaattctgtgggagttagaatacaaaagtgagtttaagaagattagagaatgaaataaaatttctcaatgttaaatagcagtttgaggctgggcatggtagctcacacctgtaatcccagcactttgggaggccaaggtgggaggatttcttgaaaccaggagttcaagaccagcctgagcaacactgggagaccctgcctgtacaaaacttcaaaaaaaaaaaaaacaaattgaaataatagagaacagtttcacatagtttttaaaggacgatgatagatcactataatattccaattccattgaatacattgaacaagtagtaatggtttcattttaaagagtcagtacttataataccctgaaattataccttttggaatgattccaaacttaatggagaaaattgtagatgttagcttaaagatgtttgaggagctacatagactttcagagtactttcagaggcatgtgaggaaaactgaaaggcactgtctttgagcatggggagaaggcccaggagaggccagccccgtgcttcctgtggagttgtgatttttctgtccttgttcctcccacttcattccttatggccatttcttttttttttcttttgcagacagagtctcactctgttgcccaggctggagtgcagtggcatgatctcggctcactgcaacctctggctctcaggttcaagtgattctccttcctcagcctcctgagtagctgggattacagacgcatgccaccacgctcggctaatttttgtatttttagtaaagatggggtttcaccatgttggccaggatggtctcgatctcctgacctcatgatccacccgcctcagcctcccaaaatgttgggattacagccgtgagccatggtgcccggcctcttacgtccatttcttttctcctggagaaagcagtgaccctgtggagcagtttttaaaggtatgtgaccttctcaggctctaaccattctatgctgcaatagcctttctgttcaaaagaaaactgagttgggccaggttttttattatatggactggtttcatttatttaggaaggagagaaggtggaaacctttatttattgaatatctgccatgtgctagatactgtgcaggatgctgtctaaataatctcatttattaacaagaaaaatcaagattattgtctgggccaggtggctcacgcctataatcccagtactttgggaggaagaggcaagcagatcatttgagctcaagaattcaagaccctcctcggcaacatggggaaaccccatctctacaaacaatacaaaaattagctgggtgtggtggtgtgtgactgtagtcccagctactcaggaggctgaggtgggaggattgcttgaacccgggaggttgaggctgcagtgagctgtgatcctgtcactgcactccagcctgggcaacggagaaagaccctgtctcaataaagaaaaaagaaaaagaaaagattatttttatatgtggttattattacactaagcatttactatgtaccagacacagagctggaaactttacataaacgatttaatcaatttctagaataatcctatgaggtgggaattatctcatttttcagataagaatataaatcttgcaagactaagcagctaggaagggacaaaaacctcatcatgatgccaaaagccacatttcgacctcaagcattgtcattcccatttaaaaatgagggtctgagagtcccaacaagttgccacgatcacaattggtaagacccaagtcagtctgagtcattcattccttattttattccagataggaaggaattaaggcactgcctgataacaacaaatagtcatgtttacctagaagtctccaagtacattattcgccttgcttctctgccaatgttataaagtgggcagaattgattttactatgttactatcccccttttttttctttttcttttttttcaaatgaaggaactgaaatacccagaaccttttctgaaacttgcatggccatcttagcatcctttcagctcctgataggagcagtctgacagctgtgaaaaccaagcctaggcagctgatgccacgaacactggggcccctgagctcccagggctggtacagctaagcaagggaaaggagccccaacacactgtcttatgcttttttggctatcactgaaggtgcgggcttctaactcacacttcccatcgtgtagggaaactgtgttcacagcctctctcctggttttgagcaggagcttttgggccctggcccaggagtgtttaggaaaggcacatgcctttaggggtcttgagaaaataatgttaatgaggaggggaggtcgtgcgaaaagagaggcctatggcctatggttaatgagtcgaggcgcttgttttgttgcttcataaagtcagggtcttcattggactttgcataagggcctgtttttactaattatgtcccaggcaatggcaaggggcagtcatttaaacataatgtaacatgtgcaaaaattctaacagtaatctgattacaagagaaccagagctgtggagcagcatgctaacgttgatagaacgggcaagagacagacccacgggaggcaggccctccccagcgaggctcttctgtgaactgcctgcaggagctccccaaattcccctgcctatcctatcccactcttcctgggaatggtgcgttccgagaaaatataaatggtctcatgtgtgtgcttttttttttttttttttaggattagagaagaagtagcttttgttttgttggggttttgttgttgttgttgttgttttcccccagttgactaaacatgacatccaacctacaggcttggaaaacaacttgactagcagtctgagcgattagacaggacaacagcctggggtattctctcgtggactaggggaagcccaggctgtcttttcttccgaaatttgtggcaatcatcaaaaaagagcagctgtcaaattcgccttctgccccagtggggccaccaggccactgcagtctttctttaattggtttattaatcttataatgcaataccatcctctgtctcattgttcctcactagctcaccaggcctggactttgtaaatatgtttaattaagctcccaaagccacaagccttctctcttcttggttacactctcccaccaggccgacaccagccagaactgaaacaaacaactggctccgtccccctgtccatctgctcccacgtgggggtggggatgcaaggggaggacattctgcaggaagaaatatcccccaccctagatgagaggtgctgccttccatggcaggaggcctgctgttctgtggggaaacatgaactaggtgttccagagagagaggagagaaaaggacaccattcaaacttgtgggtataaatctctctttcttctagacacccacatgtgtttgggaaggaaatctccctgctacaaaggagtggctcttctctaatatgttttgagctcccaccctggaaacacagggggtgcagggaagctggtgggggcatgggacatgaagtcaggaagagaaggtgaaaatatccggagacaggtctggggaagaattgcacttgaacatgttctacaactgctgtgtgcatgtctgtcttacccaataaaccccgagtcctagcagacaaggatcagtatctagcacagtgcagggtgtgtaggggacatataagtttggaaggagagaaggagggaaaaagggagcaaggggaaaagggagggagggaaggaatgaagaaaaagcaggtgggtgggtggatggacagatggatgatgtggtagggatttgaaccagggaccacattcactgttatacagaccatgtggcttcaggaaaccaaacaatggggttctttgcatagaagagtctcttcctaggaaggtattcagagagtttggtcagagatggcttatatgaagtctgcattggataccaccaaaataatgccaggaggaaggaaaaaaacccaatctctaccacttgttttgaagacaagatgctaaacaactcactgcttgggttgcagaggtttttaaaatttattcatgtatttgtttttactgaattgtgtgcacagggcactcagaatatacaaaacagtaaacattaaatctaccatccggatatttctaaacatctgggcatttggcatcacattgaccccaacatgttgtcatatgaccgtggagttggagcccagagcagacacaggcagaggaggcagcacatggagaggggtgtttagctcacccatcccagaatgcctgtggatgtccatgtctcaaatactctgatgtattgtcaggctatgtccaaatttttaatttggaaactatagtcacaatagatacagggataaaagaaacagtatgagggagagggagaaaaaaaggaagaaaagacagggaaggaaagacagcaaatgagaaagggacctggctggggcacttctttgagcagtggggatggaacagttagaaaacgcgtgccacaccgtatggcattcccaaggggtgctgtttccacagccaccgtctaacactgtcgtgggctgttctcatcccaaccccaaggctcagatggaaatagcccaagcactaggttcccctggagcttaccttacaccaggtttagtaacactgtcgttttcctcacctcagtggtgtgggacagagagggaagagtgagaatctccccattctacagacagggaagccaaggctcgggagggaacagaaggtagccagcgatgagctgaaaggtggaggtaaatctaagctccttgtttccagggtgacatctggatgtttttcccttttttttttttttttgagacagagtctccctctgtcgcccaagctggagtgcagtggcacaatctcagctcactgcagcccctacctcccaggttcaagcaattctcctgcctcagcctcccgagtagctgggactacagcatgcaccagcacacctggctaatttttatgtttttagtagaaatggggtttcaccatgttggccaggctggtcttgaactcctgaccttaagtgatccaccagcctcagccccgcaaagtgctgggattacaggtgtaagccaccgcacatgaccattttccttttctagaggtaagagctcagagaagccttaattgaaataattccccgctacccacatatagatcaaaagacaggcaagatgaggcgagactgtttggaagcctggagaagattggcaccactttaaacatggccctccacatacacacctcctcatacccaaacatacacatgttcatgccacatgtccttaagatttattttgttaaacccatgaagtgaagaccatcctctctacagagtccccctgcctgcctgcctgcctgcctagaatctccctgtgtttttatttcctgtactgggtgattgattacagaagagcaaattagctcctactgcttttaagatgcattttctttcttaaagagaagatgatgtgagcattacttctccctgtccactgtgtgccttggctttgggcctgtagtcactgcagctttggtagggccttcctggaagggaggagtacagcagcatctgctagcctgcagctaggcccaccctgggggcccagtcctgcacccactctgctttccccaggcttctcctcctgtcccaggagactctctggttactccaaactattttcacccttctctctgtaccaatacatgtgaattcagttgcctagcagagtagatgctgttgaatgaatgatgaagtaaatgatgaatgaattataaagactaaaatctcaacttaatgaaataaatgtgctctgggctccaaagcatgctcccctgcccacatctctgtctctctcctctctctgtctctctgtcctctctctctctctctctctctctctctctctcgtgggtcttgctattcaaagtagttcctggaccagcaacatcagcagtcacctgggagcaaggttccaaggagacctatatacaaattaaacctcgagaagcactgctgtgtggtcctgagtggaggcaggctggtagacaaaatgatctctcaaggcccctcattgctttacatgtctctcaacatgatctcagataaagacggttacgagaagacttagcccagggtcactgtacacctacagggccggcctggaatgtggaaggtagaatttcaactctttccctcccggtctagtccttcctgctgtaatgctcctttggaggtcccagctgacagagtcagagcacagcatggctttgctctaactaattgccaacagttggctgaagctacatttgttattcaaaaagcctcattaatctcctaatgccagtgtagccatggctgctaatggtgcctctcatctccccacacccagttccggatggggcatcctggctcctggcagcagagcagcaggaaatggcttccttctcagaaggctaagctagtatctggggtcatggagccgggctgatttagaacctggctgagttccacctgtggatggagtggtactaggaggctggactcaaatctgggtcatccttgggtcacagagccttggaatggttcaggtaggtggaagaatggattatagaataccacaagaaggcttatgtgagacctcttggatgagctgagctgagctgccccctcacagccaaatcaagaaatccactgcttgtctcttgggtatagggatgggctctatctcgcgaaaccctgcagcatgtttggacatggaagaggtgccttccagaaagctcatgatggggaagtcctgaacaactagagagcctttgccaaggttgaatcgaggttgaaacctgcatgtatcctatggcccagagaacatgatttcagtgctttcctgtcttctttctctaagttctgcctctgtgggtctgtctttccacgactataaactccgaagggacgggatctgctgtgtctttcacacctaacccctcacatccccagtactggccctgctgactcatgagccatccacctgactcacagtcagatatggaggtacaggagagagtatgagggggacgggaagaaggagaaatagcagaggagaaaaaggagcaagcaaagaagagagctggcagagcatacctctgggcagggagaatacgatgagtatcaagaataagacgctccgtgaagactcatgccactgcccccagtggctatgggtctggacccagatgggtgcaccaaccgcagcagcagggctgagacaggcgcagcctaggagggctccatcctctttttacttccaaaattttattataacattttccagacagaaaaattgaaagaatttcacagaaaatgcccaatatagatagtgggtatataggtgcccatctttttcgctgtttttgtttttccaagggaccagatcctctggcctcgatcaattcaatcccatcagtattgcccaagcttagatgcgcgggcagccttctcaccagccattattaaggtatttgttagaacaacactgccccctgctgactacaggctctcacttctctgggacggctctgccttgcaaagggctaagagagggctggatcagattgttccttcccagccctttgctttgtccctcccgtgtctacccttagctttgcctccaccctcaactttagttcctggggcacccgcccaggcacagcaatgagcacccgactgagactcagacaagctcagacaattaagcaatgtgtgaggccagcaaatgtcttttatcttcatttttctacaacttttcactgagtactgcttgtgtgaggtgcagcactagggctgtaggcagggcaaagctatgtctccaggctgtggataaataacttgaccgcctggagcctcactttcctcctctgtgaaatagggatcatcagactctttcttttctgcttcagagaggggctgtcaagcaaagaatgagaaagagcgcaatgaacctgatggtttcagacataactgggaagatctagaaagcattcaccttcttaaggctacaactgaacctatttccaggtgatttcttaacttcaggtaagtagcttgggccttagtatatgtattagtccgttttcacgctgctgataaagacatactcaagactggggaatttataaaagaaagaggtttaattgactcacagttcagcatggctggggaggcctcacaatcatggtggaagatgaaggaagagcaaaggaacatcttacgtggtggctaacaaagaaaaaatgagagtcaagcaaaaggggtttccccttataaaaccgtcagctctattgagacttattcactaccgtgagaacagtatgggggacactatcccgatgattcaattatctcccaccaggtccctcccacaacacatgggaatcatgggagctacaattcacaatgagatttgggtggggacagagccaaaccacatcagtatatttgtagttaaatattctcaggtgtttagtgtttaaatatctctaccaaaggaaagaagtaaagcagaatggatcatcccaaacactggtggcctctggggcaagctccctgtataactggaagctgacagagcgagatctcacctcaggcttctggcacctgtttcattcaagatcagttacagctgtggttctcaatcctgagctagatcagaattacctgagggagttgttaaaacccagactctggccccatcctcagcctctgattcagcttgcgtggggtgaagcctgggggtttgcatctcttaaacatcccccgatacttacctgggaccagactgggaaccactgagctcggctccgctttggcaacaactcctgaatctcagtgactttgcaccacagaagtttatttttcgcttgtgctacccatggataagaaagagtcagtgcagctctgctccacatagtcactcaagagcccaggcccacttaggtccccccacccaccttatagctgcagcttctggaacatgtgaccttcttggttgctgtggcacaggaaaagaaactgaagaatcatgtgtgggcttttaactgtctcagcccagaagtgatgatataagccacatgcactcacatttcactgaccagagctactcacatggccctgcccacattccaggcaggagtcttccatgtctctagaagaagtggattggatgttggtgaatgcttgtacttgtggggacaagaatgactttattttaaatggtaattcatcatgtaacttatgactaaccccgagtccaggaatgcctccaaaatgtctagttgatgtattactctttatgtaggaacatctattcattgtaagtttcctccaaaacaacccttgtagttgcagaaatcaaaggctgtgacacccatagccatctacacatcccttccagagcatgtatgctttttcttcaagatataagccctgggtctggggggtgttgtggtgcagagatctacctgtcttgggaccacccaagaccttgcttctgcctgtaagttccccataataaatcattcaaaactgacaaactatatttgtctgcctctttctttggttacttggctccttctgcatttggaggttgccttatacatgcggccctttcatggaacagtactgtcaaccataccaagcaagcatatctcttttcactctcaagaacatgtgtgatcacagcaaaatggggtggtggttagcgggaacaaatatctggcctcataagtcttggtcatttatgatgatgatgatgatgatgatgacagaaaataactactgagtgctcactatgttccaggaactgttctaagtagtttttgtgtatcaactcgtttaaccctcacaataaccttacatggtaagtaccatttatacccattttataaatgaggaaactgaggcaaagtgtgattacatcgctcacccaggtcatagagctataagagacacagctgggattataatccaaccagtctgacaccagagtccatgttttaaatcactgttctgctctatcactacccagctaactttatttttgaagataagaggagactatagtagaaggcctgattatgattaatagataagtgaatagttcaaacccgaataatccaaatacaaacagttctacagagaggggatccccacccaagcatctctactgatatcccaaatatgcccaaatatgtcaaaatggtttccagttccaacagaagggctttcagggtcctcaccaatgccccacaatgtctggaaggccattcaggtagtgtctgttcccccctaaaatcacactcttcctagtaaattgatcaggaggatggacaattttttttttttttttttgcatttctcaaagaattgcctcccaggtcatacagatcccccttaggaggatcttatgcccactttcctccctgtttccccctacacttccccttggccctattccatctggaaagacaaactgtagagtccccaacctgacccaagcatctactctttctctaccctggtgggccagtggcaggttcttatttgacaatatcatcataagaggatttaagtcttagcatcagtaagaaaaagcaattcttcaggttgaagcatctacacaaaaaataacctttctccctactgattgtgatagcctcttgtaacttcaataagtatgctattaaaaactctgtttctttattctgtctcaccaaataccccctccccacaataaattgagaaatgaaatatatatatatatgagatagaaaaatcccacctggactcactggattaggtgaatttgttttcctcattggtggatgcgtaggatttagctgcaaagagcctctggccagcagagggcgatagtctcatagcttcaaatgtaagctacaggtccgccctcaaaggtatctctcggggaatgtttccctcatcaagaagaatctgtttaatccgaaacacaggacgctcgatggttcttgttagtgcctcaaatggagtcaatcagtggcctcaaaatgtcagatttcctccctgggttttacgagtctctcatttcctgtttttcttaatgaaagttcaaaatctccctggcaatgattttgaatcaccacagttttcaagaaatctgaaaatcctatttcttttggttggacagccatcaaagatacatgtcagtcattccatccatctctctttctctctcagtcacacacacacacacacacacacacacacactcaactttcacaattcttgctgacagctcaaacccatgtctgcaaccagagatccagcagagaagaaggaatggttggaaattgactaagcactccaacaagttattttgcatcgatgatctgtgttatgagaggtctccaacctgctcaaaggtgtgcttagcattactaagggcacatggagtagtggctaaagcaattctttctggtccggtctccaggtggttaaaggcatgaaacatatctttcctctcttgcaagagtacctatcagatggaccagcagtggggattgtgctttggaaaaaggcaacagcaacccacctcttggctaattctgacatagaaagcatattgcacccacagctcctgaagggcaccccatgggctctgactcagattgaaagggaaaaaatggagatccaagatttcacgctgtcagcgaatccgtgtcttcagagggtggcaaaggcagacttgggatccttgggagctctccctccagcacttgagccgggaggcatgctgagccccataattccttactgcagccctgcccagccaagcaagcttcctgccccagagttcagtcttaaccatcgctgcggaggtgcgttagtttgctaggaggactgcctaacaaaggtcacaaactgggtggctaaaatgacagaaatttactgtctcacaattctggaggcaagaagtctgagatcaaggagttgtcagggttggttccttctgagggctgtgagggaggatctgttccatgtttctctcctagatttgggtgatttgcttgcaatttttggcattctttggctcatagaagcatcatcctatgatcatgaacctttatgttcatgtggcgttctgcctgtgtgtgcctgtgtccaaagttcccctttttataaggacatcagtcttactgcattaggggcccaccctccaacagtatgacctcatctaaaattaactaattacatctacaatgaccccacttccgaatgaggtcacattctgggatactggaggtttggaccccagcatataaattttggagggacacaattcacctcataatgggagggcatgcctcccctaacgaccaacccatcttccaggctagatggggatgccaatggccaggggataaagcaaacaaaaaaaagccacattcattaagtagattttatttattttattttattttattttattttattttattttattttattttatttttttagacagagtctcactctgtcacccaggctggagtgcatggtgcgatcttggctcactgcagcatctgcctcctgggttcaagtgattctcctccctcagccactcaagtagctgggattacaggcatataccaccacacccagctaatttttgtatttttagtagagatggggtttcaccgtgttggtcaggctggtcactgattggtaaaattttaaacaagagtggtatgttccatcaatccagcagggctaggaaagaatgggaatgggagaaggacaaaactagaggaaataagaccaactcagaggcttttactataatccaggtaagagatgatgaagtcaacccatgaagagatggagaagaacatgcaaatataagaaatatttaggaggtaaaatcagcgaggcttggaggcttatcagatgttgtggagtaaaggagggtggagagtgcaagtggctcagacttctaggttggatgatggagaagacgacagaggacacaggtggaggaacaggtggggaagaagatgtcagggtcattgtatggctctgctttaccccaatccttcatgctcccggcttcacttcactatctattatctaatggtttctcactccctgtttaggtgcagttgtggacatgctgaacttgaagtgactataggacaggggtggtgttcatccctgccttcaaagaattcatgttctaatagagacgacagactctattagatgtgtgatccaaatatgcctagactcaactacttacaattccacattccatgctccacaatccccaaagtgccctggaaatctaaatagaattttttaaaatataaatttgaagtaagttcatttggcagcaaaacggaactgaatggacttgagctcacgacttgactcatcaactgtgaacaggggtaggttctacacagaagttgtgatgcccattagagtcatataatggggaaatgagcattttccagcagaaagggggtagaagtgcagaaaggggaaagggtgctgtttagacctcgtagatgttctccatgctcatcagacctcactggctgtcaatattgcattattcttcctgttctatagataaaactaaggctcagattcatctgaagtcacccgacagggactcaaatttagaatggaaagctcttctatgagatgagaatttgcaaagagatgctaggacctgaactgtttgtttgtttgtttgtttgtttgtttgtttgttttgttttgttttagatggagtctcactctgttgcccagactggggtgcagtggtgcaatctcagctgacagcaaccttcgcctcccagattcaagcatttttcgtgtctcagcctcctgagtagctgggactacaggtgcgttccatcgagcccagctaattttttgtattttatagagacggagttttaccatgttgcccaggctggtatcgaactcctgagatcaggcaatctgccctcatcagcctctcaaagtgctaggattataggcatgagccaccatgcctggctctgaactgtctttatactaagatttgtgattgattgactttttcatgtcacattccagttgttttcatgttgaataaacatcacccatagacgtgttttgttttacacacctgagctctaatatacccttctccaatccatgggctagaagcagaatctctctcaggggtcttgttctgaagggagaaattttgccatactcttcttgagaagataaaaggtgcagaacagtccttgaatttaaaaactagaagtagacagccgggcacggtggctcacgcctgtaatcccaggactttgggacgctaaggcgggcggatcacgaggtcaggagatcgaggccatcctgggtaacatggtgaaaccccatctctactaaaaaatacaaaaaagccaggcgtggtggtgggcgcctgcagtcccagctactcgggaggctgaggcaggagaatgccgtgaacctgggaggcggagcttgcagtgagcccagatcgcatcactgcactccagcctgggcgacagagcaagactctgtctcaaaaaaaaataaataaataaaatttaaaaaaaaaaaaaaaactagaagtaggctttgtggaacacagaggaagcaggtaatacctgtatggatggtttatcagtgtctctgctgcaggtttcatatgtcaattctatgcacaaaatgtctaatatttaatggtggcaggaggcagagtcccttgggattccatcagacactaatgctgccttgctctggttagaaggctgggatatctgtgcctgtgttcactttttggagaaaagaggacaaagcaatggtcatgagtctggtctattgacttgttggctcattgcaccctcagctcctcagctgcaactgagagaacttcaagcctatgtacccagcagcagactcagcatggtcccaagcctggacaccaggaccgtcttcctttaaggagcctcaaagcccttttagttcaatgttcatgagactcagtcagcatccagcctttctcaggcccaatgctgaactgtgtataagtctctgccttgctcccacaaccaattctcttccctgaaccggatattcccgtgttgccatctctgcattggaactctgcccagtgtccaattccactaggacaaagacatgtccttgctttttgtttccaccctcaccttgggcttggagtattgccttgatggctcttgccctgaattctcccaccaaacctcctcatgctacctgccagcctgcttcctgaattttaatttacatttgttggatgaattagtaagttactagctgctacacactgggtagttaaagccacagaatctactctaagggctcaaaatttagtgaaatggattaatttaataattgaaaattttgcatataatggaattatgagtgaggtattgccaaatgttgtagagagagaggattattttgttatggaggatttcatagaggtggcaagatgtgagctgctcttcaagcatagttaggaatcctctaaacagaaaaggagcgaacactccaggcagaggaaatggcccatgcaaaggcacatggcctgaaaatctcagatactgtcttctcttagactccctattctatgggaacactgctgtatgcacacttgttgtgatgccccacctcgcttctcatgggaactttgggatgtgatgatttgttggacatagcacccattccctatcaccaccctctctagatacggagctccttctggtgtctaaatctttccattttcagctgggttacaggctatgataagatcaaccttaccttataaagaaatgtccaaaatgcctggcctgggcttacttcatcggctgacacttgcagagcttcctagggctataaaaggaacagggtctgccttggtgctgcccttttcttctgtctttgaaatctgccttctcgtgcccaaacccaggagaggacagaagtttctccttcctctttcccttctctccccttcacaccatcttgctgccaaacaaggggctgatttcagtatccctatttagggtcctctggacctcaaaatcaatcaataattttctggcatcaccacatcacctggccttgccaccttcacaagaatataattctttgtctctttgcaatcctttaggctggttgtttttcccaaggttttggaaaacctgggcagaccattctttctgcttccctccacctattgtggggaggtagaaatgctgaaagctaagatcaatacaatccataacaaggactggcaatgccagcctgctttctgtcctcatgctgaggacttgtctcttaacatggagcagaaggaaccagccttggatctggagtggctattgtgactcgtttccaattcactgcacagagaaggagccaggccattgtcctcagcccacagaccttgctctggaagcattgtgcctgcattccacccctatgcgaggttctttcaatccatctctggaggtcaaatgtggaccatggcactgaaagcactcagacaagagatctggtgtttaaatcaagcaattgtttcctttctcaggagtacattatttctgctgatttctacttattgtcagtcctattatttatatattccatctcaggaacaacagcagaggagaacatgtagctgatgatgatgctgctccctcagaaactctcctcctcctgaaagcctagaagaatggaagagagaaggagggtgggagtgagaggtgttcacctccctgcaggggatgcaaagggaaaggacaactggaagaaggattttcttgttgctactctccctagggccagctggggctggatgtgtccccagaaggctgtctggctcccaagccctaggcattctagttgttcaagagtcctgggtgtcaccctcccaaacccattataagggaaagattttgtctttcacttgcctccctctttctgactaagtcccctaactagggcatatattgattggactccttggagatgagagtgctcgggagctcagtagggttcctctagacagttattccaagaggtgagggtgcctgtaacctctaaatcacaggatcttaagctgagatctcactatacatggaagtcctccgtcccagacctctggcttatgggtgagcatgcgtatgcaccctctcgccttctgtgtgaacactgtcccctcctaagccccatttgctcccttcacccagatctctggagccaccttacataccatattacataccatattggtctctgccatgtctcaaactgattcagagccccaatgcgtcctcttcctgagtgcttgtcattttccaagcaattcacatacattatcttatttagcacttatgaccttcttatacccaatttatgcttgagaaactgaggctcagaaacgtttttaaaactgtctgactcagcctctgcactacagtgagtctcaaaaaggtgtgcacatattacagaatgtcaggatctatccaaggaagggattcataaaggtcatctattcactcagaagaactttccagaatgcctgtgccatgcctgctctttgatggtcactgtgggggacagaaaagactctcgggaaatccctgtgatcccattgagtcctttccacaagtggggaaggggcccagtaattgtccctgggaagtctccagaaggctgacttgctgagggaggctgcagacagctccctcttaaggcagaagagggagatgggcggtgggggagcagtgacagctcactgcctgcgcatcgccttacccacaagagacacctgcaaatatgtgttggttcttaaaatattaaagaaaaggagcagcagttactgcccatatcagaaatatgatgctccggttgtgcaatgggttaccacgtggaacttatacagaagaaatatggagaattgtgaggcaaggcagaaggaggggttggtcagacacagtgccaccaagaaaaatccgttctccccaccagtcagccagcggggaggacacaggcacctctgggcctgttgctagacagggcactgtcactagagggcagtgcagctcattcttgggagtgagggagcctggtcatcccatctcccacagagcaatcctgctgggaaatcaccgtgtcccagcggggcagggaggagcagcacctggaggtaggtagactcgagattctaggggtaggggacaaaggtgacattacttttgatgtttttatacacattctttcgttttatttgaccctgacagtagactcctggggtagactgagatctttagcccaatttgacagatgagaagacaaatcatgaaggttagactcatcccaggttacccagctaataaaggtgccaggttccctaagccccagtcccaagttgaatccagggctgtctgtaaacatgatgacatgggctagattccttagtgaaattctcactgttgtaatttctccctttgttacttgtgtattccctagagcgttgggaagaaacagaaaaacaaggttaaaatggaatcgctcaatcagatacattgtaagtagctttgataaaagtttggatgaggcaagggatacttatgactacccacttcctttgactccactcctgctggcctttccaggccacatcctggtgaccttggagagtcatcgcaaatgcctccagctctggatacagaaatgcatgtggataatgaaggatgcagaggtgaaaggtcctccagtctctgctgaggatcagaagcaggttcccataggaagtgactaggtcatagccagaaaccagaagccaagggtgatgagattcatcctgtcctccatcaactcttcctggaacatgacatagacccaggctggggtcatttgggaatatgatttaaagcagtagaggtgatagattcttttagcacatagacacaaatcacacacatgaagagagggttggatggtcagatgaaaatctacatgccttgtagacattccacctgcaagatcccctgctacccccacgtcacccaaggtacagttagacataaccaaatgcttgatgaacactggcagaggctgaggaggaggattgaagaatctgggaaagaatacataggatggagaggcccagggaaacaggagaaaagaagaaagtccaggtgggaagagttgataccttcctcctaggcagagtggccatgaggactcagccagacatcctggatgaatggcccacctctgacacacaactctctgcagcatctaaagaaattggaagatgcaacacaaaccaaacaatgggccattttgtacattcttcaggattctgtgtgtccatgggggcagcccatggtaagcctgatatggttgggctgtgtcacccaccctaatctcattttgaattgtagctcccataattctcacatgtcatgggagggacccagtggaaggtaattgaatcatgggtctttcccgtgctgttctcctgatagtgaataagtctcatgagatctgatgtttttataaagaggagttcctctgcacaaattctttctgtttgcctgctgccatccatgtaagacaggacttgctccgccttgccttctgccatgattgcaaggcctccccagtccattaaactctttcctttataaattacccagtctctggtacgtctttattagcagcgtgagaacagactaatacaaagcccatgtacttatcatccattgtcctcctcctcccttccacggtgtcaaatggcaaatggtgtcaaactcaggacacaccagaaaactctcattttcaggatttatttagcccttcaattttcaggactttgtggaagtgtagaggtcattgaagcctggtttctaccctcagagggcttatcatctaagcggcaaccctgacatgtatcataccaattttaacccaagcagagtagggcaggtgccattgcagagatgcaagagctttggaagaaaagcgaaagagtcatcatttgcctgtagacccagggaaggatgctcaggagaggagagtttttaaagatccagctcaaataaaagatgaacatctcgcaggctgcaggaacaatgttgtccaagacacagcagtgtaaaatatctattcctggaaatactagcagtctggtgtcttatttttaacataaataaatactaaagcagcgagtcttgcattgattcatgcctgtgagccagtccatctctatggtcctataatttccattccctccctccagccctcattcgctgatcaggagcacattctattctctgttatccaacctccaactggagatatgttttgaaataagtgatatttgctctttggtggcccctcttcctcctcattatctgtcctatcatgccttcaaggccttgcacgatctgctccttttcctgcctcaccagctgcatttgggctactctccccactactctacctgcccagtcacaatgcactactctgtctttccccagtaggtatggctcctttccaccccagggcctttacacatgcaattttttggttggtttttctgacttctcatgtagccaattctttctcctgattcattttcccttccctcttccaccctggcacatcaccatgtcttgtcatttggctgtccagaaagctcaatctggagatctaattaaatgtaaaagacaatacagaaaggaggtgacgcaagaatctgctcttaaacagaggaaggattgctctgtcctcatgagcgcatctcccttttctcctccttataccaaaccttaagaggaccgcttattttgtttacttggaggggtaagcgtggtcggatgggagtggtggatataactatcaggttctatatggccactttcccctttgccaatgtcacctgtgacattgcaaagctgtctcatcatttccagtatggattttgtgtaggtattcaaatctctgtgttttcttaattctaccacacccaccactgacgcctcccgaatctggtcccactcatccttaatatgcagattctactttttctataaaacctttcctgatatctccatcctgcagcaatctttccatgacccctatctctgggaacaacgtaaattaataataccttatttagctattgaatgatctccaattgttattctgtagcttgtcttgtctcctgtcctgggcacatagttgatgtttaataaatatttggtcaaattgtcagttgagaaaaacatagagataaggccattaaaagaatatgaactgggcaaaactgcaaccacgcatgtgcacgtgcatgtttttgtgtatgtgcatgcttatacctgtgctgtaatgacaaaagctctcttccacgtccttgagagacattactaaatgccccctcagttctctagtcatctgttctaatatttgagcaatgggtgctctgcctcctcgctaggcctccaatgattagcgtatggcccggtcagtgcttgcaaggagatctggcttctgacctcatcattaagaggcaatgctttctcctctggcaccagacatctgctgaacctatttctgtttccacagcagagaattcattcctggttctgtggcttgtccagcccagcagggtgcaggtgaattggagggaatgggaaagaaagaggattctggtggtctgatgacaacaggcaattcctttccgattcatcctttcatcagagtgacttacaaggtgcagagttgactgattcatacacttggacagtaaaaattgtatgaacataggaactaggtctggtttcagatccccaatacttagtatacatcagcatttgataaatatttaaggaatgagtatatgtatgtgtgtgctgggaaagtcttcagagaagcagaattcagcttcctctagagataaactttgtaatgtgtgtcaatgcccatgggcattatttggcggtcttaggctatgataaactccccagctccagagtgtgcaaacaggaggaggacagcagcttagtcggggatgctctacccagagagtctaggcaataaatgggatgttgtatcaataacaataatatgctattattcctactttcaatactaataatagttaccatttattaagtacttacttatgtaccaataattagcaaatcatatcttacttaattatgatgactatcaaaggcctttctacccctgagggctgataaatctatcaggtccaataaacatttgcagagccaggcacaggaggaggatggaggaaagaaaaaagaggggcaagaaatacaggcagaatcacagcccttaaataactatcaatttaaaaaaggagacaggaaacaaactagaattcaaggcaaaaaaagattaagaactaagaagagttgtagacaaagagttttgggaacactccaaagggaggagtgatcagttgtcagtgggggaggaaatcttagaaggctttctggatgtggaatttttgaatctatagaatttctataggtggagaatgaggaaagggcattttcttaaccaacagatcaggataaacgacggtcaggagttttggtgtgagtgttatgttagaaataagtgtttcatatggtgtggctggagtgtcacatgggtagaaagagagatgcagtggaggaaggatagctttgtgctagatcctggaatgctttcaatgatgggctgaagtgtttggattttattctgtagacaatgaatgtttttgatgaggaagcagtatgttttattgaggtaattctagaatgaaagttacattgtagactgtcttagtctattcagggtgctgtaacgaaataccataaactggcaacttgtaaacagcagaaatatatttctcaccattctggaggctggaaggcccagcaaattcaatgcctgatgagggttcacttcctaattcgtagaaggcaccttctctctgtgtccccatatagtggaaggggcagtgcagctctctagggtctcttttataagggcactgattccattcacaagggctccacactcatgacttcatcacctcccccaaaccccatgttttaataccaccacactggtgattagctgtcagcatatgaatttggatggggacataaatattcagactatggcagataaagaatgatcatagctaaaatttattaagtgcttaccatgtgcaagccactgttctaagcagctgcattctgtctcgctccaccctactagtgtgtgaaataagtttaataattatgccattttacagatggaaaaactgaggcatagaaaggttaagccactttcccaaggccacatatctgtcaatggaagacccaggaatccaacctagaagtcttccccagagccacttttaactatcctactgcaccatctcaccctatcctatcacagttctacctgggtgggaggttgaaggcctgaactgagatcatagaggtgaatatttgcttgtatttgcataaagaaataccataataaaagtggtcacctatagagggcaatgtcagggtcaagggacagataacgagtagaagggggtaacacatctcaatgtatgccttttaatatggtcattttcttgccaatatgaatatgttatttattcaaaaaacaagatgacacaaaagaaatggtacaagttaaaagctaatttcaaggaacaaatggaagtgcataagagctggatgggtttggaagacaaattaaacaggcaacacaaacaaagcaaaccctggagtttctcagttgagtgtttggaagatggtagctcattagcctagttggggaaaacaggagagtgggtttgagggtggccataacacattcagccttgtttgtaggtgacagtgggcatttggatgttgaggttgagcatgataacccaggtggggtgttgactgaggagattcttatgaagatggtaatagactctgtgagtagatggtaaccttaaggagagagtagagaaggagaaaacaagagagcagatgacagagtttaaagaaatctgcactaaagggactgccccagagaagctaagatggagactgtgaagccttggtcaagggccaggttggagagatgagtacaggaaacagagggagcacagagtttccggaggaggggaatgatttcctggggtctcataggttaggactgagacaggtttggggaccagaatgctgagcttctggaggaaaagagacatatcaaagatactgagtgagaccgtccaactgaggggccccttttgggacatccaaggccctttctcccagttctgtaagagataacaagcaaggtggacactgtcttcagtccctacatgtcagccagtgtagatgttctgtttttcagggaggtgtttttagacaacatacaccagcacctgaggggttaaaaaggaaactgaggcagacaatcctagaaagcttctctgatttttttctgtcatcacccattcaggataatgggaacttataatttgatgtttttgtttttcattcttccgttctgtctttccatcacacacatcaatgcaaacacaatttcacatacacatgcacacagactcacacacacacacaccaggcaaaaatgcacaagcatttttctggataagactcattactgcacgctgggagggagggtgagcagagccgaggaaaggaaagagagatgcaagccaccctagctacttttactcccctttaaggctgcgtggttacggtcctgggaaagaaaactcctgcattcctcccctttaattgggtttattgttaaagcactggagcagctaatcctcacagacctgtaggagctggagtgggagctcaagcaggattcttcccgagtccctgggtaagacaaccctgcttcttttcttggctttagagggtctccttgcttaatgggaagcgtgcagcacctagtgagtggatttgaagagccactttgtaagcaacttgggcatttatttcagccccagttccagtcttccctgactcttttggcatcaagggtgagttatagctgtcttggaaaataagtctgaatggggggtccgggtggagctatctgtccgtcactcctgggctggctttcatggaagcattttgcccttctgggcagggaaaagggagaaaaatatcttcatgtgcccctcctgcaatgcctgcattctagggacaaggctctgtgcctagagggaccccgtccatctgctggggtgttaggacctccaactctcttgcacccctccctcctgctcaaccttcatctcttaacctctctgaccccatccatcaaataaagtatgccctctctgccgacctctttgggttattgggaggattagcagaaccaatggcaaaaagatttgtaaacagcctgtggctctgcaagcctaaggaagacgagtcagcctcagtccctcctggaggagtctgtggacaggcttcaggctccctatcctcagccgcgttaagatgcagaacgcagccgagctatcgctgtcagtcacaagacagcttccttcaccttccccaggggcgcgtctacctcccttctgtttttaaacttccctagtttacctctcaagtgaagatcttaaaaggaggtctggattcaccaccaccaccacccccatccacaccatggccccttctctccacgtaactttgacgcacaaacattaaccctgaggctcccggagatggctgccagggctcacgctggtctggctgcagtgcttgtctggccagcctcttgctctggagtcagataaattgatcatgaggaaagcctctgagaagggtcaagagagtcaagcagattccgccttaggtaagaagatgatcttttccagatcataagctgaagacagaacaaggtccctgtcattcctgctgacccaagcttaaccagaaccaaagatgagacccacctctgaaacaggccgtaacacagagtgcttagtaatacctgatatggggaccacactttacagtttataatgtgtttataattaatcatctttaggacgatcccatcagatggtgtggcatgtattatttcccctttagtatagatgagaccattgaagcttagaagttaaagggtttgcctggggttgcaaagctggtgatggtggtggcaactgaacccagatctattgataaattcagtgaactttccacgatgctgcagattgctctggtgtgtgtgtgtgtgtggatgcttattaagagcactcttcagaaagtactctttgggaaagaagcttgaggttagagtgagaaatggtttgagctggaaccatgagaggcttggggcaatagacatgctgtccccagcctccatgctgagaaatctgagtgctttagggattcagccctttcaagattgtccgagccatgtgttcccagggtaatcagtcagacgttaggattgcattacagcatcataaggatgaaggtggagatgagtgacatcccagcaactgccaccaggaaagaggcaacaggttatgtggtacagtgacgagaagactagcgttggaatccaattcagttgtatcattcactatctgtttgatgtgggggatttttctgaacatttctacttatcagtacctcagctgtaaaatgaggttacctggaaaggtggccgtggggcattaaacaagataatgcatgcaaagtgcctagcgcggtgccccacccaggtgcgtggtccagggcagcgaatactataaagcctgggagcgattctacctggaagtgaaaaagcatccccctctaagaggagagcgcctccctcgctccctctcccccatacacacgcacacagggccagaaataaattgccacacgagtgcgcgcgagtggcggcgcccagccgcagcctgccagcgccagcgagtgaatgagctgcagcatgaatgaggggagcggctgagtctgcaaacaggctgcgtgcctggcgctacaactgcaggcgcgccgccacggtcaatggcgagggggttggggctgagtgggagcggctgccagcaagaaagcaaactgaagcaggcaggggactgcacggactatccaggattgtcaaacccatgtccttggaggaaaggctggtaactcagagcttcctgacacagcccttcgtcctgagttcagcctgtccttccttttgtactctatccctggtaggggaaagatccttcctctccccttctggaagccagagtagagtgcctagctgggaaggttaatagtaatgagcgtccaacctgtacaaaagaggcacctcttgcatgggattgggcttgaaggcggtgtgtgacacacagtcagcgttaaccttgaaaactccttagaaaagtgccccagtggagacctgcatatcacctgtgaaaaaaaccaacagaagtattatttcctctcttgttgcaacagttccctgttttcttggttgagtaccagatgaagtagttggcttgcattcaggctatgatgtacgaaaaatcatatatctttaaacactttagaaacacagtgagatgagatgctcacactgtgaaaggcacgtgggagctggaaccagctgaagaaatagcagattcctgtctcctgtcctgtgctcaccctgtttaagtccttattggtttccctttctctttctccagtgcttagtgtttaattcatgtgcctataatgtggacccactgcatggcactttagaggttgcaaactgcttttgtgcccattgaagcattagccctccaaaggaatgctgagaaatagagcagggatcattattcacattcagcagatgaggaaaccaaggcccaatatacttagcagcacactaagaataagcagcataccctgctctctacaatggcccctgtgtgttgacctctcagctaccgcatgaagccctagagaatgatcttcacatcctctgtaagaagcacacagtaaaccataagcaacacataaattatggcttgtcactgcagcaggagagattttgttaaacttaaatgtaggtttgcgccttaaataaactgcaggcaacaattgaaaaaccttctctctgggtcattttgtgaacagaaattggcatgcagagttgatctttgaaatgcagaatggcagtggcagaagggctgggaaatggagtgggtggtccttggagtttctctccagccagaagcttcctgaagcctttaggttccagagcctcagctttggcaagcagatgagccacttgggttgcaacccgtgaactggaagtgagttctgacacaggcccagccattcacctatgacaccacaaggagggtgaaactgtgactgatggcagagggctgctcgaaatttcatctatccctgcaaaatatgaagaattacaaagcattagagatggcctagtctacattttccaaaggatgctctgagaaaccctagatccccggcacactaatattagtgttacagaaggaaagaatttcactatctacgaaatttgggaaatgccagattaaacagcatgtgtctttaatggtcctcagagcctttaataagctaatgtggattgtgaatctccaagaagggggccagaggaggtagcaaatccaccctactcacagagagtcacccaggactaagatactgaaaagcaaacttcagggaactttaatctagtccaatctcctctgttcaccagagaggaggatggaaactcagggatgtatggtgatctgcctggggctgcacagctacgggatatcaaaccaagactggaacaagtctgggttctttctggtattcttttaacccagtggatgatataaagtccaatatggaccagagtctagaaaattgtgtgacccagcaagaaaacttcccaacttttgccttttacccttttgtaaccaagcgctgatagatttccatgattcggtgtgccccagcctggaggcagagagcacattcttattagagatgatccaactgataacctccctcccaaggcccataggcttttctgagtcatgctcccaaagaatggaaaatcccggaagcagagactataaagcagtcttagggtagactgattcctctaaggtcagtaaaagagccaggcatgatccagaaggctctgagcctgctcactatgccccagagtgattcatgcctccttgctcagaagcccctagtgacagcaccagcaaaaggagggtggctcttcccatgggtagaattgagaaccattatctgtgagcttaagattgcagaggtgctactcaaagatgctctccttccctgtccccatatcctctggtttgtccatgcccagtttaggaacactatgattgttcctacactgtacacacattgtaatggattggagtggattggattagagtggagcggaatggattggattggattggattggattggattggattggattggattggattggattggattggatggaaagcctttgaattataacgtgatgctcctaagactctggatcacctaattaggagcgtaacagttttaagctattagcccagctgatacgtttaataagcacctcagggcaggtccctaaatggctgcaactcaaatagaaaactttgggtaggtaggccacatggagagtggggcagacatgagagtgcagagagagaatccggccagtgtggggtcatgaggtgacctggcagttttagggtatatgaccacagtgaggaggccactgcagacaaagcttatctgcctgagagttccatctcgggctagtggtgctccctttactaaatgaagctggaaaaggaggaaggacagagaagctccatggaaggggcatctgcctcctgggctcatggcccaagcatctctcaccagcttctcataagcaagcatcacacagcctgccactgggagcatggaaagggctgcactcagaggcagcgtccaccccaaggctgtactgcctgccctttaggaagaactttcccctctgtagcctcctccacagctgcagagttttcccccaagaggccaggaagttcatccaaaaaatacaattgagccgaagctaggaggcaggtactgcactagccccaagggaggcaacaagatagccaaggtctttgtctgtgtggagttacttcctatagaggagatcaacctcaaacaagtaaacagggtgctttgagattgtcatacttattacagaagaaaggactggctcacctaagagagagtgactctgggtgtgggtagacggactctaggtagaatggtcaggaagagccttgtctagtgttgcagggaagttcttctgaccaagaaccagacacggatcccatcccaggcactagtgtccctgaaattatgatgacaagaaagaagccaagcagatttggccttctatggccattccaccctctcctgctgctgtgccatgggcagagcctcacagcaaggactatagagagggaggccctgctgcggtcccttcccagggctcaggtcttcttgctataggctcctctctaaagcaatataaagaatacatccacccaccttctatactcatcttaagtctaactcctccacaatgcgctccccaatcaacccacccccaagcttttctctcattcactcatttagcaaccatgggctggacctttagtgagtgccaggaacagtagtaggttctaggatttcaaagatgagtaatacatggtcccagtcctttaagcagactggcctgcattcttagctccttatttatatatctgtgtttggtcttcataacctgattgtaagcacttttaaggccagactcctatcatagaaatgtctatttatagcccaaattgtacacaggaagtactcaataaatgaatgttgaatgaaggagtattacctccacgactcaaagatagttccatgaataaggtaattttctttgctaggagccatttttgcagaatattcttgagaatcttgctgtgctgaggtgccaggcgcctgaaccagccacaaagccgacatcctccaagtctcctgagctatcgatgtgaaaaatcagccctaaatctcagagaaatctgtggcggctgctcctgagctttgaaacccaatggaaattgttgccagaaatagagtgagaggcggggagtactttggaaacaaatgagagcaatattggagcttcaagctccttagaatttgttttcttttcctctgaaaaggcctagattacagaagtcaagagaggagggggaggactttagcagaaagaagcaggatgcagccatggaaggaacatgggcacccctttcaaagcccactgttcagacctccaccacacagggcatgggagtcttgaggcgatcactctggttacagaggggtgggaagggaaagccaaagctatgggggtttcagatctgggcctttgagttttccacattgcactgaagatggaactaaatggttaggattcagtgtatgggaggaggagtgggcgggaaagatgttttctcatagcctagcttcggctaagaatactatgtttttaaagctatttaagagagaagggaaagaaggaacagtgagtacagatctgctaatgaggtggtgcagcctcctggagaacaggccgtcattccatggaagctccagaagagggagtgatggacgcctggctcttggtcccagagctggaaggcggctcttcattggctggaaaagcagcatccacagccctgttgttctgtggagaggggagagagcaaggatgggggaggcacataggcggctgattttgctttgttaaatgttcaaacctttgccctcatcccctacattcaagacttaccagcagcagtgtatccgcacagcaagaagtatttttgatgtcccctggagactgggtcagctaaatgaggctctttaaacctgataagattaacatgcaataatggacaatctcaaatttaaagtaccccatttttctttttgcagcctgtaaagcgagtgaaagttcatgcattctagatgccaagcaccctgataagtggaagctgctagatttgggactcaggatctacagaagggcatctggctaaagatatcccccgactcttatattctatcttcctcagagagatctccaccagtcagggtccatttgtctccaccttgtgtaccgtgccctcgaagtcaagtgtcagcccaccactgccagtgagacctcctctggccagtgcctgcatcctgaactgctgtggtccctctgggctgccaccctgccaagaggagcagatcagcaaatgggccagaagacccaaacacaagctaccagatgtatattcaactaggtcaatcatgaaggagtttctcacatgcattttttaataataaatagccaccaattattatatagccaggttgcaagttgacacattattaaaatgtacaccaaaaaaatgcagcttcctatttcttataaatctttctcatgaactcccatcccatcaggcagcaaaaaggggaatggggaactttaagtttgaatttgtccattgtagcgtataaatctccctttgggtttcaagagtctcgtttagctgaccttatgcctttcaggaaaacatagccttgagatggcctttcctcttcctgctctacttcaaagtggcattttatgttgtaaggtatttactaaaccttgttgtcatatgggagaagggggcctcagacttcttagacttatcagggaccctggattccacagtcctctggatctcatcagaagctaactagtgacctctgaagtatgactggtcccactggttcacagggatcatactgacatactctactaaaggccctgatcccagctgtttgcttacctgaaaagccctggtcatcaccgtctcttctcaccaaccccagccagctggccgtgacctgctctacatcccctcatagggccatgagccacaatgagctggtaaatgtttaatgacaggtcttggggcaagggggaggtggagtcaggcagaagccctgattgtagctgttgccactgtccatggtgtaaatattcctattaggctgggattgatggcttatacctgtaatcccagcacattggggggcgaagatgggagggtcacttgagcccaggagttcgagaccagcctgggcaacagagtaagaccctgtctttggaaaaaaaattaatatttggcaggcgtgttgatgcatgcctgtagacccaactactagggtggctgaggtgggattacttcaagcccaggaggttgaggctgcagtaaggaatgatcacaccactgcactctagcctgggtaatagagcaagactctgtctcaaaaaaaaaaaaaaaaaaaactccagtactcctactgtggtcactttctagctgccaacattaacaccagagaggtgcacagctgactctcatgagctgacatgagccggccctggggaggttttggagtctctgcccagcatgaggcactatctcaactcacctcctccctcgaggcctccccatgattcctcactgccctgttagatgtggttgcaggcagcgctcctcctaggcttaaagcctcccaccagcagggtcctggtaagccaccccatccatgtcacctgtctcccaattcctgaccagatgctctactccaagagagcgaaagcaaagatctgtgctttggaaaactacaccatctgtctccccatcccctttccaggatctacttggaccaaaagtatccatctttccacctcctcttcccctctgccaagagcctcgcttgtatcatattcttcttattcccaggggtctaagtctcatagttgtgttttaatggcagaagaggttccctctattccatgaaacctattttaccacgagtcctccagatgtggattatcaggaatgaaatacatcaacaaatgtctccagaatattatctctgttacaactgtttaagattgtagcagaacagaaaaaaaaataaaaataaaaaagaaacaactgtttatcatgtgtatcaatgtctgtatgtgcaccatttcatttaacctttaccatatcttaaagggttttgtccccactttacagataaggaaatagattcaaagaggttagtaacttgccttaggcaaggccatttaactggtaactagcaaaggtaggatttgaactcaggttggcctccaaaactatgctcttcacttaatgctgaattgtagaggaacaaactttctttttgttcaaaggctattattaatacaagaccccatagagtctacaagtagtttttctcgagtggggaaggaaggttattgcccaagaggggcagctattaggcatattttcctatttctccatcaggttcaaggagtatgagggagagggtgctggcagaaatggtgtctcactcatcactcctggacacctggtaccctgccattagctgttggagggggtagacagagtggaatatccaaaggaacagagattccctccaaccatttaaccccaccccaaaaacatgtttctctctagtattcatgagctcagtcagggcaccccccaaccaaccccagatgctcctgccagaaacatgggagtcctctcagactccgtctgccccaccatatgacacatcgcctaatactgtagataactggactcccagaatacatcccaggttcatcattctctccttcactcccatctggcccgagccaccatcatctctctcttcacatctctctcccttcacagatcagccagagtgaactctttaaaacatcaatggtgtaataactcccctgctaaaaatccttcatgtcttcccatctcacctagaagaaaatccaatgtcttaaacagagtctacaaagccctttattgtccgcccctgcctgcctcttcctacccactcaccaccccccaccatacggagctcactctgcaccagccacactgccccttcaggctctggagcacactatgctatttcctcccttccagctttggtgttttatttcctttgcacagaagctccttcacatgctctttgcttggttggctccttcccaaactttgggtctcagcttaaatatgacctgctcaagaggccttccctggactttatctaaagaagaccctccctgtttatctctatgacaaacccaggtttttgtctttcataattttccccacactttgtaactgtacacgtatgactttacttgcccattgcccgtctccttcaggctggcagccccatgaaggcagggccagcatctgtgttgttatcagtacccatcggcaccttgcacacggtaggtgctaaatacatattggttaactgttgaataaatatggtaactccagaactctgatataaatttaggaaatgattcaatttagattttcgtgaataactatttgcccagcactgtgctaaatgttgtaggagttacaaaaacagcacaatactcattcatttaacccataacaggtgggagagaggtactagtgagaaaaattgagggaggtggcagagttgtgacattagagaggaagcctggctgactctgtagtctgagtaaacattgtgactatcatttggaaaacacaggacatgcctgagctgagctcagatcccctcaaggtggatattcccaggttatctacacacgtacttactattgccctgatgtgagcatgcagcaagcatagtgcacatgtagtaggcattccatgaatttgtccttgtaatttttgaaagcaagtgtggagatagtttcactttttccagttatccaggtttgctcctaatccttccttctgataattacagaaagatgtcagtattgcagcctgtttttaatcttgtttgagaaagcagctgaccagaccttttttctcctccttctcccactccctgcaataaagcatcctcagaagcttcaactctggaggcaatgggtcgaaaggaagaagatgactgcagttcctggaagaaacagaccaccaacatccggaaaaccttcatttttatggaagtgctgggatcgtaagtcctggggctgtgaggtcgggtgggctgggctgcctgcagtgggaggttagaagagttgttctctgctggcagcaattgatgaactggactgaggctgctgtgacttcatttcgatttatttaagggacttagggagagttttgtggcctggaggatggtgggtggagtgggtggcaggtactttgggtaataccaaagatgatttaatacattggatgtagtccaagctacacgggaggctgaggcaggagaattgcttgaacccaggaggcggaggttgcagtgaaccgagattgcgctactgtattccagcctggtgacagagcgagactgtctcaaaacaaaacaaaacaaaattggagctctcagggaataccagtccctttcccccagggtttgcctccttctgacccacctgcaatgaccctctcctccttccagatctgtgccggtttgtgggttcttgcctctgtcaggagaggggagttggagagcacttctggaggtccagtgacactagaactttcccagtctcctcccccagcatcttcatttctccagcagtgaacaacctcttcttctgctctaactggaacagaagggacttttgaggcccagggagctcgtgggtcacacacacctgaatcagccccctgcaccaccatcctccctcctccatatccaagtgtttcagctgtgctctagtccatacctctgccagcctgcagggtgtctgaacctgcaccttgaagatgattacatggctggcagatcagagacagtttgtctggaactgactccccacttagtcctgcctctcccctgggaggctgcagactctgacgcaggctggcagcagctccaggccactggcccccaaacccctcagaaccccagctggtcagctaattagtctaggctaattgggcctgggcagtgaagagcagccagcgggctgtccatccgggtaagctttctctgtcgccaatgtcacttcactttgcactgactagaaggcagtgacctcgggcaatgacggctgcccacactcgtgccgtagacagagaagacacttctgggccctcctggcacaggcaggttgtcagactgtgcaagtgcaggaggatcccagcggggtgctggtcatgagagcattggccttccatcaaaggcctttaccaagatgggagtgaccagagagggggttttggcaggagaaagagacaagcctcatgagtaagtcagaaatgatttgctaagtgtcccaaaaatccagaggagaggagagaagggagcccaggttgacaagaatttaccatctgctagacttgtgctgtaggactcaaaagacagacacaccttccctcttagagctcagtgaagtctaccacacagcacctaaacctgggagaacatcggagtcatctggagagtttatgcctccaacttttaactgtgaaaatgttgaaacataaggaaaaggtgaaataatgataaaatgaacgtcagtatacccaccatctaaattcaacgttgttaacatttcactgtagagtatgtattttacatcacacacacaccacacacacacacacacacacacacacacacacacacacacgtactgagcactgaactatttgtaaataagttgcaaacatgatgacatgtcacctctgaatcttctgtttacatctcctaaaataaaggcattttcacatatatctacaatatcattgtcacactcaagaaagtgaacaacaattccttttagcctgggtgatttttgaaaaatagatccctggtctcagacccactaaactggaattttgggagataggatgcagcagccaggaatctatattttaagtgcttctggtgttctaatgtatctagctcagcagtgatttgaagggagcaggaggacaagacaggaggcatgtcacagctgttgcagaagttagagctgagcgaaagagaagacatctcagtggtgctcaggatgaagaccagtattcccaggctgcagaccccacagagttaccacagtggggcatggtggagagtggacaagtgtacaaattatgaaaaaggtgctcttttatctagcctgttgtctgctcgggcaggaaatataataattatcatatgaacatgtatgatggcttcaaggtgaatggcacctgctgaagttgttgcactgcacaacctgcccaataatagcaggggccctgtcttaagtgtccattttctgtggtctcttaatatgtcattcttatgtgttgtgtatgtatctgtctttcacattgaattccaagctagaccaatactaatcttagtgcctgacacagagaggttccatcagttcaataaacattcattgaatgcctgatagcatttttcaaatcaaatgctaattgcttacttgtccctgttcctcactattctacaggtttcttaagaatggaaacactcaccttttaatttccagtgtctaatactcagtaggtgttcaataaatcttggttgaagggatgagtaagtgaatggatgaatagagcaagctcgggcggctttgattaactcagttccaagtgcagctgggaaaacaggaaactcagacacaggaaacaccaagtaatttctaatgcagatttttaaagtccttatagaagcttgactcctattgggctttcattctagcatatccagatggcagggtcaaggatggctcagctgtttccttacttagattctctctcctcttgtactactgggtgttcttcataatggtattcaaataatagtgatgataataatagtatctaacacttatggtcttaatagatgtcagaaacacttttaagctttttacatatatcaagtcatttaatcttctaaacaaccttgtgaatcagatactattgatactcccacatttcagatgaggaaactgggaatatgggagttaagtgactttttatcccaaaagctgagattctaacgtgaacaatcaggcttcagcatctgtacctagcttaactgctggtctgatagcctgatctccagaggacccccttcaaaccaagaggcctgtaatttggaatcctgccctgtattcccattggttccacctgctctgtcttcagagcctgcctcagagactgctaggttctctcttttgtctttgcagatcctactgaatgaatgagaggttgctcagcaccctggcctctgataactggtacagagtgatcaccaccacctaaaattgagagacgagtctttttgttctcactgtatctgcactctgtcccatctgctacctccagctgccactccagtgtgccatcctgagtcacattgaaagtggcatgaattgactctgtagtggcttctttttagtgggcatgccaagtgaagaaatctctccacagctgcagtggagtctctgtctctaggggtcattcccaaactatagaaccaggaaataacacctgcctagcccctgaatctgtggtggcatctttttcttccaatatggtcttcaaattagaaagcacattagcatgtcagatacaactgccatcaacataactcagcagcgcccacatgacaatgaagagtaattatcagtccttccctcatctaccctttcactttgcctctaagtttgattgtttaactttttagttggaaataatttcaaacttagaaaaacgtttcaaaaataagaataacgccaggcttggtggctcatacctgtaatcccaacgctttggaaagccaaaggagaagagctgcttgatcccaggagttcaagaccagtttgggcaactgggttagtctatttttatactgctatgaagaacagcttgagactgggcaatttataaaggaaagaggtttaattaactcacagttcagcatggctgaggtggccccaggaaactgacaatcatggcagaaggtgaaggggaagtaaggcaccttcttcacaaggcggcaggaagaagtgccaagcaaagggggaagagccccttataaaaccatcagatcccgtgagaactcactcactatcctgagaacatcatgagggaaactgctcccatgagccaatcacctctacctggtctctcccttgacacatggggattatgaggattatggggattataattcaagatgagatttgagtggggacacaaagcctaaccagatcagcaacatagtgaagccccatctctaaaaaaataataaaataaaataagaataagaatggtagaaagaatttccaaatgccccctgcccagattcacctcttgttcacaatttctcccatctgccttctcattgctatgttctctgtagatctgtagagatagatgtagaaagatgattctttcctaatctcccaagcgttagttgtggacagacatcatgaacctttacctatcaaggacatttgtgtatatttctagggatggggtattcccttacgtgaccataggacagttctcaacttcagtcaattgaacatggacacattaagtgtacctagtctgtcttccctactccagttttatcaattgaccctattatgtgttttatagcatttttcccctccagtacaggttccagtctagggtctgatatttcacttgggtgttatctctccctagcttttttatgacattggcattttggaagaatacaggttccccccttcccttttttaaattttgtgctttcctgatgtttcttccagatttaaattcagtatatgcctttgtggtcagaggactatatagactctgttacgtcctttctgaggtcttacatctggaagtgtaagatgtctgccccctactaagttttatcttttggtgtcttctcctcagaggagctttctcagaagttttcctggtgaagcaaagactgactgggaagctctttgctctgaagtgcatcaagaagtcacctgccttccgggacagcagcctggagaatgagattgctgtgttgaaaaagtgagtgggtcttagtgttgactggatcactatgggatcacaacattttcttcacaaattaccttcaaaaaggactaggactggatttctgcacccaaaggcattgctcactttgattgagttgatgggtcagttcagtcaattagctaagtttagtcggacagctgtcctagctgatttttttttttttggcaacaaattgatgagcaaatccactggtggcagaactggtttgtgcacacaggctggagagtaattagctcaaaggaacaaaaatcaattgagggccaacatcaggaacaattaataattaaggaggcagcataagatttggaaattagcttcaatttataacaaagcaaatggaggttcagcaccccaaccatcttgatgaaggcaacactacaaatcatcatatgtttacagcaagactcttaccacagacccttgtgaaacccttatggtgtaccaaaagtgtattaggcacatgaacacaggatcagaaccacaaggatgaggcagacaaaattccttaccctcaggagcctatgctggatacagaggtgcctataagaaaatccaacaaaatcggatgtgagtcacactagcagaaggcagagtttggaggagataaagattcattttgcctatgcatatgtttttatgagtatgagttattggaccactttcaaagaagatggaccttgatagatgagtcaaactttgtgggggcaagaaaggcaatgcagaaagggaattcaggccaagactagcataaactttaatgtagaaccatgaaagtgcaagggcttttagggaagagtgagtattggaaatccctgaatttagagtatatagtattaaaagacaagtgatgaaactaaaatcatgaaatgagacctgattacaaaggatcttagatgccatgataaggaatgttggctttattctgtgggcaaaaagaagtcatcaaaggattttaagtgaggagtcacattgtctgattaacatgttggagctatccctctcaggcacagtggaggatggattggagggtgccagcctagaggacaggaggctaattataagactttcacaataatttaggcatgggttgagaatacaagcaaaggtagtggcagaatcatatgtgcctaaaaggactagatactagagatgtttcaaagagagaagtgattgggtttggggttgactagtgaaagtatacaggacaaaagcataaaaagagaccctgagattgagcaatcaagtggacaacaatataattgctaggggtggagaacaaagaggatatgtaggcttggatgaaatagaaagaaattggttggaacatactgaatatgagtatccacctatctcaacatgaaggcatttcataacatgctggagacacaggtctaggatccagggatattagggcaaagaaagactctcaactaattgagagcaaggaccatgtcatttttcttattattgccttgtatcaacagtatttgtgctcaataaatgtttgttcattgagtgaataattgataaattaatgttagcattgccagcttctgagtatagttgaattcatcaagagacaactgacacaggtctatattttctaaaatatcgtaaagaagatagctactattgattatgtgtttactattacccagacactgtattaagcattatgcattatctccttttatcctgatacaattctataagagacacatacttttcagacaaggaaagtaaagatttgaaaactggaataagccagctgaagttatctaattgcctgagctagatctgaacccaggcctgactgactctgagcctaagtccttaacccctgtgctgcacagccaggagtaaacttcctagagaatttcctcctttacaggcaggcacagcattgctgcctggcaagccgagcccctctgctaatatgattattctgcttgactcagtctctgagtaaccccaaacaagggtaatgtagggctggacttctccaccatgatgctgtcgacattttgaaacagataattatttgtggtgctgggggctgtactgtgcagtgttggatgtttagcagcatttccgcctctacccactagatgccagcagcaccttcccctgccctagtcgtgacaatcaaacatgtctcaagatattgccaaatgttccctgagaggcaaaatagtccctggttgagaatcaccatctaagtcatcccacagaactaaccctaaaaataagcatctaccttgcttctcgcaatatcttggaacactaatttttcaacacttcctccgtggttatccatgaggaggctcagctcacctgaatccctcccattgtatacaggttgagcattcctaatctaaaatccaaaatgctccaaaatcagaaacatttttagcattgacaggatgccacaaatagaaaattccacagctgacctcatgtgacaggtcacagtcaaaatgtattcaaagctttatttcatgcacaaaattattttttaaatattatgtaaaattacctaccagctatgtgtataaggtacacatgagacataaatgaacttcatgtttagacttgggtcccatctctaaggtatatcattatgtatatgcaaatattccaaaaactcttctagtctcaaacattttagatatgggatactcaacctgtactttttaagtgcaatatttctaacatctgaatacctactttgtagaataacctagtgttgggtactcccttagccttcctccattcttctaactgtctcttttagtaaagtttaaagagattcctggcataacaaggaagtgtttgtttcctgaaggaaggatgatcaattattcattcagtaaacacggaccgagcacctccacaggaatcttttctctgggattaatttgtgacagtctacttattatgaaggcttcacagagacctagtcttcctaaacattgccatcaaaagcctccaacagaaacttttgggctaggtctgcattatttgtctgtacaacctgaaccacatacctttcatcatgcactttatttttcccaggatcaagcatgaaaacattgtgaccctggaggacatctatgagagcaccacccactactacctggtcatgcagctgtaagtaaaaggtgacttgcttccttcacagaggcctggtggggcctgggaggcctacaggaggttggtcgctggtgagggatggacagcaattcaaagacaaaaatgaagacttcattcaggaggctcagaaatgccaagggtccttttagactgagtgtgcctctggtcatgggcaggttctatgtgggccacagggtaaacctgggctgattattaagctcctttgcaccaggacaaccctatcagtggttagaatatttcctaccagtcagcaaatagctgcgaccccaacccatccatgtccttctgctgccccacccctcctcgccccttgcaaccaaacgaccgaaggggtaatgccagactaacaaggagtcaccagaacccttttctggcactgtggcttcagtccttttggaccaatacatgttaatcaaattggatagtaaatgttttaacatcactccagcataaaattactgtaggagcagaagccacaggtagatatatttcaatataaagaaagattttcccaaaacagagtcacccaaagttaaagacaggctgtgattgtatgtgtccatctgtaggacagaagtggtgcttcattagcaaggtctctgcgtcattgctgcatccaaacaacagctctgcctgacagcaactgctgtagaggttggtcagtgttcataagggctcagcattgggctcaaagacacttctgtttataaagtacacacacacacacaaatacacacacttgcacatgcacagacatacacgcactcacacaccactctgcaatctcagcgtggctggacacaacgtggtttttggctgatgggctgattactaagaaactttaacgagataactcaggcctaagcacctgattaactgggggcttatctgaggacctctaaagagaggtagcaggcagtcaagggagtgaggtggaaagggcccctagtggcactaacaagtgatcagggaccatcaggcactacaatgaggccaagccaacaggattagcacacagtaattcagaaggcagggaggcaaggagatgggaagagttggatgtccctgaggagacactaagcctcctcagagcatagctccaggactcaggcccctcagggttaagaattcatagctctgtacaaagagtgtatttcctacaggccttcagctactataggagcgcccaaagcctaaagaccaaaagccaaagagcaggatgaggtaaattttgatcggagaaattcttttccctcatcactagttgggcatggatggccacatctaatgtcatgggacagtggtaagggggactaggagggtgagaggactggaacccctggctcacggttgaaatagtctgggaatgcttatctcagagaaggcaagccctccggagagcatttcactatctttgtatacttgaaaggtgcacaggaaaggctgttattcttgttctgtgcagtcccgatgggtggaattggtagcactggtagaaatgacagggcatcaggtttcaacataatgtgcagaaacattaggaatattccaaaaaacactctgtcttaggaggaagagtgttctctgacacttatgtgttctagtactcactagaaaacttctaggcaaaaatagagtagatggtattcatgagatagaagtcagacttgatctttaagctcccctgggtcctaagatactctaccatttgagtctcaaagcacccctaaagccctacctgtaattccagctttatgaagcccaatttcccaaaatgagaagagaaatcttgatataagtacggttagtctcacttgcatgccttctctttcctactttgcttaatttctgtgttacctctctctttcatcatggcttcctgtgtcataggccaggaaggaaggtggctaagaagtgtgtccacactaagggattattggcttcttgtgcagccccaaccagaagcatttacattttaaaggaaattttcagagagagaggtcaatgcttggcccaaagagatgactgcctagtgatatcactccaggtacccagagtgggtgttctgagactggagtagtctgctggcagcccttcaatcacagttcatgggggcctgcctgttccactgcagctgtgtcccccaaggcttcaaagcaaacaccttctcattactttgagtgaaatgagaactgaattcctgtcttgatcctatgcccacagtgtttctggtggggagctctttgaccggatcctggagcggggtgtctacacagagaaggatgccagtctggtgatccagcaggtcttgtcggcagtgaaatacctacatgagaatggcatcgtccacagagacttaaaggtgtcaaggcgggagtcctgggtgggaaacagataatgacccttaaggaagctgcatgggtcatgggacatctaagctccataaagtaagagggttggactagtgactcccaaggccccttctgcccttagattccttaaacagaccttggatgagtgaacaatggttactaatttgcacctcaatttttcctgaaactttcactatgccttaccctatttcaataccaaaataatacacacatttcttacatccagccacagtcaaaacccaatgcaacaaaatatttactgagggcctacattgtactaggccttgcactggactctggagagatcagacaccattaaccaaagattggttctctcaaaacacaaacagggtagttggaagtgagggaaggaaagggtgagtatattaatgtttacaagaccgagcaaagtcagagccataagcaaggtgcagactactggggtgcaggtgtgagggtgcaggtaggaggaagaagggaagaatgggggaggatgagctggccttgaagtcaggagcccagggctcatcttcctgtgaaattttgtatcaaggggaaggaaaatacttgcttcaggtggttatatcctacactacatattttttctcctacagcccgaaaacctgctttaccttacccctgaagagaactctaagatcatgatcactgactttggtctgtccaagatggaacagaatggcatcatgtccactgcctgtgggaccccaggctacgtgggtaagtctgggagcaggaggaaggttgaccagttggctacattcaaccacatgcctatcattcatagaaaggggtgattctgagaacatagggttcaacttcaggggctatcctggtaggacatccacttataaagtttagggccagccaattcatccgtctcaggatctatctctactgctcccaaattctacatttttaaagaccgcctacccctgagccctccagagtccctgcctatgctttggctctcatttctgcggatagacttccagcctttgtctttccagctggaccaggggcagacaagatcacaaacccagagatcagcagagagctgaggggttgagcaccaaccctactcctttctcccactctgaaggagacattagcccccagttcttgggagcctggaagcacaggatattgaatttgggcctgaggaacccagaaatgggctcctaaatgtccacccaaggaagcactctgggaaattggcaaaggtggagcttcctgcttgaacatccctgtaggtttccagggcccccaaaaagagtgagagcaggcagggcacctggaagagtattcctgggatctacaggagacaaagttgaaactcagagcatccgctcctgtcccttcccacttcaccagtcgtcacctgtttgcagttgggtaatgggaagcagagctctgactcaagagagggaatctaccctcccagaaaggcaggtcattcactcctcagtaggtattgagtcccaccaggtacaaagctctggtagctgcaagaaatacagaaggcagctctctacaccccacagggctcctaatcagaggaagtcatggctcatgccctctgaaagctctctatctgatgggacagactaaatgcctcccctcagaaggtacccacagtctgatgggagacatactgcctttgctctgggagctcctaatctaatgggggtgactcagtctctgcctcccagaggctcccagtctagtggaggagagacagtcctacactgaagatctcccagtctgaagggagagagatcacacttgccctgggaatcccctatctgagtggggagacaggccctgccttcagagaatactcagtgtaatcagggagacatagccctggagatccccagtccaacaggtgagtcatcgaccctgtcatggggaaactcacaaactaatgcagaagtcacagtctctcttttcaggaagatttagtcttcccagaagagacacagcccctacccttagggagttttcagtctcctagaaaccccaccatcccacagtcttccccagacctcttctctaagcctggccttcagctcccaccccaaagccctctcctcttgccaccagccctgactctgcccttggtctgctgcagctccagaagtgctggcccagaaaccctacagcaaggctgtggattgctggtccatcggcgtcatcacctacatattgtgagtagacgctggcctgggttcagctgatgagaagtctcggagcctgcttaccccttctcgttccctcccctgtctcagctcctcctccaagcacgtgcatgtgcacaaacagacacaggcacaggggcacacgggtacacacacacacacacacacacacatacatgtacacacagcttaaaagcttcctctctcttcacctccaccaagttttaaaacagagctcagcgtgaatccactgcacagccacctggcactccctcctcttgcacccctgccccttttactgcctataaatgtgttgcatcccactcagtggaatgaacagtgaaaaagagagaggccaagccaggcttggaagccaggtggaccctctgttcctacatctcagctttcccaaggcaaaactgtggcctggataaagctggtcagggcctttccttttcgctcctctccacacattcccaagctctccctttcctaacctgcccatcacctcttccccaccatccctcctcacctatagcaaactttgctcttaggaaaataacctggtttcaattatctcctccatttatgttgaataagtactctcatttatttacccaaccaatattcactgcctatctactctgcatctatccctgtgctaggttctaaagaatcccgcagtgaacaacacagcctaaataaatccttcatcaagcttacattcaagtagacagtctaccaactttcttaaacatattttatatagcattcataagtcagggaaagtataaggagaggtggcaagggttggagggcataatgtacacagggaccttctagggatgactgtgccaaggcttggcacttgcataaacactttacgggacctgtaccattctggaatttgaggcagatctgcgtcctggtcacaccactgttctgggaccttcagtgcccacctgtgtatacagtgggagcttgggggagcagagaggctggctcagggaggacaggtttgttcagtagtatgcctccttcctgtcctgctgcaggctctgtggataccccccattctatgaagaaacggagtctaagcttttcgagaagatcaaggagggctactatgagtttgagtctccattctgggatgacatttctgagtcaggtaaggccagtaggcatgaaggagagataacaggctcaaggtagaggctgccaagagaaatgagcttaacaaaggatgacagtcccggctcttcaaagtccctggggatcttacagaacaggctgccaaggaaagtggagaaatcttcgtctgcttcaaagacccgataaattatttacatctaagagctatgcttttgtttttctggaggcagaaccacagtatgcctgaaaaggagatgggggggcagtttagaagatgggccaggagggcagagccaggtggtgaggggagagttccagacaagagcagcaaaactaaggggttagatatcaagaaagagcatgcaatgtctatgtgtgagagaagatgatcattacgtgattgttggacctgttaagtggaaatcagagctggaacaatccagcactcacatgcgtgcccatgtgccagcgctggcccctgtttcagtgcaaacacatgcacatgtgatgtgcaggcacatgggggccttgtgactagtgacgcttctgcagtgctgtgtgggtgaggcggcttggagcttcccttctttggtggtttatggtgggttgttgttaggtggtttggattttttttcctcttctaagactcttaaaattgcctctttggaagatcagctaaagatgcattcactgccacccaccagcccggaagtgaatttccatcagtcatgaaatctggtccttagtcgtcgtgtctttgattactccccttagccaaggactttatttgccacttgcttgagaaggatccgaacgagcggtacacctgtgagaaggccttgagtcatccctggtgagtgagacatggagtggactctagaccccagccctgtagttcccaaaaccatgctgactcattcatattgcatttccctggaatctgcttgtcctgatccatttaatgatgtgacaagcaaggaccatgccattgttcatcaggcccaggtagagattcaaacattatacagtggagcaggttctaatgcaagggtagcagctgggttgtctggtcaagtgcaggaggcaggtgcagccgagcgctgtcattcagcactgggatagggtgcaatgttcagtactagttattcaagaagtgccaggaatgggcaactctgtcctgtcctttagagctttgttttactcagggatcaataactgttccaggtgaagtgatttaagaacaactgcaaagcaaccgattggcaaaataggaaaaagaaaatcttaaaatattttttcatgttacttcccaactcaatttacaatagctccccacttcataccccagcaagcctggattcctttgtctttccagtttccctaatatggcctgaccctttatctccagcctcttttcccaggattgccagcatacctcctctgtccagataggcctgggagctatatctgtctcaccccacataccaagctctgtcttaactacaccttggatcagatcattcccactacctacaatgtcctgcctcttgccctgtatgtagcatctttcaggatccagatcagtctctgcccttccctgaagcccctgacccctgcagcccaacccaatctccccagttctctttattaatcacacggcttaccatgtattactgcagtccatagcatgaacaacacccgacatctagactgtaggctcctaggggtcagggcccatgtctcttacttctcgaggtctattagtagttggcacagtactatgcacacagcaagtgtctgaggaacacttagagaccagtgaataagaggctaaggggataggaaaggggcagcctgtaccagacggccttactgaggaaggcttccaggaggaggtgggttctaggcaaggttttgagctaaaggatggtgactaccccagtgccctgtgatgctgggtccaataaactttttctataaagcatcagatagtaaatattttaggctttcctggccatactgtctttatggcaactactcaaccctgctgtcacagtgcaaaaggagccacagacaatacctgaattaatgggcaccactgtgtccccataaaaccttatttacaaaaacaggcaataggtcgatttggcctataggctatagcttgttgaccttgctctaaaagtttttgattttgccaaagctaaagccgactcttacttgcccaggggcacagaggagacctgcttgcagccctttcctgctgtgggctgtctttcctctgcacactctacccagctctcacctagacctgcaggcaccctgcccactccctggatgagtgtaaatagccacccagtagccaggtgtcccctcttacatccacaggattgacggaaacacagccctccaccgggacatctacccatcagtcagcctccagatccagaagaactttgctaagagcaagtggagggtaagctgtcctctccagggggtgggaaagctgttctgggcccctggaggctgggctggcaggggctgacataagggctttccttgggatgtcccagaaggcattggagctccgtgtaccctctctgaaatgagaagtgggcacccaggtttcaagaggccacaaggcaaagggaaagtttaagctccaaggccctctgaggttgcagaaggccagaggctgctcttgtgtctccttagcaagccttcaacgcagcagctgtggtgcaccacatgaggaagctacacatgaacctgcacagcccgggcgtccgcccagaggtggagaacaggccgcctgaaactcaagcctcagaaacctctagacccagctcccctgagatcaccatcaccgaggcacctgtcctggaccacagtgtagcactccctgccctgacccaattaccctgccagcatggccgccggcccactgcccctggtggcaggtccctcaactgcctggtcaatggctccctccacatcagcagcagcctggtgcccatgcatcaggggtccctggccgccgggccctgtggctgctgctccagctgcctgaacattgggagcaaaggaaagtcctcctactgctctgagcccacactcctcaaaaaggccaacaaaaaacagtacgtatttttagccaaagatggagccccagcttgggtctgaaagaaatcggtcaacaggactgaaagaaatggacacaaaggcctctcccactcataggcagctatatagggagggatgagttctggatgagggggcaaggaaaatgcttccagccctgtccccatcacttactagttgtgaggccattgacgagtcaatgtctctgcttctattttgctcatctgtaaactgggttgaagtctatttacccaattagcctgcctcacagaggtactataaaaggttagtgaagcaacagagatgataaaagcccacagaaaactgggtatttgcaagtggtgagcatcagattccccgagtttccagggcctctgtgctttgcagaaggaagtgtaggggggcttggttatctttatcttttctgcaggttcttgaactttctgaacctcatgaggggagagctgagtggatgccacaggcacagagaactaccacctctgccctgccccatcgactcttcttccctcaaatacttcaaaggttgttgcttcatttcctttctaggaacttcaagtcggaggtcatggtaccagttaaagccagtggcagctcccactgccgggcagggcagactggagtctgtctcattatgtgattcctggagcctgtgcctatgtcactgcaattttcaggttaggagggtcacagtgtgaggcttggggagagtttctgtcccagaggagagccccatgccagagtgtgagtgatggggtctgaggtggcaaccccctcctcactctgagcccctttctcttgcaggagacatattcaactcctctgctcttccaaacctggtgtctatccggcagagggaggaaggcagagcaagtggagcagggcttagcaggagcagtttctggccagaagcaccagcctgctgccagcggggcagcccctcataggaggcccaggagggagccccaaggcgtagaagccttgttgaagctgtgagcaggagaagcggtgcccaccagcttccaggtctccctgacctgcctgctctatgccccacaccctacgtgccgtggctctgtgcagtgtacgtagatagctctcgcctgggtctgtgctgtttgtcgtgaaaagcttaatgggctggccaggctgtgtcaccttctccaagcaaagccatatggagcatctacccagactcccactctgcacacactcactcccacctctcaagcctccaacctcttggccagattgggctcattaatgtcgttgcctgcccatctgcatgaatgacaggcagctccccatggtggtctgcctgtgagctcttcaagttctaatccttaactccaggattagctcccaagtgcgctgagacccagccagcacacttctggcccttctccctgcctcaatctaaaagcagtgccacaccctccaaagtggaatagaaagaagttcatgagtaagggctgcaaggaattcttatcctggccacatgtcctccgtgcacacacccaatggagttaaccttggaagttgactattttaatgtctgccaggagttctaatcctgcctctgttcccttttctctccttgaaagtccagcacaccattcttgtccttccccagtttcctcgccctccacccctccagcttcatgctcagtgttgtgcttaataaaatggacatatttttctctatgaggcttcatttggttctttcaaagtcaggagcagttcagggacttctgagcatgtctggagtagagagtgcatggatggctggggtcagcaaggaaccagcagttgtagctgatgtgcatacagatgtgcatatagctgagtatacagtgctccaggcactggacatgagttacatcattaactcattacttctcacaacaaccccttgggacaggttctgttggaaccgaggcagtctggctttagtggccattctcttaatggctctattagatgagcatgagttcgagccccagctctgccccgacctgccatatctgtgaccttggggaagttacttagtctttcgtgccttggtttcctcacttatccaatgggaatagcagtgccacctgcatcatgaaatgatgcagatgatgtgcccagcctcgtggccaataaataatcaaaatgattaacaatgatcaagaagttaatgtagataaaatgtctagcacagtgcctgcttgggccatggttaacacacaaatgtccatgccattccatatcatcctccaccccaccccccacacatatccatttacaacttctctttaagaccccaagagacatagattctagtcttagtccttcctctggaagctgcatgaatttgaacgttccttcccttccctggatttctatttccccatctgtaagcaagaagttgaatcgctgcttagagatgaaaactgcttctctggtctctcaaggatagaatccatcaggttctagggcagaggggcccagactccctaaatctgggtgcccagaaagccgggtggaacagaggagagcaaaatatgggcttgaatagaggagtccccacaaaccccaaatgggagacagaaacagtggcaagagtttacccagtgaggggacagactgtttttcctcgttgaacctccaggctcttttccaaagatttttatttggcttttcatttttacatcacttttgttaagtttttgtgcttggtccaggattcctccccagtggagaactaaaggcgggggatactgcccagcccagcttccttgacttgagagctcttagtttcaatggcatgccaatctccaccatctttgtctgtcaagtgtaactgtcccattggctcaggctcagctgcagctcagggtaatcttactagctacacaccaggggtggtccaggctgtactttaggctgcatgaaagtctcctggagatggaaagcattccaacccaatctgcccccaaccaaaagcaaaggcggcagatgagtggggcaacgggctggaagctgtagcatcacttgcaggtggcatagtagagcacgcgcccattgatgtggtcacggatctgctccacacgcttctccagtcctgtcaggtccgctgagcgcagcatgatggcctggctgccccgcagcagctccaactccatgtctgaggcaaatggaacagcagcaggaggaggagttgatggtgagactccccaagactcccaacccttccttcccctcctgctaactggaggtagcatgtgtagaggaatcccctccagaaaaatctggcctctaaagcagtggtgcccctccagcttcatgcctctccagccccatcccatgcttagtaacctggcaggggccaaacccagcaggctcccactcctaattatccctgggcccctgaccctctgggctctcacagttgtccaaggctaatgaaggaggtcacaaacacccatcatcatatcttagcgatgactctagttcccaggagcccctgttctcctttctaaggaatttttggaacccttttgaaagctactttcattatgtttctgtcatattcaaagcccttggatgtgccctaccgcctcctgagctatctaccctcctcatcctcacactagaagccaggcacagggacaagtcatgttgacctcatgccacttctttcctaccagctcctcagcaaccctcttctcctgtccctatgtcagaacatatcttgttggggcttcccaacacccttatcctaatgtccctcctcttcctcacagcttctcccaggcctacacatccttccaggcccatctcaaatcttcttttcttaccttcactgaacatgtctctctcaaattatttattccctgaaccccagaactcttattattcaacccctatcagagaccacctcatcctgttaaatataattctatgcatcttaagcataaagcaataggaataaaaaatatgaccccactgtttgcaaaaacagagaaaatggtcccattaattgcaagctttttgaggccaggacaagtcatgggaccatctctgcattccccagcctcatacatgctagatgcccagaaaaaatctcatttgatctcactcccatagcacggctagctccaataagaacgggcttcagaagccttgggttgggtgggaccagccttcatgaatgcccaatagtccatgcccctaaaccattccctcacctttcatcctgtccatcatctccatggtctccccaaacagctcctctgcctctgtcttcacactctggatccgggcaccctgctcacccagcatggaactctgacccaaccggtccttcaactcagcatacttttgttttattctctcaaatccctgaaaaaggtagaatagtctcagtgtcattgtcatcatgcaaggtcctaaagacctgtggccaaagcacttgatatcacccaaatcaatacacagtgggaaggtgcctcctattagcccccaatagcattggttttgcctattgaaacccctgagccacagactatgacttaaacttctgtgtttccctcttcccatctccagctcttgacatgacccacctcactcccaggacaatgtcagtcacatcataggatattaatagtaggctgtcacttaatagcatacttagcaacttggtcactgtgccaggattgaaagtgccaatgttcctggccatagcgggagcaggtagtgtgcttgagacactgagaatgggctctttgatcttctctctcatctaattctgacaatcatggagcccaggttaggttaagcaaagagcatctgtccaggctgggaatccaagagacaagctctggatccagtgtcaggggacctgctttgtagatgattcttaggcctgtgaacgagtcaccacctcatctgtaaaaggaaagggactggactaggtcatctccagtgtccctgagccagctataccaaaagcagtggagggagaaatcagatgcctggccaggtttgggttgactctcaagcctcttctggttctgttattctaagcacttttgagtttgtggtcttataaagtgtgcaaagtcctcctctgctcaggaccccctcactggccgtacatcattgagctaactccgccttctctgtacctcttgggcactcaatgcctgctcgctggcaccttccgcaagctgctgggcctggactgcctctgccccctgctgccgggcttggtggcggagctcctccatccgtgtccagaagtcacccagctgcttggtcatgcttgtcaccagcttttctgctggccgcagtacctgctgaacctttgtggaaagagggagatctggcctttgtggtgggtctcataggtcggggggttcatccccattttttctccaactcatcaggcagcaaaaaccctctccaaccagaacaaacacaattgcccacctctcacccctccttctgcatcccagccacaagaccagaaaatccaaggcccatagcctgcccaaagcttgtcactctcctctagaactccacctcaggttttatctagtcactttctggcattttctatgcctggtgcccaaattttcctgtttatgcccaaatatgggcggaggaagccataatgcctcacctcagcaaccctgtcctggataagccgaagggagcggctggtgccttgcatggtgtcctgagcttcctgcagtgccactgtcccctgccgcaggttcccaaccacatcttccacctggccctccactgcatgggctcggctcctgggtgagagaagcagcagggagaggagagagagaatgagtgaacagtgaatcaatgtgtggttcgactcacacaccagtccaaaagaacacccttcccagccaaggcaaagagcaaggaaagtattaactcagtagctttgatgtcccaggaccccaccaagcagacgtgatgaatgttcccattatgcccaagacaccttctgggcggtttccaatacggaagacacagtcatgcaaattaagtttaacctgcactctggactctgtgtaccactctccaccatggtaagcaccccctcctgtctcccagccctctgtacccctctcccagcaacggggttgaggagcaaaacgccagcttaatcctgggcagagagaagttcagggcccaaggccatacctggcttcctcagcctcagcctgcaaccggcgggcacgcgcaatgtcctgcttggtctgggacagcaccaagtccacgttggggagcctggctgcaatggcctggatctcattcatcttctgcagaacagtagctgagtctgtgggcagccacagggccagcacggcctcgctgacctcctggatagtggctgcatcagtgtcggggtctggaagacaacacgcatttgactgtaggagcccactaacctccccagagatacgaggcctctcctagctgagcagcacttgtggaaggcacccacagtggcccctctaccgtggtgtcccaaggagctgctgccccctttcaaccagactcaggcgcctgttctccagggatttctgggaatgctgtaggaggatgactatcctgtgctccatgctgcagcagggaggaggaggggtgtcgtcacagctgtgccaactaagtgtctggtgccacaggaagctgcgggctggggagaaggaggtggtgccagacagctggtactgaacaatgcctgcagcacatgcacaatcagccctaaattgggaagagtggccagaaactggagctgcttccttggcaaaaagaccccttccctgcattccccctcaggcaagattgctcatttgtgcaaaggcaaataagaatgatcaagtctctgctggaatatcaccttctcagggaggccaaccccagccatccaatctagaattgcaacctccaccctggactgtagcacaccttatcgtccctccctgccttatctttctgtctagcacttgcaccacccatgtgccaaacactgtactcatttaactggtttgtttggtctcttttcctctagaatgtaagctctacaagggcagggcctgggttttgtccacagctatatccccagaccccagcacagtgcctggcacttggaggtcctcatgtgcgtagagtgaatgaatgattgacagggcttcaggttcagatctctgctttactattcaagccacctgggcaagtcaagtggttcctctaagctcagtttcctgatcggtgaaaaaggaatatcaatccccaatctgaaggtacaacatgaaacaatccatttgcaagagttgttacaaaccaactctgggtcagagggtaaggtcagaagtacagagatgacatctcctccccatctctcccaatttgcttttacagatttcactggtattgaggtattaaggaggggggcacccactcaccagatgatttgccctggaatctgaactcctctggggccccaggcccagtttcactctacaactagaggatactagctggtcacacaggtctcattccctgccccatcccatgccaggtggaaggacactcctggagggtggaagccatgcttgttcattgctgtgtccctcggggcttgcatgcagcataactgtggttgagtttcattaagtgggtttatcccagcagcaagctttcataaggaccttcctaatagagctcgtcaacctctcttctaacccagagtcccactctcaaaacaaggcttcatgtgcaccttctcaagaaagccttccaggatgagcacatccccacctagatcttgcccttcctttactctgcaccacttcagctgcatcctcagggtcaatcggtatgacattcagtcgcagagccttctgggtctcaggatgggctcagtaaatatttactgctttgagcaaaagcatgggaggagttgagactgcaaatcaaattaaactcagtccctgcctttatggagctggaaatctaatgggagacaggcacatgaacaaggaagtacagctttgagtgagcggagtaaacacaggtgctatgatggccatggaaggggcccccagcgccatctgataggtccagggaggctgcctggagagtgggatgcctagattcctagaggatgagcaggagctagacaggtcagcagaggtgggagaggggagacgatccagagagaggaaaaacattcgcaaaggcaagaggcaagagaaagtgtgtttgtgtatgcacactcgcatgtgtgtatgtatttgtgtgtctatgccgtaaagcaatagggtgtgaatgacagagtcagacaattaggcttgaagcctacctgcaccactgccaagctagatgcctttaggcaagttgcttaacctctctgtgcctagattacttgtgtgcaaagtggaagtaataagagtacttagttcatgtagtcaggaataaatgatgtaatacatgcaaagcacttggaacagtgcctggactcagttagaacttaataagccttaactattcattttgtcaattccatttgaagaactgggaaaggcgatgtttcctcctccctccacagactttccactctccctcccacactgtgaccctatatcggacagcagccagcgctcagtggcttctgcaccaggactctggagaccagagtccaagcccaccgctacagaaaattttcctgggtttccccctggggccaggcctctctcaggccctgctttttctctctgaccctggactcatcataagacttgacctgagatacaagtataagagagaggacaaaaggggcctggcagctccctgtctgttcacatgagcctcagctgcacccggcaggagagaattccaactcacctcccacccactgaggccagagccctggctgagattaatgcaggatgagtggccattcagcctcactggacttgactcaggggcaagaatcccttatccatgagcctaagccccacaccttgacctcccccatcatccacgtttgtggggcacaatctccctacatccccaggtaatattttccccaggtgagtgcctcaaggccttatgcaagtcatgagacctcagaagaactttcctgcccctctctgacctgccacctgcactcaggaggggacttcggcctatctggggcaaaaacactgtaatagtagcaagaggagccactgtcattacaccaagccctgggctcgtattaactgggctcccagccaagcaaataacctggcaggaaccagaaatctaacaatatcaactcttgttcctgaggcacttttcctttttcaaaacacattttacatcagttatttgatcctcagaataaacttgtgaaatactaccgtccccatttatagaagcaaaagatgaggcaaagagaaaagcagcctgcccaatgtcacacgagtctcccgggtcctgtttagggatctttccattgtacagcccttcctcactccccagagcagtgaggaagggtgtctggttcatttattcatgccaccaatgtcagatgtctttctgtgtctggccttatgttaggtgctggtgacacagtagtcaacaagacagacaaccatccttgccctcctggagtccccattctagtggagtgacaatccacttgttgacaactgaatgagggtgagtagtgcaaggaaagtaagatgatgggggacaaacagatcggatggtcaggaaacacctctctggggagatgacccagaggacaagaagcagcagccctggaaagaggacttcaggcagacaaagagcaaataaacatccctgaagcaggcaagagcctgacatgttcaaggacagccagtgaacaaagcagagaagagcccaaggtcactcccaagagatggacagaggccaggctctgtagccacgatgggagttgagagctcattccaagaacaaggtgaagccactgaaggattttaagcaggagtgagatgtgatctgacttatgcattttaaaagagatatttcagctgttgtgttgagaatgagaatgggtcacaagggattggagtgggtggggagggcagacgggagaacgagaaggtcatggccatggtgccaaggagaagtggaggcctggatgttgcaggcctgggactagggagggagggctggctgatgcactgaacatgggaatgcgggacaggcaggactggaacagcagccaaggtggggtggagactgggctcacctgttaggaagtcccggacctgctggattaggagccgtgtgcgtctgacatcttcctccatctgggagcggctggcgctcacctgggtctccaagcgctgggcactggattgaatctgtgaggcagattcctcggctgccctaatctgttgacatacactctaggtcagaaggggtaaggccccaagggaacctctcagcagccccacgattctgcggatcatcctctatgccaagacctacctaggaagcatttgtaaccaacccagcttacatgtggatgttcagtaaaagagaaaaaaaactatcttgcacttgctgtggcaccttaagcaggtcacctaaaatctctggactttagtgtaaaatgggaactctgacttgcccgggggatctatcctgtctgcctcctcctacctgtgcccaccccgcctcggctgcacttaccatctgcctggtccgctggagctgggcattgaagccccgcagctgctcagccacctgccccgccatcaagaaggccccaccggccctgggaaggacacccctgcagcgggagccacaggctgtgccattgtcttggggacatagctcaccagggcatgatattggggtgcaagccatctgcctggagttgccacagagctgtggacagatggcggtgttaaagaggctacccaaagcccctcaataaccaatcccaccccacacctgggaaaccaacagccagacatctccatgacaaccaagcaccagaaacagccagacatctccatgagagctaaggaccagaaactggtttccttggcaaccactgagctcacagtgagcagtacaagggtcgggatggctgggggagtggggttctcacaggggcagatc

SEQ ID NO: 2 - DNA, 1383 bp, Homo sapiens
atgggtcgaaaggaagaagatgactgcagttcctggaagaaacagaccaccaacatccggaaaaccttcatttttatggaagtgctgggatcaggagctttctcagaagttttcctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcatgcagcttgtttctggtggggagctctttgaccggatcctggagcggggtgtctacacagagaaggatgccagtctggtgatccagcaggtcttgtcggcagtgaaatacctacatgagaatggcatcgtccacagagacttaaagcccgaaaacctgctttaccttacccctgaagagaactctaagatcatgatcactgactttggtctgtccaagatggaacagaatggcatcatgtccactgcctgtgggaccccaggctacgtggctccagaagtgctggcccagaaaccctacagcaaggctgtggattgctggtccatcggcgtcatcacctacatattgctctgtggataccccccattctatgaagaaacggagtctaagcttttcgagaagatcaaggagggctactatgagtttgagtctccattctgggatgacatttctgagtcagccaaggactttatttgccacttgcttgagaaggatccgaacgagcggtacacctgtgagaaggccttgagtcatccctggattgacggaaacacagccctccaccgggacatctacccatcagtcagcctccagatccagaagaactttgctaagagcaagtggaggcaagccttcaacgcagcagctgtggtgcaccacatgaggaagctacacatgaacctgcacagcccgggcgtccgcccagaggtggagaacaggccgcctgaaactcaagcctcagaaacctctagacccagctcccctgagatcaccatcaccgaggcacctgtcctggaccacagtgtagcactccctgccctgacccaattaccctgccagcatggccgccggcccactgcccctggtggcaggtccctcaactgcctggtcaatggctccctccacatcagcagcagcctggtgcccatgcatcaggggtccctggccgccgggccctgtggctgctgctccagctgcctgaacattgggagcaaaggaaagtcctcctactgctctgagcccacactcctcaaaaaggccaacaaaaaacagtacgtatttttagccaaagatggagccccagcttgggtctga

SEQ ID NO: 3 - protein, 460 aa, Homo sapiens
MGRKEEDDCSSWKKQTTNIRKTFIFMEVLGSGAFSEVFLVKQRLTGKLFALKCIKKSPAFRDSSLENEIAVLKKIKHENIVTLEDIYESTTHYYLVMQLVSGGELFDRILERGVYTEKDASLVIQQVLSAVKYLHENGIVHRDLKPENLLYLTPEENSKIMITDFGLSKMEQNGIMSTACGTPGYVAPEVLAQKPYSKAVDCWSIGVITYILLCGYPPFYEETESKLFEKIKEGYYEFESPFWDDISESAKDFICHLLEKDPNERYTCEKALSHPWIDGNTALHRDIYPSVSLQIQKNFAKSKWRQAFNAAAVVHHMRKLHMNLHSPGVRPEVENRPPETQASETSRPSSPEITITEAPVLDHSVALPALTQLPCQHGRRPTAPGGRSLNCLVNGSLHISSSLVPMHQGSLAAGPCGCCSSCLNIGSKGKSSYCSEPTLLKKANKKQYVFLAKDGAPAWV

SEQ ID NO: 4 - DNA, 1738 bp, Homo sapiens
cttcaactctggaggcaatgggtcgaaaggaagaagatgactgcagttcctggaagaaacagaccaccaacatccggaaaaccttcatttttatggaagtgctgggatcaggagctttctcagaagttttcctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcatgcagcttgtttctggtggggagctctttgaccggatcctggagcggggtgtctacacagagaaggatgccagtctggtgatccagcaggtcttgtcggcagtgaaatacctacatgagaatggcatcgtccacagagacttaaagcccgaaaacctgctttaccttacccctgaagagaactctaagatcatgatcactgactttggtctgtccaagatggaacagaatggcatcatgtccactgcctgtgggaccccaggctacgtggctccagaagtgctggcccagaaaccctacagcaaggctgtggattgctggtccatcggcgtcatcacctacatattgctctgtggataccccccattctatgaagaaacggagtctaagcttttcgagaagatcaaggagggctactatgagtttgagtctccattctgggatgacatttctgagtcagccaaggactttatttgccacttgcttgagaaggatccgaacgagcggtacacctgtgagaaggccttgagtcatccctggattgacggaaacacagccctccaccgggacatctacccatcagtcagcctccagatccagaagaactttgctaagagcaagtggaggcaagccttcaacgcagcagctgtggtgcaccacatgaggaagctacacatgaacctgcacagcccgggcgtccgcccagaggtggagaacaggccgcctgaaactcaagcctcagaaacctctagacccagctcccctgagatcaccatcaccgaggcacctgtcctggaccacagtgtagcactccctgccctgacccaattaccctgccagcatggccgccggcccactgcccctggtggcaggtccctcaactgcctggtcaatggctccctccacatcagcagcagcctggtgcccatgcatcaggggtccctggccgccgggccctgtggctgctgctccagctgcctgaacattgggagcaaaggaaagtcctcctactgctctgagcccacactcctcaaaaaggccaacaaaaaacagaacttcaagtcggaggtcatggtaccagttaaagccagtggcagctcccactgccgggcagggcagactggagtctgtctcattatgtgattcctggagcctgtgcctatgtcactgcaattttcaggagacatattcaactcctctgctcttccaaacctggtgtctatccggcagagggaggaaggcagagcaagtggagcagggcttagcaggagcagtttctggccagaagcaccagcctgctgccagcggggcagcccctcataggaggcccaggagggagccccaaggcgtagaagccttgttgaagctgtgagcaggagaagcggtgcccaccagcttccaggtctccctgacctgcctgctctatgccccac

SEQ ID NO: 5 - protein, 476 aa, Homo sapiens [sequence omitted]

SEQ ID NO: 6 - DNA, 386 bp, Homo sapiens
  misc_feature: n = a or g or c or t/u
gncactgcaattttcaggagacatattcaactcctctgctcttccaaacctggtgtctatccggcagagggaggaaggcaganaagtggagcagggcttagcaggagcagtttctggccagaagcaccagcctgctgccagcggggcancgcctcataggaggcccaggagggagccccaaggctnagaagccttgttgaagctgtgagcaggagaagcggtgcccaccagcttccaggtctccctgacctggcctgctctatgccccacaccctnacgtggccgtggntctgtngcagtgttacgttagatagcttttcgcctggggtnttgttgttgttttgttcgtgaaaagctttaatggggttnggccagggtngttttca

SEQ ID NO: 7 - DNA, 501 bp, Homo sapiens
ctgcaattttcaggagacatattcaactcctctgctcttccaaacctggtgtctatccggcagagggaggaaggcagagcaagtggagcagggcttagcaggagcagtttctggccagaagcaccagcctgctgccagcggggcagcccctcataggaggcccaggagggagccccaaggcgtaaaagccttgttgaagctgtgagcaggagaagcggtgcccaccagcttccaggtctccctgacctgcctgctctatgccccacaccctacgtgccgtggctctgtgcagtgtacgtagatagctctcgcctgggtctgtgctgtttgtcgtgaaaagcttaatgggctggccaggctgtgtcaccttctccaagcaaagccatatggagcatctacccagactcccactctgcacacactcactcccacctctcaagcctccaacctcttggccagattgggctcattaatgtcgttgcctgcccatctgcatgaatg

SEQ ID NO: 8 - DNA, 20 bp, Artificial Sequence
  DNA primer
agaagggaagaatgggggag

SEQ ID NO: 9 - DNA, 20 bp, Artificial Sequence
  DNA primer
gagacggatgaattggctgg

SEQ ID NO: 10 - DNA, 22 bp, Artificial Sequence
  DNA primer
cagtccaacaggtgagtcatcg

SEQ ID NO: 11 - DNA, 20 bp, Artificial Sequence
  DNA primer
gggaacgagaaggggtaagc

SEQ ID NO: 12 - DNA, 18 bp, Artificial Sequence
  DNA primer
tgggagcttgggggagca

SEQ ID NO: 13 - DNA, 21 bp, Artificial Sequence
  DNA primer
actttccttggcagcctgttc

SEQ ID NO: 14 - DNA, 20 bp, Artificial Sequence
  DNA primer
cctgcccactccctggatga

SEQ ID NO: 15 - DNA, 20 bp, Artificial Sequence
  DNA primer
gctgcgttgaaggcttgcta

SEQ ID NO: 16 - DNA, 22 bp, Artificial Sequence
  DNA primer
cacaaggcaaagggaaagttta

SEQ ID NO: 17 - DNA, 20 bp, Artificial Sequence
  DNA primer
ccattgaccaggcagttgag

SEQ ID NO: 18 - DNA, 20 bp, Artificial Sequence
  DNA primer
cctgacccaattaccctgcc

SEQ ID NO: 19 - DNA, 21 bp, Artificial Sequence
  DNA primer
ccccctcatccagaactcatc

SEQ ID NO: 20 - DNA, 24 bp, Artificial Sequence
  DNA primer
caaaaagtaggattgtagccctgc

SEQ ID NO: 21 - DNA, 30 bp, Artificial Sequence
  DNA primer
gtttcttctaccatccccactttcagaacc

SEQ ID NO: 22 - DNA, 23 bp, Artificial Sequence
  DNA primer
cctctctgtgaaatggcattgac

SEQ ID NO: 23 - DNA, 30 bp, Artificial Sequence
  DNA primer
gtttcttaatgcctggtcaaataccgtagg

SEQ ID NO: 24 - DNA, 22 bp, Artificial Sequence
  DNA primer
agccaaaactgacaccaggaag

SEQ ID NO: 25 - DNA, 28 bp, Artificial Sequence
  DNA primer
gtttcttggaaatggcttggtcttggtc

SEQ ID NO: 26 - DNA, 20 bp, Artificial Sequence
  DNA primer
gatgggcactgtgttactgg

SEQ ID NO: 27 - DNA, 31 bp, Artificial Sequence
  DNA primer
gtttcttgctttgatggaaatagtattatgc

SEQ ID NO: 28 - DNA, 23 bp, Artificial Sequence
  DNA primer
tgaaataaatgtgctctgggctc

SEQ ID NO: 29 - DNA, 26 bp, Artificial Sequence
  DNA primer
gtttcttccagcctgcctccactcag

SEQ ID NO: 30 - DNA, 20 bp, Artificial Sequence
  DNA primer
cacaggacggtcgatggttc

SEQ ID NO: 31 - DNA, 31 bp, Artificial Sequence
  DNA primer
gtttcttgctgtcagcaagaantgtgaaagt

SEQ ID NO: 32 - DNA, 23 bp, Artificial Sequence
  DNA primer
caaagatgctctccttccctgtc

SEQ ID NO: 33 - DNA, 27 bp, Artificial Sequence
  DNA primer
gtttcttcagccatttagggacctgcc

SEQ ID NO: 34 - DNA, 22 bp, Artificial Sequence
  DNA primer
ttacccctttctcgttccctcc

SEQ ID NO: 35 - DNA, 31 bp, Artificial Sequence
  DNA primer
gtttcttagatgtaggaacagagggtccacc

SEQ ID NO: 36 - protein, 317 aa, Homo sapiens
KQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQEG

SEQ ID NO: 37 - DNA, 21 bp, Homo sapiens
actacatattgtttctcctac

SEQ ID NO: 38 - DNA, 21 bp, Homo sapiens
acctcttctccaagcctggcc

SEQ ID NO: 39 - DNA, 21 bp, Homo sapiens
gataccccccgttctatgaag

SEQ ID NO: 40 - DNA, 21 bp, Homo sapiens
gggtgggaaatctgttctggg

SEQ ID NO: 41 - DNA, 21 bp, Homo sapiens
ttggagctccctgtaccctct

SEQ ID NO: 42 - DNA, 21 bp, Homo sapiens
cagcccgggaatccgcccaga

SEQ ID NO: 43 - DNA, 23 bp, Artificial Sequence
  DNA Primer
tggagaatgagattgctgtgttg

SEQ ID NO: 44 - DNA, 23 bp, Artificial Sequence
  DNA Primer
catctatgagagcaccacccact

SEQ ID NO: 45 - DNA, 27 bp, Artificial Sequence
  DNA probe
tcaagcatgaaaacattgtgaccctgg

SEQ ID NO: 46 - DNA, 480 bp, Homo sapiens
  misc_feature: n=a or g or c or t/u
catcctcagaagcttcanctctggaggcaatgggtcgaaaggaagaagatgactgcagttcctggaagaaacagaccaccaacatccggaaaaccttcatttttatggaagtgctgggatcaggagctttctcagaagttttcctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcatgcagcttgtttctggtggggagctctttgaccggatcctggagcggggtgtctacacagagaaggatgccagtctggtgatccagcaggtcttgtcggcagtgaaatacctacatgagaatggcatcgtccacagagacttaaagcccgaaaacctgcttt

SEQ ID NO: 47 - DNA, 467 bp, Homo sapiens
  misc_feature: n=a or g or c or t/u
catcctcagaagcttcaactctggaggcaatgggtcgaaaggaagaagatgactgcagttcctggaagaaacagaccaccaacatccggaaaaccttcatttttatggaagtgctgggatcaggagctttctcanaagttttcctggtgaagcaaagactgactgggaagctctttgctctgaagtgcatcaagaagtcacctgccttccgggacagcagcctgnagaatgagattgctgtgttgaaaaagatcaagcatgaaaacattgtgaccctggaggacatctatgagagcaccacccactactacctgntcatgcaacttgtttctggtggggagctctttgacccggatcctggagcggngtgtctacacagagaaggatgccagnctgggtgatccacangtcttgtcngcagtgaaatacctacatgagaatggcatcgtncacagagacttaaagcc

SEQ ID NO: 48 - DNA, 470 bp, Homo sapiens
  misc_feature: n=a or g or c or t/u
catcctcagaagcttcaactctggaggcaatgggtcgaaaggaagaagatgactgcagttcctggaagaaacagaccaccaacatcccggaaaaccttcatttttatggaagtgctgggatcaggagctttctcagaaagttttccctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcatgcagcttgtttctggtggggagctctttgacccggatcctggagcggngtgtctacacagagaaggatgccagnctgggtgatccacangtcttgtcngcagtgaaatacctacatgagaatggcatcgtncacagagacttaaagcc

SEQ ID NO: 49 - DNA, 356 bp, Homo sapiens
  misc_feature: n=a or g or c or t/u
catcctcagaagcttcaactctggaggcaatgggtcgaaaggaagaagatgactgcagttcctggaagaaacagaccaccaacatcccggaaaaccttcatttttatggaagtgctgggatcaggagctttctcagaaagttttccctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcatgcagcttgnttctggtggggagctctttgacccg

SEQ ID NO: 50 - DNA, 319 bp, Homo sapiens
catcctcagaagcttcaactctggaggcaatgggtcgaaaggaagaagatgactgcagttcctggaagaaacagaccaccaacatccggaaaaccttcatttttatggaagtgctgggatcaggagctttctcagaagttttcctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcat

SEQ ID NO: 51 - DNA, 24 bp, Artificial Sequence
  DNA primer
tgtcatcacccattcaggataatg

SEQ ID NO: 52 - DNA, 24 bp, Artificial Sequence
  DNA primer
ttaagcaaggagaccctctaaagc

SEQ ID NO: 53 - DNA, 21 bp, Artificial Sequence
  DNA primer
ctgggtctgtgctgtttgtcg

SEQ ID NO: 54 - DNA, 21 bp, Artificial Sequence
  DNA primer
gaagtgtgctggctgggtctc

SEQ ID NO: 55 - DNA, 18 bp, Artificial Sequence
  DNA primer
tcgttgcctgcccatctg

SEQ ID NO: 56 - DNA, 22 bp, Artificial Sequence
  DNA primer
ggtgtgctggactttcaaggag

SEQ ID NO: 57 - DNA, 24 bp, Artificial Sequence
  DNA primer
ggagtcatctggagagtttatgcc

SEQ ID NO: 58 - DNA, 26 bp, Artificial Sequence
  DNA primer
tgttcactttcttgagtgtgacaatg

SEQ ID NO: 59 - DNA, 26 bp, Artificial Sequence
  DNA primer
tcccagttctgtaagagataacaagc

SEQ ID NO: 60 - DNA, 18 bp, Artificial Sequence
  DNA primer
cggctctgctcaccctcc

SEQ ID NO: 61 - DNA, 21 bp, Artificial Sequence
  DNA primer
agagggagcacagagtttccg

SEQ ID NO: 62 - DNA, 25 bp, Artificial Sequence
  DNA primer
cagaacatctacactggctgacatg

SEQ ID NO: 63 - DNA, 26 bp, Artificial Sequence
  DNA primer
ggcttggttatctttatcttttctgc

SEQ ID NO: 64 - DNA, 22 bp, Artificial Sequence
  DNA primer
catcactcacactctggcatgg

SEQ ID NO: 65 - DNA, 21 bp, Artificial Sequence
  DNA primer
agtggactctagaccccagcc

SEQ ID NO: 66 - DNA, 21 bp, Artificial Sequence
  DNA primer
tcaggacaagcagattccagg

SEQ ID NO: 67 - DNA, 20 bp, Artificial Sequence
  DNA primer
cagtacccatcggcaccttg

SEQ ID NO: 68 - DNA, 20 bp, Artificial Sequence
  DNA primer
cacagtgctgggcaaatagt

SEQ ID NO: 69 - DNA, 20 bp, Artificial Sequence
  DNA primer
ctatttgcccagcactgtgc

SEQ ID NO: 70 - DNA, 22 bp, Artificial Sequence
  DNA primer
acctctctcccacctgttatgg

SEQ ID NO: 71 - DNA, 25 bp, Artificial Sequence
  DNA primer
ctctgttcctttggatattccactc

SEQ ID NO: 72 - DNA, 24 bp, Artificial Sequence
  DNA primer
aaatggtgtctcactcatcactcc

SEQ ID NO: 73 - DNA, 26 bp, Artificial Sequence
  DNA primer
gaggaacaaactttctttttgttcaa

SEQ ID NO: 74 - DNA, 21 bp, Artificial Sequence
  DNA primer
ataaccttccttccccactcg

SEQ ID NO: 75 - DNA, 21 bp, Artificial Sequence
  DNA primer
tcaggttggcctccaaaacta

SEQ ID NO: 76 - DNA, 19 bp, Artificial Sequence
  DNA primer
ccttccttccccactcgag

SEQ ID NO: 77 - DNA, 21 bp, homo sapiens
atgcacaagcgtttttctgga

SEQ ID NO: 78 - DNA, 21 bp, homo sapiens
acaggcagcttcccatggtgg

SEQ ID NO: 79 - DNA, 21 bp, homo sapiens
aatagaaagatgttcatgagt

SEQ ID NO: 80 - DNA, 21 bp, homo sapiens
caatttcacacacacatgcac

SEQ ID NO: 81 - DNA, 21 bp, homo sapiens
aggactgagaaaggtttgggg

SEQ ID NO: 82 - DNA, 21 bp, homo sapiens
gacatatcaaggatactgagt

SEQ ID NO: 83 - DNA, 21 bp, homo sapiens
aaggccctttttcccagttct

SEQ ID NO: 84 - DNA, 21 bp, homo sapiens
ctgccccatcaactcttcttc

SEQ ID NO: 85 - DNA, 21 bp, homo sapiens
ccaaaaccatactgactcatt

SEQ ID NO: 86 - DNA, 21 bp, homo sapiens
tgctaaatacgtattggttaa

SEQ ID NO: 87 - DNA, 21 bp, homo sapiens
acaaaaacagtacaatactca

SEQ ID NO: 88 - DNA, 21 bp, homo sapiens
gccattagctattggaggggg

SEQ ID NO: 89 - DNA, 21 bp, homo sapiens
caagaccccacagagtctaca

SEQ ID NO: 90 - DNA, 21 bp, homo sapiens
attgtagaggtacaaactttc